(19) United States
(12) Patent Application Publication
(10) Pub. No.: US 2012/0088663 A1
(43) Pub. Date: Apr. 12, 2012

(54) Triazole Compounds Carrying a Sulfur Substituent

(75) Inventors: Sarah Uluschneider, Dirmstein (DE); Jochen Dietz, Karlsruhe (DE); Jens Renner, Dirmstein (DE); Thomas Grote, Wachenheim (DE); Wassilios Grammenos, Ludwigshafen (DE); Bernd Mueller, Frankenthal (DE); Jan Klaas Lohmann, Lambsheim (DE); Marianna Vrettou-Schultes, Mannheim (DE); Richard Riggs, Mannheim (DE)

(73) Assignee: BASF SE, Ludwigshafen (DE)

(21) Appl. No.: 13/378,152

(22) PCT Filed: Jun. 17, 2010
(86) PCT No.: PCT/EP10/58539

§ 371 Date: Dec. 14, 2011

(30) Foreign Application Priority Data
Jun. 18, 2009 (EP) 09163170.5
May 12, 2010 (EP) 10162690.1

(51) Int. Cl. A01C 1/06; C07D 405/14; A01N 43/653; A61P 31/10; A01P 3/00; A61P 35/00; A61P 31/12; C07D 405/06; A61K 31/4196

(52) U.S. Cl. 504/100; 548/263.2; 548/266.6; 514/384

(57) ABSTRACT

The present invention relates to novel triazole compounds of the formulae I and II as defined below which carry a sulfur substituent, to agricultural compositions containing them, to their use as fungicides and to intermediate compounds used in the method of producing them.

The present invention relates to novel triazole compounds of the formulae I and II as defined below which carry a sulfur substituent, to agricultural compositions containing them, to their use as fungicides and to intermediate compounds used in the method of producing them.

The control of plant diseases caused by phythopathogenic fungi is extremely important for achieving high crop efficiency. Plant disease damage to ornamental, vegetable, field, cereal, and fruit crops can cause significant reduction in productivity and thereby result in increased costs to the consumer.

WO 96/41804, WO 96/16048, WO 97/41107, WO 97/43269 and WO 97/44331 describe sulfurized triazolyl derivatives. The compounds are used for combating harmful fungi.

There is a continuous need for new compounds which are more effective, less costly, less toxic, environmentally safer and/or have different modes of action.

Accordingly, it is an object of the present invention to provide compounds having a better fungicidal activity and/or a better crop plant compatibility.

Surprisingly, these objects are achieved by triazole compounds of the general formulae I and II, defined below, and by the agriculturally acceptable salts of the compounds I and II.

Accordingly, the present invention relates to triazole compounds of the formulae I and II and to agriculturally useful salts thereof

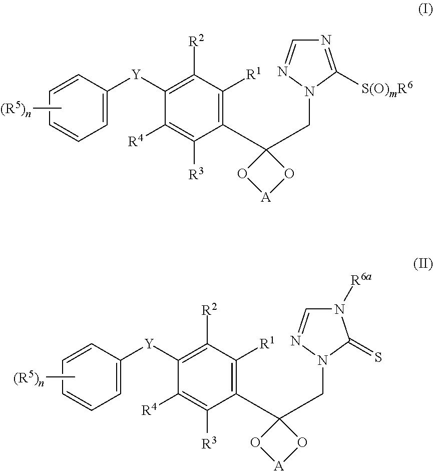

wherein
A is a linear C₁-C₅-alkylene bridge which may be substituted by 1, 2, 3, 4, 5 or 6, preferably 1, 2, 3 or 4 substituents R⁷;
Y is O, S or NR⁸;
R¹, R², R³ and R⁴, independently of each other, are selected from hydrogen, halogen, OH, SH, NO₂, CN, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₈-cycloalkyl, C₃-C₈-halocycloalkyl, C₁-C₄-alkoxy, C₁-C₄-haloalkoxy, C₁-C₄-alkenyloxy, C₁-C₄-haloalkenyloxy, C₁-C₄-alkynyloxy, C₁-C₄-haloalkynyloxy, C₃-C₈-cycloalkoxy, C₃-C₈-halocycloalkoxy, C₁-C₄-alkylthio, C₁-C₄-haloalkylthio, C₁-C₄-alkenylylthio, C₁-C₄-haloalkynylylthio, phenyl, phenyl-C₁-C₄-alkyl, phenyl-C₁-C₄-alkoxy, phenoxy, phenylthio, where the phenyl moiety in the 5 last-mentioned radicals may carry 1, 2, 3, 4 or 5 substituents R⁹; 3-, 4-, 5-, 6- or 7-membered saturated, partially unsaturated or maximum unsaturated heterocyclic ring containing 1, 2 or 3 heteroatoms selected from N, O and S as ring members, where the heterocyclic ring may carry 1, 2 or 3 substituents R⁹; COR¹⁰, COOR¹⁰, CONR¹⁵R¹⁶, NR¹⁵R¹⁶ and S(O)ₚR¹⁰, where the aliphatic moieties in the above radicals may carry 1, 2 or 3 substituents R¹⁸ and where the cycloaliphatic moieties in the above radicals may carry 1, 2 or 3 substituents R¹⁹; or
   R¹ and R² or R³ and R⁴, together with the carbon atom to which they are bound, form a partly unsaturated or maximum unsaturated 5-, 6- or 7-membered carbocyclic ring or a partly unsaturated or maximum unsaturated 5-, 6- or 7-membered heterocyclic ring containing 1, 2 or 3 heteroatoms selected from O, S and N as ring members; where the carbocyclic or heterocyclic ring may carry 1, 2 or 3 substituents R⁹;
each R⁵ is independently selected from halogen, OH, SH, NO₂, CN, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₈-cycloalkyl, C₃-C₈-halocycloalkyl, C₁-C₄-alkoxy, C₁-C₄-haloalkoxy, C₁-C₄-alkenyloxy, C₁-C₄-haloalkenyloxy, C₁-C₄-alkynyloxy, C₁-C₄-haloalkynyloxy, C₃-C₈-cycloalkoxy, C₃-C₈-halocycloalkoxy, C₁-C₄-alkylthio, C₁-C₄-haloalkylthio, C₁-C₄-alkenylylthio, C₁-C₄-haloalkynylylthio, phenyl, phenyl-C₁-C₄-alkyl, phenyl-C₁-C₄-alkoxy, phenoxy, phenylthio, where the phenyl moiety in the 5 last-mentioned radicals may carry 1, 2, 3, 4 or 5 substituents R⁹; 3-, 4-, 5-, 6- or 7-membered saturated, partially unsaturated or maximum unsaturated heterocyclic ring containing 1, 2 or 3 heteroatoms selected from N, O and S as ring members, where the heterocyclic ring may carry 1, 2 or 3 substituents R⁹; COR¹⁰, COOR¹⁰, CONR¹⁵R¹⁶, NR¹⁵R¹⁶ and S(O)ₚR¹⁰, where the aliphatic moieties in the above radicals may carry 1, 2 or 3 substituents R¹⁸ and where the cycloaliphatic moieties in the above radicals may carry 1, 2 or 3 substituents R¹⁹; or two radicals R⁵ bound on adjacent carbon atoms, together with the carbon atom to which they are bound, form a partly unsaturated or maximum unsaturated 5-, 6- or 7-membered carbocyclic ring or a partly unsaturated or maximum unsaturated 5-, 6- or 7-membered heterocyclic ring containing 1, 2 or 3 heteroatoms selected from O, S and N as ring members; where the carbocyclic or heterocyclic ring may carry 1, 2 or 3 substituents R⁹;
R⁶ is selected from hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, where the phenyl moiety in the 2 last-mentioned radicals may carry 1, 2, 3, 4 or 5 substituents R¹¹, and a 5- or 6-membered saturated, partially unsaturated or aromatic heterocyclic ring containing 1, 2 or 3 heteroatoms selected from N, O and S as ring members, where the heterocyclic ring may carry 1, 2 or 3 substituents R¹¹; or, in case m is 0, may also be selected from —C(═O)R¹², —C(═S)R¹², —S(O)₂R¹², —CN, —P(═O)R¹³R¹⁴, M and a group of the formula III

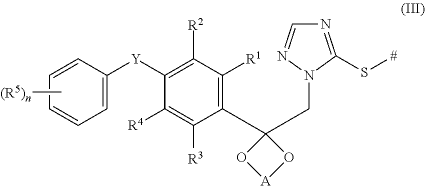

wherein
A, Y, R¹, R², R³, R⁴, R⁵ and n are as defined for formulae I and II; and
# is the attachment point to the remainder of the molecule;
R^{6a} is selected from hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, where the phenyl moiety in the 2 last-mentioned radicals may carry 1, 2, 3, 4 or 5 substituents R¹¹, a 5- or 6-membered saturated, partially unsaturated or aromatic heterocyclic ring containing 1, 2 or 3 heteroatoms selected from N, O and S as ring members, where the heterocyclic ring may carry 1, 2 or 3 substituents R¹¹, —C(═O)R¹², —C(═S)R¹², —S(O)₂R¹², —CN, —P(═O)R¹³R¹⁴ and M;
each R⁴⁷ is independently selected from halogen, OH, SH, NR¹⁵R¹⁶, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, C₁-C₄-alkylthio and C₁-C₄-haloalkylthio, where the aliphatic moieties in the above radicals may carry 1, 2 or 3 substituents R¹⁸; or
   two radicals R⁷ bound on two adjacent carbon atoms, together with the carbon atoms to which they are bound, form a 3-, 4-, 5-, 6- or 7-membered saturated, partly unsaturated or maximum unsaturated carbocyclic ring or a 3-, 4-, 5-, 6- or 7-membered saturated, partly unsaturated or maximum unsaturated heterocyclic ring containing 1, 2, or 3 heteroatoms selected from O, S and N as ring members, where the carbocyclic or heterocyclic ring may carry 1, 2 or 3 substituents R⁹;
R⁸ is selected from hydrogen, CN, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, phenyl, phenyl-C₁-C₄-alkyl, where the phenyl moiety in the 2 last-mentioned radicals may carry 1, 2, 3, 4 or 5 substituents R⁹; COR¹⁰, COOR¹⁰, CONR¹⁵R¹⁶ and S(O)ₚR¹⁰;
each R⁹ is independently selected from halogen, OH, SH, NR¹⁵R¹⁶, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, C₁-C₄-alkylthio and C₁-C₄-haloalkylthio, where the aliphatic moieties in the above radicals may carry 1, 2 or 3 substituents R¹⁸;
each R¹⁰ is independently selected from hydrogen, C₁-C₄-alkyl, C₁-C₄-haloalkyl, C₂-C₄-alkenyl, C₂-C₄-haloalkenyl, C₁-C₄-aminoalkyl, phenyl, phenyl-C₁-C₄-alkyl, where the phenyl moiety in the 2 last-mentioned radicals may carry 1, 2, 3, 4 or 5 substituents R⁹, and a 5- or 6-membered saturated, partially unsaturated or aromatic heterocyclic ring containing 1, 2 or 3 heteroatoms selected from N, O and S as ring members, where the heterocyclic ring may carry 1, 2 or 3 substituents R⁹;
each R¹¹ is independently selected from halogen, OH, SH, NR¹⁵R¹⁶, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, C₁-C₄-alkylthio and C₁-C₄-haloalkylthio, where the aliphatic moieties in the above radicals may carry 1, 2 or 3 substituents R¹⁸;
R¹² is selected from hydrogen, C₁-C₁₀-alkyl, C₁-C₁₀-haloalkyl, C₁-C₁₀-alkoxy, C₁-C₁₀-haloalkoxy, C₁-C₁₀-aminoalkyl, C₃-C₁₀-cycloalkyl, C₃-C₁₀-halocycloalkyl, phenyl, phenyl-C₁-C₄-alkyl, where the phenyl moiety in the 2 last-mentioned radicals may carry 1, 2, 3, 4 or 5 substituents R¹¹, a 5- or 6-membered saturated, partially unsaturated or aromatic heterocyclic ring containing 1, 2 or 3 heteroatoms selected from N, O and S as ring members, where the heterocyclic ring may carry 1, 2 or 3 substituents R¹¹, and NR¹⁵R¹⁶;
R¹³ and R¹⁴, independently of each other, are selected from C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, C₁-C₁₀-alkoxy, C₁-C₁₀-haloalkoxy, C₁-C₄-alkoxy-C₁-C₁₀-alkyl, C₁-C₄-alkoxy-C₁-C₁₀-alkoxy, C₁-C₁₀-alkylthio, C₁-C₁₀-haloalkylthio, C₂-C₁₀-alkenyloxy, C₂-C₁₀-alkenylthio, C₂-C₁₀-alkynyloxy, C₂-C₁₀-alkynylthio, C₃-C₁₀-cycloalkoxy, C₃-C₁₀-cycloalkylthio, phenyl, phenyl-C₁-C₄-alkyl, phenylthio, phenyl-C₁-C₄-alkoxy, and NR¹⁵R¹⁶;
each R¹⁵ is independently selected from hydrogen and C₁-C₈-alkyl;
each R¹⁶ is independently selected from hydrogen, C₁-C₈-alkyl, phenyl, and phenyl-C₁-C₄-alkyl;
   or R¹⁵ and R¹⁶ together form a linear C₄- or C₅-alkylene bridge or a group —CH₂CH₂OCH₂CH₂— or —CH₂CH₂NR¹⁷CH₂CH₂—;
each R¹⁷ is independently selected from hydrogen and C₁-C₄-alkyl;
each R¹⁸ is independently selected from nitro, CN, OH, SH, COR¹⁰, COOR¹⁰, CONR¹⁵R¹⁶; NR¹⁵R¹⁶, C₃-C₆-cycloalkyl, C₃-C₆-halocycloalkyl, C₁-C₄-alkoxy, C₁-C₄-haloalkoxy, C₃-C₆-cycloaloxy, phenyl and phenoxy;
each R¹⁹ is independently selected from nitro, CN, OH, SH, COR¹⁰, COOR¹⁰, CONR¹⁵R¹⁶; NR¹⁵R¹⁶, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy, C₃-C₆-cycloaloxy, phenyl and phenoxy;
Q is O or S;
M is a metal cation equivalent or an ammonium cation of formula (NR^{a}R^{b}R^{c}R^{d})⁺, wherein R^{a}, R^{b}, R^{c} and R^{d}, independently of each other, are selected from hydrogen, C₁-C₁₀-alkyl, phenyl and benzyl, where the phenyl moiety in the 2 last-mentioned radicals may carry 1, 2 or 3 substituents independently selected from halogen, CN, nitro, C₁-C₄-alkyl, C₁-C₄-haloalkyl, C₁-C₄-alkoxy, C₁-C₄-haloalkoxy and NR¹⁵R¹⁶;
m is 0, 1 or 2;
n is 0, 1, 2, 3, 4 or 5; and
p is 1 or 2;
   with the proviso that R¹ is not Cl if R², R³ and R⁴ are hydrogen, Y is O, (R⁵)ₙ is 4-Cl, relative to the 1-position of the attachment point of the phenyl ring to the group Y, and A is a linear C₂-alkylene group which may be substituted by 1 or 2 C₁-C₄-alkyl groups or is —(CH₂)₃; and with the proviso that R³ is not Cl if R¹, R² and R⁴ are hydrogen, Y is O, (R⁵)ₙ is 4-Cl, relative to the 1-position of the attachment point of the phenyl ring to the group Y, and A is a linear C₂-alkylene group which may be substituted by 1 or 2 C₁-C₄-alkyl groups or is —(CH₂)₃.

The present invention also relates to triazole compounds of the formulae I and II as defined above and to agriculturally useful salts thereof, with the proviso that R¹ is not Cl if R², R³ and R⁴ are hydrogen, Y is O and (R⁵)ₙ is 4-Cl, relative to the 1-position of the attachment point of the phenyl ring to the group Y; and with the proviso that R³ is not Cl if R¹, R² and R⁴ are hydrogen, Y is O and (R⁵)ₙ is 4-Cl, relative to the 1-position of the attachment point of the phenyl ring to the group Y.

The present invention also provides the use of triazole compounds of the formulae I and II and/or their agriculturally useful salts for controlling harmful fungi.

The invention further provides fungicidal compositions comprising these triazole compounds of the formulae I and/or II (and/or also of the formula IV; see below) and/or their agriculturally acceptable salts and suitable carriers. Suitable agriculturally acceptable carriers are described below.

The compounds I and II can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, e.g. a racemate, individual stereoisomers, or as an optically active form.

Compounds I and II can be understood as positional/double bond isomers of each other, at least in case the radicals R⁶/R^{6a} are identical. In case R⁶ (and of course also R^{6a}) is hydrogen, the respective compounds I and II are tautomers.

Suitable agriculturally useful salts are especially the salts of those cations or the acid addition salts of those acids whose cations and anions, respectively, have no adverse effect on the fungicidal action of the compounds I and II. Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and also the anions of C₁-C₄-alkanoic acids, preferably formate, acetate, propionate and butyrate. They can be formed by reacting I or II with an acid of the corresponding anion, preferably hydrochloric acid, hydrobromic acid, sulfuric acid, phosphoric acid or nitric acid.

In the definitions of the variables given in the formulae above, collective terms are used which are generally representative for the substituents in question. The term Cₙ—Cₘ indicates the number of carbon atoms possible in each case in the substituent or substitutent moiety in question:

Halogen: fluorine, chlorine, bromine and iodine;

Alkyl and the alkyl moieties in alkoxy, alkoxyalkyl, alkoxyalkoxy, alkylcarbonyl, alkylthiocarbonyl, aminoalkyl, alkylamino, dialkylamino, alkylaminocarbonyl, dialkylaminocarbonyl, alkylthio, alkylsulfonyl and the like: saturated straight-chain or branched hydrocarbon radicals having 1 to 2 (C₁-C₂-alkyl), 2 or 3 (C₂-C₃-alkyl), 1 to 4 (C₁-C₄-alkyl), 1 to 6 (C₁-C₆-alkyl), 1 to 8 (C₁-C₈-alkyl) or 1 to 10 (C₁-C₁₀-alkyl) carbon atoms. C₂-C₃-Alkyl is ethyl, n-propyl or isopropyl. C₁-C₂-Alkyl is methyl or ethyl. C₁-C₄-Alkyl is methyl, ethyl, propyl, isopropyl, butyl, 1-methylpropyl (sec-butyl), 2-methylpropyl (isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl. C₁-C₈-Alkyl is additionally also, for example, heptyl, octyl, 2-ethylhexyl and positional isomers thereof. C₁-C₁₀-Alkyl is additionally also, for example, nonyl, decyl, 2-propylheptyl, 3-propylheptyl and positional isomers thereof.

Haloalkyl: straight-chain or branched alkyl groups having 1 to 2 (C₁-C₂-haloalkyl), 1 to 3 (C₁-C₃-haloalkyl), 1 to 4 (C₁-C₄-haloalkyl), 1 to 6 (C₁-C₆-haloalkyl), 1 to 8 (C₁-C₈-haloalkyl), 1 to 10 (C₁-C₁₀-haloalkyl) or 2 to 10 (C₂-C₁₀-haloalkyl) carbon atoms (as mentioned above), where some or all of the hydrogen atoms in these groups may be replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, or pentafluoroethyl. C₁-C₃-Haloalkyl is additionally, for example, 1,1,1-trifluoroprop-2-yl, 3,3,3-trifluoropropyl or heptafluoropropyl. C₁-C₄-Haloalkyl is additionally, for example, 1-chlorobuty, 2-chlorobutyl, 3-chlorobutyl or 4-chlorobutyl.

C₁-C₁₀-Hydroxyalkyl: straight-chain or branched alkyl groups having 1 to 2 (C₁-C₂-hydroxyalkyl), 1 to 4 (C₁-C₄-hydroxyalkyl), 2 to 4 (C₂-C₄-hydroxyalkyl), 1 to 6 (C₁-C₆-hydroxyalkyl), 2 to 6 (C₂-C₆-hydroxyalkyl), 1 to 8 (C₁-C₈-hydroxyalkyl), 2 to 8 (C₂-C₈-hydroxyalkyl), 1 to 10 (C₁-C₁₀-hydroxyalkyl) or 2 to 10 (C₂-C₁₀-hydroxyalkyl) carbon atoms (as mentioned above), where at least one of the hydrogen atoms is replaced by a hydroxyl group, such as in 2-hydroxyethyl or 3-hydroxypropyl.

Alkenyl and the alkenyl moieties in alkenyloxy, alkenylthio, alkenylcarbonyl and the like: monounsaturated straight-chain or branched hydrocarbon radicals having 2 to 4 (C₂-C₄-alkenyl), 2 to 6 (C₂-C₆-alkenyl), 2 to 8 (C₂-C₈-alkenyl), 3 to 8 (C₃-C₈-alkenyl), 2 to 10 (C₂-C₁₀-alkenyl) or 3 to 10 (C₃-C₁₀-alkenyl) carbon atoms and a double bond in any position, for example C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl, or, for example, C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like;

Haloalkenyl and the haloalkenyl moieties in haloalkenyloxy, haloalkenylcarbonyl and the like: unsaturated straight-chain or branched hydrocarbon radicals having 2 to 4 (C₂-C₄-haloalkenyl), 2 to 6 (C₂-C₆-haloalkenyl), 2 to 8 (C₂-C₈-haloalkenyl) or 2 to 10 (C₂-C₁₀-haloalkenyl) carbon atoms and a double bond in any position (as mentioned above), where some or all of the hydrogen atoms in these groups may be replaced by halogen atoms as mentioned above, in particular fluorine, chlorine and bromine, for example chlorovinyl, chloroallyl and the like;

Alkynyl and the alkynyl moieties in alkynyloxy, alkynylthio, alkynylcarbonyl and the like: straight-chain or branched hydrocarbon groups having 2 to 4 (C₂-C₄-alkynyl), 2 to 6 (C₂-C₆-alkynyl), 2 to 8 (C₂-C₈-alkynyl), 3 to 8 (C₃-C₈-alkynyl), 2 to 10 (C₂-C₁₀-alkynyl) or 3 to 10 (C₃-C₁₀-alkynyl) carbon atoms and one or two triple bonds in any position, for example C₂-C₄-alkynyl, such as ethynyl, 1-propynyl, 2-propynyl, 1-butynyl, 2-butynyl, 3-butynyl, or 1-methyl-2-propynyl, or, for example, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

Haloalkynyl and the haloalkynyl moieties in haloalkynyloxy, haloalkynylcarbonyl and the like: unsaturated straight-chain or branched hydrocarbon radicals having 2 to 4 (C₂-C₄-haloalkynyl), 2 to 6 (C₂-C₆-haloalkynyl), 2 to 8 (C₂-C₈-haloalkynyl) or 2 to 10 (C₂-C₁₀-haloalkynyl) carbon atoms and one or two triple bonds in any position (as mentioned above), where some or all of the hydrogen atoms in these groups may be replaced by halogen atoms as mentioned above, in particular fluorine, chlorine and bromine;

Cycloalkyl and the cycloalkyl moieties in cycloalkoxy, cycloalkylcarbonyl and the like; monocyclic saturated hydrocarbon groups having 3 to 6 (C₃-C₆-cycloalkyl), 3 to 8 (C₃-C₈-cycloalkyl) or 3 to 10 (C₃-C₁₀-cycloalkyl) carbon ring members, such as cyclopropyl, cyclobutyl, cyclopentyl, cyclohexyl, cycloheptyl, cyclooctyl, cyclononyl and cyclodecyl;

Halocycloalkyl and the halocycloalkyl moieties in halocycloalkoxy, halocycloalkylcarbonyl and the like: monocyclic saturated hydrocarbon groups having 3 to 6 (C₃-C₆-halocycloalkyl), 3 to 8 (C₃-C₈-halocycloalkyl) or 3 to 10 (C₃-C₁₀-halocycloalkyl) carbon ring members (as mentioned above) in which some or all of the hydrogen atoms may be replaced by halogen atoms as mentioned above, in particular fluorine, chlorine and bromine;

Cycloalkenyl and the cycloalkenyl moieties in cycloalkenyloxy, cycloalkenylcarbonyl and the like; monocyclic monounsaturated hydrocarbon groups having 3 to 6 (C₃-C₆-cycloalkenyl), 3 to 8 (C₃-C₈-cycloalkenyl) or 3 to 10 (C₃-C₁₀-cycloalkenyl) carbon ring members, such as cyclopropenyl, cyclobutenyl, cyclopentenyl, cyclohexenyl, cycloheptenyl, cyclooctenyl, cyclononenyl and cyclodecenyl;

Halocycloalkenyl and the halocycloalkenyl moieties in halocycloalkenyloxy, halocycloalkenylcarbonyl and the like: monocyclic monounsaturated hydrocarbon groups having 3 to 6 (C₃-C₆-halocycloalkenyl), 3 to 8 (C₃-C₈-halocycloalkenyl) or 3 to 10 (C₃-C₁₀-halocycloalkenyl) carbon ring members (as mentioned above) in which some or all of the hydrogen atoms may be replaced by halogen atoms as mentioned above, in particular fluorine, chlorine and bromine; C₃-C₆-cycloalkyl-C₁-C₂-alkyl: a C₁-C₂-alkyl residue, as described above, wherein one of the hydrogen atoms is replaced by a C₃-C₆-cycloalkyl group. Examples are cyclopropylmethyl, cyclobutylmethyl, cyclopentylmethyl, cyclohexylmethyl, cyclopropyl-1-ethyl, cyclobutyl-1-ethyl, cyclopentyl-1-ethyl, cyclohexyl-1-ethyl, cyclopropyl-2-ethyl, cyclobutyl-2-ethyl, cyclopentyl-2-ethyl, cyclohexyl-2-ethyl and the like. C₃-C₁₀-cycloalkyl-C₁-C₄-alkyl is a C₁-C₄-alkyl residue, as described above, wherein one of the hydrogen atoms is replaced by a C₃-C₁₀-cycloalkyl group. Examples are, apart those mentioned above for C₃-C₆-cycloalkyl-C₁-C₄-alkyl, cycloheptylmethyl, cyclooctylmethyl, cyclononylmethyl, cyclodecylmethyl, cycloheptyl-1-ethyl, cyclooctyl-1-ethyl, cyclononyl-1-ethyl, cyclodecyl-1-ethyl, cycloheptyl-2-ethyl, cyclooctyl-2-ethyl, cyclononyl-2-ethyl, cyclodecyl-2-ethyl, cyclopropyl-1-propyl, cyclopropyl-2-propyl, cyclopropyl-3-propyl, cyclobutyl-1-propyl, cyclobutyl-2-propyl, cyclobutyl-3-propyl, cyclopentyl-1-propyl, cyclopentyl-2-propyl, cyclopentyl-3-propyl, cyclohexyl-1-propyl, cyclohexyl-2-propyl, cyclohexyl-3-propyl, cycloheptyl-1-propyl, cycloheptyl-2-propyl, cycloheptyl-3-propyl, cyclooctyl-1-propyl, cyclooctyl-2-propyl, cyclooctyl-3-propyl, cyclononyl-1-propyl, cyclononyl-2-propyl, cyclononyl-3-propyl, cyclodecyl-1-propyl, cyclodecyl-2-propyl, cyclodecyl-3-propy, cyclopropyl-1-butyl, cyclopropyl-2-butyl, cyclopropyl-3-butyl, cyclopropyl-4-butyl, cyclobutyl-1-butyl, cyclobutyl-2-butyl, cyclobutyl-3-butyl, cyclobutyl-4-butyl, cyclopentyl-1-butyl, cyclopentyl-2-butyl, cyclopentyl-3-butyl, cyclopentyl-4-butyl, cyclohexyl-1-butyl, cyclohexyl-2-butyl, cyclohexyl-3-butyl, cyclohexyl-4-butyl, cycloheptyl-1-butyl, cycloheptyl-2-butyl, cycloheptyl-3-butyl, cycloheptyl-4-butyl, cyclooctyl-1-butyl, cyclooctyl-2-butyl, cyclooctyl-3-butyl, cyclooctyl-4-butyl, cyclononyl-1-butyl, cyclononyl-2-butyl, cyclononyl-3-butyl, cyclononyl-4-butyl, cyclodecyl-1-butyl, cyclodecyl-2-butyl, cyclodecyl-3-butyl, cyclodecyl-4-butyl, and the like.

C₃-C₆-halocycloalkyl-C₁-C₂-alkyl: a C₁-C₂-alkyl residue, as described above, wherein one of the hydrogen atoms is replaced by a C₃-C₆-halocycloalkyl group. Examples are 1-chlorocyclopropylmethyl, 1-chlorocyclobutylmethyl, 1-chlorocyclopentylmethyl, 1-chlorocyclohexylmethyl, 1-chlorocyclopropyl-1-ethyl, 1-chlorocyclobutyl-1-ethyl, 1-chlorocyclopentyl-1-ethyl, 1-chlorocyclohexyl-1-ethyl, 1-chlorocyclopropyl-2-ethyl, 1-chlorocyclobutyl-2-ethyl, 1-chlorocyclopentyl-2-ethyl, 1-chlorocyclohexyl-2-ethyl, 2-chlorocyclopropylmethyl, 2-chlorocyclobutylmethyl, 2-chlorocyclopentylmethyl, 2-chlorocyclohexylmethyl, 2-chlorocyclopropyl-1-ethyl, 2-chlorocyclobutyl-1-ethyl, 2-chlorocyclopentyl-1-ethyl, 2-chlorocyclohexyl-1-ethyl, 2-chlorocyclopropyl-2-ethyl, 2-chlorocyclobutyl-2-ethyl, 2-chlorocyclopentyl-2-ethyl, 2-chlorocyclohexyl-2-ethyl, 1-fluorocyclopropylmethyl, 1-fluorocyclobutylmethyl, 1-fluorocyclopentylmethyl, 1-fluorocyclohexylmethyl, 1-fluorocyclopropyl-1-ethyl, 1-fluorocyclobutyl-1-ethyl, 1-fluorocyclopentyl-1-ethyl, 1-fluorocyclohexyl-1-ethyl, 1-fluorocyclopropyl-2-ethyl, 1-fluorocyclobutyl-2-ethyl, 1-fluorocyclopentyl-2-ethyl, 1-fluorocyclohexyl-2-ethyl, 2-fluorocyclopropylmethyl, 2-fluorocyclobutylmethyl, 2-fluorocyclopentylmethyl, 2-fluorocyclohexylmethyl, 2-fluorocyclopropyl-1-ethyl, 2-fluorocyclobutyl-1-ethyl, 2-fluorocyclopentyl-1-ethyl, 2-fluorocyclohexyl-1-ethyl, 2-fluorocyclopropyl-2-ethyl, 2-fluorocyclobutyl-2-ethyl, 2-fluorocyclopentyl-2-ethyl, 2-fluorocyclohexyl-2-ethyl, and the like. C₃-C₁₀-halocycloalkyl-C₁-C₄-alkyl is a C₁-C₄-alkyl residue, as described above, wherein one of the hydrogen atoms is replaced by a C₃-C₁₀-halocycloalkyl group.

Alkoxy: an alkyl group attached via oxygen. C₁-C₂-Alkoxy is methoxy or ethoxy. C₁-C₃-Alkoxy is additionally, for example, n-propoxy or 1-methylethoxy (isopropoxy). C₁-C₄-Alkoxy is additionally, for example, butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof. C₂-C₁₀-Alkoxy is like C₁-C₁₀-alkoxy with the exception of methoxy.

Haloalkoxy: an alkoxy radical as mentioned above which is partially or fully substituted by fluorine, chlorine, bromine and/or iodine, preferably by fluorine. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅.C₁-C₄-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

Alkenyloxy: alkenyl as mentioned above which is attached via an oxygen atom, for example C₂-C₁₀-alkenyloxy, such as 1-ethenyloxy, 1-propenyloxy, 2-propenyloxy, 1-methylethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyl, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyloxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy and the like;

Haloalkenyloxy: an alkenyloxy radical as mentioned above which is partially or fully substituted by fluorine, chlorine, bromine and/or iodine, preferably by fluorine.

Alkynyloxy: alkynyl as mentioned above which is attached via an oxygen atom, for example C₂-C₁₀-alkynyloxy, such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy and the like;

Haloalkynyloxy: an alkynyloxy radical as mentioned above which is partially or fully substituted by fluorine, chlorine, bromine and/or iodine, preferably by fluorine.

Cycloalkoxy: cycloalkyl as mentioned above which is attached via an oxygen atom, for example C₃-C₁₀-cycloalkoxy or C₃-C₈-cycloalkoxy, such as cyclopropoxy, cyclopentoxy, cyclohexoxy, cycloheptoxy, cyclooctoxy, cyclononyloxy, cyclodecyloxy and the like;

Halocycloalkoxy: a cycloalkoxy radical as mentioned above which is partially or fully substituted by fluorine, chlorine, bromine and/or iodine, preferably by fluorine.

Cycloalkenyloxy: cycloalkenyl as mentioned above which is attached via an oxygen atom, for example C₃-C₁₀-cycloalkenyloxy, C₃-C₈-cycloalkenyloxy or, preferably, C₅-C₆-cycloalkenyloxy, such as cyclopent-1-enoxy, cyclopent-2-enoxy, cyclohex-1-enoxy and cyclohex-2-enoxy;

Alkoxyalkyl: alkyl as defined above having 1 to 10, 1 to 8, 1 to 6 or 1 to 4, in particular 1 to 3, carbon atoms, in which one hydrogen atom is replaced by an alkoxy group having 1 to 8, 1 to 6, 1 to 4 or 1 to 3 carbon atoms, for example methoxymethyl, 2-methoxyethyl, ethoxymethyl, 3-methoxypropyl, 3-ethoxypropyl and the like.

Alkoxyalkoxy: alkoxy as defined above having 1 to 10, 1 to 8, 1 to 6 or 1 to 4, in particular 1 to 3, carbon atoms, in which one hydrogen atom is replaced by an alkoxy group having 1 to 8, 1 to 6 or in particular 1 to 4 carbon atoms, for example 2-methoxyethoxy, 2-ethoxyethoxy, 3-methoxypropoxy, 3-ethoxypropoxy and the like.

Alkylcarbonyl: group of the formula R—CO— in which R is an alkyl group as defined above, for example C₁-C₁₀-alkyl, C₁-C₈-alkyl, C₁-C₆-alkyl, C₁-C₄-alkyl, C₁-C₂-alkyl or C₃-C₄-alkyl. Examples are acetyl, propionyl and the like. Examples for C₃-C₄-alkylcarbonyl are propylcarbonyl, isopropylcarbonyl, n-butylcarbonyl, sec-butylcarbonyl, isobutylcarbonyl and tert-butylcarbonyl.

Haloalkylcarbonyl: group of the formula R—CO— in which R is a haloalkyl group as defined above, for example C₁-C₁₀-haloalkyl, C₁-C₈-haloalkyl, C₁-C₆-haloalkyl, C₁-C₄-haloalkyl, C₁-C₂-haloalkyl or C₃-C₄-haloalkyl. Examples are difluoromethylcarbonyl, trifluoromethylcarbonyl, 2,2-difluoroethylcarbony, 2,2,3-trifluoroethylcarbonyl and the like.

Alkoxycarbonyl: group of the formula R—CO— in which R is an alkoxy group as defined above, for example C₁-C₁₀-alkoxy, C₁-C₈-alkoxy, C₁-C₆-alkoxy, C₁-C₄-alkoxy or C₁-C₂-alkoxy. Examples for C₁-C₄-alkoxycarbonyl are methoxycarbonyl, ethoxycarbonyl, propoxycarbonyl, isopropoxycarbonyl, n-butoxycarbonyl, sec-butoxycarbonyl, isobutoxycarbonyl and tert-butoxycarbonyl.

Haloalkoxycarbonyl: group of the formula R—CO— in which R is a haloalkoxy group as defined above, for example C₁-C₁₀-haloalkoxy, C₁-C₈-haloalkoxy, C₁-C₆-haloalkoxy, C₁-C₄-haloalkoxy or C₁-C₂-haloalkoxy. Examples for C₁-C₄-haloalkoxycarbonyl are difluoromethoxycarbonyl, trifluoromethoxycarbonyl, 2,2-difluoroethoxycarbony, 2,2,3-trifluoroethoxycarbonyl and the like.

Alkylaminocarbonyl: group of the formula R—NH—CO— in which R is an alkyl group as defined above, for example C₁-C₁₀-alkyl, C₁-C₈-alkyl, C₁-C₆-alkyl, C₁-C₄-alkyl, C₁-C₂-alkyl or C₃-C₄-alkyl. Examples for C₁-C₄-alkylaminocarbonyl are methylaminocarbonyl, ethylaminocarbonyl, propylaminocarbonyl, isopropylaminocarbonyl, butylaminocarbonyl, sec-butylaminocarbonyl, isobutylaminocarbonyl and tert-butylaminocarbonyl.

Dialkylaminocarbonyl: group of the formula RR^{′}N—CO— in which R and R′, independently of each other, are an alkyl group as defined above, for example C₁-C₁₀-alkyl, C₁-C₈-alkyl, C₁-C₆-alkyl, C₁-C₄-alkyl, C₁-C₂-alkyl or C₃-C₄-alkyl. Examples for di-(C₁-C₄-alkyl)aminocarbonyl are dimethylaminocarbonyl, diethylaminocarbonyl, dipropylaminocarbonyl, diisopropylaminocarbonyl and dibutylaminocarbonyl.

Aminoalkyl: group of the formula R—NH₂ in which R is an alkyl group as defined above, for example C₁-C₁₀-alkyl, C₁-C₈-alkyl, C₁-C₆-alkyl, C₁-C₄-alkyl, C₁-C₂-alkyl or C₃-C₄-alkyl. Examples are aminomethyl, 1- and 2-aminoethyl, 1-, 2- and 3-aminopropyl, 1- and 2-amino1-methylethyl, 1-, 2-, 3- and 4-aminobutyl and the like.

Alkylsulfonyl: group of the formula R—S(O)₂— in which R is an alkyl group as defined above, for example C₁-C₁₀-alkyl, C₁-C₈-alkyl, C₁-C₆-alkyl, C₁-C₄-alkyl or C₁-C₂-alkyl.

Examples for C₁-C₄-alkylsulfonyl are methylsulfonyl, ethylsulfonyl, propylsulfonyl, isopropylsulfonyl, n-butylsulfonyl, sec-butylsulfonyl, isobutylsulfonyl and tert-butylsulfonyl.

Alkylthio: alkyl as defined above which is attached via a sulfur atom.

Haloalkylthio: haloalkyl as defined above which is attached via a sulfur atom.

Alkenylthio: alkenyl as defined above which is attached via a sulfur atom.

Haloalkenylthio: haloalkenyl as defined above which is attached via a sulfur atom.

Alkynylthio: alkynyl as defined above which is attached via a sulfur atom.

Haloalkynylthio: haloalkynyl as defined above which is attached via a sulfur atom.

Cycloalkylthio: cycloalkyl as defined above which is attached via a sulfur atom.

Aryl is a carbocyclic aromatic monocyclic or polycyclic ring containing 6 to 16 carbon atoms as ring members. Examples are phenyl, naphthyl, anthracenyl, phenanthrenyl, fluorenyl and azulenyl. Preferably, aryl is phenyl or naphthyl, and especially phenyl.

Phenyl-C₁-C₄-alkyl: C₁-C₄-alkyl (as defined above), where a hydrogen atom is replaced by a phenyl group, such as benzyl, phenethyl and the like.

Phenyl-C₁-C₄-alkoxy: C₁-C₄-alkoxy (as defined above), where one hydrogen atom is replaced by a phenyl group, such as benzyloxy, phenethyloxy and the like.

3-, 4-, 5-, 6- or 7-membered saturated, partially unsaturated or maximum unsaturated carbocyclic radical: cyclopropyl, cyclobutyl, cyclopentyl, cyclohexyl, cycloheptyl, cyclopropenyl, cyclobutenyl, cyclopentenyl, cyclohexenyl, cycloheptenyl, cyclobutadienyl, cyclopentadienyl, cyclohexadienyl, cycloheptadienyl or cycloheptatrienyl. Formally, phenyl is also included in this definition, but as it is also encompassed in the term aryl, it is not listed here.

3-, 4-, 5-, 6- or 7-membered saturated, partially unsaturated or maximum unsaturated heterocycle which contains 1, 2 or 3 heteroatoms or heteroatom containing groups selected from oxygen, nitrogen (as N or NR) and sulfur (as S, SO or SO₂) and optionally 1 or 2 groups selected from C(═O) and C(═S) as ring members:
three- or four-membered saturated or partially unsaturated heterocycle (hereinbelow also referred to as heterocyclyl) which contains 1, 2 or 3 heteroatoms from the group consisting of oxygen, nitrogen (as N or NR) and sulfur (as S, SO or SO₂) and optionally 1 or 2 groups selected from C(═O) and C(═S) as ring members: for example monocyclic saturated or partially unsaturated heterocycles which, in addition to carbon ring members, contain one to three nitrogen atoms and/or one oxygen or sulfur atom or one or two oxygen and/or sulfur atoms and optionally 1 or 2 groups selected from C(═O) and C(═S), for example 2-oxiranyl, 2-thiiranyl, 1- or 2-aziridinyl, 1-, 2- or 3-azetidinyl,
five- or six-membered saturated or partially unsaturated heterocycle (hereinbelow also referred to as heterocyclyl) which contains 1, 2 or 3 heteroatoms from the group consisting of oxygen, nitrogen (as N or NR) and sulfur (as S, SO or SO₂) and optionally 1 or 2 groups selected from C(═O) and C(═S) as ring members: for example monocyclic saturated or partially unsaturated heterocycles which, in addition to carbon ring members, contain one to three nitrogen atoms and/or one oxygen or sulfur atom or one or two oxygen and/or sulfur atoms and optionally 1 or 2 groups selected from C(═O) and C(═S), for example 2-tetrahydrofuranyl, 3-tetrahydrofuranyl, 3-tetrahydrofuran-2-onyl, 4-tetrahydrofuran-2-onyl, 5-tetrahydrofuran-2-onyl, 2-tetrahydrofuran-3-onyl, 4-tetrahydrofuran-3-onyl, 5-tetrahydrofuran-3-onyl, 2-tetrahydrothienyl, 3-tetrahydrothienyl, 3-tetrahydrothien-2-onyl, 4-tetrahydrothien-2-onyl, 5-tetrahydrothien-2-onyl, 2-tetrahydrothien-3-onyl, 4-tetrahydrothien-3-onyl, 5-tetrahydrothien-3-onyl, 2-pyrrolidinyl, 3-pyrrolidinyl, 1-pyrrolidin-2-onyl, 3-pyrrolidin-2-onyl, 4-pyrrolidin-2-onyl, 5-pyrrolidin-2-onyl, 1-pyrrolidin-3-onyl, 2-pyrrolidin-3-onyl, 4-pyrrolidin-3-onyl, 5-pyrrolidin-3-onyl, 1-pyrrolidin-2,5-dionyl, 3-pyrrolidin-2,5-dionyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals;
a seven-membered saturated or partially unsaturated heterocycle which contains 1, 2 or 3 heteroatoms from the group consisting of oxygen, nitrogen and sulfur as ring members: for example mono- and bicyclic heterocycles having 7 ring members which, in addition to carbon ring members, contain one to three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals.
a five- or six-membered aromatic (=maximum unsaturated) heterocycle (=heteroaromatic radical) which contains 1, 2 or 3 heteroatoms from the group consisting of oxygen, nitrogen and sulfur, for example 5-membered heteroaryl which is attached via carbon and contains one to three nitrogen atoms or one or two nitrogen atoms and one sulfur or oxygen atom as ring members, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 5-membered heteroaryl which is attached via nitrogen and contains one to three nitrogen atoms as ring members, such as pyrrol-1-yl, pyrazol-1-yl, imidazol-1-yl, 1,2,3-triazol-1-yl and 1,2,4-triazol-1-yl; 6-membered heteroaryl, which contains one, two or three nitrogen atoms as ring members, such as pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;

Linear C₂- or C₃-alkylene: divalent unbranched chains having 2 or 3 carbon atoms, namely CH₂CH₂ and CH₂CH₂CH₂.

Linear C₁-C₅-alkylene: divalent unbranched chains having 1 to 5 carbon atoms, namely CH₂, CH₂CH₂, CH₂CH₂CH₂, CH₂CH₂CH₂CH₂ and CH₂CH₂CH₂CH₂CH₂.

C₂-C₅-Alkylene: divalent branched or preferably unbranched chains having 2 to 5 carbon atoms, for example CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂.

C₄-C₅-Alkylene: divalent branched or preferably unbranched chains having 4 to 5 carbon atoms, for example CH₂CH₂CH₂CH₂ or CH₂CH₂CH₂CH₂CH₂.

The group —SM is more correctly spoken a group —S⁻M⁺, where M⁺ is a metal cation equivalent or an ammonium cation as defined above. A metal cation equivalent is more correctly spoken 1/a M^{a+}, where a is the valence of the metal and is in general 1, 2 or 3.

The statements made below with respect to suitable and preferred features of the compounds according to the invention, especially with respect to their substituents A, Y, R¹, R², R³, R⁴, R⁵, R⁶, R^{6a}, R⁷, R⁸, R⁹, R¹⁰, R¹¹, R¹², R¹³, R¹⁴, R¹⁵, R¹⁶, R¹⁷, R¹⁸, R¹⁹, R^{a}, R^{b}, R^{c}, R^{d}, M, Q and the indices m, n and p, and to their use, are valid both per se and, in particular, in every possible combination with one another.

In one embodiment of the invention (embodiment A), R¹ is different from chlorine if R², R³ and R⁴ are hydrogen; or R³ is different from chlorine if R¹, R² and R⁴ are hydrogen; and (R⁵)ₙ can have any of the above-given meanings, but has preferably one of the below-given preferred meanings. Especially, R¹ is different from chlorine if R², R³ and R⁴ are hydrogen, or R³ is different from chlorine if R¹, R² and R⁴ are hydrogen, if simultaneously (R⁵)ⁿ is 4-Cl, relative to the 1-position of the attachment point of the phenyl ring to the group Y and A is a linear C₂-alkylene group which may be substituted by 1 or 2 C₁-C₄-alkyl groups or is —(CH₂)₃.

In a preferred embodiment (embodiment A.1), R², R³ and R⁴ are hydrogen and R¹ is selected from fluorine, bromine, OH, SH, NO₂, CN, C₁-C₄-alkyl, C₁-C₄-haloalkyl, C₁-C₄-alkoxy-C₁-C₄-alkyl, C₁-C₄-alkoxy, C₁-C₄-alkoxy-C₁-C₄-alkoxy, and C₁-C₄-haloalkoxy, and preferably from fluorine, bromine, C₁-C₄-alkyl, C₁-C₄-haloalkyl, C₁-C₄-alkoxy and C₁-C₄-haloalkoxy, especially if simultaneously (R⁵)ⁿ is 4-Cl, relative to the 1-position of the attachment point of the phenyl ring to the group Y and A is a linear C₂-alkylene group which may be substituted by 1 or 2 C₁-C₄-alkyl groups or is —(CH₂)₃. In a more preferred embodiment (embodiment A.1.1), R², R³ and R⁴ are hydrogen and R¹ is selected from fluorine and bromine, especially if simultaneously (R⁵)ⁿ is 4-Cl, relative to the 1-position of the attachment point of the phenyl ring to the group Y and A is a linear C₂-alkylene group which may be substituted by 1 or 2 C₁-C₄-alkyl groups or is —(CH₂)₃. In another more preferred embodiment (embodiment A.1.2), R², R³ and R⁴ are hydrogen and R¹ is selected from C₁-C₄-alkyl, C₁-C₄-haloalkyl, C₁-C₄-alkoxy and C₁-C₄-haloalkoxy and preferably from methyl, ethyl, CHF₂, CF₃, methoxy, ethoxy, OCHF₂ and OCF₃, especially if simultaneously (R⁵)ⁿ is 4-Cl, relative to the 1-position of the attachment point of the phenyl ring to the group Y and A is a linear C₂-alkylene group which may be substituted by 1 or 2 C₁-C₄-alkyl groups or is —(CH₂)₃.

In another preferred embodiment (embodiment A.2), R¹ at, R², R³ and R⁴ are hydrogen.

In another preferred embodiment (embodiment A.3), at least two of R¹, R², R³ and R⁴ are not hydrogen. In a more preferred embodiment (embodiment A.3.1), at least two of R¹, R², R³ and R⁴ are not hydrogen and these at least two radicals of R¹, R², R³ and R⁴ which are not hydrogen are selected from fluorine, chlorine, bromine, C₁-C₄-alkyl, C₁-C₄-haloalkyl, C₁-C₄-alkoxy and C₁-C₄-haloalkoxy and preferably from fluorine, chlorine, bromine, methyl, ethyl, CHF₂, CF₃, methoxy, ethoxy, OCHF₂ and OCF₃.

Specifically, the combined meaning of R¹, R², R³ and R⁴ in embodiment A, especially if simultaneously (R⁵)ⁿ is 4-Cl, relative to the 1-position of the attachment point of the phenyl ring to the group Y and A is a linear C₂-alkylene group which may be substituted by 1 or 2 C₁-C₄-alkyl groups or is —(CH₂)₃, is selected from following combinations compiled in Table 1:

TABLE 1

| No. | R¹ | R² | R³ | R⁴ |
|---|---|---|---|---|
| 1. | H | H | H | H |
| 2. | H | F | H | H |
| 3. | H | Cl | H | H |
| 4. | H | Br | H | H |
| 5. | H | CH₃ | H | H |
| 6. | H | CH₂CH₃ | H | H |
| 7. | H | CHF₂ | H | H |
| 8. | H | CF₃ | H | H |
| 9. | H | OCH₃ | H | H |
| 10. | H | OCH₂CH₃ | H | H |
| 11. | H | OCHF₂ | H | H |
| 12. | H | OCF₃ | H | H |
| 13. | F | H | H | H |
| 14. | Br | H | H | H |
| 15. | CH₃ | H | H | H |
| 16. | CH₂CH₃ | H | H | H |
| 17. | CHF₂ | H | H | H |
| 18. | CF₃ | H | H | H |
| 19. | OCH₃ | H | H | H |
| 20. | OCH₂CH₃ | H | H | H |
| 21. | OCHF₂ | H | H | H |
| 22. | OCF₃ | H | H | H |
| 23. | F | F | H | H |
| 24. | H | F | H | F |
| 25. | H | F | F | H |
| 26. | F | H | F | H |
| 27. | Cl | Cl | H | H |
| 28. | H | Cl | H | Cl |
| 29. | H | Cl | Cl | H |
| 30. | Cl | H | Cl | H |
| 31. | F | Cl | H | H |
| 32. | Cl | F | H | H |
| 33. | H | F | H | Cl |
| 34. | F | H | Cl | H |
| 35. | H | Cl | F | H |
| 36. | H | F | Cl | H |
| 37. | CH₃ | CH₃ | H | H |
| 38. | H | CH₃ | H | CH₃ |
| 39. | H | CH₃ | CH₃ | H |
| 40. | CH₃ | H | CH₃ | H |
| 41. | F | CH₃ | H | H |
| 42. | CH₃ | F | H | H |
| 43. | H | F | H | CH₃ |
| 44. | F | H | CH₃ | H |
| 45. | H | CH₃ | F | H |
| 46. | H | F | CH₃ | H |
| 47. | F | OCH₃ | H | H |
| 48. | OCH₃ | F | H | H |
| 49. | H | F | H | OCH₃ |
| 50. | F | H | OCH₃ | H |
| 51. | H | OCH₃ | F | H |
| 52. | H | F | OCH₃ | H |
| 53. | F | F | F | H |
| 54. | F | F | H | F |

In another embodiment of the invention (embodiment B), R⁵ is different from 4-Cl, relative to the 1-position of the attachment point of the phenyl ring to the group Y, if n is 1, especially if simultaneously A is a linear C₂-alkylene group which may be substituted by 1 or 2 C₁-C₄-alkyl groups or is —(CH₂)₃—, and R¹, R², R³ and R⁴ can have any of the above-given general meanings, but have preferably one of the above-given preferred meanings.

In a preferred embodiment (embodiment B.1), n is 1 and R⁵ is selected from 2-Cl and 3-Cl, relative to the 1-position of the attachment point of the phenyl ring to the group Y, especially if simultaneously A is a linear C₂-alkylene group which may be substituted by 1 or 2 C₁-C₄-alkyl groups or is —(CH₂)₃.

In another preferred embodiment (embodiment B.2), R⁵ is selected from fluorine, bromine, OH, SH, NO₂, CN, C₁-C₄-alkyl, C₁-C₄-haloalkyl, C₁-C₄-alkoxy-C₁-C₄-alkyl, C₁-C₄-alkoxy, C₁-C₄-alkoxy-C₁-C₄-alkoxy, and C₁-C₄-haloalkoxy, preferably from fluorine, bromine, C₁-C₄-alkyl, C₁-C₄-haloalkyl, C₁-C₄-alkoxy and C₁-C₄-haloalkoxy and more preferably from fluorine, bromine, methyl, ethyl, CHF₂, CF₃, methoxy, ethoxy, OCHF₂ and OCF₃, especially if simultaneously A is a linear C₂-alkylene group which may be substituted by 1 or 2 C₁-C₄-alkyl groups or is —(CH₂)₃.

In a more preferred embodiment (embodiment B.2.1), n is 1 and R⁵ is selected from fluorine, bromine, OH, SH, NO₂, CN, C₁-C₄-alkyl, C₁-C₄-haloalkyl, C₁-C₄-alkoxy-C₁-C₄-alkyl, C₁-C₄-alkoxy, C₁-C₄-alkoxy-C₁-C₄-alkoxy, and C₁-C₄-haloalkoxy, preferably from fluorine, bromine, C₁-C₄-alkyl, C₁-C₄-haloalkyl, C₁-C₄-alkoxy and C₁-C₄-haloalkoxy and C₁-C₄-haloalkoxy and more preferably from fluorine, bromine, methyl, ethyl, CHF₂, CF₃, methoxy, ethoxy, OCHF₂ and OCF₃, especially if simultaneously A is a linear C₂₋alkylene group which may be substituted by 1 or 2 C₁-C₄-alkyl groups or is —(CH₂)₃.

In another preferred embodiment (embodiment B.3), n is 0.

In another preferred embodiment (embodiment B.4), n is 2, 3, 4 or 5, preferably 2, 3 or 4 and more preferably 2 or 3. In a more preferred embodiment (embodiment B.4.1), n is 2, 3, 4 or 5, preferably 2, 3 or 4, more preferably 2 or 3 and specifically 2, and R⁵ is selected from fluorine, chlorine, bromine, OH, SH, NO₂, CN, C₁-C₄-alkyl, C₁-C₄-haloalkyl, C₁-C₄-alkoxy-C₁-C₄-alkyl, C₁-C₄-alkoxy, C₁-C₄-alkoxy-C₁-C₄-alkoxy, and C₁-C₄-haloalkoxy, preferably from fluorine, chlorine, bromine, C₁-C₄-alkyl, C₁-C₄-haloalkyl, C₁-C₄-alkoxy and C₁-C₄-haloalkoxy and C₁-C₄-haloalkoxy, more preferably from fluorine, chlorine, bromine, methyl, ethyl, CHF₂, CF₃, methoxy, ethoxy, OCHF₂ and OCF₃ and in particular from fluorine, bromine, methyl, ethyl, CHF₂, CF₃, methoxy, ethoxy, OCHF₂ and OCF₃.

Specifically, in embodiment B, the phenyl ring carrying n substituents R⁵ can be depicted as follows:

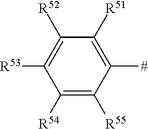

and the combination of the variables R⁵¹, R⁵², R⁵³, R⁵⁴ and R⁵⁵ is selected from the definition given in each line of Table A below, with the exception of line no. A-7.

In another embodiment of the invention (embodiment C), (R⁵)ₙ is 4-Cl, relative to the 1-position of the attachment point of the phenyl ring to the group Y (i.e. n is 1 and R⁵ is Cl bound on the 4-position), except for compounds I and II wherein simultaneously A is a linear C₂-alkylene group which may be substituted by 1 or 2 C₁-C₄-alkyl groups or is —(CH₂)₃, R¹ is Cl and R², R³ and R⁴ are hydrogen.

R¹² in the groups —C(═O)R¹² and —S(O)₂R¹² is preferably selected from C₁-C₄-alkyl, C₁-C₂-haloalkyl, C₁-C₄-alkoxy, C₁-C₂-haloalkoxy, phenyl, phenoxy and NR¹⁵R¹⁶, more preferably from C₁-C₄-alkyl, C₁-C₂-haloalkyl, C₁-C₄-alkoxy, C₁-C₂-haloalkoxy and NR¹⁵R¹⁶ and even more preferably from C₁-C₄-alkyl, C₁-C₄-alkoxy and NR¹⁵R¹⁶. In the group —C(═O)R¹², R¹² is specifically C₁-C₄-alkyl, such as methyl, ethyl, propyl, isopropyl, n-butyl, sec-butyl, isobutyl or tert-butyl, preferably methyl, or is C₁-C₄-alkoxy, such as methoxy, ethoxy, propoxy, isopropoxy, n-butoxy, sec-butoxy, isobutoxy or tert-butoxy, preferably methoxy, and is more specifically methyl, and in the group —S(O)₂R¹², R¹² is specifically methyl. Preferably, R¹⁵ is hydrogen and R¹⁶ is selected from hydrogen, C₁-C₄-alkyl and phenyl, preferably from hydrogen and C₁-C₄-alkyl; or R¹⁵ and R¹⁶ are both C₁-C₄-alkyl, preferably methyl or ethyl.

M is preferably selected from an alkali metal cation, an earth alkaline metal cation equivalent, a cation equivalent of Cu, Zn, Fe or Ni or an ammonium cation of formula (NR^{a}R^{b}R^{c}R^{d})⁺, wherein one of R^{a}, R^{b}, R^{c} and R^{d} is hydrogen and three of R^{a}, R^{b}, R^{c} and R^{d}, independently of each other, are selected from C₁-C₁₀-alkyl. More preferably, M is selected from Li⁺, Na⁺, K⁺, % Mg²⁺, a cation equivalent of Cu, Zn, Fe or Ni and an ammonium cation of formula (NR^{a}R^{b}R^{c}R^{d})⁺, wherein one of R^{a}, R^{b}, R^{c} and R^{d} is hydrogen and three of R^{a}, R^{b}, R^{c} and R^{d}, independently of each other, are selected from C₁-C₁₀-alkyl. Even more preferably, M is selected from Na⁺, K⁺, ½Mg²⁺, ½Cu²⁺, ½Zn²⁺, ½Fe²⁺, ½Ni²⁺, triethylammonium and trimethylammonium.

In the group of formula III, the variables preferably have the same meanings as in the remainder of the molecule I. Thus, the remarks made above as to preferred meanings of the radicals apply to this moiety, too.

R⁶ is preferably selected from hydrogen, C₁-C₄-alkyl, —C(═O)R¹², —S(O)₂R¹², —CN, M and a group of the formula III, where R¹² has one of the above general meanings or, in particular, one of the above preferred meanings and M has one of the above general meanings or, in particular, one of the above-given preferred meanings.

R⁶ is more preferably selected from hydrogen, C₁-C₄-alkyl, C₃-C₄-alkylcarbonyl, C₁-C₄-alkoxycarbonyl, —C(═O)N(H)C₁-C₄-alkyl, —C(═O)N(C₁-C₄-alkyl)₂, C₁-C₄-alkylsulfonyl, CN, M and a group of the formula III, where M has one of the above general meanings or, in particular, one of the above preferred meanings. In particular, R⁶ is selected from hydrogen, methyl, ethyl, propyl, isopropyl, methylcarbonyl, methoxycarbonyl, —C(═O)N(CH₃)₂, CN, M and a group of the formula III, where M has one of the above general meanings or, in particular, one of the preferred meanings and is preferably an alkaline metal cation or ½Cu²⁺. Specifically, R⁶ is hydrogen, methyl, methylcarbonyl, methoxycarbonyl, Na⁺ or a group of the formula III. Very specifically, R⁶ is hydrogen.

R^{6a} is preferably selected from hydrogen, C₁-C₁₀-alkyl, C₁-C₄-haloalkyl, phenyl, phenyl-C₁-C₄-alkyl, —C(═O)R¹² and —S(O)₂R¹², where R¹² has one of the above given general or, in particular, one of the above-given preferred meanings. More preferably, R^{6a} is selected from hydrogen, C₁-C₄-alkyl, C₁-C₄-haloalkyl, phenyl, benzyl, —C(═O)R¹² and —S(O)₂R¹², where R¹² has one of the above given general or, in particular, one of the above-given preferred meanings, and more preferably from hydrogen, C₁-C₄-alkyl, C₁-C₄-haloalkyl, —C(═O)R¹² and —S(O)₂R¹², where R¹² has one of the above given general or, in particular, one of the above-given preferred meanings. In particular, R^{6a} is hydrogen, C₁-C₄-alkyl, preferably methyl, or —C(═O)R¹², more particularly hydrogen, C₁-C₄-alkyl, preferably methyl, methylcarbonyl or methoxycarbonyl, even more particularly hydrogen or C₁-C₄-alkyl, preferably methyl, and is specifically hydrogen.

Y is preferably O or NR⁸ and in particular O.

Preferably, each R⁷ is independently selected from C₁-C₄-alkyl, C₁-C₄-haloalkyl, C₁-C₄-alkoxy-C₁-C₄-alkyl, C₁-C₄-alkoxy, C₁-C₄-alkoxy-C₁-C₄-alkoxy, C₁-C₄-haloalkoxy and phenyl, or two substituents R⁷ bound on adjacent carbon atoms, together with the carbon atoms to which they are bound, form a cyclopentyl, cyclohexyl or phenyl ring. More preferably, each R⁷ is independently selected from C₁-C₄-alkyl, methoxy, ethoxy, methoxymethyl or phenyl or two substituents R⁷ bound on adjacent carbon atoms, together with the carbon atoms to which they are bound, form a cyclopentyl, cyclohexyl or phenyl ring. Even more preferably, each R⁷ is independently selected from methyl, ethyl, propyl, isopropyl, tert-butyl, methoxy, ethoxy, methoxymethyl and phenyl or two substituents R⁷ bound on adjacent carbon atoms, together with the carbon atoms to which they are bound, form a cyclopentyl, cyclohexyl or phenyl ring.

If A is a linear C₂-alkyene bridge, it is clear that it can carry at most 4 substituents R⁷.

A is preferably a linear C₂- or C₃-alkyene bridge, where 1, 2, 3, 4, 5 or 6, preferably 1, 2, 3 or 4 hydrogen atoms of the alkylene bridge may be replaced by 1, 2, 3, 4, 5 or 6, preferably 1, 2, 3 or 4 substituents R⁷, where each R⁷ is independently selected from C₁-C₄-alkyl, C₁-C₄-haloalkyl, C₁-C₄-alkoxy-C₁-C₄-alkyl, C₁-C₄-alkoxy, C₁-C₄-alkoxy-C₁-C₄-alkoxy, C₁-C₄-haloalkoxy and phenyl, preferably from C₁-C₄-alkyl, methoxy, ethoxy, methoxymethyl or phenyl or two substituents R⁷ bound on adjacent carbon atoms, together with the carbon atoms to which they are bound, form a cyclopentyl, cyclohexyl or phenyl ring, more preferably from methyl, ethyl, propyl, isopropyl, tert-butyl, methoxy, ethoxy, methoxymethyl and phenyl or two substituents R⁷ bound on adjacent carbon atoms, together with the carbon atoms to which they are bound, form a cyclopentyl, cyclohexyl or phenyl ring, and specifically from methyl, ethyl, propyl, methoxymethyl and phenyl. More preferably, in compounds I and II A is a linear C₂- or C₃-alkyene bridge, where 1 hydrogen atom of the alkylene bridge may be replaced by 1 substituent R⁷, where R⁷ is C₁-C₄-alkyl or methoxymethyl. Specifically, in compounds I and II A is a linear C₂-alkyene bridge or a linear C₃-alkyene bridge, where 1 hydrogen atom of the alkylene bridge may be replaced by 1 substituent R⁷, where R⁷ is C₁-C₄-alkyl, preferably methyl, ethyl or propyl, or is methoxymethyl. Very specifically, in compounds I and II A is —CH(R⁷)—CH₂— or —CH(R⁷)—CH₂—CH₂—, where R⁷ is C₁-C₄-alkyl, preferably methyl, ethyl or propyl, or is methoxymethyl.

If m is 1, the oxygen atom is preferably bound via a double bond to the sulfur atom, the radical —S(O)ₘ—R⁶ thus resulting in a group —S(═O)—R⁶. If m is 2, the two oxygen atoms are preferably both bound via a double bond to the sulfur atom, the radical —S(O)ₘ—R⁶ thus resulting in a group —S(═O)₂—R⁶. If m is 3, the radical —S(O)ₘ—R⁶ is a group —S(═O)₂—O—R⁶.

m is preferably 0 or 2 and more preferably 0.

n is preferably 0, 1, 2 or 3, more preferably 0, 1, 2 or 3 and in particular 0, 1 or 2.

In a particularly preferred embodiment, in compounds I, m is 0 and R⁶ is H (or, alternatively, in compounds II, R^{6a} is H).

Particular compounds I/II are the following compounds of formula I.A, in which the combination of R¹, R², R³, R⁴, R⁵¹, R⁵², R⁵³, R⁵⁴ and R⁵⁵ for a compound corresponds in each case to one row of Table 2 and R⁷¹ is selected from hydrogen, methyl, ethyl, propyl and methoxymethyl:

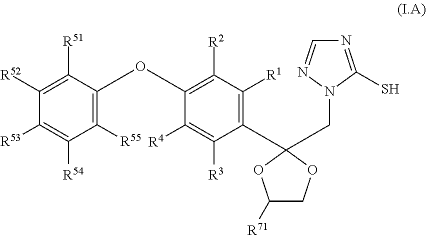

TABLE 2

| No. | R¹ | R² | R³ | R⁴ | R⁵¹ | R⁵² | R⁵³ | R⁵⁴ | R⁵⁵ |
|---|---|---|---|---|---|---|---|---|---|
| 1. | H | F | F | H | H | H | Cl | H | H |
| 2. | H | F | F | H | F | H | Cl | H | H |
| 3. | H | F | H | F | H | H | H | H | H |
| 4. | H | F | H | F | H | H | Cl | H | H |
| 5. | H | F | H | F | F | H | Cl | H | H |
| 6. | H | F | Cl | H | H | H | H | H | H |
| 7. | H | F | Cl | H | H | H | Cl | H | H |
| 8. | H | F | Cl | H | F | H | Cl | H | H |
| 9. | F | H | F | H | H | H | H | H | H |
| 10. | F | H | F | H | H | H | Cl | H | H |
| 11. | F | H | F | H | F | H | Cl | H | H |
| 12. | F | H | H | H | H | H | H | H | H |
| 13. | F | H | H | H | H | H | Cl | H | H |
| 14. | F | H | H | H | F | H | Cl | H | H |
| 15. | H | F | H | H | H | H | H | H | H |
| 16. | H | F | H | H | H | H | Cl | H | H |
| 17. | H | F | H | H | F | H | Cl | H | H |
| 18. | Cl | H | H | H | H | H | H | H | H |
| 19. | Cl | H | H | H | F | H | Cl | H | H |
| 20. | Cl | H | H | H | F | H | F | H | H |
| 21. | Cl | H | H | H | F | H | H | F | H |
| 22. | Cl | H | H | H | F | H | H | H | Cl |
| 23. | CH₃ | H | H | H | F | H | F | H | H |
| 24. | CH₃ | H | H | H | F | H | Cl | H | H |
| 25. | CH₃ | H | H | H | F | H | H | F | H |
| 26. | CH₃ | H | H | H | F | H | H | H | F |
| 27. | CH₃ | H | H | H | F | H | H | H | Cl |
| 28. | CH₃ | H | H | H | Cl | H | Cl | H | H |
| 29. | CH₃ | H | H | H | H | H | Cl | H | H |
| 30. | CH₃ | H | H | H | Cl | H | H | H | H |
| 31. | CH₃ | H | H | H | Cl | H | H | Cl | H |
| 32. | CH₃ | H | H | H | H | Cl | H | Cl | H |
| 33. | CF₃ | H | H | H | F | H | F | H | H |
| 34. | CF₃ | H | H | H | F | H | Cl | H | H |
| 35. | CF₃ | H | H | H | F | H | H | F | H |
| 36. | CF₃ | H | H | H | F | H | H | H | F |
| 37. | CF₃ | H | H | H | F | H | H | H | Cl |
| 38. | CF₃ | H | H | H | Cl | H | Cl | H | H |
| 39. | CF₃ | H | H | H | H | H | Cl | H | H |
| 40. | CF₃ | H | H | H | Cl | H | H | Cl | H |
| 41. | CF₃ | H | H | H | H | Cl | H | Cl | H |
| 42. | H | H | H | H | F | H | F | H | H |
| 43. | H | H | H | H | F | H | Cl | H | H |
| 44. | H | H | H | H | F | H | H | F | H |
| 45. | H | H | H | H | F | H | H | H | F |
| 46. | H | H | H | H | F | H | H | H | Cl |
| 47. | Cl | H | H | H | H | H | Cl | H | H |
| 48. | F | H | H | F | H | H | H | H | H |
| 49. | F | H | H | F | H | H | Cl | H | H |

except for compounds wherein the combination of R¹, R², R³, R⁴, R⁵¹, R⁵², R⁵³, R⁵⁴ and R⁵⁵ corresponds to row of 47 Table 2 and simultaneously R⁷¹ is selected from hydrogen, methyl, ethyl and propyl.

Particular compounds I/II are moreover the following compounds of formula I.B, in which the combination of R¹, R², R³, R⁴, R⁵¹, R⁵², R⁵³, R⁵⁴ and R⁵⁵ for a compound corresponds in each case to one row of Table 2 above and R⁷¹ is selected from hydrogen, methyl, ethyl, propyl and methoxymethyl:

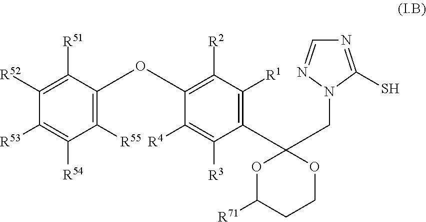

except for the compound wherein the combination of R¹, R², R³, R⁴, R⁵¹, R⁵², R⁵³, R⁵⁴ and R⁵⁵ corresponds to row 47 of Table 2 and simultaneously R⁷¹ is hydrogen.

In sum, particular compounds I/II are the following compounds of formula I.C, in which the combination of R¹, R², R³, R⁴, R⁵¹, R⁵², R⁵³, R⁵⁴ and R⁵⁵ for a compound corresponds in each case to one row of Table 2 above, R⁷¹ is selected from hydrogen, methyl, ethyl, propyl and methoxymethyl and a is 0 or 1:

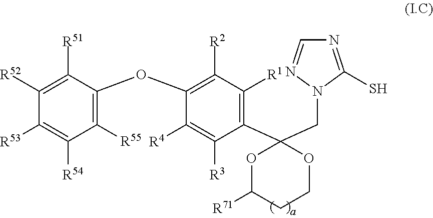

except for the compound wherein a is 1, the combination of R¹, R², R³, R⁴, R⁵¹, R⁵², R⁵³, R⁵⁴ and R⁵⁵ corresponds to row 47 of Table 2 and simultaneously R⁷¹ is hydrogen; and
except for compounds wherein a is 0, the combination of R¹, R², R³, R⁴, R⁵¹, R⁵², R⁵³, R⁵⁴ and R⁵⁵ corresponds to row 47 of Table 2 and simultaneously R⁷¹ is selected from hydrogen, methyl, ethyl and propyl.

Specific compounds I/II are the following compounds of formula I.C1, in which the combination of R¹, R², R³ and R⁴ for a compound corresponds in each case to one row of Table 3, R⁵³ is hydrogen or Cl, R⁷¹ is selected from hydrogen, methyl, ethyl, propyl and methoxymethyl and a is 0 or 1:

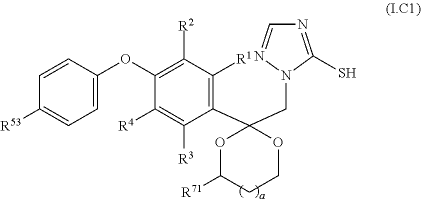

TABLE 3

| No. | R¹ | R² | R³ | R⁴ |
|---|---|---|---|---|
| 1. | H | F | F | H |
| 2. | H | F | H | F |
| 3. | H | F | Cl | H |
| 4. | F | H | F | H |
| 5. | F | H | H | H |
| 6. | H | F | H | H |
| 7. | Cl | H | H | H |
| 8. | CH₃ | H | H | H |
| 9. | CF₃ | H | H | H |
| 10. | H | H | H | H |
| 11. | F | H | H | F |

except for the compound wherein R⁵³ is Cl, a is 1, the combination of R¹, R², R³ and R⁴ corresponds to row of 7 Table 3 and simultaneously R⁷¹ is hydrogen; and
except for compounds wherein R⁵³ is Cl, a is 1, the combination of R¹, R², R³ and R⁴ corresponds to row of 7 Table 3 and simultaneously R⁷¹ is selected from hydrogen, methyl, ethyl and propyl.

Very specific compounds are compounds of formula I.C1, wherein
a is 1, R¹ is Cl, R², R³ and R⁴ are H, R⁵³ is Cl and R⁷¹ is methyl (compound I.C1.1);
a is 0, R¹ and R⁴ are F, R² and R³ and are H, R⁵³ is Cl and R⁷¹ is methyl (compound I.C1.2);
a is 0, R¹ and R⁴ are F, R² and R³ and are H, R⁵³ is H and R⁷¹ is methyl (compound I.C1.3);
a is 0, R¹ is Cl, R², R³ and R⁴ are H, R⁵³ is Cl and R⁷¹ is n-propyl (compound I.C1.4);
a is 0, R¹ is Cl, R², R³ and R⁴ are H, R⁵³ is Cl and R⁷¹ is methoxymethyl (compound I.C1.5);
a is 0, R² is F, R¹, R³ and R⁴ are H, R⁵³ is H and R⁷¹ is methyl (compound I.C1.6);
a is 0, R² and R⁴ are F, R¹ and R³ and are H, R⁵³ is H and R⁷¹ is methyl (compound I.C1.7);
a is 0, R² is F, R¹, R³ and R⁴ are H, R⁵³ is Cl and R⁷¹ is methyl (compound I.C1.8);
a is 0, R¹ is F, R², R³ and R⁴ are H, R⁵³ is Cl and R⁷¹ is methyl (compound I.C1.9);
a is 0, R¹ is methyl, R², R³ and R⁴ are H, R⁵³ is Cl and R⁷¹ is methyl (compound I.C1.10).

Examples for preferred compounds I and II are compounds of formulae I.1 to I.96 and II.1 to II.48, where the variables have one of the general or, in particular, one of the preferred meanings given above. Examples of preferred compounds are the individual compounds compiled in the tables 1 to 87648 below. Moreover, the meanings mentioned below for the individual variables in the tables are per se, independently of the combination in which they are mentioned, a particularly preferred embodiment of the substituents in question.

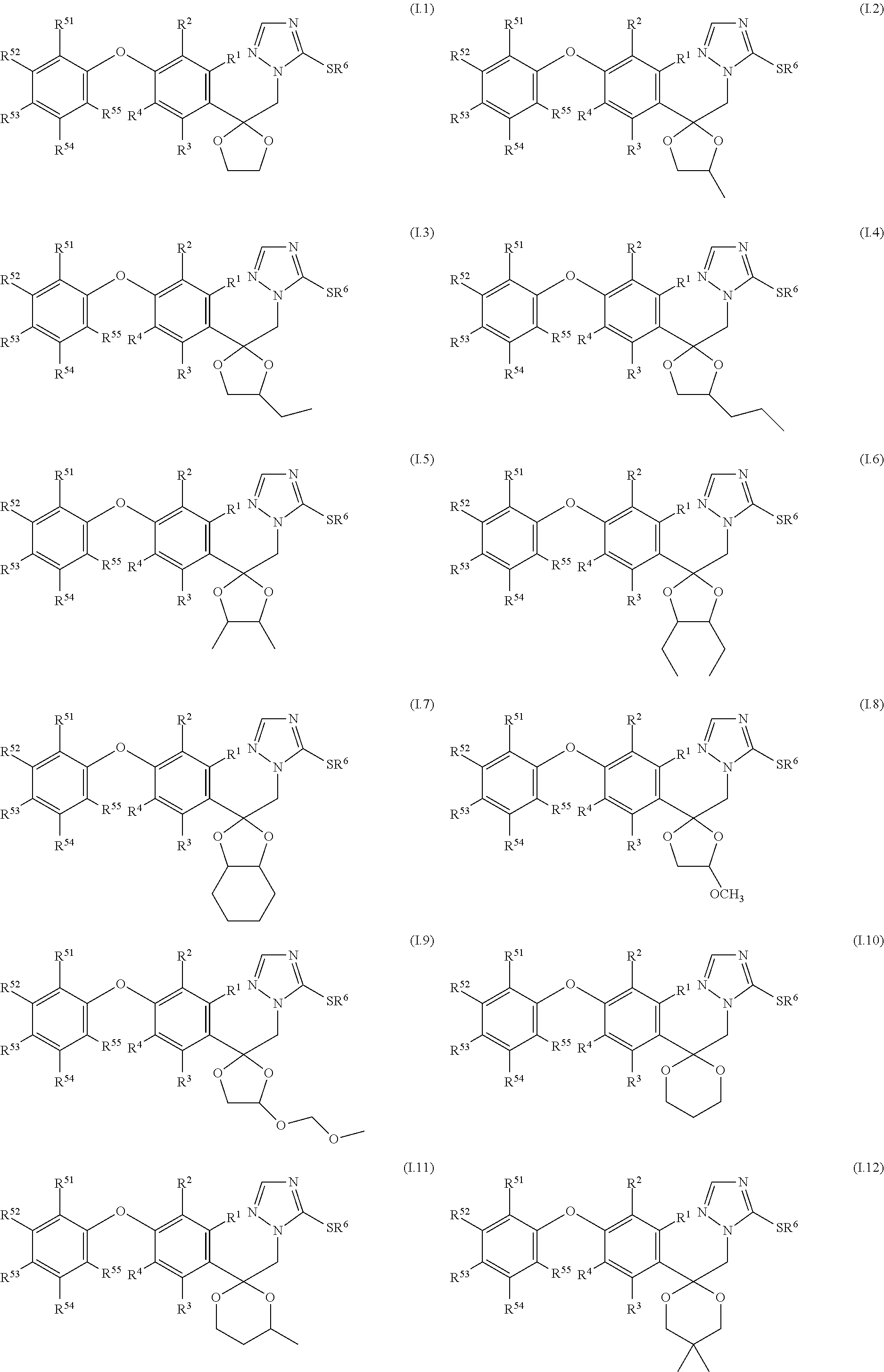

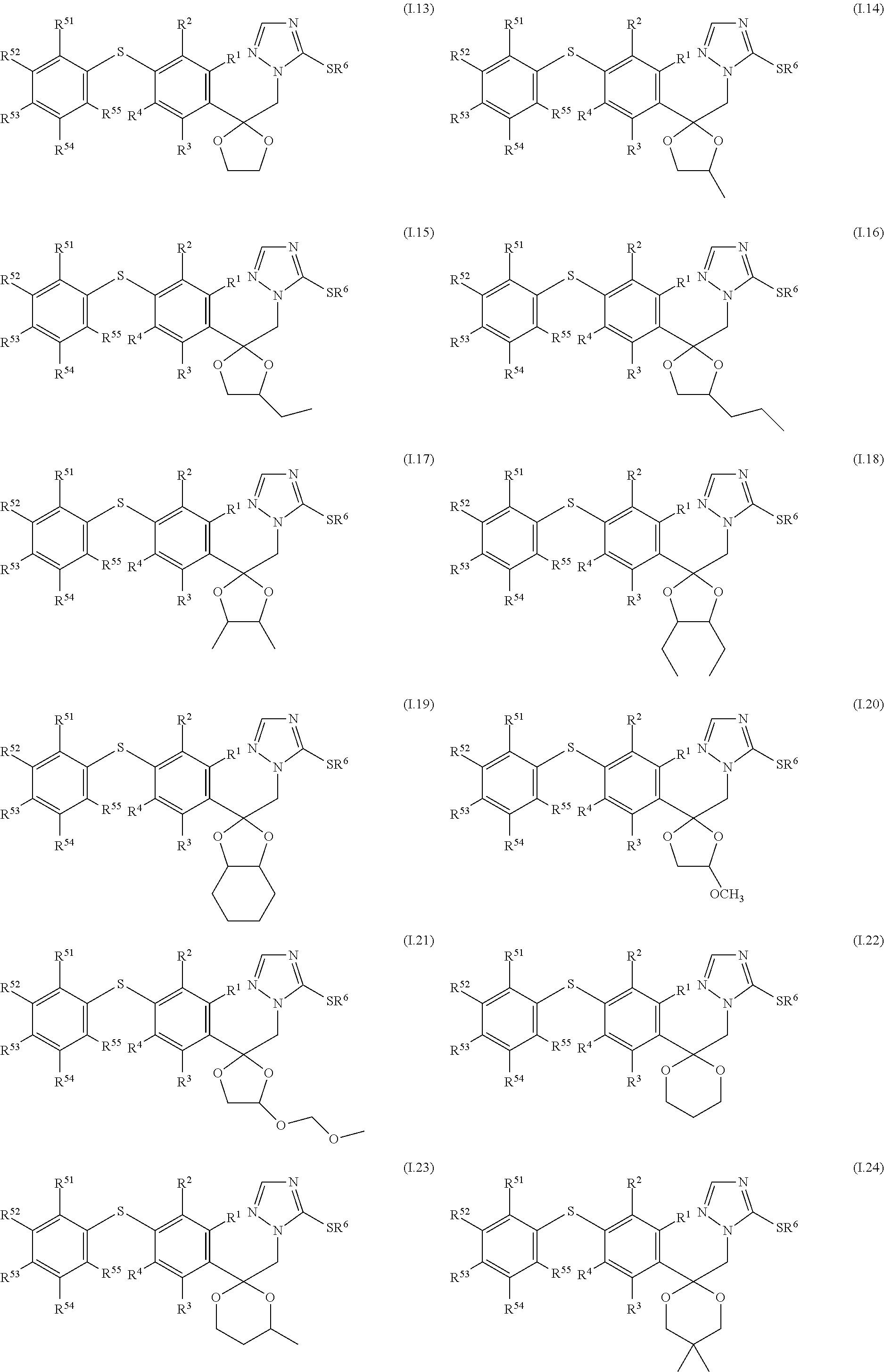

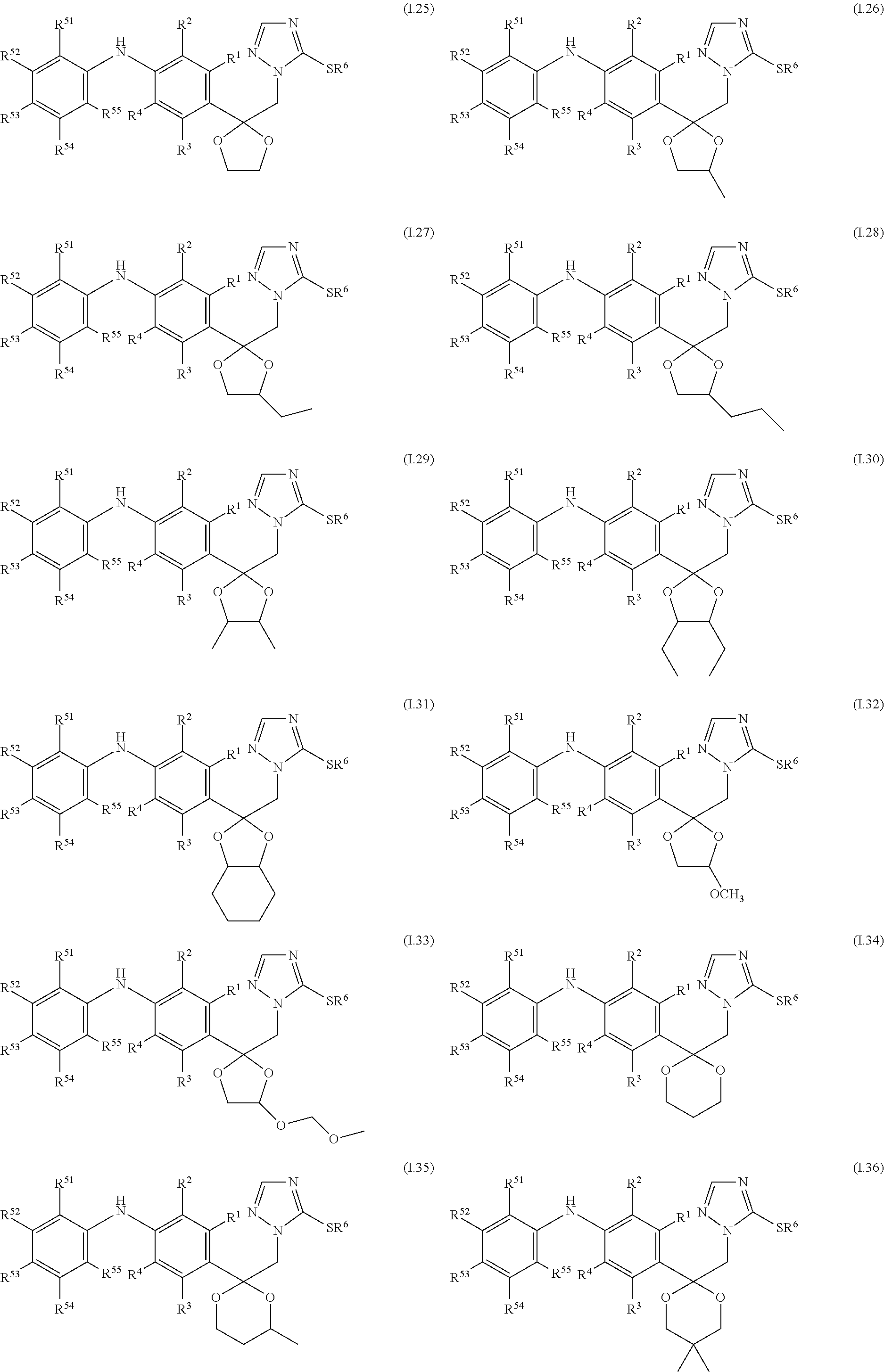

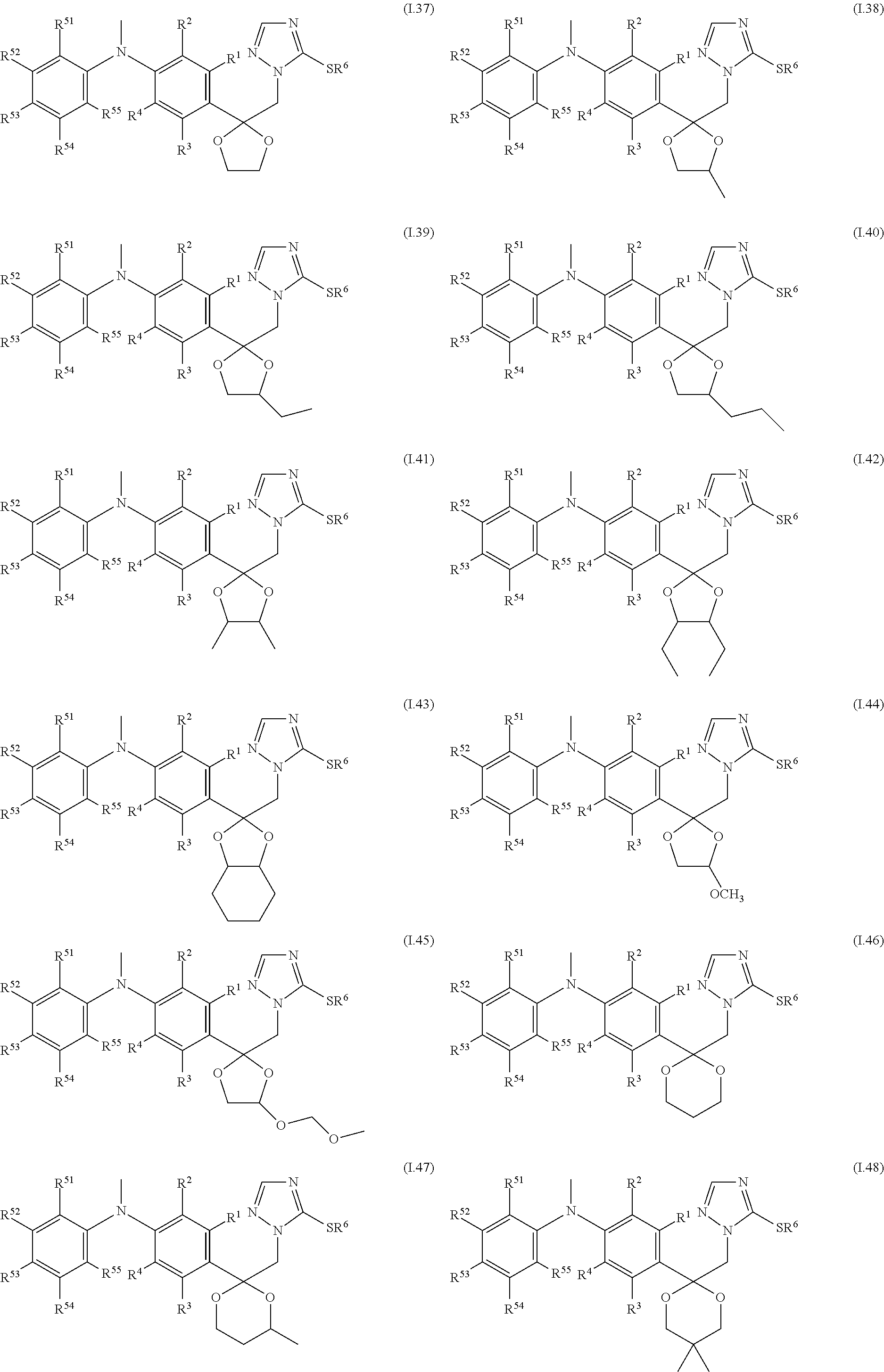

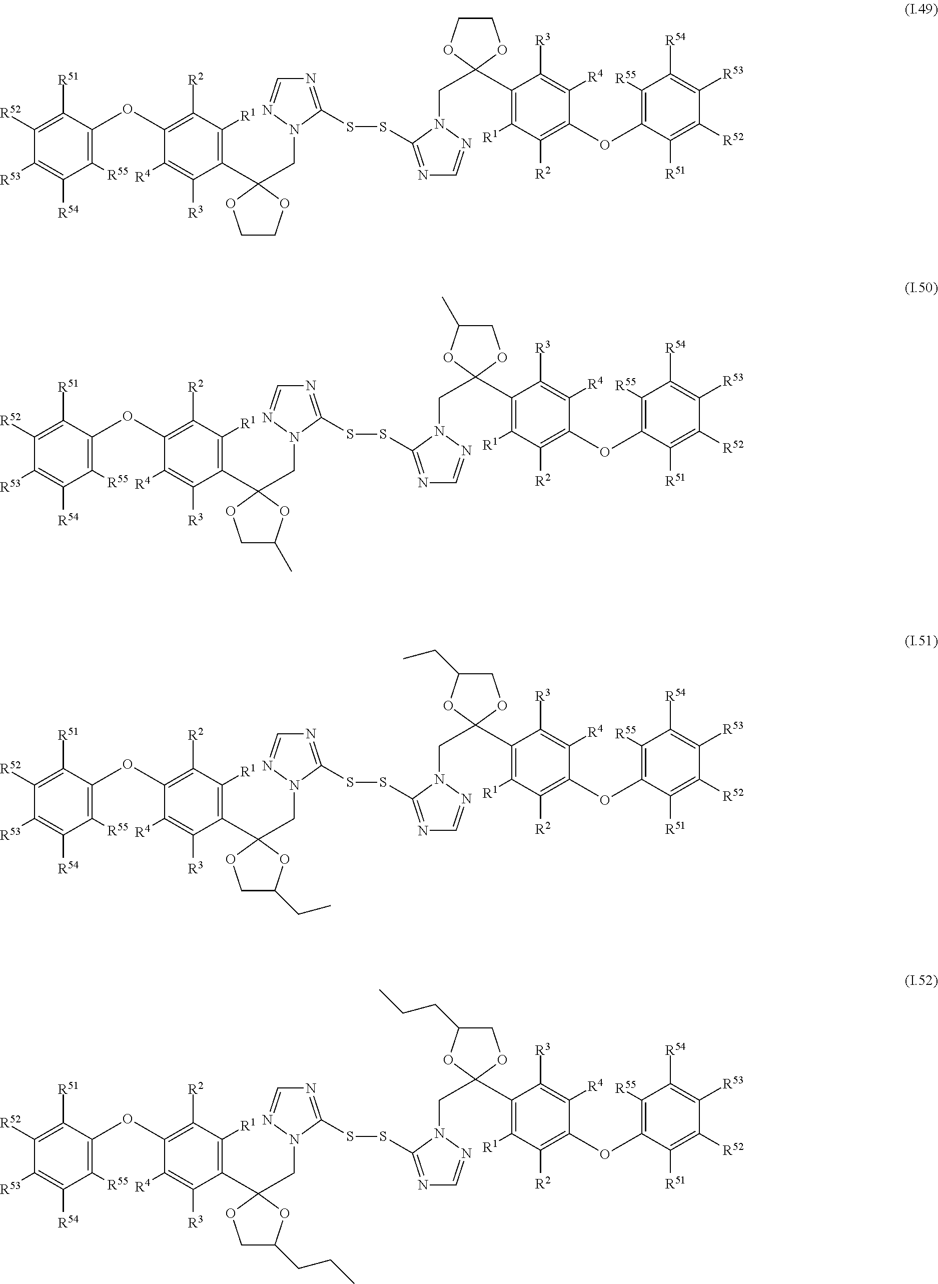

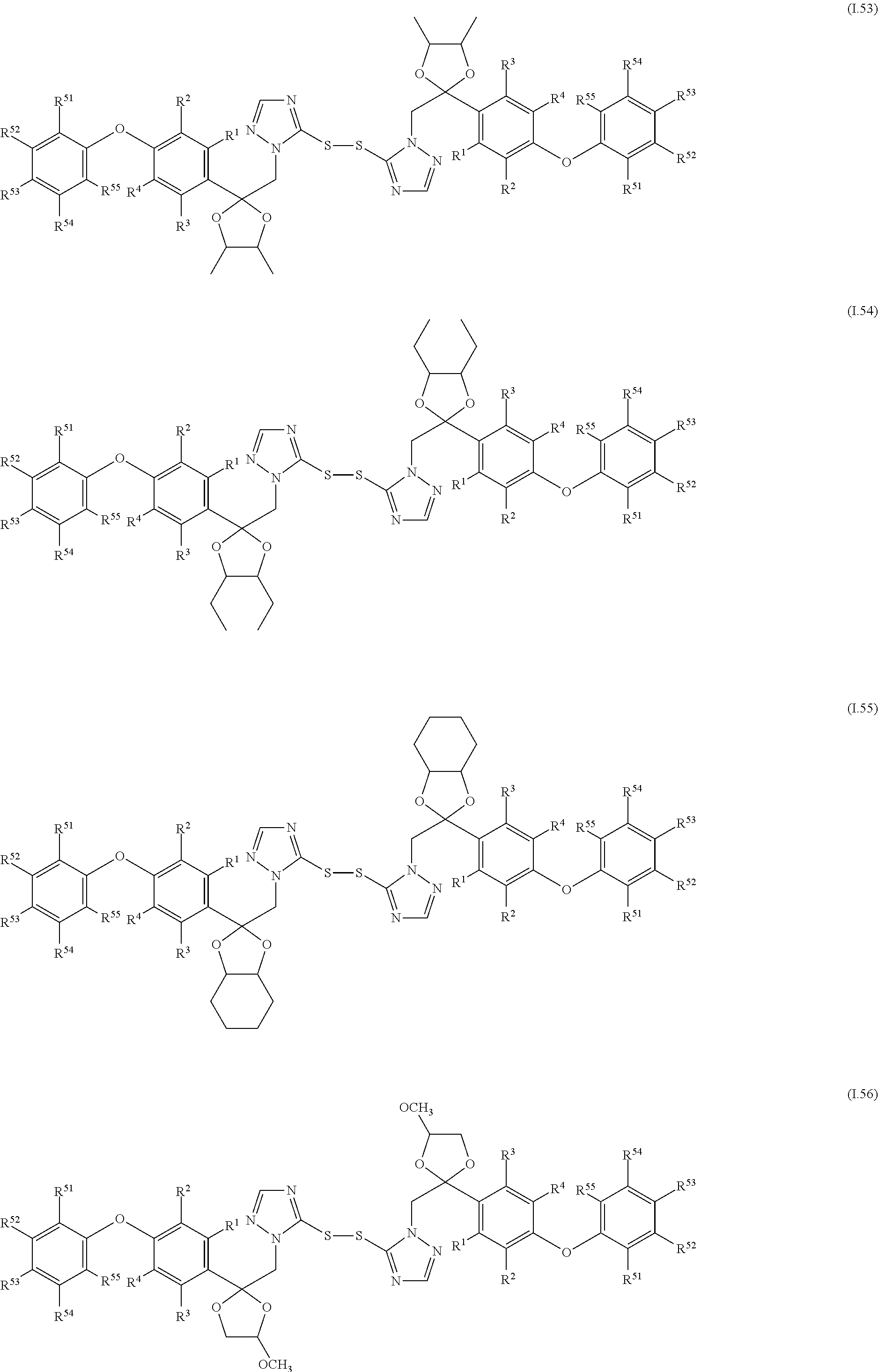

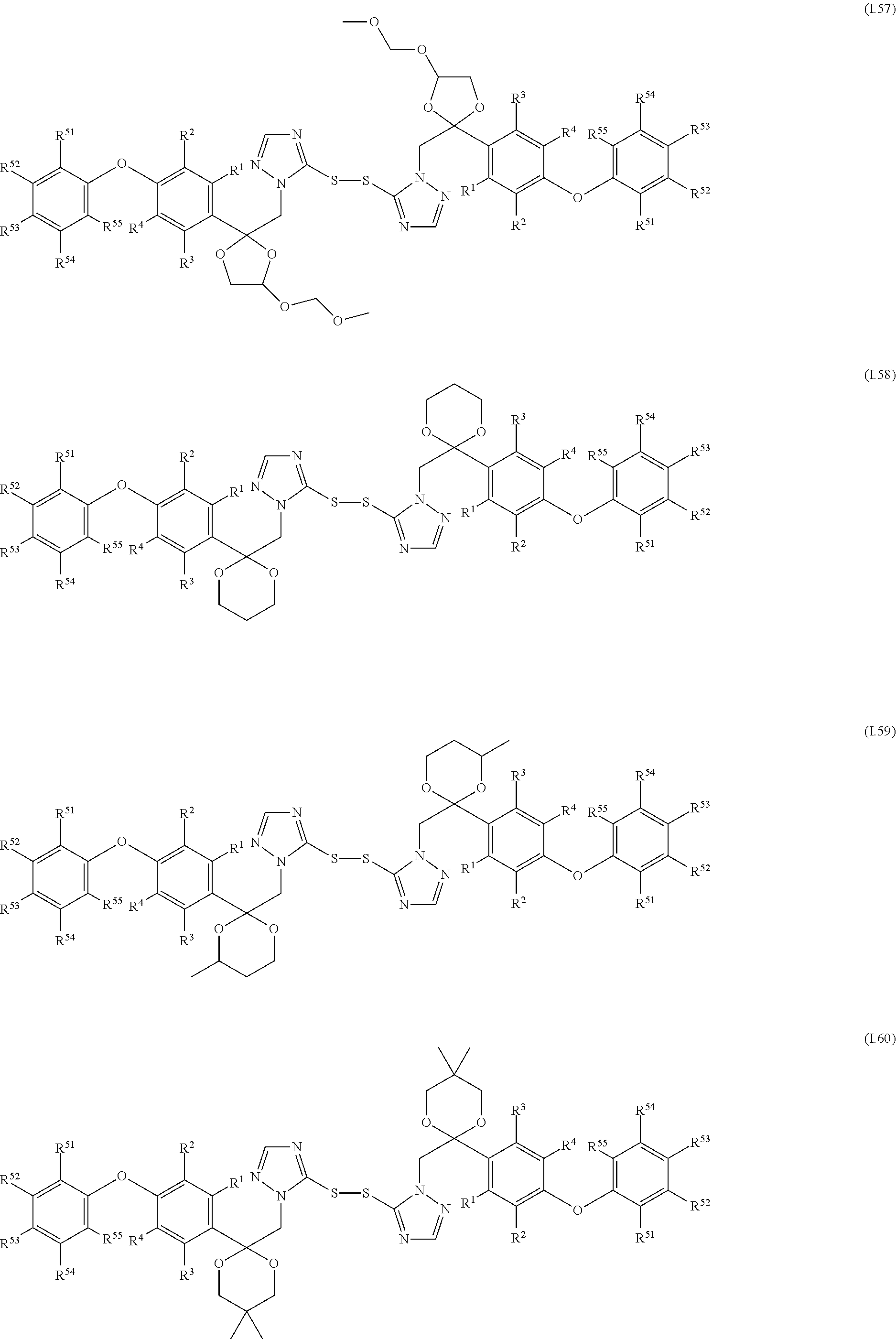

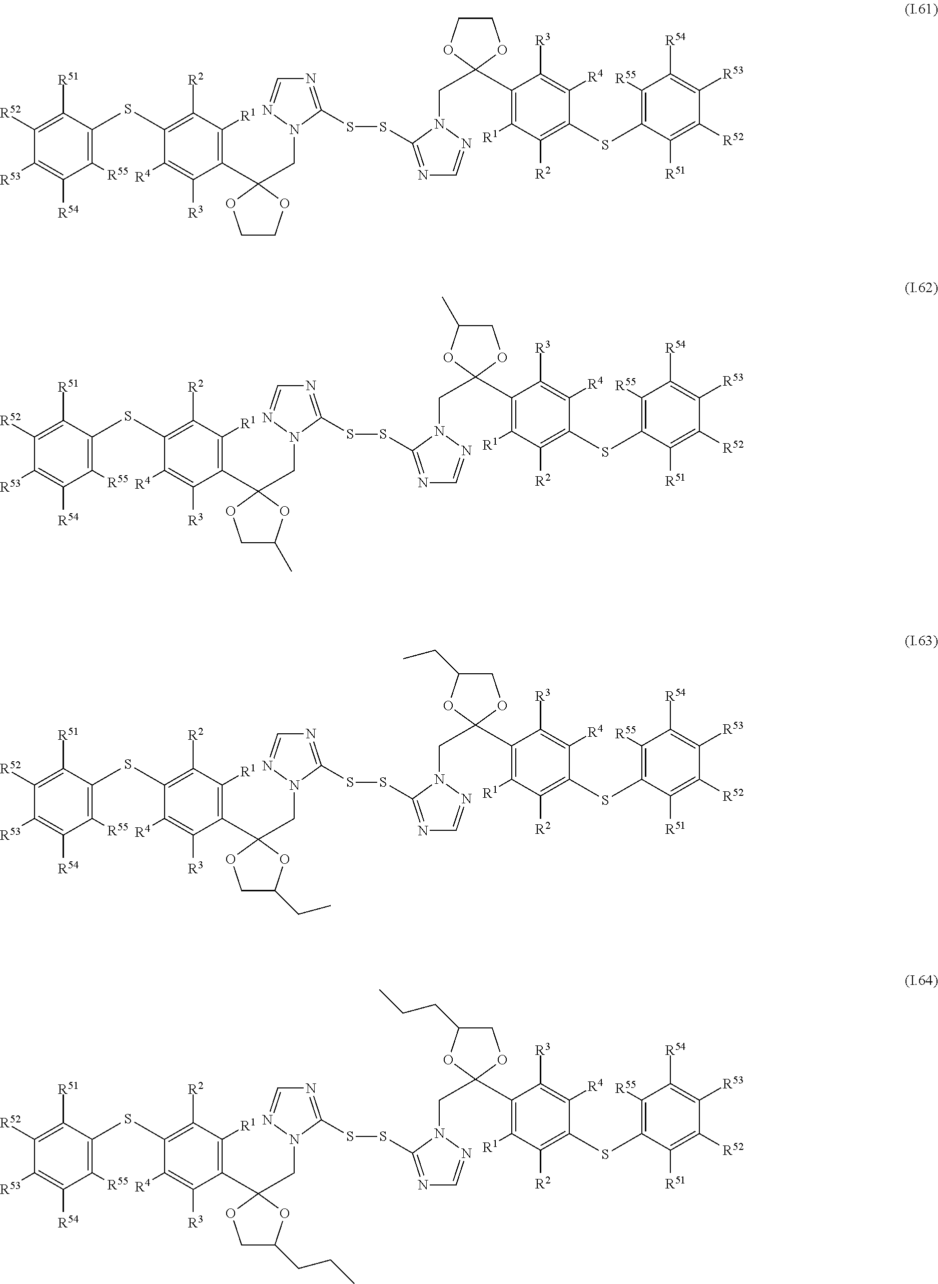

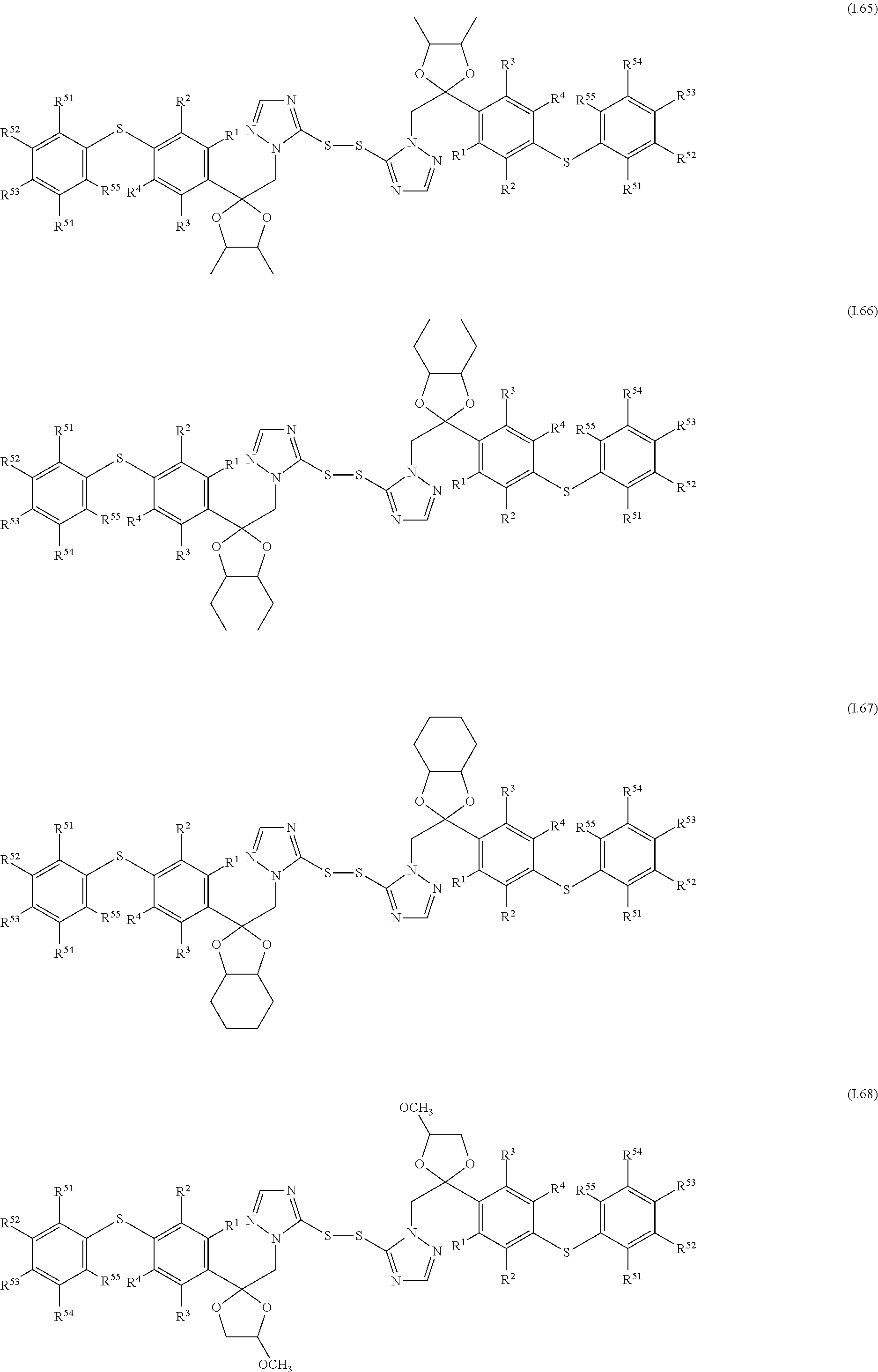

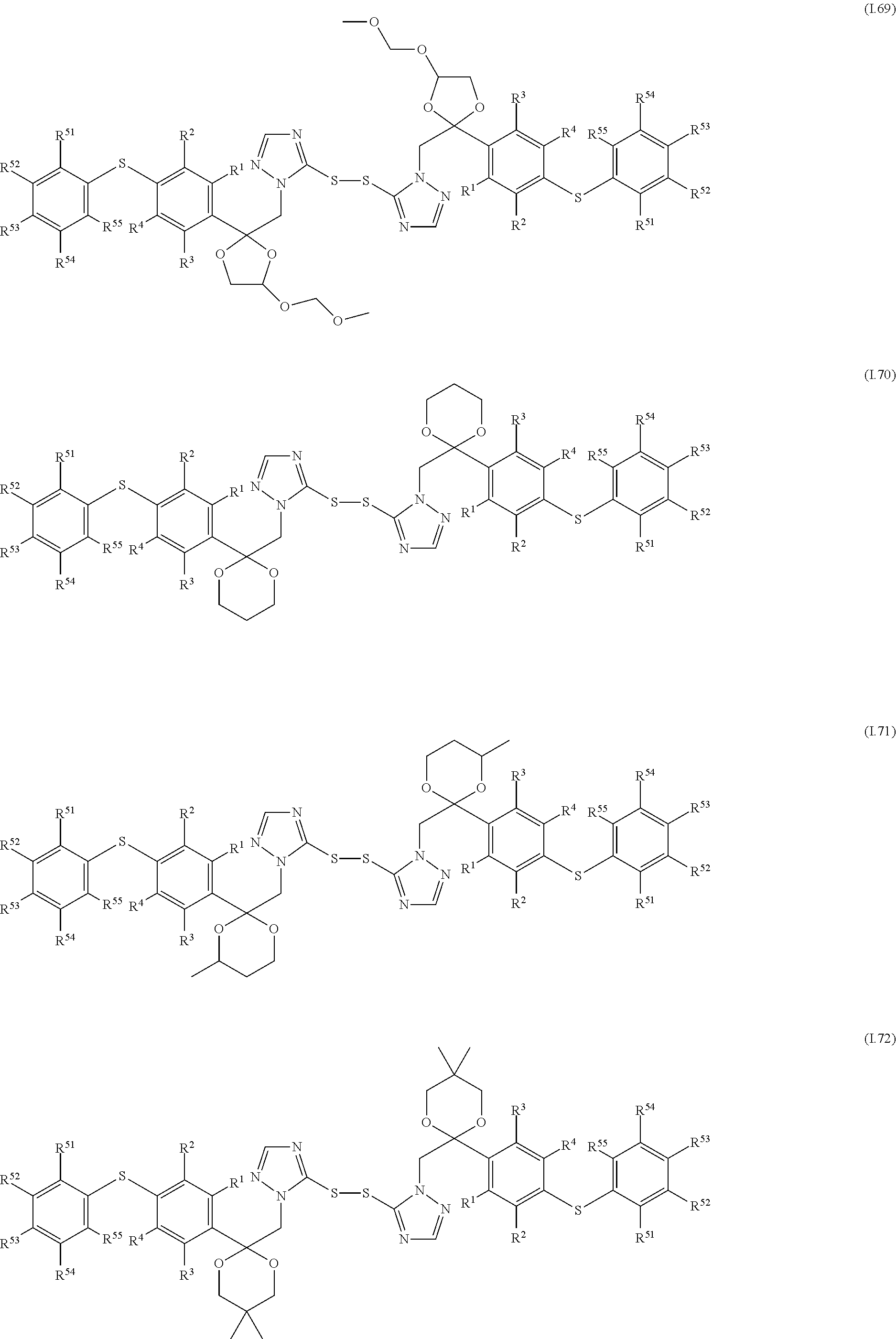

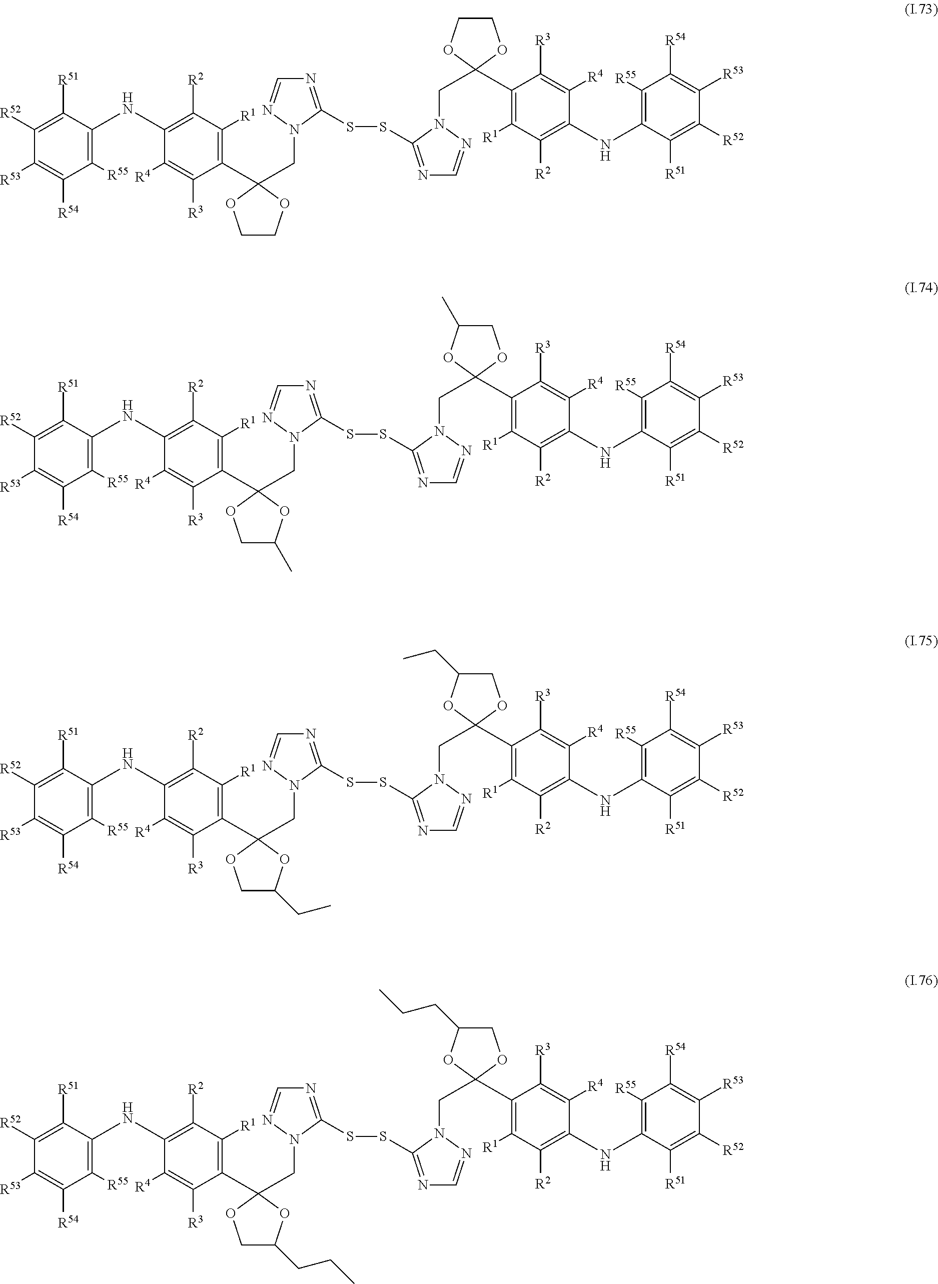

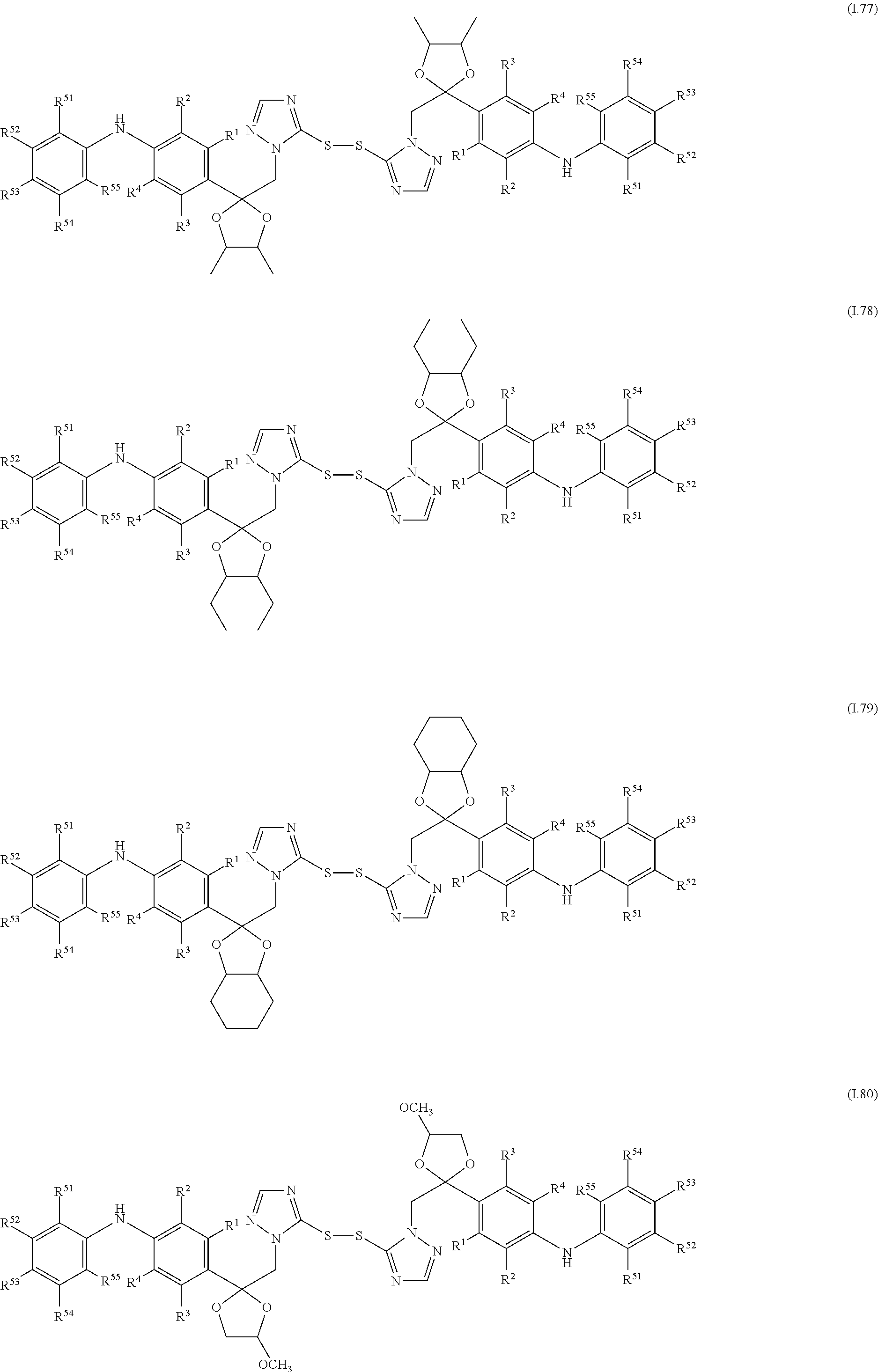

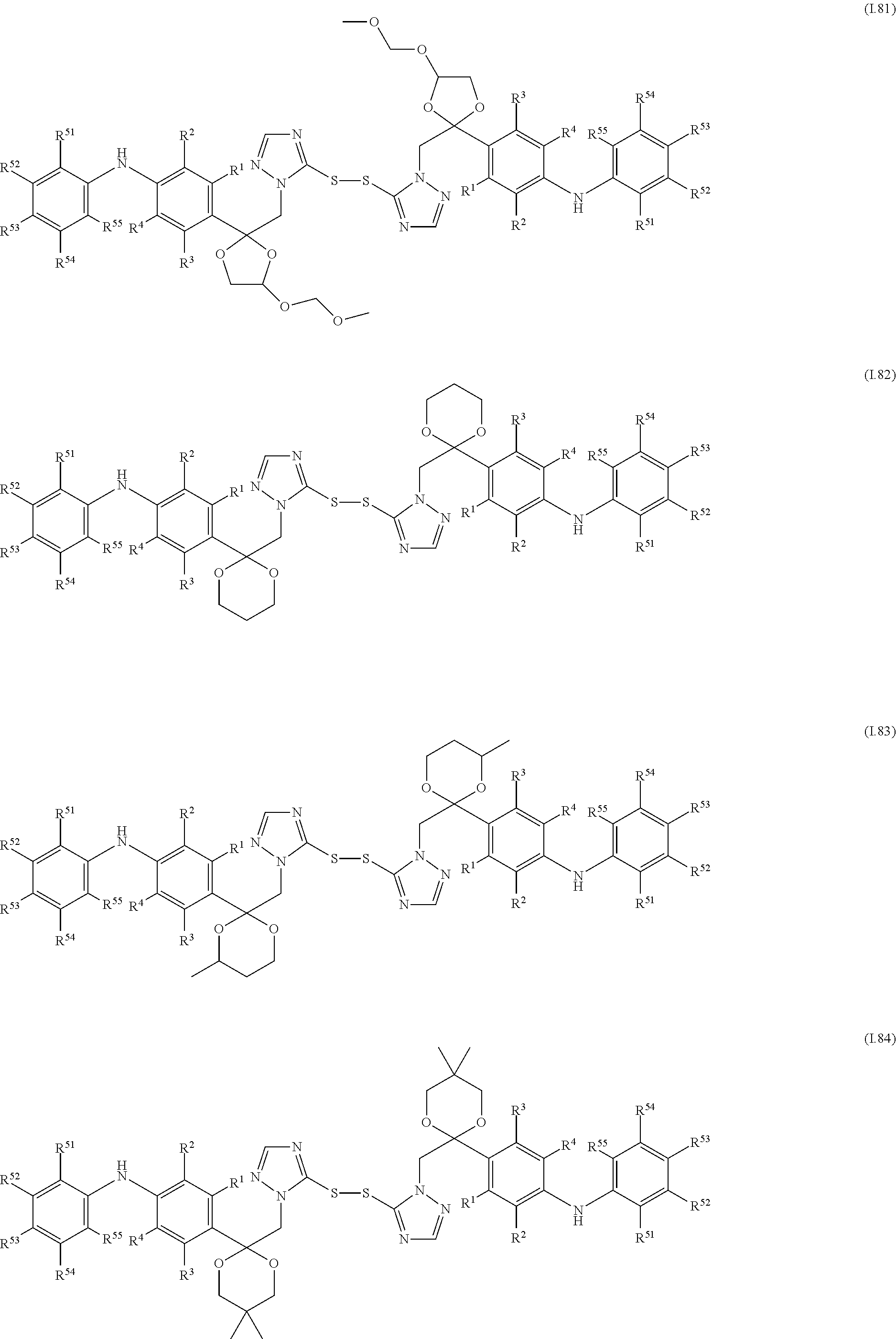

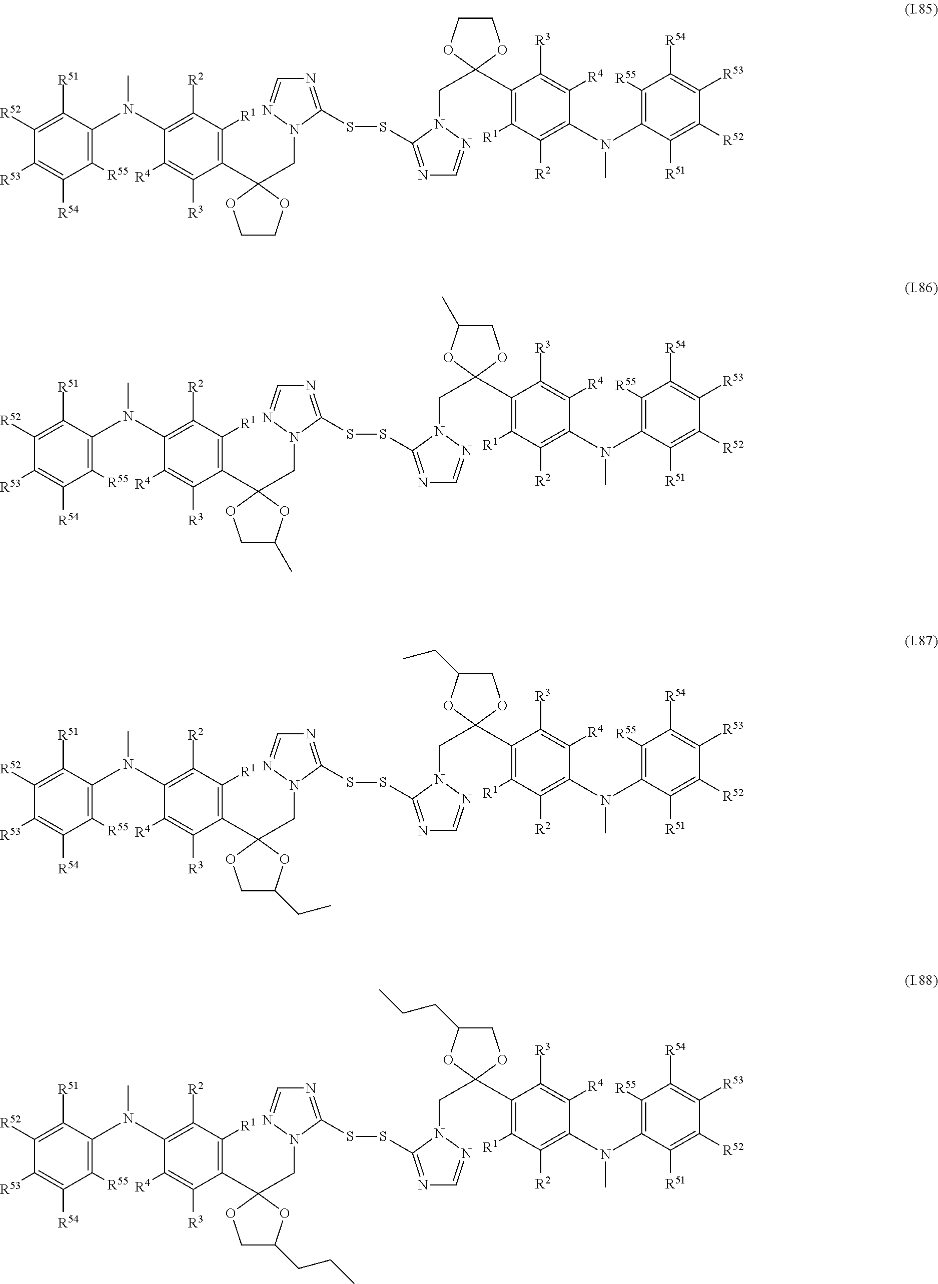

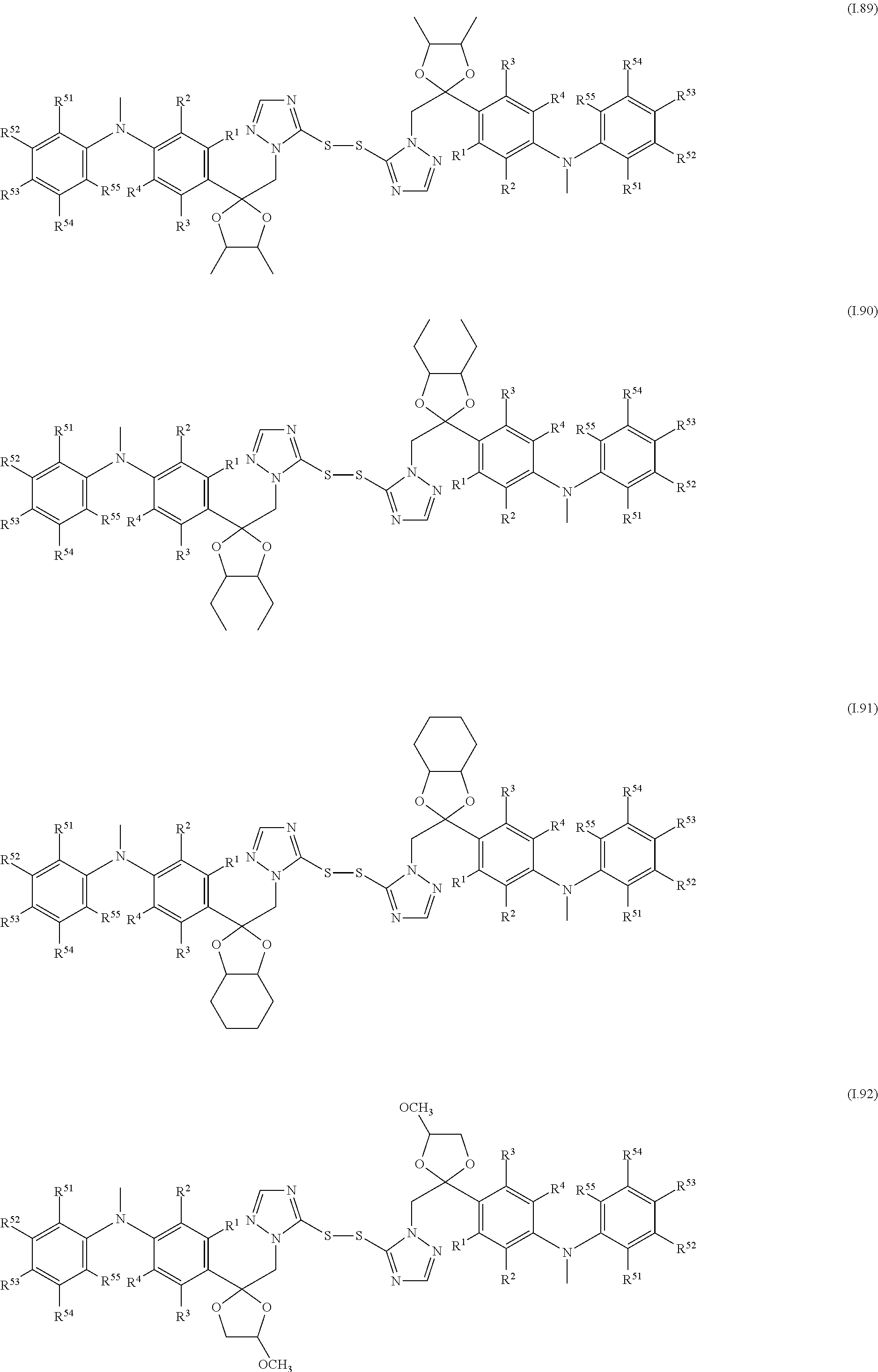

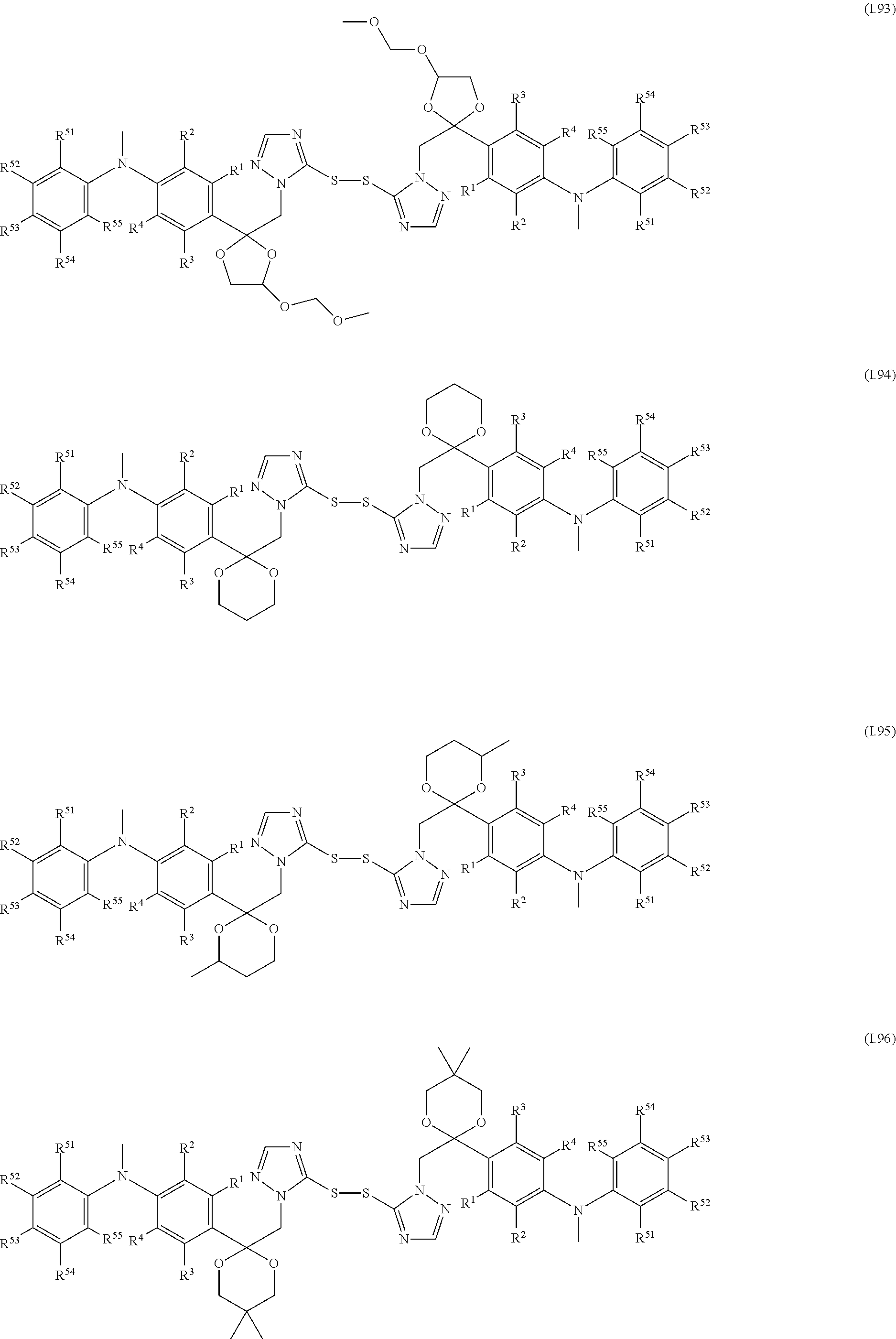

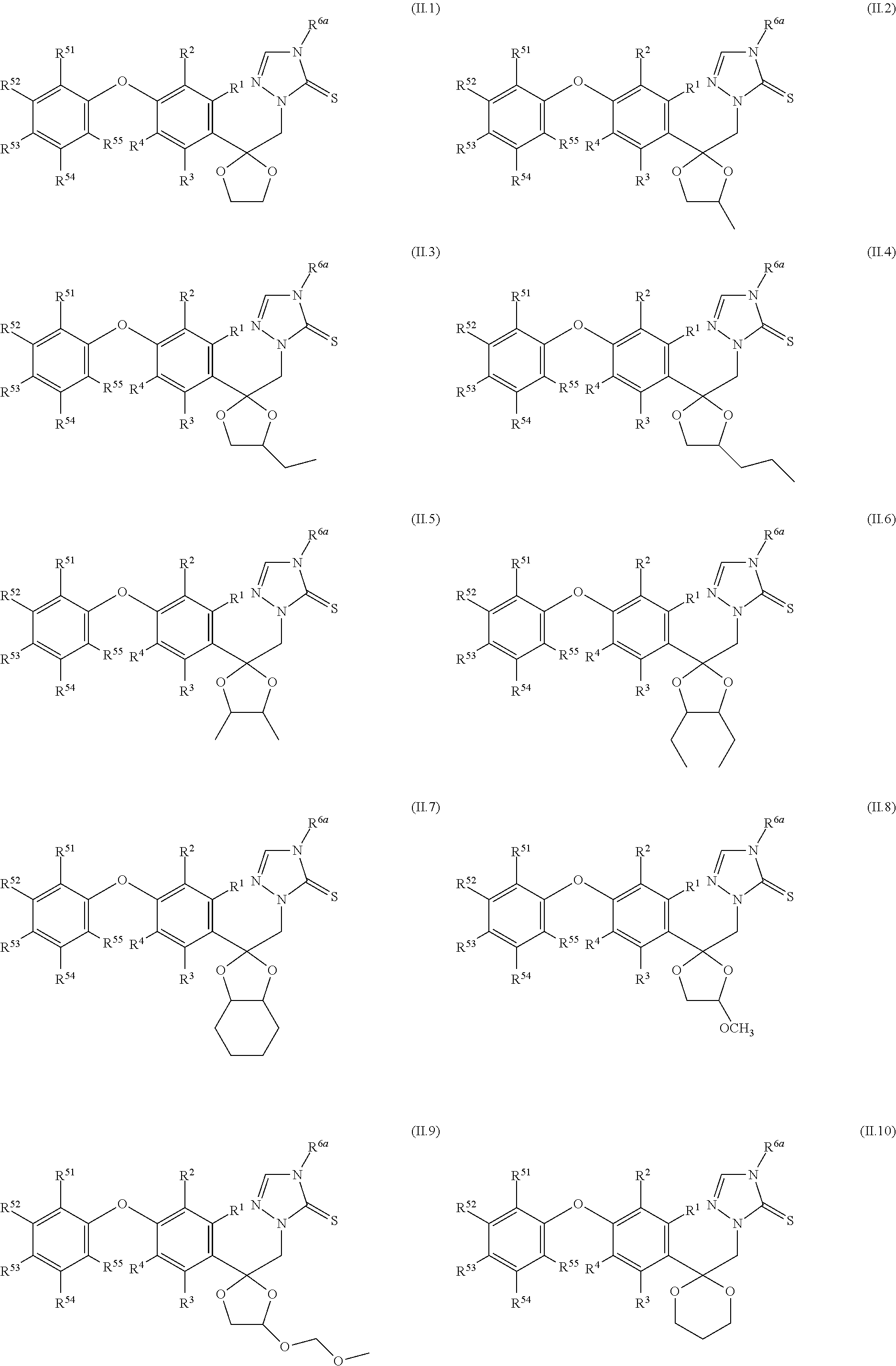

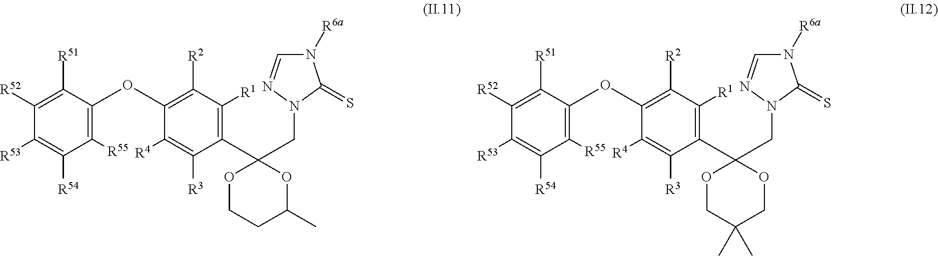

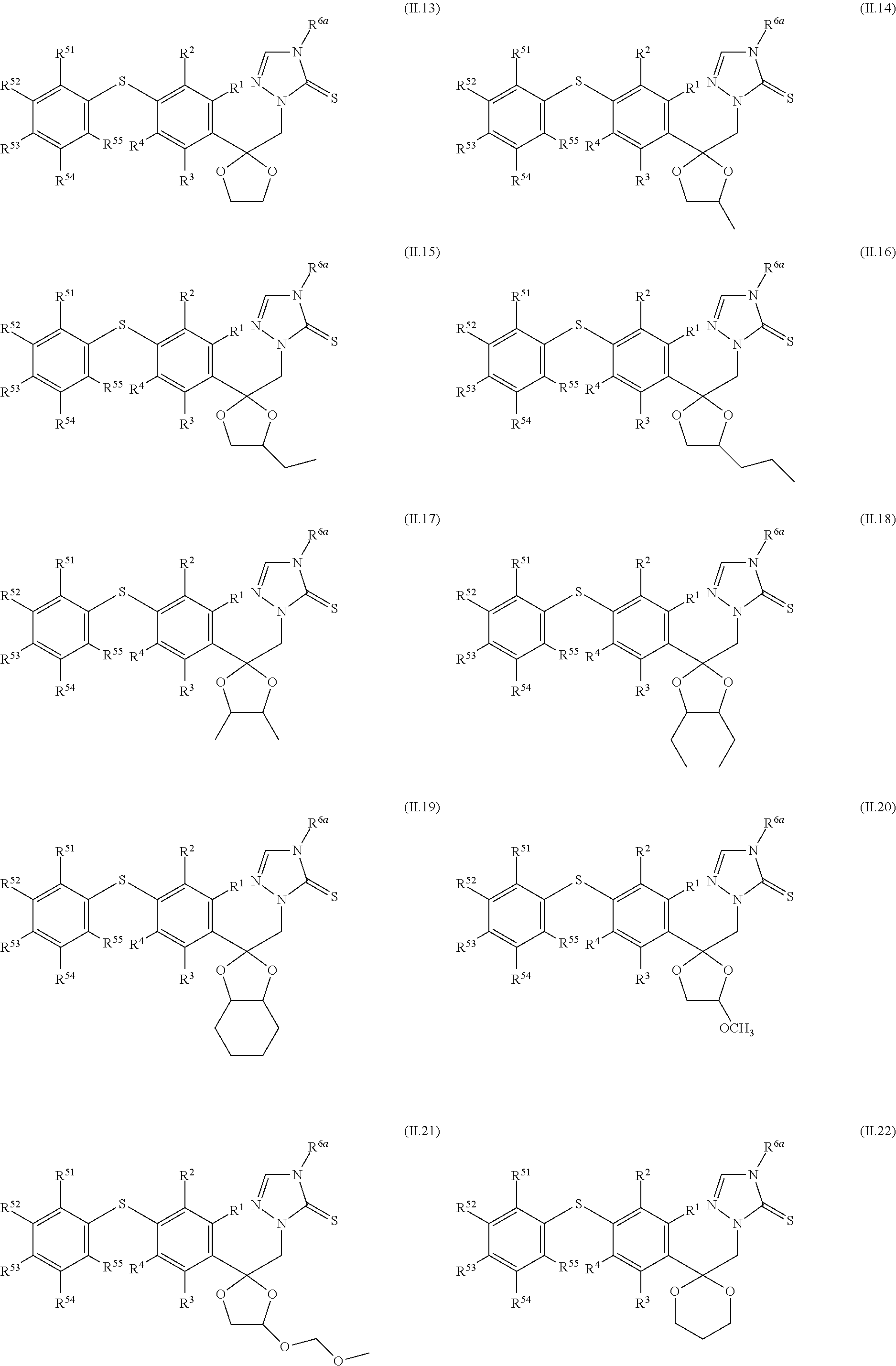

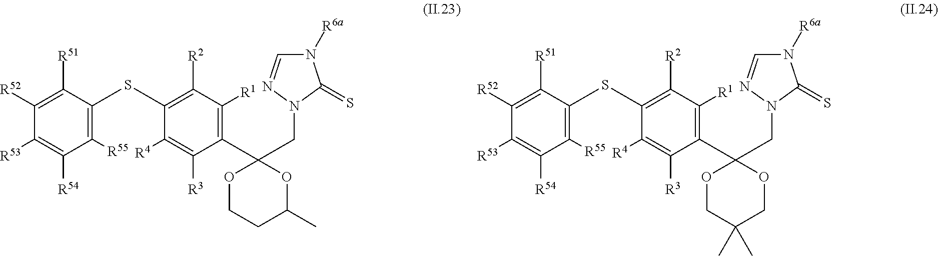

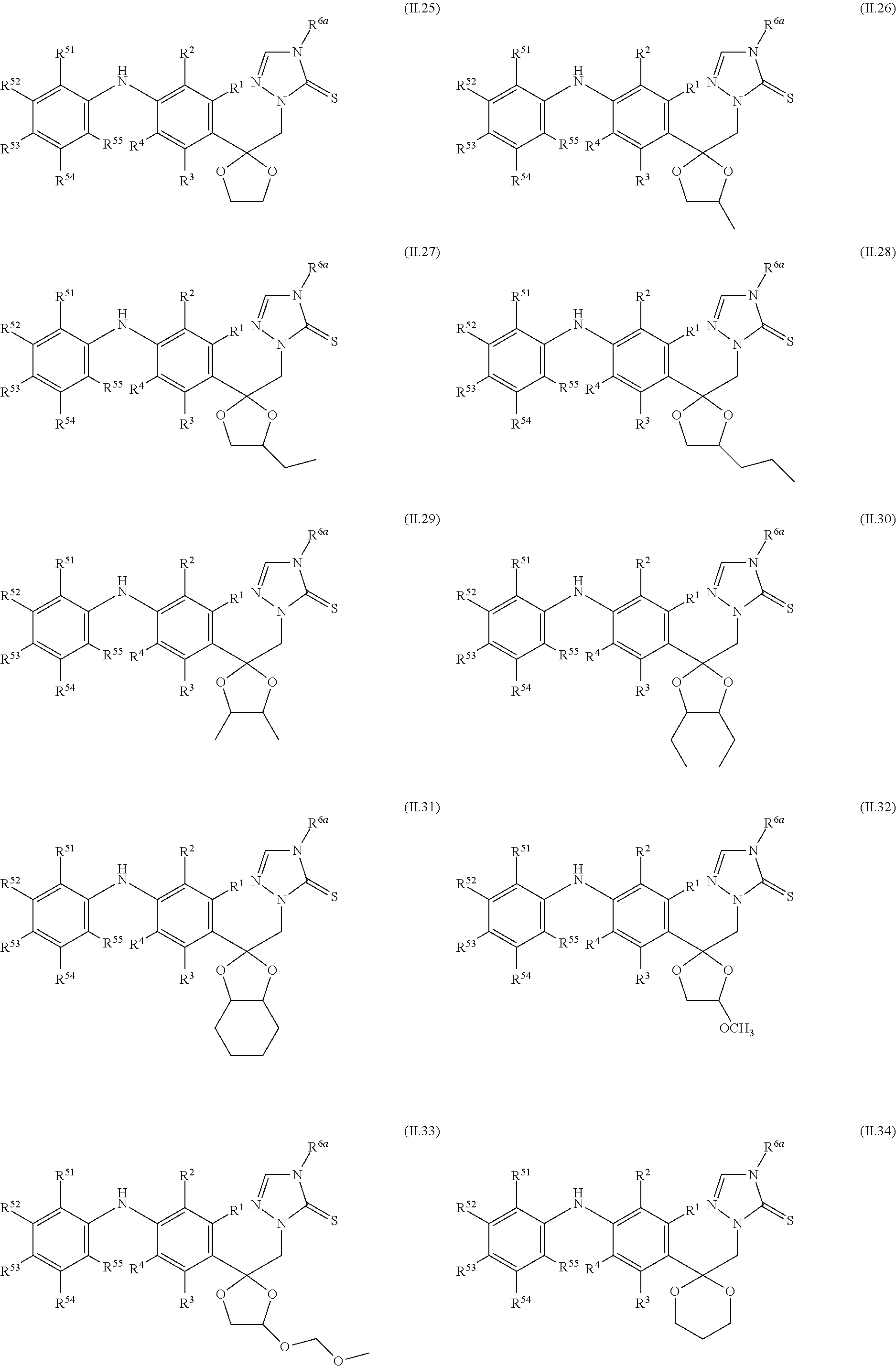

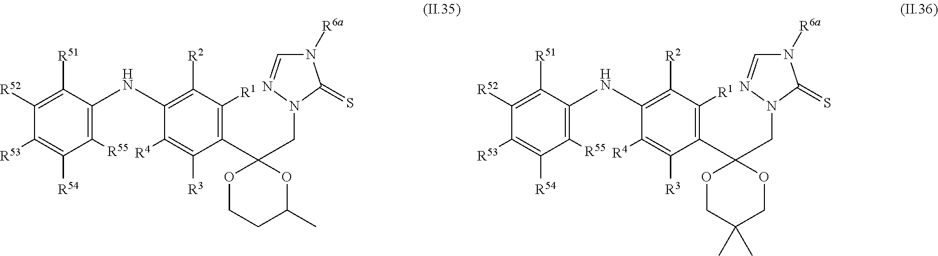

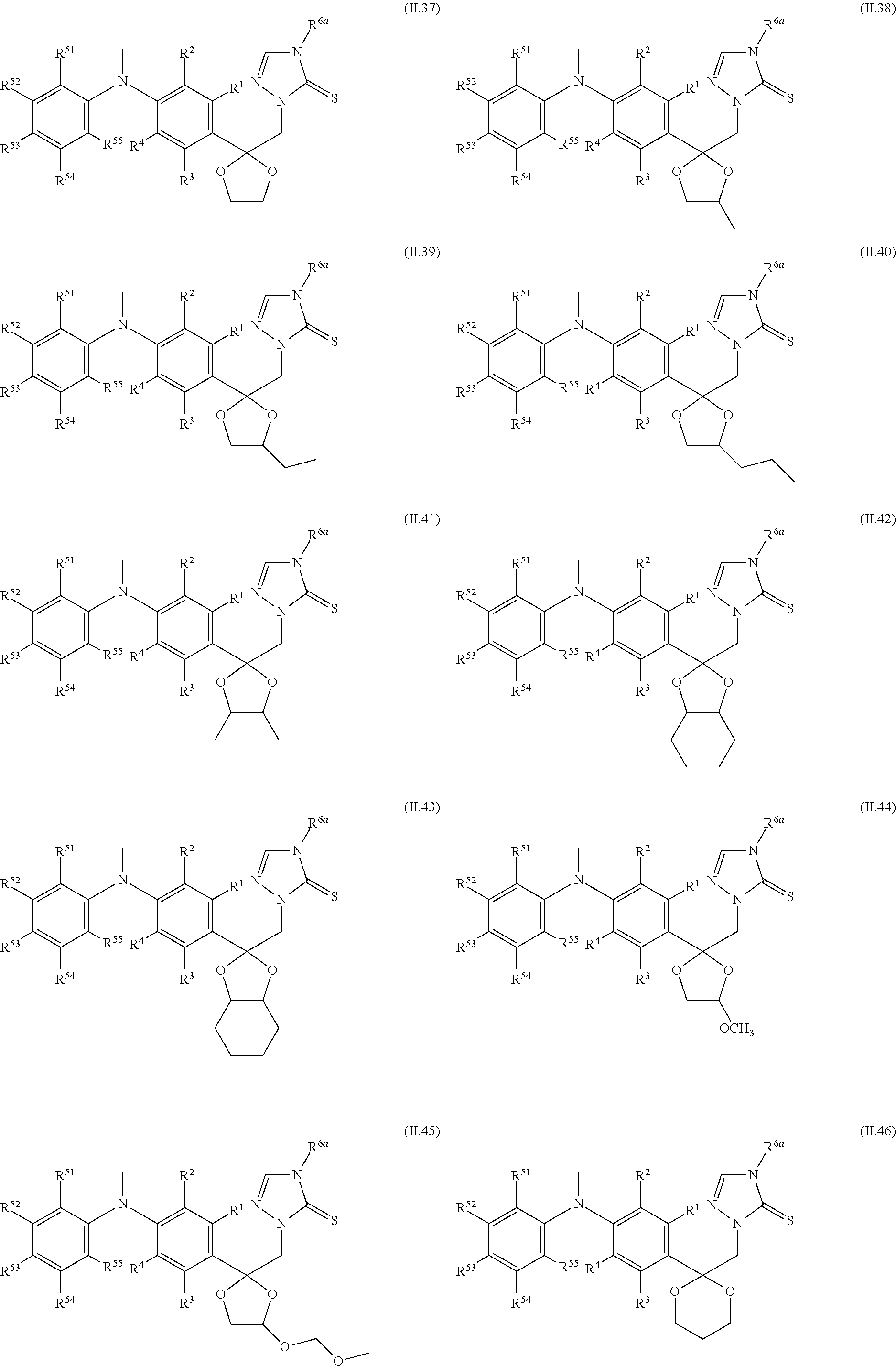

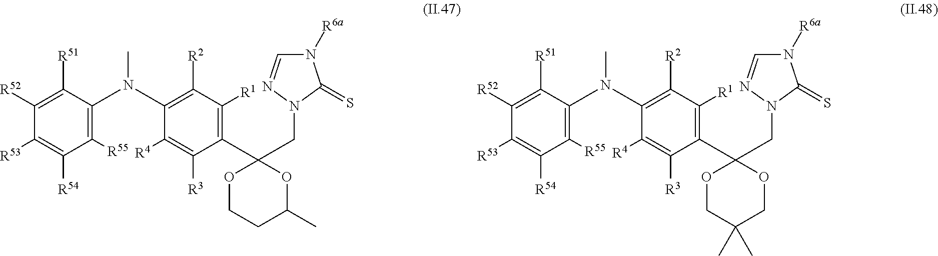

### Table 1

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is H

### Table 2

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is methyl

### Table 3

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is ethyl

### Table 4

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is propyl

### Table 5

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is isopropyl

### Table 6

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is n-butyl

### Table 7

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is sec-butyl

### Table 8

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is isobutyl

### Table 9

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is tert-butyl

### Table 10

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is phenyl

### Table 11

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is benzyl

### Table 12

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is Li⁺

### Table 13

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is Na⁺

### Table 14

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is K⁺

### Table 15

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is ½Mg²⁺

### Table 16

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is ½Cu²⁺

### Table 17

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is ½Zn²⁺

### Table 18

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is ½Fe²⁺

### Table 19

Compounds of the formula I.1 in which the combination of R⁹¹, R⁹², R⁹³, R⁹⁴ and R⁹⁵ for a compound corresponds in each case to one row of Table A, R¹, R², R³ and R⁴ are H and R⁶ is ½Ni²⁺

### Table 20

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is NH(CH₃)₃⁺

### Table 21

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is NH(C₂H₅)₃⁺

### Table 22

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is NH(CH₂CH₂CH₂)₃⁺

### Table 23

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is NH(CH(CH₃)₂)₃⁺

### Table 24

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is NH(CH₂CH₂CH₂CH₂)₃⁺

### Table 25

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is methylcarbonyl

### Table 26

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is ethylcarbonyl

### Table 27

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is propylcarbonyl

### Table 28

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is isopropylcarbonyl

### Table 29

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is methoxycarbonyl

### Table 30

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is ethoxycarbonyl

### Table 31

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is propoxycarbonyl

### Table 32

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is isopropoxycarbonyl

### Table 33

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is phenoxycarbonyl

### Table 34

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is methylaminocarbonyl

### Table 35

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is ethylaminocarbonyl

### Table 36

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is propylaminocarbonyl

### Table 37

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is isopropylaminocarbonyl

### Table 38

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is phenylaminocarbonyl

### Table 39

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is methylsulfonyl

### Table 40

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is ethylsulfonyl

### Table 41

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is propylsulfonyl

### Table 42

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is isopropylsulfonyl

### Table 43

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is phenylsulfonyl

### Table 44

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is methoxysulfonyl

### Table 45

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is ethoxysulfonyl

### Table 46

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is propoxysulfonyl

### Table 47

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is isopropoxysulfonyl

### Table 48

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is phenoxysulfonyl

### Table 49

Compounds of the formula I.1 in which the combination of R⁵¹, R⁵², R⁵³, R⁵⁴ and R⁵⁵ for a compound corresponds in each case to one row of Table A, R¹, R², R³ and R⁴ are H and R⁶ is CN

### Tables 50 to 98

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R¹ is F and R², R³ and R⁴ are H

### Tables 99 to 147

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R¹ is Cl and R², R³ and R⁴ are H

### Tables 148 to 196

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R¹ is Br and R², R³ and R⁴ are H

### Tables 197 to 245

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R¹ is CH₃ and R², R³ and R⁴ are H

### Tables 246 to 294

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R¹ is CF₃ and R², R³ and R⁴ are H

### Tables 295 to 343

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R¹ is CHF₂ and R², R³ and R⁴ are H

### Tables 344 to 392

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R¹ is OCH₃ and R², R³ and R⁴ are H

### Tables 393 to 441

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R¹ is OCF₃ and R², R³ and R⁴ are H

### Tables 442 to 490

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R² is F and R¹, R³ and R⁴ are H

### Tables 491 to 539

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R² is Cl and R¹, R³ and R⁴ are H

### Tables 540 to 588

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R² is Br and R¹, R³ and R⁴ are H

### Tables 589 to 637

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R² is CH₃ and R¹, R³ and R⁴ are H

### Tables 638 to 686

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R² is CF₃ and R¹, R³ and R⁴ are H

### Tables 687 to 735

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R² is OCH₃ and R¹, R³ and R⁴ are H

### Tables 736 to 784

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R² is OCF₃ and R¹, R³ and R⁴ are H

### Tables 785 to 833

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R¹ and R² are F and R³ and R⁴ are H

### Tables 834 to 882

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R¹ and R³ are F and R² and R⁴ are H

### Tables 883 to 931

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R¹ and R⁴ are F and R³ and R⁴ are H

### Tables 932 to 980

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R² and R⁴ are F and R³ and R⁴ are H

### Tables 981 to 1029

Compounds of the formula I.1 in which the combination of R⁵¹, R⁵², R⁵³, R⁵⁴ and R⁵⁵ for a compound corresponds in each case to one row of Table A, R⁶ is as defined in any of tables 1 to 49 and R¹ is Cl, and R⁴ is F and R² and R³ are H

### Tables 1030 to 2058

Compounds of the formula I.2 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 2059 to 3087

Compounds of the formula I.3 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 3088 to 4116

Compounds of the formula I.4 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 4117 to 5145

Compounds of the formula I.5 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 5146 to 6174

Compounds of the formula I.6 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 6175 to 7203

Compounds of the formula I.7 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 7204 to 8232

Compounds of the formula I.8 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 8233 to 9261

Compounds of the formula I.9 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 9262 to 10290

Compounds of the formula I.10 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 10291 to 11319

Compounds of the formula I.11 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 11320 to 12348

Compounds of the formula I.12 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 12349 to 13377

Compounds of the formula I.13 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 13378 to 14406

Compounds of the formula I.14 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 14407 to 15435

Compounds of the formula I.15 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 15436 to 16464

Compounds of the formula I.16 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 16465 to 17493

Compounds of the formula I.17 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 17494 to 18522

Compounds of the formula I.18 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 18523 to 19551

Compounds of the formula I.19 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 19552 to 20580

Compounds of the formula I.20 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 20581 to 21609

Compounds of the formula I.21 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 21610 to 22638

Compounds of the formula I.22 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 22639 to 23667

Compounds of the formula I.23 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 23668 to 24696

Compounds of the formula I.24 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 24697 to 25725

Compounds of the formula I.25 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 25726 to 26754

Compounds of the formula I.26 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 26755 to 27783

Compounds of the formula I.27 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 27784 to 28812

Compounds of the formula I.28 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 28813 to 29841

Compounds of the formula I.29 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 29842 to 30870

Compounds of the formula I.30 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 30871 to 31899

Compounds of the formula I.31 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 31900 to 32928

Compounds of the formula I.32 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 32929 to 33957

Compounds of the formula I.33 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 33958 to 34986

Compounds of the formula I.34 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 34987 to 36015

Compounds of the formula I.35 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 36016 to 37044

Compounds of the formula I.36 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 37045 to 38073

Compounds of the formula I.37 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 38074 to 39102

Compounds of the formula I.38 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 39103 to 40131

Compounds of the formula I.39 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 40132 to 41160

Compounds of the formula I.40 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 41161 to 42189

Compounds of the formula I.41 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 42190 to 43218

Compounds of the formula I.42 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 43219 to 44247

Compounds of the formula I.43 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 44248 to 45276

Compounds of the formula I.44 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 45277 to 46305

Compounds of the formula I.45 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 46306 to 47334

Compounds of the formula I.46 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 47335 to 48363

Compounds of the formula I.47 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 48364 to 49392

Compounds of the formula I.48 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Table 49393

Compounds of the formula I.49 in which the combination of R⁵¹, R⁵², R⁵³, R⁵⁴ and R⁵⁵ for a compound corresponds in each case to one row of Table A and R¹, R², R³ and R⁴ are H

### Table 49394

Compounds of the formula I.49 in which the combination of R⁵¹, R⁵², R⁵³, R⁵⁴ and R⁵⁵ for a compound corresponds in each case to one row of Table A, R¹ is F and R², R³ and R⁴ are H

### Table 49395

Compounds of the formula I.49 in which the combination of R⁵¹, R⁵², R⁵³, R⁵⁴ and R⁵⁵ for a compound corresponds in each case to one row of Table A, R¹ is Cl and R², R³ and R⁴ are H

### Table 49396

Compounds of the formula I.49 in which the combination of R⁵¹, R⁵², R⁵³, R⁵⁴ and R⁵⁵ for a compound corresponds in each case to one row of Table A, R¹ is Br and R², R³ and R⁴ are H

### Table 49397

Compounds of the formula I.49 in which the combination of R⁵¹, R⁵², R⁵³, R⁵⁴ and R⁵⁵ for a compound corresponds in each case to one row of Table A, R¹ is CH₃ and R², R³ and R⁴ are H

### Table 49398

Compounds of the formula I.49 in which the combination of R⁵¹, R⁵², R⁵³, R⁵⁴ and R⁵⁵ for a compound corresponds in each case to one row of Table A, R¹ is CF₃ and R², R³ and R⁴ are H

### Table 49399

Compounds of the formula I.49 in which the combination of R⁵¹, R⁵², R⁵³, R⁵⁴ and R⁵⁵ for a compound corresponds in each case to one row of Table A, R¹ is OCH₃ and R², R³ and R⁴ are H

### Table 49400

Compounds of the formula I.49 in which the combination of R⁵¹, R⁵², R⁵³, R⁵⁴ and R⁵⁵ for a compound corresponds in each case to one row of Table A, R¹ is OCF₃ and R², R³ and R⁴ are H

### Table 49401

Compounds of the formula I.49 in which the combination of R⁵¹, R⁵², R⁵³, R⁵⁴ and R⁵⁵ for a compound corresponds in each case to one row of Table A, R² is F and R¹, R³ and R⁴ are H

### Table 49402

Compounds of the formula I.49 in which the combination of R⁵¹, R⁵², R⁵³, R⁵⁴ and R⁵⁵ for a compound corresponds in each case to one row of Table A, R² is Cl and R¹, R³ and R⁴ are H

### Table 49403

Compounds of the formula I.49 in which the combination of R⁵¹, R⁵², R⁵³, R⁵⁴ and R⁵⁵ for a compound corresponds in each case to one row of Table A, R² is Br and R¹, R³ and R⁴ are H

### Table 49404

Compounds of the formula I.49 in which the combination of R⁵¹, R⁵², R⁵³, R⁵⁴ and R⁵⁵ for a compound corresponds in each case to one row of Table A, R² is CH₃ and R¹, R³ and R⁴ are H

### Table 49405

Compounds of the formula I.49 in which the combination of R⁵¹, R⁵², R⁵³, R⁵⁴ and R⁵⁵ for a compound corresponds in each case to one row of Table A, R² is CF₃ and R¹, R³ and R⁴ are H

### Table 49406

Compounds of the formula I.49 in which the combination of R⁵¹, R⁵², R⁵³, R⁵⁴ and R⁵⁵ for a compound corresponds in each case to one row of Table A, R² is OCH₃ and R¹, R³ and R⁴ are H

### Table 49407

Compounds of the formula I.49 in which the combination of R⁵¹, R⁵², R⁵³, R⁵⁴ and R⁵⁵ for a compound corresponds in each case to one row of Table A, R² is OCF₃ and R¹, R³ and R⁴ are H

### Table 49408

Compounds of the formula I.49 in which the combination of R⁵¹, R⁵², R⁵³, R⁵⁴ and R⁵⁵ for a compound corresponds in each case to one row of Table A, R¹ and R² are F and R³ and R⁴ are H

### Table 49409

Compounds of the formula I.49 in which the combination of R⁵¹, R⁵², R⁵³, R⁵⁴ and R⁵⁵ for a compound corresponds in each case to one row of Table A, R¹ and R³ are F and R² and R⁴ are H

### Table 49410

Compounds of the formula I.49 in which the combination of R⁵¹, R⁵², R⁵³, R⁵⁴ and R⁵⁵ for a compound corresponds in each case to one row of Table A, R¹ and R⁴ are F and R² and R³ are H

### Table 49411

Compounds of the formula I.49 in which the combination of R⁵¹, R⁵², R⁵³, R⁵⁴ and R⁵⁵ for a compound corresponds in each case to one row of Table A, R² and R⁴ are F and R¹ and R³ are H

### Table 49412

Compounds of the formula I.49 in which the combination of R⁵¹, R⁵², R⁵³, R⁵⁴ and R⁵⁵ for a compound corresponds in each case to one row of Table A, R¹ is Cl, R⁴ is F and R² and R³ are H

### Tables 49413 to 49432

Compounds of the formula I.50 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49433 to 49452

Compounds of the formula I.51 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49453 to 49472

Compounds of the formula I.52 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49473 to 49492

Compounds of the formula I.53 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49493 to 49512

Compounds of the formula I.54 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49513 to 49532

Compounds of the formula I.55 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49533 to 49552

Compounds of the formula I.56 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49553 to 49572

Compounds of the formula I.57 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49573 to 49592

Compounds of the formula I.58 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49593 to 49612

Compounds of the formula I.59 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49613 to 49632

Compounds of the formula I.60 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49633 to 49652

Compounds of the formula I.61 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49653 to 49672

Compounds of the formula I.62 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49673 to 49692

Compounds of the formula I.63 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49693 to 49712

Compounds of the formula I.64 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49713 to 49732

Compounds of the formula I.65 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49733 to 49752

Compounds of the formula I.66 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49753 to 49772

Compounds of the formula I.67 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49773 to 49792

Compounds of the formula I.68 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49793 to 49812

Compounds of the formula I.69 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49813 to 49832

Compounds of the formula I.70 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49833 to 49852

Compounds of the formula I.71 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49853 to 49872

Compounds of the formula I.72 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49873 to 49892

Compounds of the formula I.73 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49893 to 49912

Compounds of the formula I.74 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49913 to 49932

Compounds of the formula I.75 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49933 to 49952

Compounds of the formula I.76 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49953 to 49972

Compounds of the formula I.77 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49973 to 49992

Compounds of the formula I.78 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 49993 to 50012

Compounds of the formula I.79 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50013 to 50032

Compounds of the formula I.80 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50033 to 50052

Compounds of the formula I.81 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50053 to 50072

Compounds of the formula I.82 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50073 to 50092

Compounds of the formula I.83 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50093 to 50112

Compounds of the formula I.84 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50113 to 50132

Compounds of the formula I.85 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50133 to 50152

Compounds of the formula I.86 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50153 to 50172

Compounds of the formula I.87 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50173 to 50192

Compounds of the formula I.88 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50193 to 50212

Compounds of the formula I.89 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50213 to 50232

Compounds of the formula I.90 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50233 to 50252

Compounds of the formula I.91 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50253 to 50272

Compounds of the formula I.92 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50273 to 50292

Compounds of the formula I.93 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50293 to 50312

Compounds of the formula I.94 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50313 to 50332

Compounds of the formula I.95 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 50333 to 50352

Compounds of the formula I.96 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Table 50353

Compounds of the formula II.1 in which the combination R⁵¹, R⁵², R⁵³, R⁵⁴ and R⁵⁵ for a compound corresponds in each case to one row of Table A, R¹, R², R³ and R⁴ are H and R^{6a} is H

### Table 50354

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is methyl

### Table 50355

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is ethyl

### Table 50356

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is n-propyl

### Table 50357

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is isopropyl

### Table 50358

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is n-butyl

### Table 50359

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is sec-butyl

### Table 50360

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is isobutyl

### Table 50361

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is tert-butyl

### Table 50362

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is phenyl

### Table 50363

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is benzyl

### Table 50364

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is methylcarbonyl

### Table 50365

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is ethylcarbonyl

### Table 50366

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is propylcarbonyl

### Table 50367

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is isopropylcarbonyl

### Table 50368

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is phenylcarbonyl

### Table 50369

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is methoxycarbonyl

### Table 50370

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is ethoxycarbonyl

### Table 50371

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is propoxycarbonyl

### Table 50372

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is isopropoxycarbonyl

### Table 50373

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is phenoxycarbonyl

### Table 50374

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is methylaminocarbonyl

### Table 50375

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is ethylaminocarbonyl

### Table 50376

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is propylaminocarbonyl

### Table 50377

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is isopropylaminocarbonyl

### Table 50378

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is phenylaminocarbonyl

### Table 50379

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is methylsulfonyl

### Table 50380

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is ethylsulfonyl

### Table 50381

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is propylsulfonyl

### Table 50382

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is isopropylsulfonyl

### Table 50383

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is phenylsulfonyl

### Table 50384

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is methoxysulfonyl

### Table 50385

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is ethoxysulfonyl

### Table 50386

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is propoxysulfonyl

### Table 50387

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is isopropoxysulfonyl

### Table 50388

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is phenoxysulfonyl

### Table 50389

Compounds of the formula II.1 in which the combination of R⁵¹, R⁵², R⁵³, R⁵⁴ and R⁵⁵ for a compound corresponds in each case to one row of Table A, R¹, R², R³ and R⁴ are H and R^{6a} is CN

### Tables 50390 to 50426

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R¹ is F and R², R³ and R⁴ are H

### Tables 50427 to 50463

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R¹ is Cl and R², R³ and R⁴ are H

### Tables 50464 to 50500

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R¹ is Br and R², R³ and R⁴ are H

### Tables 50501 to 50537

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R¹ is CH₃ and R², R³ and R⁴ are H

### Tables 50538 to 50574

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R¹ is CF₃ and R², R³ and R⁴ are H

### Tables 50575 to 50611

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R¹ is CHF₂ and R², R³ and R⁴ are H

### Tables 50612 to 50648

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R¹ is OCH₃ and R², R³ and R⁴ are H

### Tables 50649 to 50685

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R¹ is OCF₃ and R², R³ and R⁴ are H

### Tables 50686 to 50722

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R² is F and R¹, R³ and R⁴ are H

### Tables 50723 to 50759

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R² is Cl and R¹, R³ and R⁴ are H

### Tables 50760 to 50796

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R² is Br and R¹, R³ and R⁴ are H

### Tables 50797 to 50833

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R² is CH₃ and R¹, R³ and R⁴ are H

### Tables 50834 to 50870

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R² is CF₃ and R¹, R³ and R⁴ are H

### Tables 50871 to 50907

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R² is OCH₃ and R¹, R³ and R⁴ are H

### Tables 50908 to 50944

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R² is OCF₃ and R¹, R³ and R⁴ are H

### Tables 50945 to 50981

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R¹ and R² are F and R³ and R⁴ are H

### Tables 50982 to 51018

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R¹ and R³ are F and R² and R⁴ are H

### Tables 51019 to 51055

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R¹ and R⁴ are F and R³ and R⁴ are H

### Tables 51056 to 51092

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R² and R⁴ are F and R¹ and R³ are H

### Tables 51093 to 51129

Compounds of the formula II.1 in which the combination of R⁵¹, R⁵², R⁵³, R⁵⁴ and R⁵⁵ for a compound corresponds in each case to one row of Table A, R^{6a} is as defined in any of tables 50353 to 50389 and R¹ is Cl, and R⁴ is F and R² and R³ are H

### Tables 51130 to 51905

Compounds of the formula II.2 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 51906 to 52683

Compounds of the formula II.3 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 52684 to 53460

Compounds of the formula II.4 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 53461 to 54237

Compounds of the formula II.5 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 54238 to 55014

Compounds of the formula II.6 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 55015 to 55791

Compounds of the formula II.7 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 55792 to 56568

Compounds of the formula II.8 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 56569 to 57345

Compounds of the formula II.9 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 57346 to 58122

Compounds of the formula II.10 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 58123 to 58899

Compounds of the formula II.11 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 58900 to 59676

Compounds of the formula II.12 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 59677 to 60453

Compounds of the formula II.13 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 60454 to 61230

Compounds of the formula II.14 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 61231 to 62007

Compounds of the formula II.15 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 62008 to 62784

Compounds of the formula II.16 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 62785 to 63561

Compounds of the formula II.17 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 63562 to 64338

Compounds of the formula II.18 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 64339 to 65115

Compounds of the formula II.19 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 65116 to 65892

Compounds of the formula II.20 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 65893 to 66669

Compounds of the formula II.21 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 66670 to 67446

Compounds of the formula II.22 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 67447 to 68223

Compounds of the formula II.23 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 68224 to 69000

Compounds of the formula II.24 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 69001 to 69777

Compounds of the formula II.25 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 69778 to 70554

Compounds of the formula II.26 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 70555 to 71331

Compounds of the formula II.27 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 71332 to 72108

Compounds of the formula II.28 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 72109 to 72885

Compounds of the formula II.29 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 72886 to 73662

Compounds of the formula II.30 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 73663 to 74439

Compounds of the formula II.31 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 74440 to 75216

Compounds of the formula II.32 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 75217 to 75993

Compounds of the formula II.33 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 75994 to 76770

Compounds of the formula II.34 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 76771 to 77547

Compounds of the formula II.35 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 77548 to 78324

Compounds of the formula II.36 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 78325 to 79101

Compounds of the formula II.37 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 79102 to 79878

Compounds of the formula II.38 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 79879 to 80655

Compounds of the formula II.39 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 80656 to 81432

Compounds of the formula II.40 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 81433 to 82209

Compounds of the formula II.41 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 82209 to 82986

Compounds of the formula II.42 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 82987 to 83763

Compounds of the formula II.43 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 83764 to 84540

Compounds of the formula II.44 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 84541 to 85317

Compounds of the formula II.45 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 85318 to 86094

Compounds of the formula II.46 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 86095 to 86871

Compounds of the formula II.47 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

### Tables 86872 to 87648

Compounds of the formula II.48 in which the combination of R⁵¹, R⁵², R⁵³, R⁵⁴ and R⁵⁵ for a compound corresponds in each case to one row of Table A and the combination of R¹, R², R³, R⁴ and R^{6a} is as defined in any of tables 50353 to 51129

TABLE A

| No. | R⁵¹ | R⁵² | R⁵³ | R⁵⁴ | R⁵⁵ |
|---|---|---|---|---|---|
| A-1 | H | H | H | H | H |
| A-2 | F | H | H | H | H |
| A-3 | H | F | H | H | H |
| A-4 | H | H | F | H | H |
| A-5 | Cl | H | H | H | H |
| A-6 | H | Cl | H | H | H |
| A-7 | H | H | Cl | H | H |
| A-8 | Br | H | H | H | H |
| A-9 | H | Br | H | H | H |
| A-10 | H | H | Br | H | H |
| A-11 | CH₃ | H | H | H | H |
| A-12 | H | CH₃ | H | H | H |
| A-13 | H | H | CH₃ | H | H |
| A-14 | CHF₂ | H | H | H | H |
| A-15 | H | CHF₂ | H | H | H |
| A-16 | H | H | CHF₂ | H | H |
| A-17 | CF₃ | H | H | H | H |
| A-18 | H | CF₃ | H | H | H |
| A-19 | H | H | CF₃ | H | H |
| A-20 | OCH₃ | H | H | H | H |
| A-21 | H | OCH₃ | H | H | H |
| A-22 | H | H | OCH₃ | H | H |
| A-23 | OCHF₂ | H | H | H | H |
| A-24 | H | OCHF₂ | H | H | H |
| A-25 | H | H | OCHF₂ | H | H |
| A-26 | OCF₃ | H | H | H | H |
| A-27 | H | OCF₃ | H | H | H |
| A-28 | H | H | OCF₃ | H | H |
| A-29 | F | F | H | H | H |
| A-30 | F | H | F | H | H |
| A-31 | F | H | H | F | H |
| A-32 | F | H | H | H | F |
| A-33 | H | F | F | H | H |
| A-34 | H | F | H | F | H |
| A-35 | Cl | Cl | H | H | H |
| A-36 | Cl | H | Cl | H | H |
| A-37 | Cl | H | H | Cl | H |
| A-38 | Cl | H | H | H | Cl |
| A-39 | H | Cl | Cl | H | H |
| A-40 | H | Cl | H | Cl | H |
| A-41 | Br | Br | H | H | H |
| A-42 | Br | H | Br | H | H |
| A-43 | Br | H | H | Br | H |
| A-44 | Br | H | H | H | Br |
| A-45 | H | Br | Br | H | H |
| A-46 | H | Br | H | Br | H |
| A-47 | CH₃ | CH₃ | H | H | H |
| A-48 | CH₃ | H | CH₃ | H | H |
| A-49 | CH₃ | H | H | CH₃ | H |
| A-50 | CH₃ | H | H | H | CH₃ |
| A-51 | H | CH₃ | CH₃ | H | H |
| A-52 | H | CH₃ | H | CH₃ | H |
| A-53 | CHF₂ | CHF₂ | H | H | H |
| A-54 | CHF₂ | H | CHF₂ | H | H |
| A-55 | CHF₂ | H | H | CHF₂ | H |
| A-56 | CHF₂ | H | H | H | CHF₂ |
| A-57 | H | CHF₂ | CHF₂ | H | H |
| A-58 | H | CHF₂ | H | CHF₂ | H |
| A-59 | CF₃ | CF₃ | H | H | H |
| A-60 | CF₃ | H | CF₃ | H | H |
| A-61 | CF₃ | H | H | CF₃ | H |
| A-62 | CF₃ | H | H | H | CF₃ |
| A-63 | H | CF₃ | CF₃ | H | H |
| A-64 | H | CF₃ | H | CF₃ | H |
| A-65 | OCH₃ | OCH₃ | H | H | H |
| A-66 | OCH₃ | H | OCH₃ | H | H |
| A-67 | OCH₃ | H | H | OCH₃ | H |
| A-68 | OCH₃ | H | H | H | OCH₃ |
| A-69 | H | OCH₃ | OCH₃ | H | H |
| A-70 | H | OCH₃ | H | OCH₃ | H |
| A-71 | OCHF₂ | OCHF₂ | H | H | H |
| A-72 | OCHF₂ | H | OCHF₂ | H | H |
| A-73 | OCHF₂ | H | H | OCHF₂ | H |
| A-74 | OCHF₂ | H | H | H | OCHF₂ |
| A-75 | H | OCHF₂ | OCHF₂ | H | H |
| A-76 | H | OCHF₂ | H | OCHF₂ | H |
| A-77 | OCF₃ | OCF₃ | H | H | H |
| A-78 | OCF₃ | H | OCF₃ | H | H |
| A-79 | OCF₃ | H | H | OCF₃ | H |
| A-80 | OCF₃ | H | H | H | OCF₃ |
| A-81 | H | OCF₃ | OCF₃ | H | H |
| A-82 | H | OCF₃ | H | OCF₃ | H |
| A-83 | F | Cl | H | H | H |
| A-84 | F | H | Cl | H | H |
| A-85 | F | H | H | Cl | H |
| A-86 | F | H | H | H | Cl |
| A-87 | H | F | Cl | H | H |
| A-88 | H | F | H | Cl | H |
| A-89 | Cl | F | H | H | H |
| A-90 | Cl | H | F | H | H |
| A-91 | Cl | H | H | F | H |
| A-92 | H | Cl | F | H | H |
| A-93 | F | Br | H | H | H |
| A-94 | F | H | Br | H | H |
| A-95 | F | H | H | Br | H |
| A-96 | F | H | H | H | Br |
| A-97 | H | F | Br | H | H |
| A-98 | H | F | H | Br | H |
| A-99 | Br | F | H | H | H |
| A-100 | Br | H | F | H | H |
| A-101 | Br | H | H | F | H |
| A-102 | H | Br | F | H | H |
| A-103 | F | CH₃ | H | H | H |
| A-104 | F | H | CH₃ | H | H |
| A-105 | F | H | H | CH₃ | H |
| A-106 | F | H | H | H | CH₃ |
| A-107 | H | F | CH₃ | H | H |
| A-108 | H | F | H | CH₃ | H |
| A-109 | CH₃ | F | H | H | H |
| A-110 | CH₃ | H | F | H | H |
| A-111 | CH₃ | H | H | F | H |
| A-112 | H | CH₃ | F | H | H |
| A-113 | F | CHF₂ | H | H | H |
| A-114 | F | H | CHF₂ | H | H |
| A-115 | F | H | H | CHF₂ | H |
| A-116 | F | H | H | H | CHF₂ |
| A-117 | H | F | CHF₂ | H | H |
| A-118 | H | F | H | CHF₂ | H |
| A-119 | CHF₂ | F | H | H | H |
| A-120 | CHF₂ | H | F | H | H |
| A-121 | CHF₂ | H | H | F | H |
| A-122 | H | CHF₂ | F | H | H |
| A-123 | F | CF₃ | H | H | H |
| A-124 | F | H | CF₃ | H | H |
| A-125 | F | H | H | CF₃ | H |
| A-126 | F | H | H | H | CF₃ |
| A-127 | H | F | CF₃ | H | H |
| A-128 | H | F | H | CF₃ | H |
| A-129 | CF₃ | F | H | H | H |
| A-130 | CF₃ | H | F | H | H |
| A-131 | CF₃ | H | H | F | H |
| A-132 | H | CF₃ | F | H | H |
| A-133 | F | OCH₃ | H | H | H |
| A-134 | F | H | OCH₃ | H | H |
| A-135 | F | H | H | OCH₃ | H |
| A-136 | F | H | H | H | OCH₃ |
| A-137 | H | F | OCH₃ | H | H |
| A-138 | H | F | H | OCH₃ | H |
| A-139 | OCH₃ | F | H | H | H |
| A-140 | OCH₃ | H | F | H | H |
| A-141 | OCH₃ | H | H | F | H |
| A-142 | H | OCH₃ | F | H | H |
| A-143 | F | OCHF₂ | H | H | H |
| A-144 | F | H | OCHF₂ | H | H |
| A-145 | F | H | H | OCHF₂ | H |
| A-146 | F | H | H | H | OCHF₂ |
| A-147 | H | F | OCHF₂ | H | H |
| A-148 | H | F | H | OCHF₂ | H |
| A-149 | OCHF₂ | F | H | H | H |
| A-150 | OCHF₂ | H | F | H | H |
| A-151 | OCHF₂ | H | H | F | H |
| A-152 | H | OCHF₂ | F | H | H |
| A-153 | F | OCF₃ | H | H | H |
| A-154 | F | H | OCF₃ | H | H |
| A-155 | F | H | H | OCF₃ | H |
| A-156 | F | H | H | H | OCF₃ |
| A-157 | H | F | OCF₃ | H | H |
| A-158 | H | F | H | OCF₃ | H |
| A-159 | OCF₃ | F | H | H | H |
| A-160 | OCF₃ | H | F | H | H |
| A-161 | OCF₃ | H | H | F | H |
| A-162 | H | OCF₃ | F | H | H |
| A-163 | Cl | Br | H | H | H |
| A-164 | Cl | H | Br | H | H |
| A-165 | Cl | H | H | Br | H |
| A-166 | Cl | H | H | H | Br |
| A-167 | H | Cl | Br | H | H |
| A-168 | H | Cl | H | Br | H |
| A-169 | Br | Cl | H | H | H |
| A-170 | Br | H | Cl | H | H |
| A-171 | Br | H | H | Cl | H |
| A-172 | H | Br | Cl | H | H |
| A-173 | Cl | CH₃ | H | H | H |
| A-174 | Cl | H | CH₃ | H | H |
| A-175 | Cl | H | H | CH₃ | H |
| A-176 | Cl | H | H | H | CH₃ |
| A-177 | H | Cl | CH₃ | H | H |
| A-178 | H | Cl | H | CH₃ | H |
| A-179 | CH₃ | Cl | H | H | H |
| A-180 | CH₃ | H | Cl | H | H |
| A-181 | CH₃ | H | H | Cl | H |
| A-182 | H | CH₃ | Cl | H | H |
| A-183 | Cl | CHF₂ | H | H | H |
| A-184 | Cl | H | CHF₂ | H | H |
| A-185 | Cl | H | H | CHF₂ | H |
| A-186 | Cl | H | H | H | CHF₂ |
| A-187 | H | Cl | CHF₂ | H | H |
| A-188 | H | Cl | H | CHF₂ | H |
| A-189 | CHF₂ | Cl | H | H | H |
| A-190 | CHF₂ | H | Cl | H | H |
| A-191 | CHF₂ | H | H | Cl | H |
| A-192 | H | CHF₂ | Cl | H | H |
| A-193 | Cl | CF₃ | H | H | H |
| A-194 | Cl | H | CF₃ | H | H |
| A-195 | Cl | H | H | CF₃ | H |
| A-196 | Cl | H | H | H | CF₃ |
| A-197 | H | Cl | CF₃ | H | H |
| A-198 | H | Cl | H | CF₃ | H |
| A-199 | CF₃ | Cl | H | H | H |
| A-200 | CF₃ | H | Cl | H | H |
| A-201 | CF₃ | H | H | Cl | H |
| A-202 | H | CF₃ | Cl | H | H |
| A-203 | Cl | OCH₃ | H | H | H |
| A-204 | Cl | H | OCH₃ | H | H |
| A-205 | Cl | H | H | OCH₃ | H |
| A-206 | Cl | H | H | H | OCH₃ |
| A-207 | H | Cl | OCH₃ | H | H |
| A-208 | H | Cl | H | OCH₃ | H |
| A-209 | OCH₃ | Cl | H | H | H |
| A-210 | OCH₃ | H | Cl | H | H |
| A-211 | OCH₃ | H | H | Cl | H |
| A-212 | H | OCH₃ | Cl | H | H |
| A-213 | Cl | OCHF₂ | H | H | H |
| A-214 | Cl | H | OCHF₂ | H | H |
| A-215 | Cl | H | H | OCHF₂ | H |
| A-216 | Cl | H | H | H | OCHF₂ |
| A-217 | H | Cl | OCHF₂ | H | H |
| A-218 | H | Cl | H | OCHF₂ | H |
| A-219 | OCHF₂ | Cl | H | H | H |
| A-220 | OCHF₂ | H | Cl | H | H |
| A-221 | OCHF₂ | H | H | Cl | H |
| A-222 | H | OCHF₂ | Cl | H | H |
| A-223 | Cl | OCF₃ | H | H | H |
| A-224 | Cl | H | OCF₃ | H | H |
| A-225 | Cl | H | H | OCF₃ | H |
| A-226 | Cl | H | H | H | OCF₃ |
| A-227 | H | Cl | OCF₃ | H | H |
| A-228 | H | Cl | H | OCF₃ | H |
| A-229 | OCF₃ | Cl | H | H | H |
| A-230 | OCF₃ | H | Cl | H | H |
| A-231 | OCF₃ | H | H | Cl | H |
| A-232 | H | OCF₃ | Cl | H | H |
| A-233 | Br | CH₃ | H | H | H |
| A-234 | Br | H | CH₃ | H | H |
| A-235 | Br | H | H | CH₃ | H |
| A-236 | Br | H | H | H | CH₃ |
| A-237 | H | Br | CH₃ | H | H |
| A-238 | H | Br | H | CH₃ | H |
| A-239 | CH₃ | Br | H | H | H |
| A-240 | CH₃ | H | Br | H | H |
| A-241 | CH₃ | H | H | Br | H |
| A-242 | H | CH₃ | Br | H | H |
| A-243 | Br | CHF₂ | H | H | H |
| A-244 | Br | H | CHF₂ | H | H |
| A-245 | Br | H | H | CHF₂ | H |
| A-246 | Br | H | H | H | CHF₂ |
| A-247 | H | Br | CHF₂ | H | H |
| A-248 | H | Br | H | CHF₂ | H |
| A-249 | CHF₂ | Br | H | H | H |
| A-250 | CHF₂ | H | Br | H | H |
| A-251 | CHF₂ | H | H | Br | H |
| A-252 | H | CHF₂ | Br | H | H |
| A-253 | Br | CF₃ | H | H | H |
| A-254 | Br | H | CF₃ | H | H |
| A-255 | Br | H | H | CF₃ | H |
| A-256 | Br | H | H | H | CF₃ |
| A-257 | H | Br | CF₃ | H | H |
| A-258 | H | Br | H | CF₃ | H |
| A-259 | CF₃ | Br | H | H | H |
| A-260 | CF₃ | H | Br | H | H |
| A-261 | CF₃ | H | H | Br | H |
| A-262 | H | CF₃ | Br | H | H |
| A-263 | Br | OCH₃ | H | H | H |
| A-264 | Br | H | OCH₃ | H | H |
| A-265 | Br | H | H | OCH₃ | H |
| A-266 | Br | H | H | H | OCH₃ |
| A-267 | H | Br | OCH₃ | H | H |
| A-268 | H | Br | H | OCH₃ | H |
| A-269 | OCH₃ | Br | H | H | H |
| A-270 | OCH₃ | H | Br | H | H |
| A-271 | OCH₃ | H | H | Br | H |
| A-272 | H | OCH₃ | Br | H | H |
| A-273 | Br | OCHF₂ | H | H | H |
| A-274 | Br | H | OCHF₂ | H | H |
| A-275 | Br | H | H | OCHF₂ | H |
| A-276 | Br | H | H | H | OCHF₂ |
| A-277 | H | Br | OCHF₂ | H | H |
| A-278 | H | Br | H | OCHF₂ | H |
| A-279 | OCHF₂ | Br | H | H | H |
| A-280 | OCHF₂ | H | Br | H | H |
| A-281 | OCHF₂ | H | H | Br | H |
| A-282 | H | OCHF₂ | Br | H | H |
| A-283 | Br | OCF₃ | H | H | H |
| A-284 | Br | H | OCF₃ | H | H |
| A-285 | Br | H | H | OCF₃ | H |
| A-286 | Br | H | H | H | OCF₃ |
| A-287 | H | Br | OCF₃ | H | H |
| A-288 | H | Br | H | OCF₃ | H |
| A-289 | OCF₃ | Br | H | H | H |
| A-290 | OCF₃ | H | Br | H | H |
| A-291 | OCF₃ | H | H | Br | H |
| A-292 | H | OCF₃ | Br | H | H |
| A-293 | CH₃ | CHF₂ | H | H | H |
| A-294 | CH₃ | H | CHF₂ | H | H |
| A-295 | CH₃ | H | H | CHF₂ | H |
| A-296 | CH₃ | H | H | H | CHF₂ |
| A-297 | H | CH₃ | CHF₂ | H | H |
| A-298 | H | CH₃ | H | CHF₂ | H |
| A-299 | CHF₂ | CH₃ | H | H | H |
| A-300 | CHF₂ | H | CH₃ | H | H |
| A-301 | CHF₂ | H | H | CH₃ | H |
| A-302 | H | CHF₂ | CH₃ | H | H |
| A-303 | CH₃ | CF₃ | H | H | H |
| A-304 | CH₃ | H | CF₃ | H | H |
| A-305 | CH₃ | H | H | CF₃ | H |
| A-306 | CH₃ | H | H | H | CF₃ |
| A-307 | H | CH₃ | CF₃ | H | H |
| A-308 | H | CH₃ | H | CF₃ | H |
| A-309 | CF₃ | CH₃ | H | H | H |
| A-310 | CF₃ | H | CH₃ | H | H |
| A-311 | CF₃ | H | H | CH₃ | H |
| A-312 | H | CF₃ | CH₃ | H | H |
| A-313 | CH₃ | OCH₃ | H | H | H |
| A-314 | CH₃ | H | OCH₃ | H | H |
| A-315 | CH₃ | H | H | OCH₃ | H |
| A-316 | CH₃ | H | H | H | OCH₃ |
| A-317 | H | CH₃ | OCH₃ | H | H |
| A-318 | H | CH₃ | H | OCH₃ | H |
| A-319 | OCH₃ | CH₃ | H | H | H |
| A-320 | OCH₃ | H | CH₃ | H | H |
| A-321 | OCH₃ | H | H | CH₃ | H |
| A-322 | H | OCH₃ | CH₃ | H | H |
| A-323 | CH₃ | OCHF₂ | H | H | H |
| A-324 | CH₃ | H | OCHF₂ | H | H |
| A-325 | CH₃ | H | H | OCHF₂ | H |
| A-326 | CH₃ | H | H | H | OCHF₂ |
| A-327 | H | CH₃ | OCHF₂ | H | H |
| A-328 | H | CH₃ | H | OCHF₂ | H |
| A-329 | OCHF₂ | CH₃ | H | H | H |
| A-330 | OCHF₂ | H | CH₃ | H | H |
| A-331 | OCHF₂ | H | H | CH₃ | H |
| A-332 | H | OCHF₂ | CH₃ | H | H |
| A-333 | CH₃ | OCF₃ | H | H | H |
| A-334 | CH₃ | H | OCF₃ | H | H |
| A-335 | CH₃ | H | H | OCF₃ | H |
| A-336 | CH₃ | H | H | H | OCF₃ |
| A-337 | H | CH₃ | OCF₃ | H | H |
| A-338 | H | CH₃ | H | OCF₃ | H |
| A-339 | OCF₃ | CH₃ | H | H | H |
| A-340 | OCF₃ | H | CH₃ | H | H |
| A-341 | OCF₃ | H | H | CH₃ | H |
| A-342 | H | OCF₃ | CH₃ | H | H |
| A-343 | CHF₂ | CF₃ | H | H | H |
| A-344 | CHF₂ | H | CF₃ | H | H |
| A-345 | CHF₂ | H | H | CF₃ | H |
| A-346 | CHF₂ | H | H | H | CF₃ |
| A-347 | H | CHF₂ | CF₃ | H | H |
| A-348 | H | CHF₂ | H | CF₃ | H |
| A-349 | CF₃ | CHF₂ | H | H | H |
| A-350 | CF₃ | H | CHF₂ | H | H |
| A-351 | CF₃ | H | H | CHF₂ | H |
| A-352 | H | CF₃ | CHF₂ | H | H |
| A-353 | CHF₂ | OCH₃ | H | H | H |
| A-354 | CHF₂ | H | OCH₃ | H | H |
| A-355 | CHF₂ | H | H | OCH₃ | H |
| A-356 | CHF₂ | H | H | H | OCH₃ |
| A-357 | H | CHF₂ | OCH₃ | H | H |
| A-358 | H | CHF₂ | H | OCH₃ | H |
| A-359 | OCH₃ | CHF₂ | H | H | H |
| A-360 | OCH₃ | H | CHF₂ | H | H |
| A-361 | OCH₃ | H | H | CHF₂ | H |
| A-362 | H | OCH₃ | CHF₂ | H | H |
| A-363 | CHF₂ | OCHF₂ | H | H | H |
| A-364 | CHF₂ | H | OCHF₂ | H | H |
| A-365 | CHF₂ | H | H | OCHF₂ | H |
| A-366 | CHF₂ | H | H | H | OCHF₂ |
| A-367 | H | CHF₂ | OCHF₂ | H | H |
| A-368 | H | CHF₂ | H | OCHF₂ | H |
| A-369 | OCHF₂ | CHF₂ | H | H | H |
| A-370 | OCHF₂ | H | CHF₂ | H | H |
| A-371 | OCHF₂ | H | H | CHF₂ | H |
| A-372 | H | OCHF₂ | CHF₂ | H | H |
| A-373 | CHF₂ | OCF₃ | H | H | H |
| A-374 | CHF₂ | H | OCF₃ | H | H |
| A-375 | CHF₂ | H | H | OCF₃ | H |
| A-376 | CHF₂ | H | H | H | OCF₃ |
| A-377 | H | CHF₂ | OCF₃ | H | H |
| A-378 | H | CHF₂ | H | OCF₃ | H |
| A-379 | OCF₃ | CHF₂ | H | H | H |
| A-380 | OCF₃ | H | CHF₂ | H | H |
| A-381 | OCF₃ | H | H | CHF₂ | H |
| A-382 | H | OCF₃ | CHF₂ | H | H |
| A-383 | CF₃ | OCH₃ | H | H | H |
| A-384 | CF₃ | H | OCH₃ | H | H |
| A-385 | CF₃ | H | H | OCH₃ | H |
| A-386 | CF₃ | H | H | H | OCH₃ |
| A-387 | H | CF₃ | OCH₃ | H | H |
| A-388 | H | CF₃ | H | OCH₃ | H |
| A-389 | OCH₃ | CF₃ | H | H | H |
| A-390 | OCH₃ | H | CF₃ | H | H |
| A-391 | OCH₃ | H | H | CF₃ | H |
| A-392 | H | OCH₃ | CF₃ | H | H |
| A-393 | CF₃ | OCHF₂ | H | H | H |
| A-394 | CF₃ | H | OCHF₂ | H | H |
| A-395 | CF₃ | H | H | OCHF₂ | H |
| A-396 | CF₃ | H | H | H | OCHF₂ |
| A-397 | H | CF₃ | OCHF₂ | H | H |
| A-398 | H | CF₃ | H | OCHF₂ | H |
| A-399 | OCHF₂ | CF₃ | H | H | H |
| A-400 | OCHF₂ | H | CF₃ | H | H |
| A-401 | OCHF₂ | H | H | CF₃ | H |
| A-402 | H | OCHF₂ | CF₃ | H | H |
| A-403 | CF₃ | OCF₃ | H | H | H |
| A-404 | CF₃ | H | OCF₃ | H | H |
| A-405 | CF₃ | H | H | OCF₃ | H |
| A-406 | CF₃ | H | H | H | OCF₃ |
| A-407 | H | CF₃ | OCF₃ | H | H |
| A-408 | H | CF₃ | H | OCF₃ | H |
| A-409 | OCF₃ | CF₃ | H | H | H |
| A-410 | OCF₃ | H | CF₃ | H | H |
| A-411 | OCF₃ | H | H | CF₃ | H |
| A-412 | H | OCF₃ | CF₃ | H | H |
| A-413 | OCH₃ | OCHF₂ | H | H | H |
| A-414 | OCH₃ | H | OCHF₂ | H | H |
| A-415 | OCH₃ | H | H | OCHF₂ | H |
| A-416 | OCH₃ | H | H | H | OCHF₂ |
| A-417 | H | OCH₃ | OCHF₂ | H | H |
| A-418 | H | OCH₃ | H | OCHF₂ | H |
| A-419 | OCHF₂ | OCH₃ | H | H | H |
| A-420 | OCHF₂ | H | OCH₃ | H | H |
| A-421 | OCHF₂ | H | H | OCH₃ | H |
| A-422 | H | OCHF₂ | OCH₃ | H | H |
| A-423 | OCH₃ | OCF₃ | H | H | H |
| A-424 | OCH₃ | H | OCF₃ | H | H |
| A-425 | OCH₃ | H | H | OCF₃ | H |
| A-426 | OCH₃ | H | H | H | OCF₃ |
| A-427 | H | OCH₃ | OCF₃ | H | H |
| A-428 | H | OCH₃ | H | OCF₃ | H |
| A-429 | OCF₃ | OCH₃ | H | H | H |
| A-430 | OCF₃ | H | OCH₃ | H | H |
| A-431 | OCF₃ | H | H | OCH₃ | H |
| A-432 | H | OCF₃ | OCH₃ | H | H |
| A-433 | OCHF₂ | OCF₃ | H | H | H |
| A-434 | OCHF₂ | H | OCF₃ | H | H |
| A-435 | OCHF₂ | H | H | OCF₃ | H |
| A-436 | OCHF₂ | H | H | H | OCF₃ |
| A-437 | H | OCHF₂ | OCF₃ | H | H |
| A-438 | H | OCHF₂ | H | OCF₃ | H |
| A-439 | OCF₃ | OCHF₂ | H | H | H |
| A-440 | OCF₃ | H | OCHF₂ | H | H |
| A-441 | OCF₃ | H | H | OCHF₂ | H |
| A-442 | H | OCF₃ | OCHF₂ | H | H |
| A-443 | F | F | F | H | H |
| A-444 | F | F | H | F | H |
| A-445 | F | F | H | H | F |
| A-446 | F | H | F | F | H |
| A-447 | F | H | F | H | F |
| A-448 | H | F | F | F | H |
| A-449 | Cl | Cl | Cl | H | H |
| A-450 | Cl | Cl | H | Cl | H |
| A-451 | Cl | Cl | H | H | Cl |
| A-452 | Cl | H | Cl | Cl | H |
| A-453 | Cl | H | Cl | H | Cl |
| A-454 | H | Cl | Cl | Cl | H |
| A-455 | Br | Br | Br | H | H |
| A-456 | Br | Br | H | Br | H |
| A-457 | Br | Br | H | H | Br |
| A-458 | Br | H | Br | Br | H |
| A-459 | Br | H | Br | H | Br |
| A-460 | H | Br | Br | Br | H |
| A-461 | CH₃ | CH₃ | CH₃ | H | H |
| A-462 | CH₃ | CH₃ | H | CH₃ | H |
| A-463 | CH₃ | CH₃ | H | H | CH₃ |
| A-464 | CH₃ | H | CH₃ | CH₃ | H |
| A-465 | CH₃ | H | CH₃ | H | CH₃ |
| A-466 | H | CH₃ | CH₃ | CH₃ | H |
| A-467 | CF₃ | CF₃ | CF₃ | H | H |
| A-468 | CF₃ | CF₃ | H | CF₃ | H |
| A-469 | CF₃ | CF₃ | H | H | CF₃ |
| A-470 | CF₃ | H | CF₃ | CF₃ | H |
| A-471 | CF₃ | H | CF₃ | H | CF₃ |
| A-472 | H | CF₃ | CF₃ | CF₃ | H |
| A-473 | F | H | Cl | H | F |
| A-474 | F | H | F | H | Cl |
| A-475 | F | H | Cl | F | H |
| A-476 | F | H | Cl | H | Cl |
| A-477 | Cl | H | F | H | Cl |
| A-478 | Cl | H | Cl | F | H |
| A-479 | Cl | H | Cl | H | F |
| A-480 | Cl | H | F | F | H |
| A-481 | Cl | F | H | F | H |
| A-482 | F | H | H | F | Cl |
| A-483 | F | Cl | H | Cl | H |
| A-484 | F | Cl | H | H | Cl |
| A-485 | H | F | F | H | Cl |
| A-486 | Cl | F | H | H | F |
| A-487 | F | H | Cl | Cl | H |
| A-488 | F | H | CH₃ | H | F |
| A-489 | F | H | F | H | CH₃ |
| A-490 | F | H | CH₃ | F | H |
| A-491 | F | H | CH₃ | H | CH₃ |
| A-492 | CH₃ | H | F | H | CH₃ |
| A-493 | CH₃ | H | CH₃ | F | H |
| A-494 | CH₃ | H | CH₃ | H | F |
| A-495 | CH₃ | H | F | F | H |
| A-496 | CH₃ | F | H | F | H |
| A-497 | F | H | H | F | CH₃ |
| A-498 | F | CH₃ | H | CH₃ | H |
| A-499 | F | CH₃ | H | H | CH₃ |
| A-500 | H | F | F | H | CH₃ |
| A-501 | CH₃ | F | H | H | F |
| A-502 | F | H | CH₃ | CH₃ | H |
| A-503 | F | H | CF₃ | H | F |
| A-504 | F | H | F | H | CF₃ |
| A-505 | F | H | CF₃ | F | H |
| A-506 | F | H | CF₃ | H | CF₃ |
| A-507 | CF₃ | H | F | H | CF₃ |
| A-508 | CF₃ | H | CF₃ | F | H |
| A-509 | CF₃ | H | CF₃ | H | F |
| A-510 | CF₃ | H | F | F | H |
| A-511 | CF₃ | F | H | F | H |
| A-512 | F | H | H | F | CF₃ |
| A-513 | F | CF₃ | H | CF₃ | H |
| A-514 | F | CF₃ | H | H | CF₃ |
| A-515 | H | F | F | H | CF₃ |
| A-516 | CF₃ | F | H | H | F |
| A-517 | F | H | CF₃ | CF₃ | H |
| A-518 | Cl | H | CH₃ | H | Cl |
| A-519 | Cl | H | Cl | H | CH₃ |
| A-520 | Cl | H | CH₃ | Cl | H |
| A-521 | Cl | H | CH₃ | H | CH₃ |
| A-522 | CH₃ | H | Cl | H | CH₃ |
| A-523 | CH₃ | H | CH₃ | Cl | H |
| A-524 | CH₃ | H | CH₃ | H | Cl |
| A-525 | CH₃ | H | Cl | Cl | H |
| A-526 | CH₃ | Cl | H | Cl | H |
| A-527 | Cl | H | H | Cl | CH₃ |
| A-528 | Cl | CH₃ | H | CH₃ | H |
| A-529 | Cl | CH₃ | H | H | CH₃ |
| A-530 | H | Cl | Cl | H | CH₃ |
| A-531 | CH₃ | Cl | H | H | Cl |
| A-532 | Cl | H | CH₃ | CH₃ | H |
| A-533 | Cl | H | CF₃ | H | Cl |
| A-534 | Cl | H | Cl | H | CF₃ |
| A-535 | Cl | H | CF₃ | Cl | H |
| A-536 | Cl | H | CF₃ | H | CF₃ |
| A-537 | CF₃ | H | Cl | H | CF₃ |
| A-538 | CF₃ | H | CF₃ | Cl | H |
| A-539 | CF₃ | H | CF₃ | H | Cl |
| A-540 | CF₃ | H | Cl | Cl | H |
| A-541 | CF₃ | Cl | H | Cl | H |
| A-542 | Cl | H | H | Cl | CF₃ |
| A-543 | Cl | CF₃ | H | CF₃ | H |
| A-544 | Cl | CF₃ | H | H | CF₃ |
| A-545 | H | Cl | Cl | H | CF₃ |
| A-546 | CF₃ | Cl | H | H | Cl |
| A-547 | Cl | H | CF₃ | CF₃ | H |
| A-548 | CF₃ | H | Cl | H | F |
| A-549 | Cl | H | CF₃ | F | H |
| A-550 | CF₃ | H | Cl | F | H |
| A-551 | CF₃ | F | H | Cl | H |
| A-552 | Cl | H | H | F | CF₃ |
| A-553 | Cl | H | CF₃ | F | H |
| A-554 | Cl | H | CH₃ | F | H |
| A-555 | CH₃ | F | H | Cl | H |
| A-556 | CH₃ | H | Cl | F | H |
| A-557 | Cl | H | H | F | CH₃ |
| A-558 | Cl | H | CH₃ | F | H |

Among the above compounds, preference is given to compounds of formulae I.1 to I.24, I.49 to I.72 and II.1 to II.24. Among these, preference is given to compounds I.1, I.2, I.13, I.14, I.49, I.50, I.61, I.62, II.1, II.2, II.13 and II.14. More preference is given to compounds I.1, I.2, I.49, I.50, II.1 and II.2 and even more preference to compounds I.2, I.50 and II.2.

Further examples for preferred compounds I and II are compounds of formulae I.97 to I.172, where the variables have one of the general or, in particular, one of the preferred meanings given above. Examples of preferred compounds are the individual compounds compiled in the tables 87649 to 129528 below.

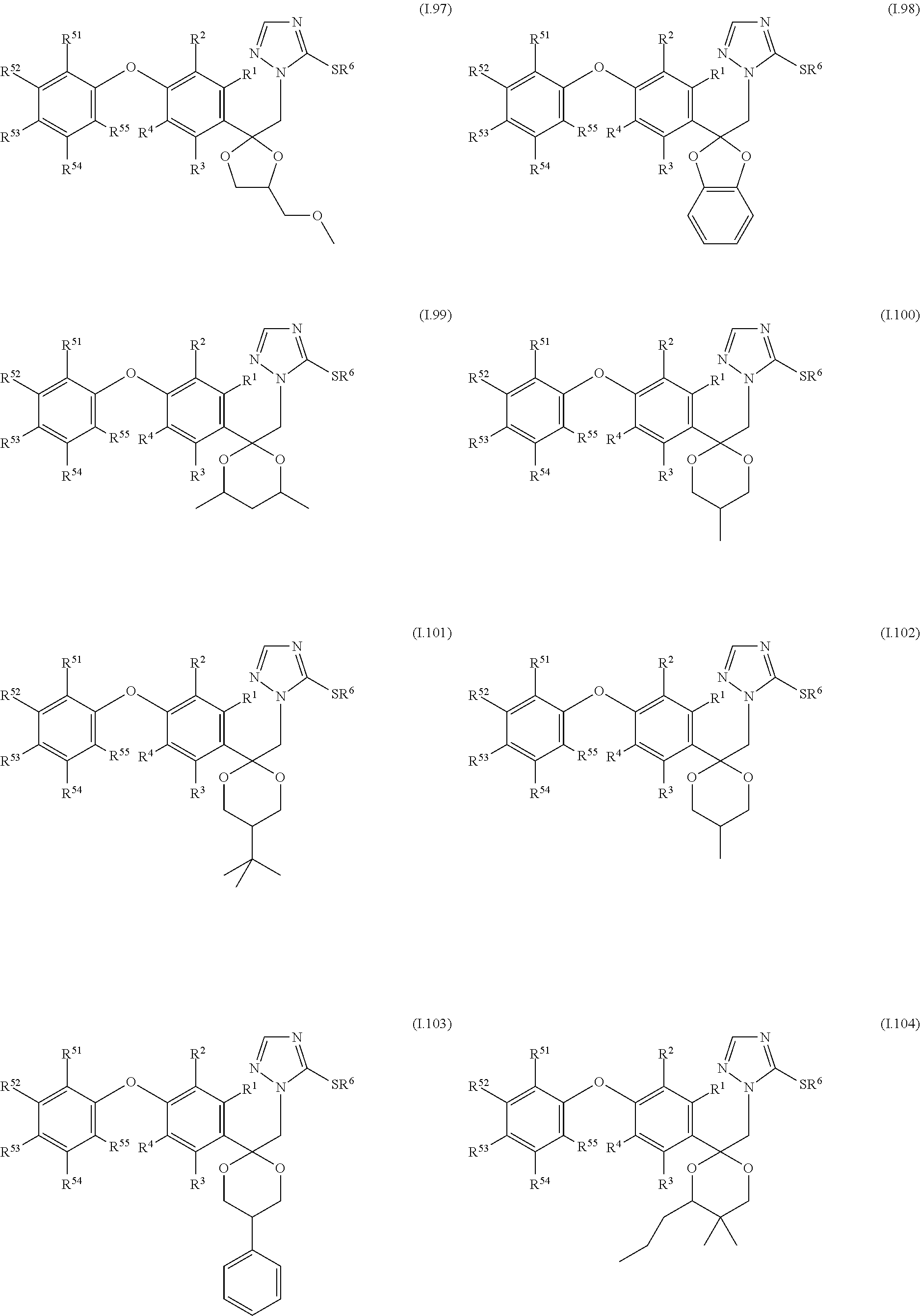

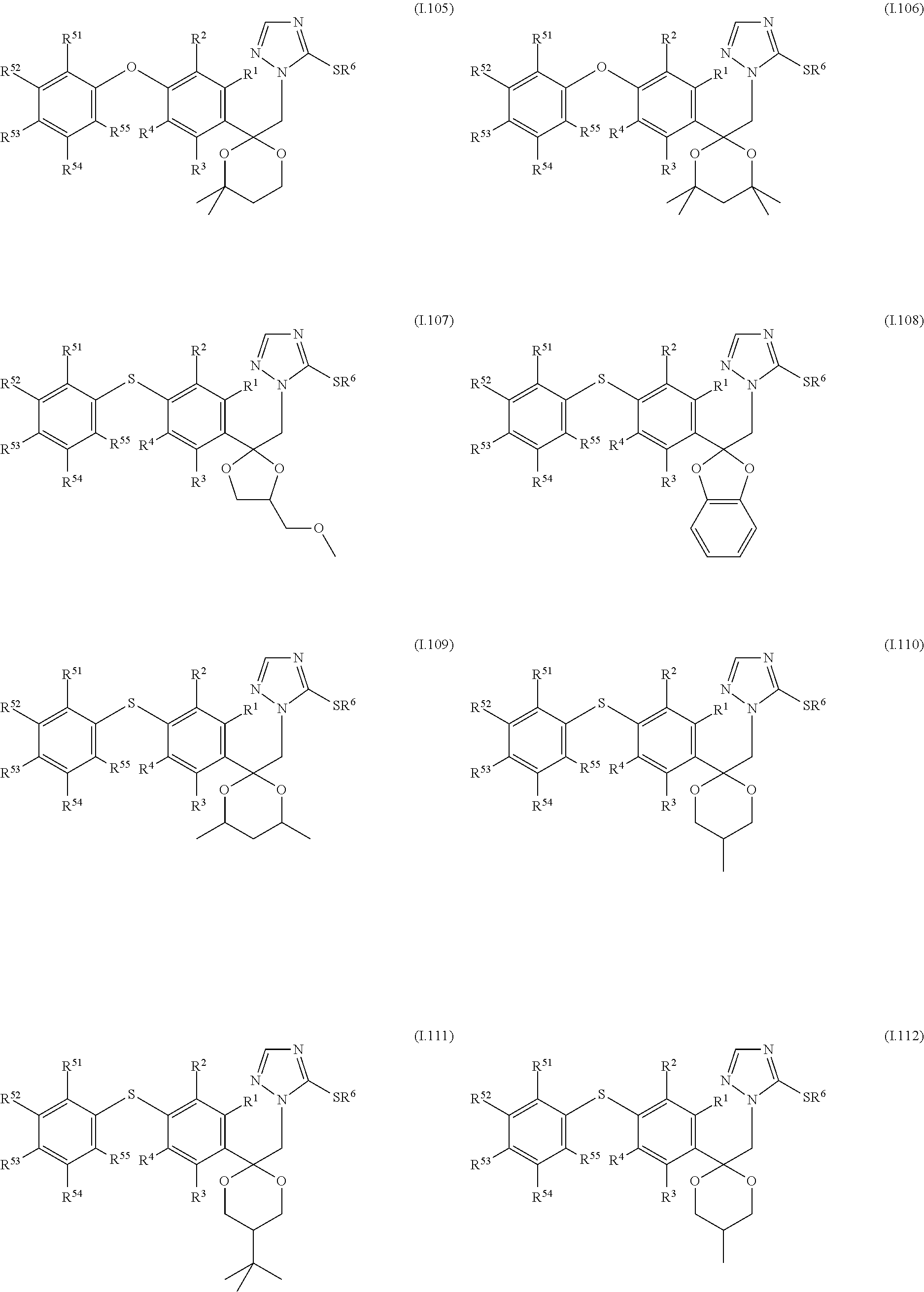

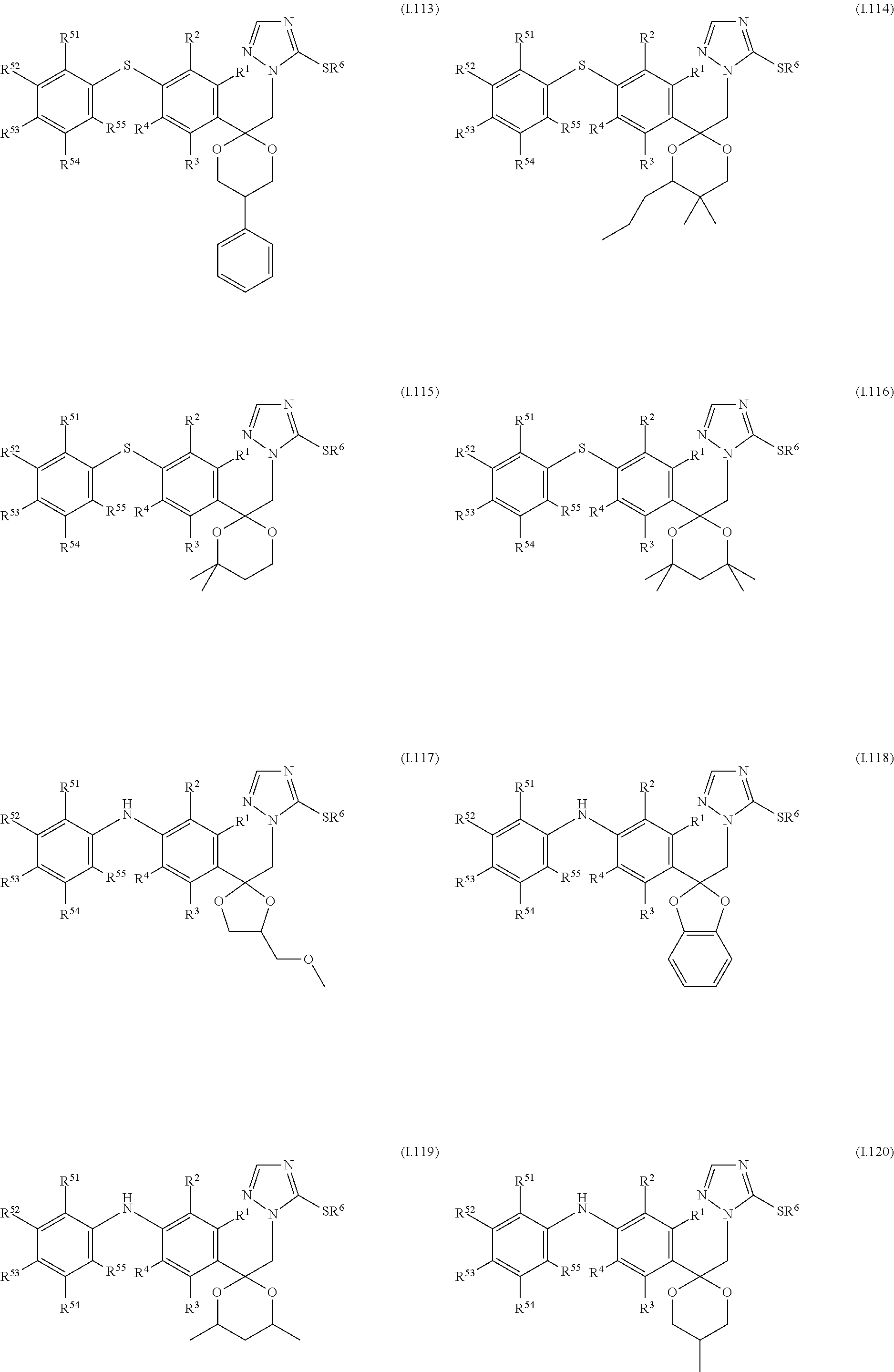

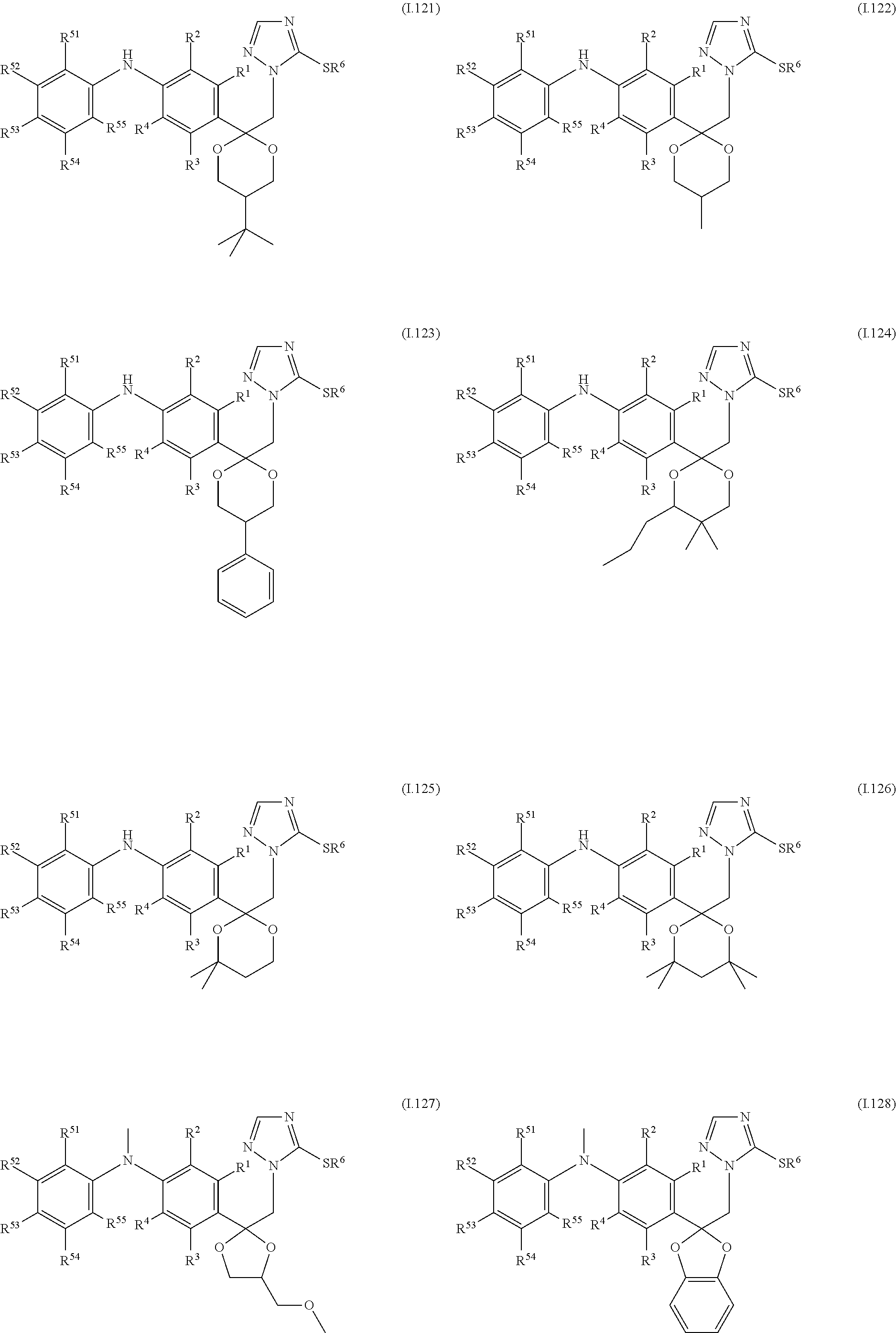

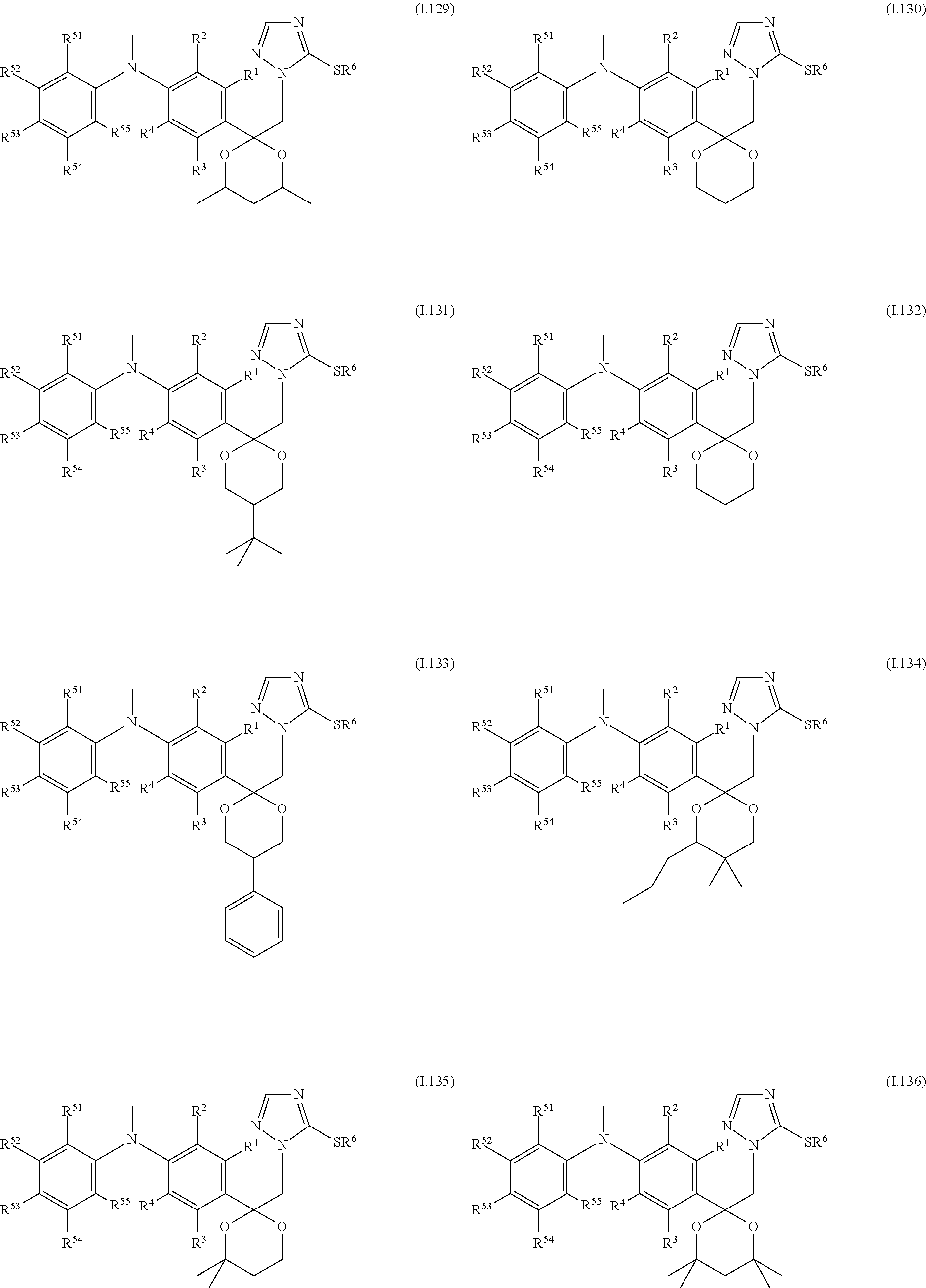

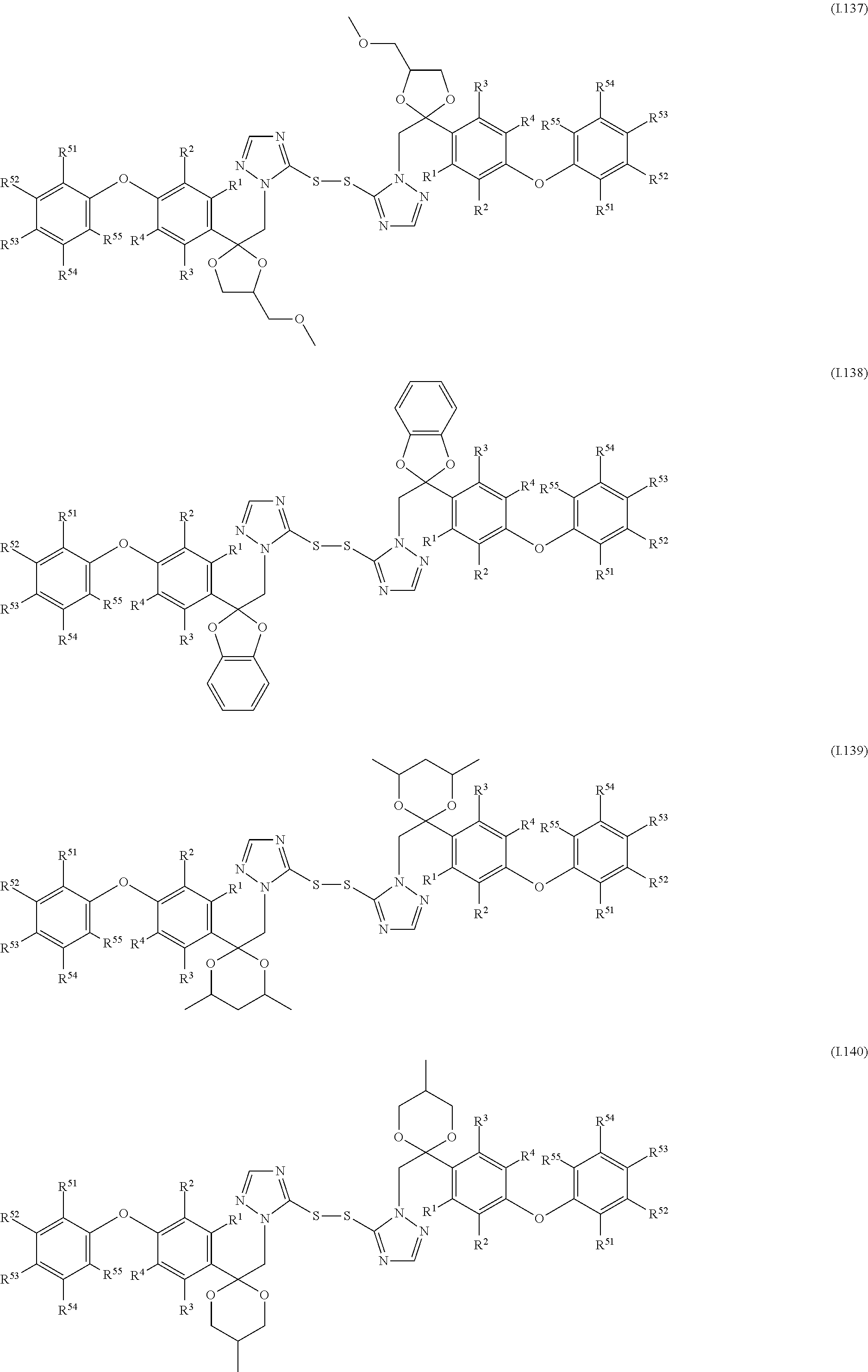

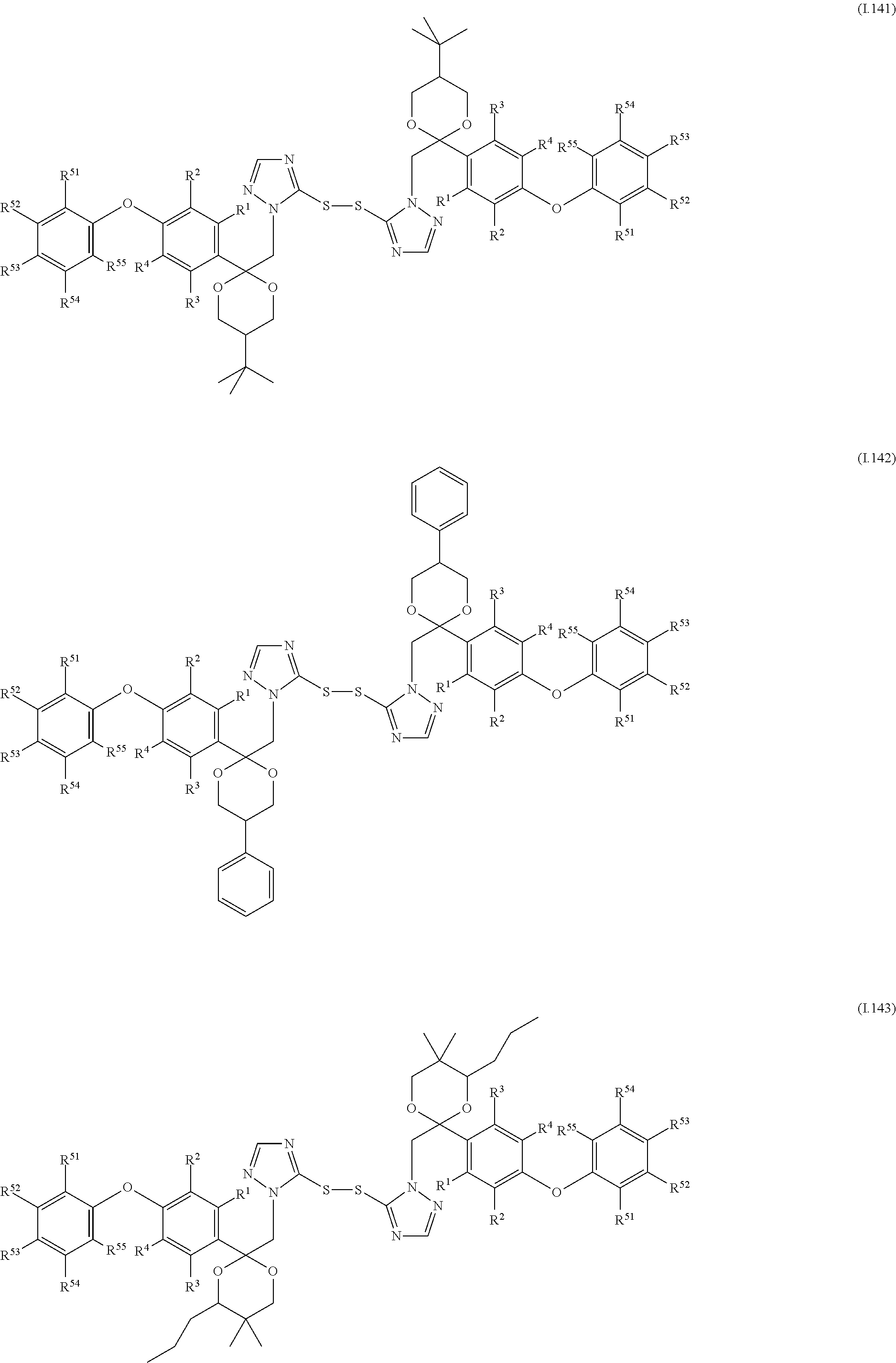

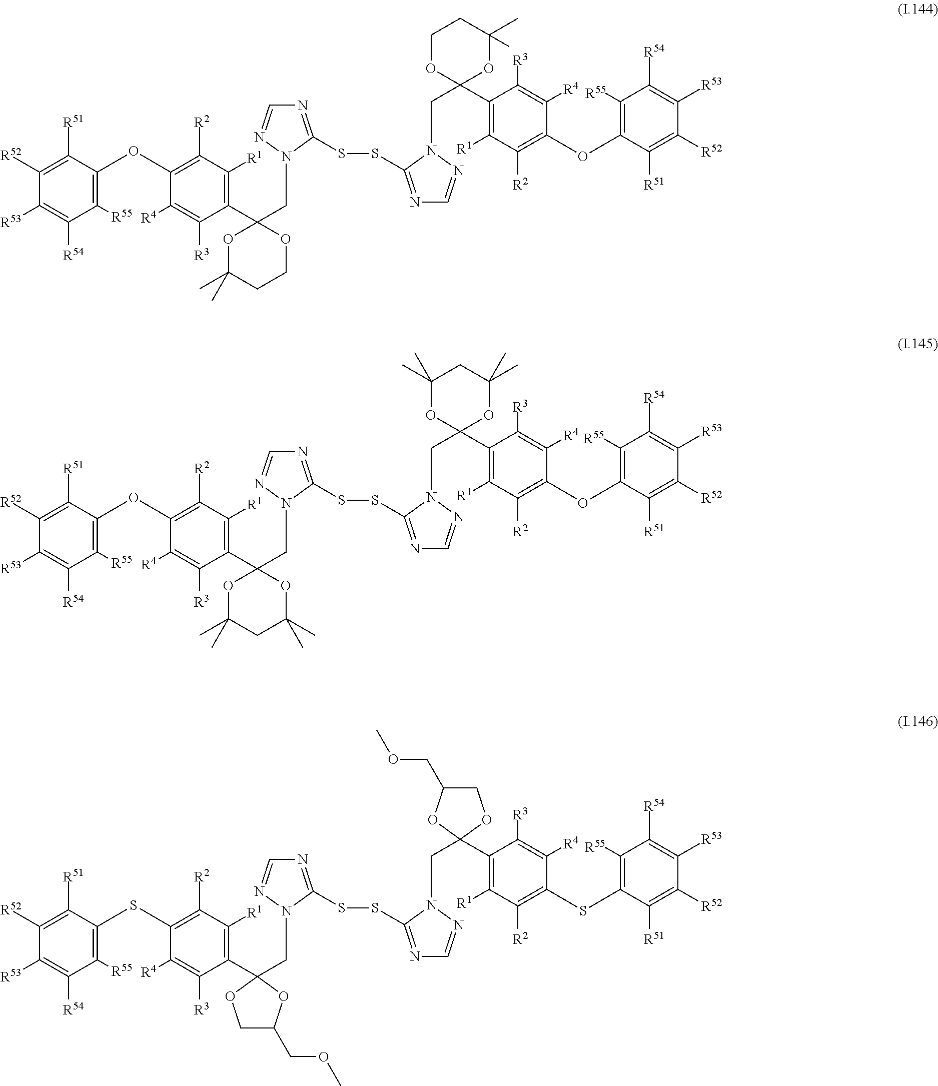

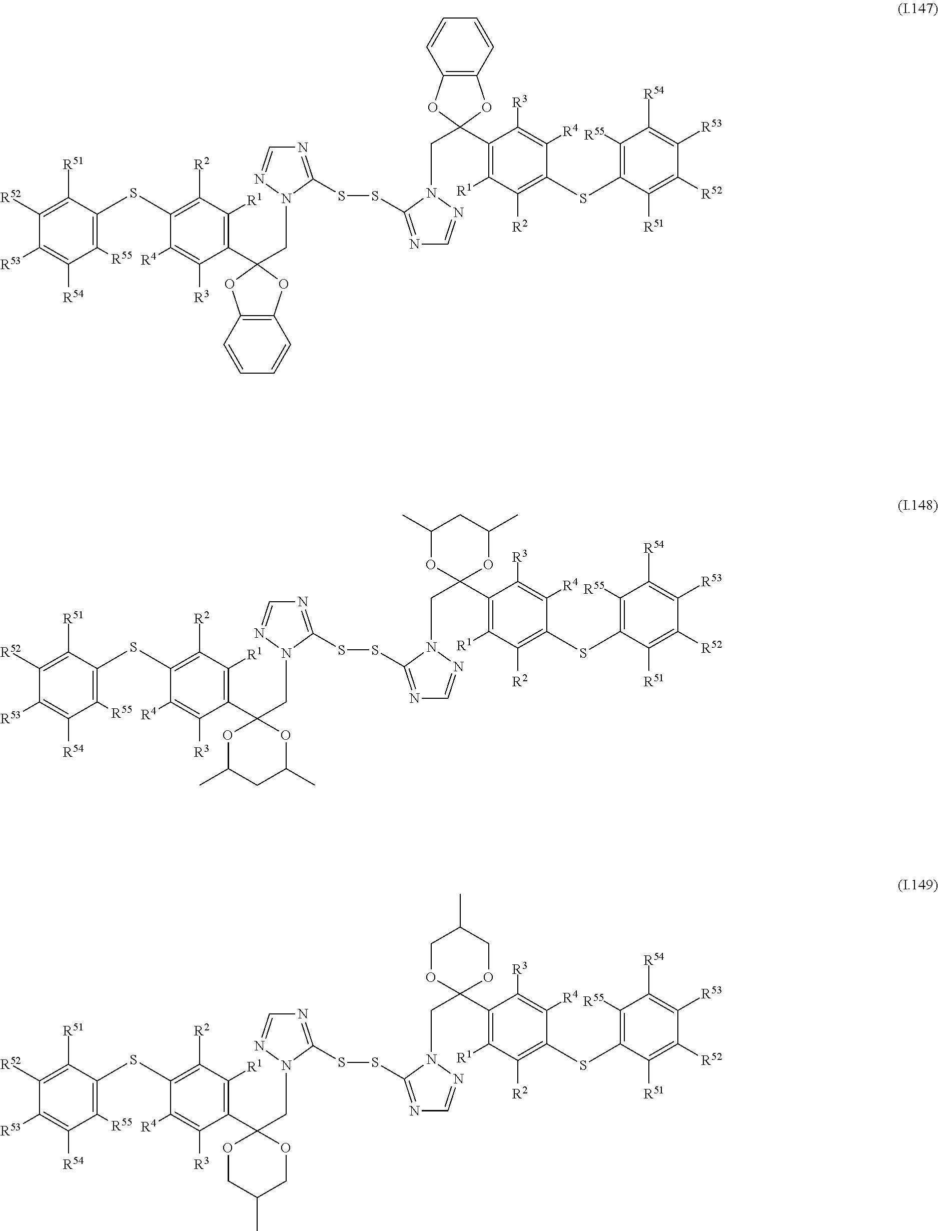

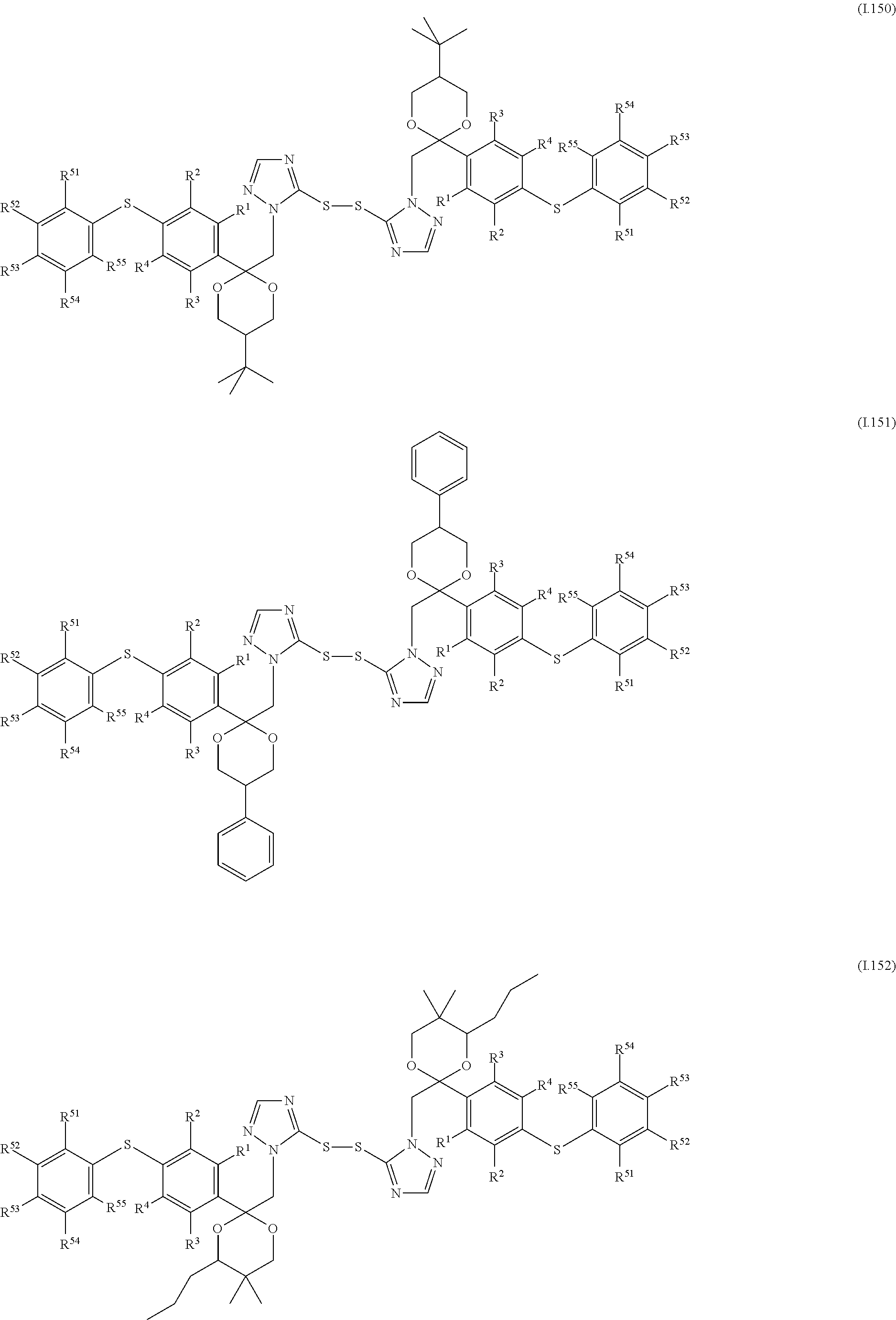

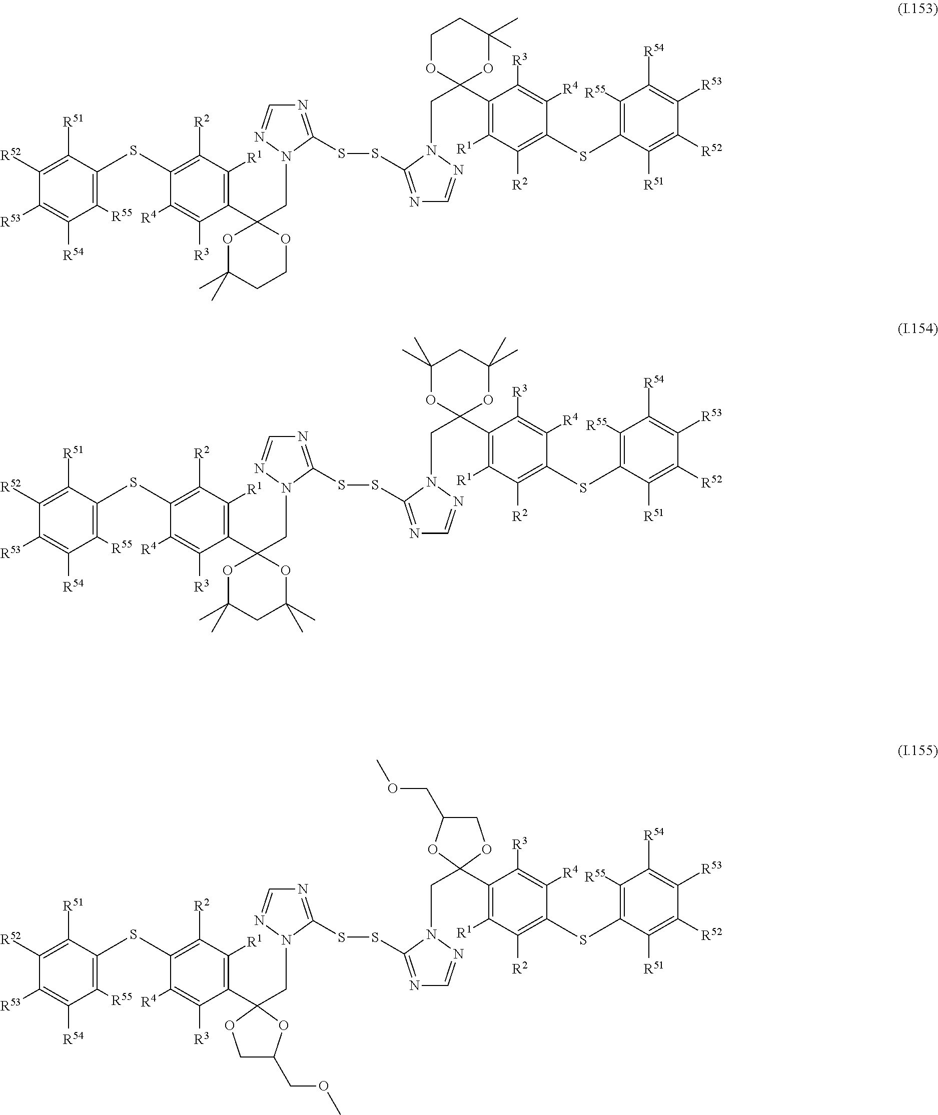

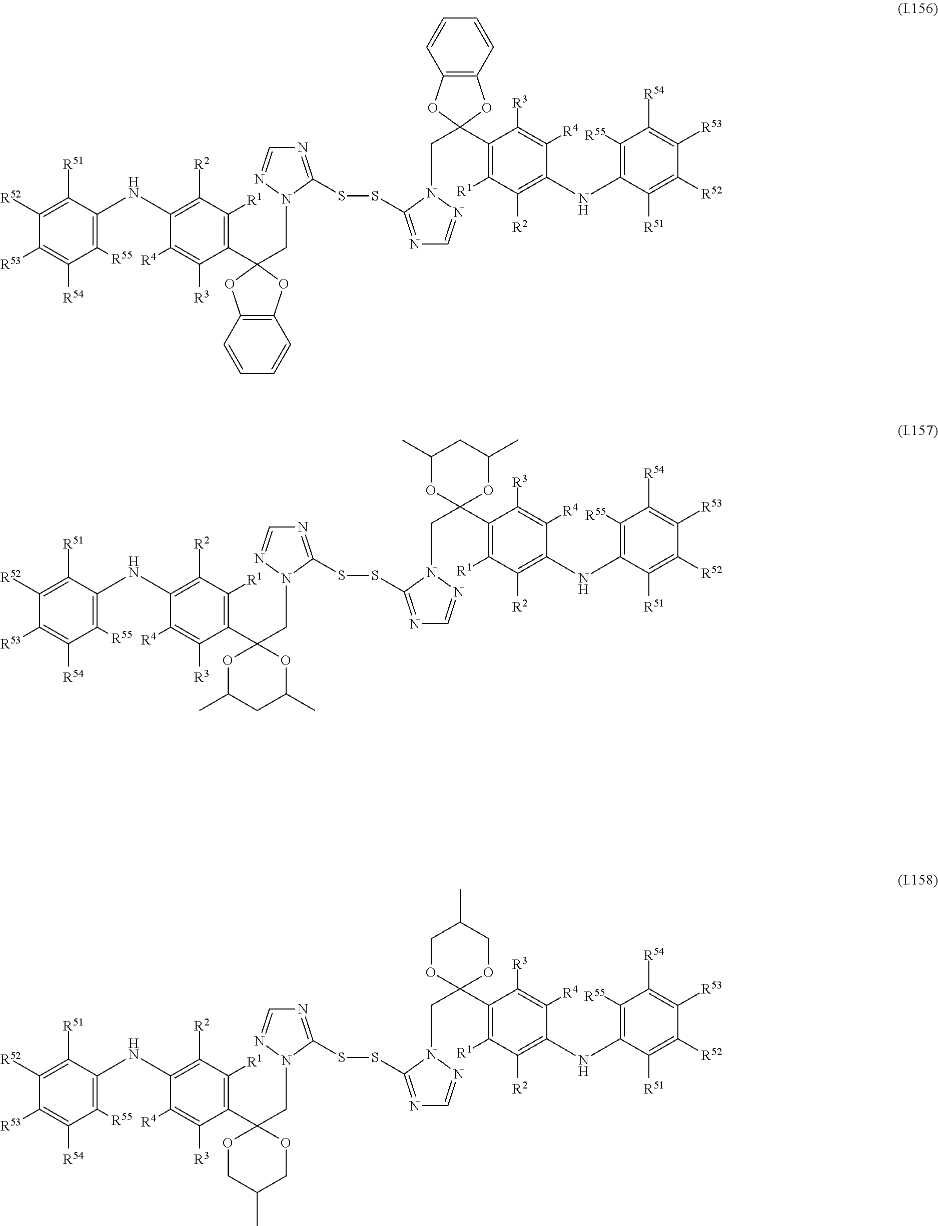

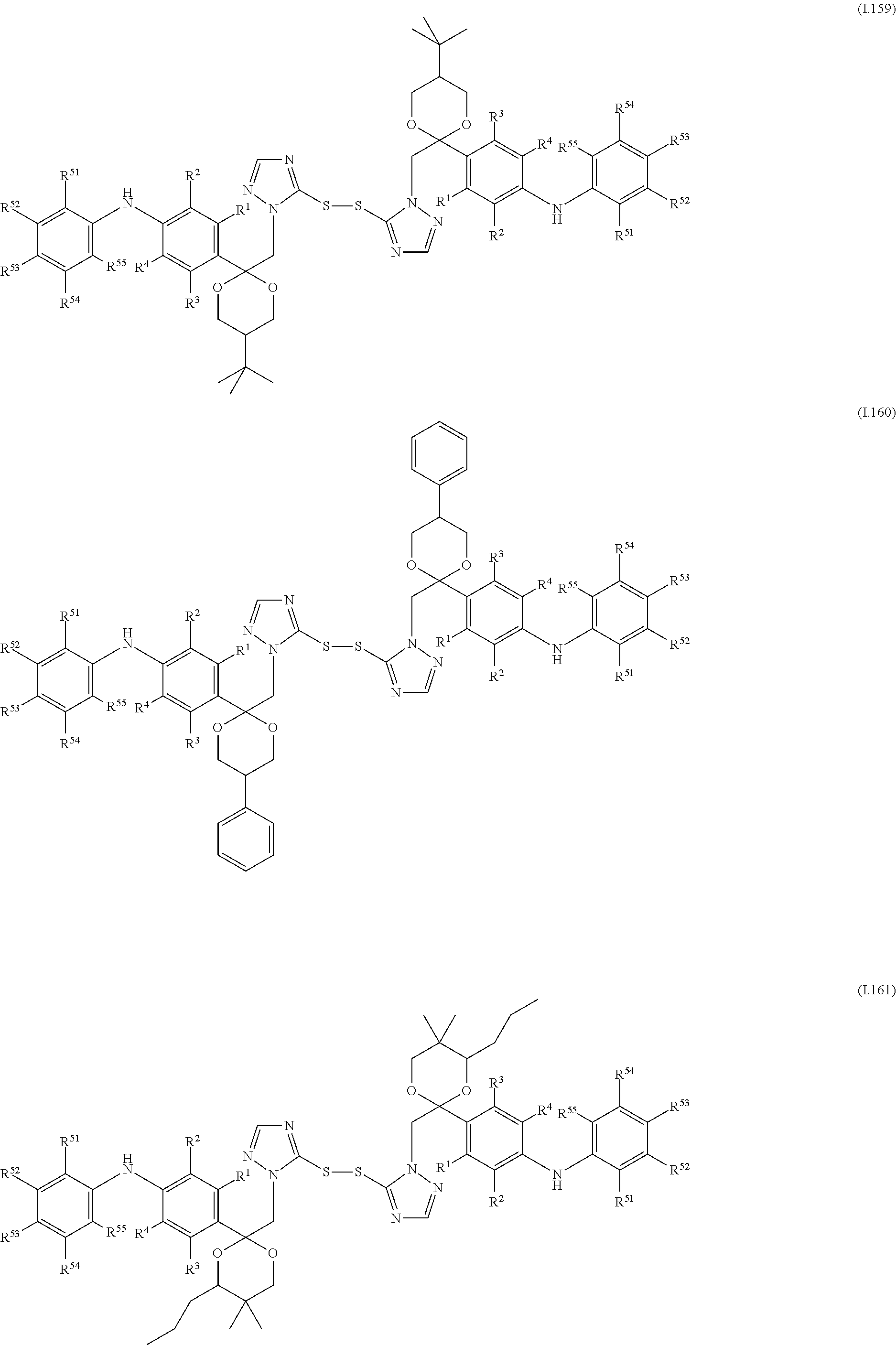

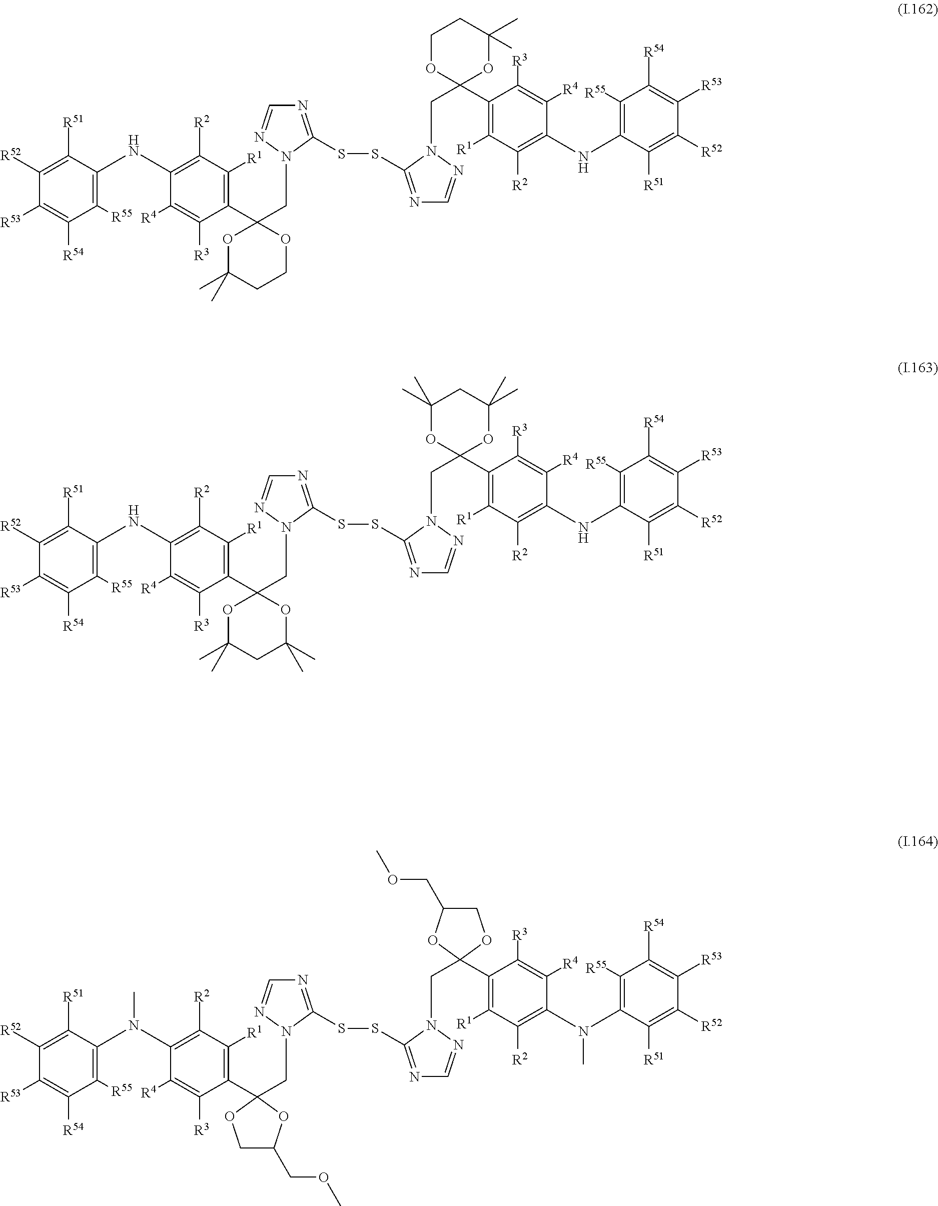

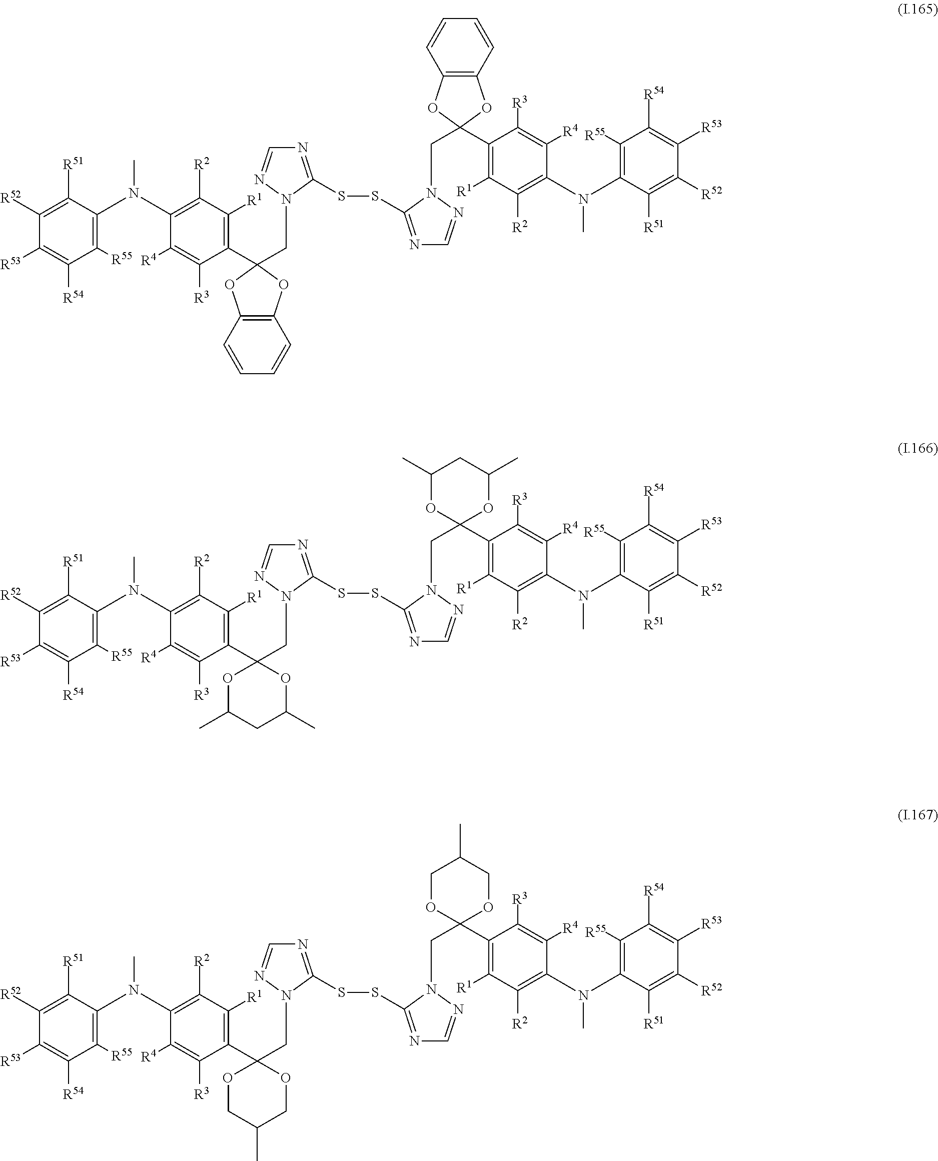

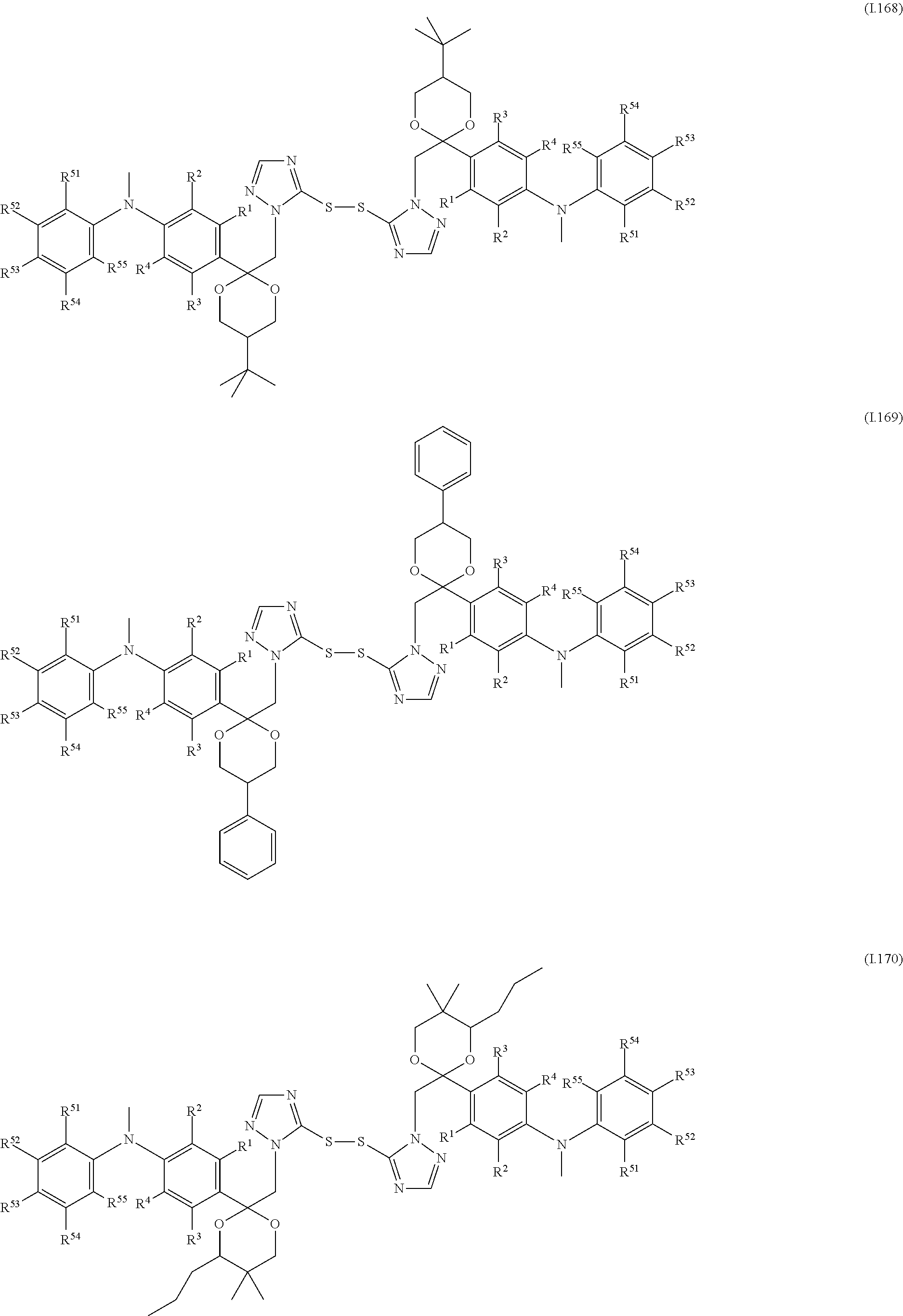

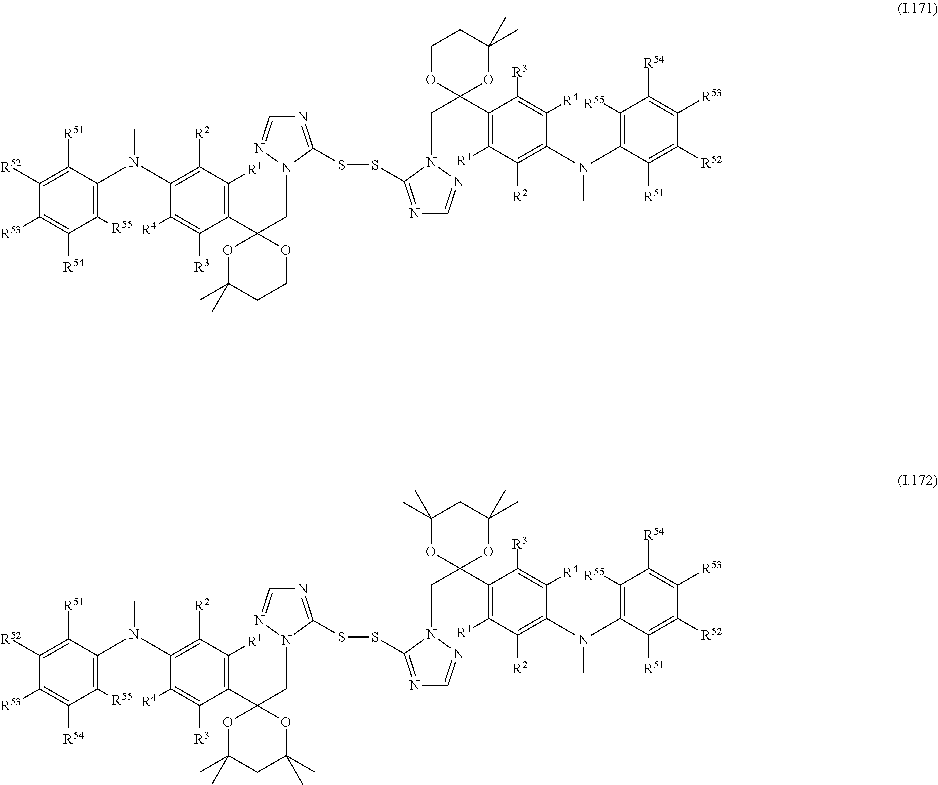

### Tables 87649 to 88677

Compounds of the formula I.97 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 88678 to 89706

Compounds of the formula I.98 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 89707 to 90735

Compounds of the formula I.99 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 90736 to 91764

Compounds of the formula I.100 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 91765 to 92793

Compounds of the formula I.101 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 92794 to 93822

Compounds of the formula I.102 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 93823 to 94851

Compounds of the formula I.103 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 94852 to 95880

Compounds of the formula I.104 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 95881 to 96909

Compounds of the formula I.105 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 96910 to 97938

Compounds of the formula I.106 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 97939 to 98967

Compounds of the formula I.107 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 98968 to 99996

Compounds of the formula I.108 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 99997 to 101025

Compounds of the formula I.109 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 101026 to 102054

Compounds of the formula I.110 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 102055 to 103083

Compounds of the formula I.111 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 103084 to 104112

Compounds of the formula I.112 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 104113 to 105141

Compounds of the formula I.113 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 105142 to 106170

Compounds of the formula I.114 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 106171 to 107199

Compounds of the formula I.115 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 107200 to 108228

Compounds of the formula I.116 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 108229 to 109257

Compounds of the formula I.117 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 109258 to 110286

Compounds of the formula I.118 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 110287 to 111315

Compounds of the formula I.119 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 111316 to 112344

Compounds of the formula I.120 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 112345 to 113373

Compounds of the formula I.121 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 113374 to 114402

Compounds of the formula I.122 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 114403 to 115431

Compounds of the formula I.123 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 115432 to 116460

Compounds of the formula I.124 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 116461 to 117489

Compounds of the formula I.125 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 117490 to 118518

Compounds of the formula I.126 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 118519 to 119547

Compounds of the formula I.127 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 119548 to 120576

Compounds of the formula I.128 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 120577 to 121605

Compounds of the formula I.129 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 121606 to 122634

Compounds of the formula I.130 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 122635 to 123663

Compounds of the formula I.131 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 123664 to 124692

Compounds of the formula I.132 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 124693 to 125721

Compounds of the formula I.133 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 126722 to 126750

Compounds of the formula I.134 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 126751 to 127779

Compounds of the formula I.135 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 127780 to 128808

Compounds of the formula I.136 in which the combination of R⁵¹, R⁵², R⁵³, R⁵⁴ and R⁵⁵ for a compound corresponds in each case to one row of Table A and the combination of R¹, R², R³, R⁴ and R⁶ is as defined in any of tables 1 to 1029

### Tables 128809 to 128828

Compounds of the formula I.137 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 128829 to 128848

Compounds of the formula I.138 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 128849 to 128868

Compounds of the formula I.139 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 128869 to 128888

Compounds of the formula I.140 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 128889 to 128908

Compounds of the formula I.141 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 128909 to 128928

Compounds of the formula I.142 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 128929 to 128948

Compounds of the formula I.143 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 128949 to 128968

Compounds of the formula I.144 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 128969 to 128988

Compounds of the formula I.145 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 128989 to 129008

Compounds of the formula I.146 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129009 to 129028

Compounds of the formula I.147 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129029 to 129048

Compounds of the formula I.148 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129049 to 129068

Compounds of the formula I.149 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129069 to 129088

Compounds of the formula I.150 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129089 to 129108

Compounds of the formula I.151 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129109 to 129128

Compounds of the formula I.152 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129129 to 129148

Compounds of the formula I.153 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129149 to 129168

Compounds of the formula I.154 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129169 to 129188

Compounds of the formula I.155 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129189 to 129208

Compounds of the formula I.156 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129209 to 129228

Compounds of the formula I.157 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129229 to 129248

Compounds of the formula I.158 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129249 to 129268

Compounds of the formula I.159 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129269 to 129288

Compounds of the formula I.160 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129289 to 129308

Compounds of the formula I.161 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129309 to 129328

Compounds of the formula I.162 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129329 to 129348

Compounds of the formula I.163 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129349 to 129368

Compounds of the formula I.164 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129369 to 129388

Compounds of the formula I.165 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129389 to 129408

Compounds of the formula I.166 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129409 to 129428

Compounds of the formula I.167 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129429 to 129448

Compounds of the formula I.168 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129449 to 129468

Compounds of the formula I.169 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129469 to 129488

Compounds of the formula I.170 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129489 to 129508

Compounds of the formula I.171 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

### Tables 129509 to 129528

Compounds of the formula I.172 in which the combination of R⁵¹, R⁵², R⁵³, R⁵⁴ and R⁵⁵ for a compound corresponds in each case to one row of Table A and the combination of R¹, R², R³ and R⁴ is as defined in any of tables 49393 to 49412

Among the above compounds, preference is given to compounds of formulae I.97 to I.106.

Compounds of formulae I and II can be prepared by one or more of the following methods and variations as described in schemes 1 to 9 and in the syntheses descriptions below. The variables are as defined above for formulae I and II.

Compounds of formula I, wherein R⁶ is H and m is 0 (or compounds II, wherein R^{6a} is H), can be prepared by sulfurizing the corresponding triazole derivative IV as outlined in scheme 1. Sulfurization can be carried out in analogy to known processes, for example as described in WO 96/41804. For instance, the triazolyl ring can be first deprotonated with a strong base, e.g. an organolithium base, such as n-butyllithium, tert-butyllithium or sec-butyllithium, lithium diisopropyl amide, sodium hydride, sodium amide or potassium tert-butylate mixed with tetramethylethylene diamine (TMEDA), and then the resulting anion is reacted with elemental sulfur. Sulfur is generally used in powdered form. The reaction is generally carried out in an inert solvent, such as ethers, e.g. diethylether, methyl-tert-butylether, tetrahydrofuran or dioxane, dimethoxyethane, liquid ammonia, dimethylsulfoxide or dimethylformamide. The reaction temperature is not very critical and can range, for example, from −70 to +50° C., preferably from −70 to 0° C. Alternatively, sulfurization can be carried out in the absence of a base by reacting 7 with elemental sulfur in a high-boiling solvent, such as N-methylpyrrolidinone, dioxane or N,N-dimethylformamide, while heating, e.g. to 160 to 250° C. After completion of the reaction, the resulting mixture is hydrolyzed, e.g. by the addition of water or an aqueous acid, such as a mineral acid (e.g. dilute sulfuric acid or hydrochloric acid), acetic acid or ammoniumchloride, to give compound I.

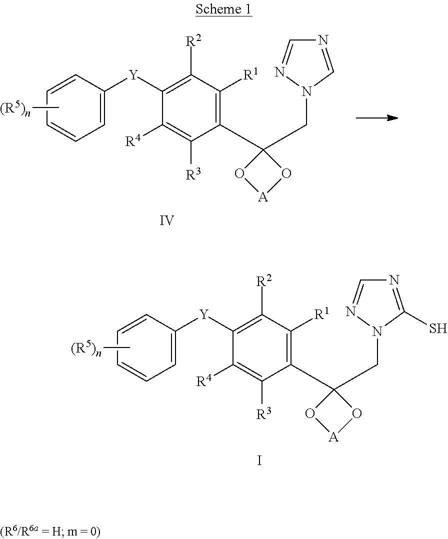

The triazole compound IV can be prepared in analogy to known methods, such as described, for example, in EP-A-0065485, as outlined in scheme 2. For instance, the compound 1, wherein X is a good leaving group, such as a halogen atom, in particular Cl, Br or I, phenylsulfonyloxy, p-tosyloxy, trifluoroacetyloxy or alkylsulfonyloxy, such as mesyloxy, can be reacted with the [1,2,4]-1H-triazole compound 2, wherein M is a hydrogen atom or a metal atom, in particular an alkali metal atom, such as Li, Na or K. In case M is H, the reaction is suitably carried out in the presence of a base, such as an alkali metal hydride (e.g. sodium hydride, potassium hydride), an alkali metal hydroxide (e.g. sodium hydroxide, potassium hydroxide), an alkali metal carbonate (e.g. sodium carbonate, potassium carbonate, caesium carbonate) or a suitable amine (e.g. triethylamine, triethylenediamine, piperidine, pyridine, 4-dimethylaminopyridine, 4-pyrrolidylpyridine). If X is Cl or Br, the reaction can be accelerated by the addition of an alkali metal iodide, such as NaI or KI. The reaction is suitably carried out in a solvent. Suitable solvents are inert versus the reacants and products and are rather polar, for example, N,N-dimethylformamide, N,N-dimethylacetamide, dimethylsulfoxide, acetonitrile, benzonitrile, ethers, such as diethylether, dipropylether, methyl-tert-butyl ether, tetrahydrofuran or dioxane, and the like, and can be used in combination with other inert, less polar solvents, such as benzene, toluene, the xylenes, chlorobenzene, nitrobenzene, hexane, heptane, petrolether and the like. The reaction temperature is not very critical and can range, for example from 0 to 220° C. and preferably from 80 to 170° C. Suitably, the reaction is carried out at reflux temperature of the reaction mixture.

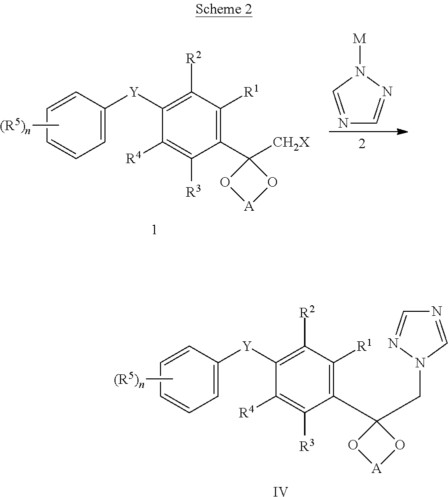

Compound 1 in turn can be prepared in analogy to known methods, such as described, for example, in EP-A-0065485 or in Synthesis, 1974, I, 23, as outlined in scheme 3 below. For instance, the ketone 3 may be reacted with a diol HO-A-OH for several hours preferably in the presence of an azeotrope-forming compound, such as benzene, toluene, xylene, chloroform or tetrachloromethane, which can also serve as reaction solvents. The ketalization reaction is accelerated by the presence of a strong acid, such p-toluoenesulfonic acid. Subsequent halogenation of the resulting ketale 4 yields ketale 1, wherein X is a halogen atom, which, if desired, can be converted into a compound 1, wherein X is a leaving group X other than halogen.

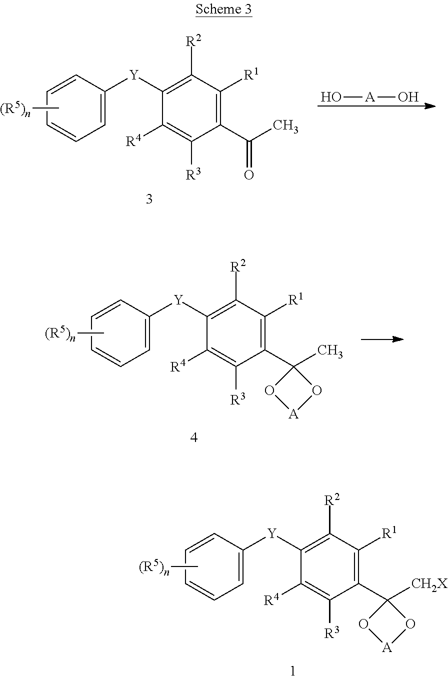

Alternatively, the ketone 3 may be first halogenated at the CH₃ group and then reacted with the diol HO-A-OH.

The ketone 3 can be obtained in analogy to known methods, such as described, for example, in EP-A-0065485, from the condensation of compounds 5 and 6, wherein X¹ is a group Y—H or Y-M, wherein M is a metal atom, in particular an alkali metal atom, such as Li, Na or K, and X² is a good leaving group, such as a halogen atom, such as F, Cl, Br or I, phenylsulfonyloxy, p-tosyloxy, trifluoroacetyloxy or alkylsulfonyloxy, such as mesyloxy, or, vice versa, wherein X¹ is a good leaving group and X² is a group Y—H or Y-M, as outlined in scheme 4 below. In case X¹ or X² is Y—H, the reaction is suitably carried out in the presence of a base, such as an alkali metal hydride (e.g. sodium hydride, potassium hydride), an alkali metal hydroxide (e.g. sodium hydroxide, potassium hydroxide), an alkali metal carbonate (e.g. sodium carbonate, potassium carbonate, caesium carbonate) or a suitable amine (e.g. triethylamine, triethylenediamine, piperidine, pyridine, 4-dimethylaminopyridine, 4-pyrrolidylpyridine). If the leaving group X¹ or X² is Cl or Br, the reaction can be accelerated by the addition of an alkali metal iodide, such as NaI or KI. The reaction is suitably carried out in a solvent. Suitable solvents are inert versus the reacants and products and are rather polar, for example, N,N-dimethylformamide, N,N-dimethylacetamide, dimethylsulfoxide, acetonitrile, benzonitrile, ethers, such as diethyl ether, dipropyl ether, methyl-tert-butyl ether, tetrahydrofuran or dioxane, and the like, and can be used in combination with other inert, less polar solvents, such as benzene, toluene, the xylenes, chlorobenzene, nitrobenzene, hexane, heptane, petrolether and the like. The reaction temperature is not very critical and can range, for example from 0 to 220° C. and preferably from 80 to 170° C. Suitably, the reaction is carried out at reflux temperature of the reaction mixture.

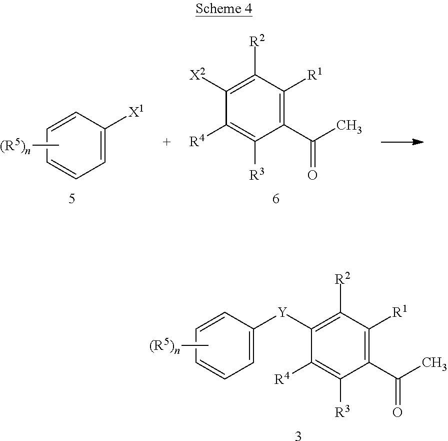

As an alternative to the process described in scheme 4, the ketone 3, wherein Y is O (=3′), can be prepared in analogy to the method described in EP-A-0065485 as outlined in scheme 5 below by decarboxylation of the carbonic ester 7. This in turn can be prepared from the phenol 8 which is reacted with a difunctional derivative 9 of carbonic acid, such as phosgene, a halogenoformic acid diester, a dialkylcarbonic diester or a diphenylcarbonic acid diester and further reaction with the phenol 10. Decarboxylation is carried out by heating 7 in substance or in a high-boiling inert solvent, such as diphenylether or ethyleneglycoldimethylether, to a temperature in the range of from 120 to 220° C.

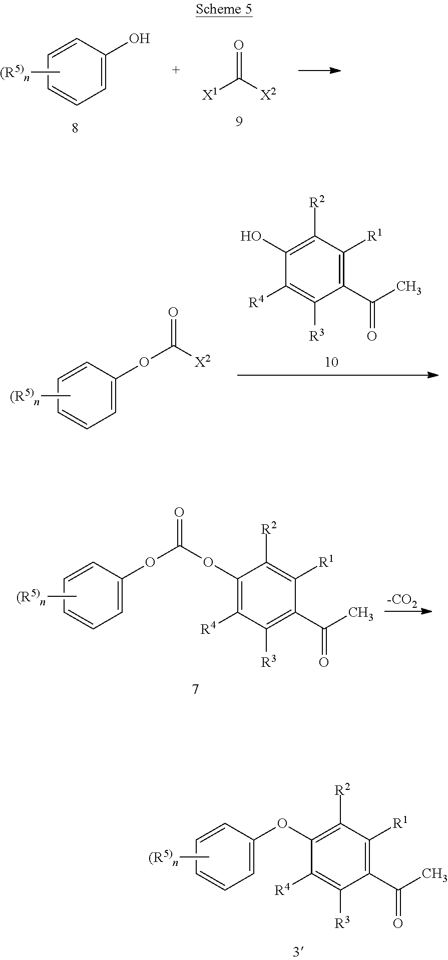

As an alternative to the process described in scheme 2, compound IV can be prepared in analogy to the method described in EP-A-0065485 as outlined in scheme 6 below by a ketalization reaction of the ketone 11 with a diol HO-A-OH. The ketalization reaction can be carried out under reaction conditions described for scheme 3.

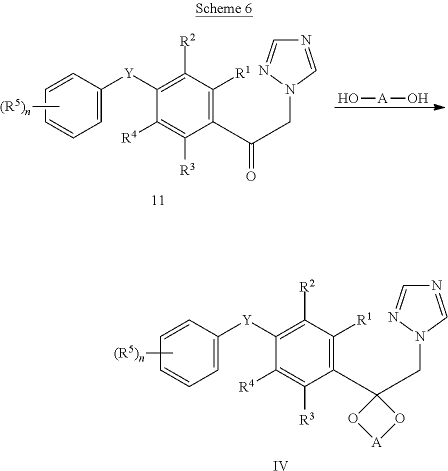

The ketone 11 in turn can be prepared in analogy to the method described in EP-A-0065485 as outlined in scheme 7 below by reacting the compound 12, wherein X is a good leaving group, such as a halogen atom, in particular Cl, Br or I, phenylsulfonyloxy, p-tosyloxy, trifluoroacetyloxy or alkylsulfonyloxy, such as mesyloxy, with the triazole compound 2 under reaction conditions described for the reaction in scheme 2. Compound 12, in turn, can be obtained from the halogenation of the ketone 3.

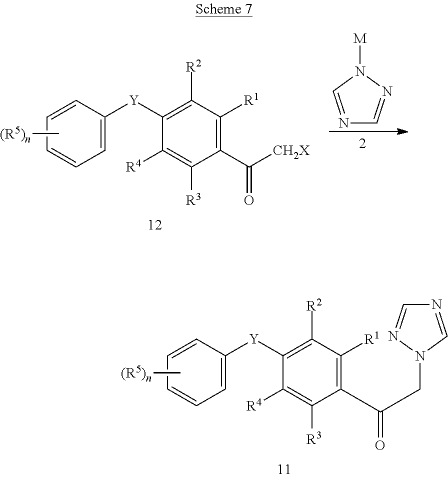

As an alternative to the process described in scheme 2, compound IV can be prepared in analogy to the method described in EP-A-0065485 as outlined in scheme 8 below by condensation of compounds 13 and 14, wherein X¹ is a group Y—H or Y-M, wherein M is a metal atom, in particular an alkali metal atom, such as Li, Na or K, and X² is a good leaving group, such as a halogen atom, in particular Cl, Br or I, phenylsulfonyloxy, p-tosyloxy, trifluoroacetyloxy or alkylsulfonyloxy, such as mesyloxy, or, vice versa, wherein X¹ is a good leaving group and X² is a group Y—H or Y-M, under reaction conditions as described for scheme 4.

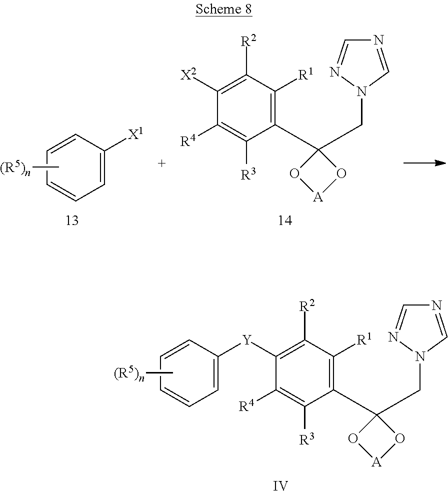

As an alternative to the process described in scheme 2, compounds IV, wherein Y is O (═IV′), can be prepared in analogy to the method described in EP-A-0065485 as outlined in scheme 9 below by decarboxylation of the carbonic ester 15 under reaction conditions as described for scheme 5. The carbonic ester 15, in turn, can be prepared under reaction conditions described for the condensation reaction in scheme 5.

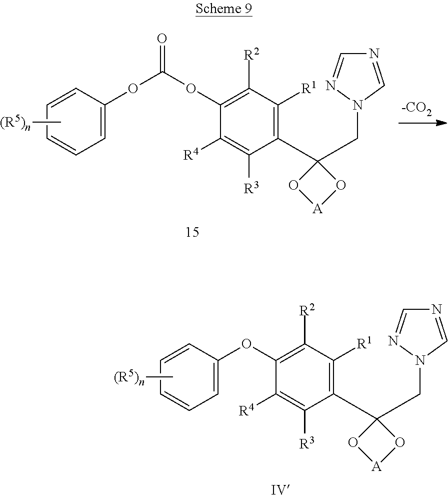

Compounds 5, 6, 8, 9 and 10 used in the above reactions are either commercially available or can be produced by standard methods known to the skilled person.

The radicals R¹, R², R³, R⁴ and R⁵ in the above reactants, if inert towards the respective reaction, can be present in the above reaction steps or can be introduced at a later stage, e.g. into compound IV.

Compounds of formula I, wherein R⁶ is different from hydrogen and m is 0, can be prepared from compounds I, wherein R⁶═H and m=0.

Compounds of formula I, wherein m is 0 and R⁶ is C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, where the phenyl moiety in the 2 last-mentioned radicals may be substituted as described above, and a 5- or 6-membered saturated, partially unsaturated or aromatic heterocyclic ring containing 1, 2 or 3 heteroatoms selected from N, O and S as ring members, where the heterocyclic ring may be substituted as described above, may be prepared in analogy to the method described in DE-A-19520098 or WO 96/41804 by reacting a compound I, wherein m is 0 and R⁶ is H, with a compound R⁶-LG, where R⁶ has one of the above meanings and LG is a leaving group, such as a halide (e.g. Cl, Br, I), a tosylate or a mesylate, in the presence of a base. Suitable bases are, for example, alkali metal hydrides (e.g. sodium hydride, potassium hydride), alkali metal hydroxides (e.g. sodium hydroxide, potassium hydroxide), alkali metal carbonates (e.g. sodium carbonate, potassium carbonate, caesium carbonate), alkali metal alkoxides (e.g. sodium methoxide, potassium methoxide, sodium ethoxide, potassium ethoxide, potassium tert-butoxide) or organolithium bases (e.g. n-butyl lithium, sec-butyl lithium, tert-butyl lithium and lithium diisopropylamine.). The reaction is generally carried out in a suitable solvent. Suitable solvents are, for example, toluene, N-methylpyrrolidinone, ethers (e.g. diethyl ether, tetrahydrofuran, dioxane, 1,2-dimethoxyethane), acetonitrile, N,N-dimethylformamide or dimethylsulfoxide.

Alternatively, compounds of formula I, wherein m is 0 and R⁶ is C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, where the phenyl moiety in the 2 last-mentioned radicals may be substituted as described above, and a 5- or 6-membered saturated, partially unsaturated or aromatic heterocyclic ring containing 1, 2 or 3 heteroatoms selected from N, O and S as ring members, where the heterocyclic ring may be substituted as described above, may be prepared in analogy to the method described in Heterocycles, 23(7), 1645-1649, 1985 by reacting compound IV with a disulfide R⁶—S—S—R⁶ in the presence of a strong base under conditions similar to those described for scheme 1.

Compounds of formulae I, wherein m is 0 and R⁶ is —C(═O)R¹² or —C(═S)R¹², may be prepared in analogy to the method described in DE-A-19617461 by reacting a compound I, wherein m is 0 and R⁶ is H, with a compound R¹²—C(═O)—W, R¹²—C(═S)—W, R^{12′}—N═C═O or R^{12′}—N═C═S, wherein R¹² has one of the above meanings, R^{12′} is C₁-C₁₀-alkyl or C₁-C₁₀-haloalkyl and W is a good leaving group, such as a halide (e.g. Cl, Br, I), an alkoxide (e.g. methoxide, ethoxide) or pentafluorophenoxide, in the presence of a base. Suitable bases are, for example, alkali metal hydrides (e.g. sodium hydride, potassium hydride), alkali metal hydroxides (e.g. sodium hydroxide, potassium hydroxyides), alkali metal carbonates (e.g. sodium carbonate, potassium carbonate, caesium carbonate), alkali metal alkoxides (e.g. sodium methoxide, potassium methoxide, sodium ethoxide, potassium ethoxide, potassium tert-butoxide) or organolithium bases (e.g. n-butyl lithium, sec-butyl lithium, tert-butyl lithium, lithium diisopropylamine). The reaction is generally carried out in a suitable solvent. Suitable solvents are, for example, toluene, N-methylpyrrolidinone, ethers (e.g. diethyl ether, tetrahydrofuran, dioxane, 1,2-dimethoxyethane), acetonitrile, N,N-dimethylformamide or dimethylsulfoxide.

Compounds of formula I, wherein m is 0 and R⁶ is —SO₂R¹², may be prepared in analogy to the method described in DE-A-19620590 by reacting a compound I, wherein m is 0 and R⁶ is H, with a compound R¹²—SO₂—W, wherein R¹² has one of the above meanings and W is a good leaving group, such as a halide (e.g. Cl, Br, I), an alkoxide (e.g. methoxide, ethoxide) or pentafluorophenoxide, in the presence of a base. Suitable bases are, for example, alkali metal hydrides (e.g. sodium hydride, potassium hydride), alkali metal hydroxides (e.g. sodium hydroxide, potassium hydroxide), alkali metal carbonates (e.g. sodium carbonate, potassium carbonate, caesium carbonate), alkali metal alkoxides (e.g. sodium methoxide, potassium methoxide, sodium ethoxide, potassium ethoxide, potassium tert-butoxide) or organolithium bases (e.g. n-butyl lithium, sec-butyl lithium, tert-butyl lithium, lithium diisopropylamine). The reaction is generally carried out in a suitable solvent. Suitable solvents are, for example, toluene, N-methylpyrrolidinone, ethers (e.g. diethyl ether, tetrahydrofuran, dioxane, 1,2-dimethoxyethane), acetonitrile, N,N-dimethylformamide or dimethylsulfoxide.

Compounds of formula I, wherein m is 0 and R⁶ is —CN, may be prepared in analogy to the method described in DE-A-19620407 by reacting a compound I, wherein m is 0 and R⁶ is H, with a compound CN—W, wherein W is a good leaving group, such as a halide (e.g. Cl, Br, I), in the presence of a base. Suitable bases are, for example, alkali metal hydrides (e.g. sodium hydride, potassium hydride), alkali metal hydroxides (e.g. sodium hydroxide, potassium hydroxide), alkali metal carbonates (e.g. sodium carbonate, potassium carbonate, caesium carbonate), alkali metal alkoxides (e.g. sodium methoxide, potassium methoxide, sodium ethoxide, potassium ethoxide, potassium tert-butoxide) or organolithium bases (e.g. n-butyl lithium, sec-butyl lithium, tert-butyl lithium, lithium diisopropylamine). The reaction is generally carried out in a suitable solvent. Suitable solvents are, for example, toluene, N-methylpyrrolidinone, ethers (e.g. diethyl ether, tetrahydrofuran, dioxane, 1,2-dimethoxyethane), acetonitrile, N,N-dimethylformamide or dimethylsulfoxide.

Compounds of formula I, wherein m is 0 and R⁶ is M, may be prepared in analogy to the method described in DE-A-19617282 by reacting a compound I, wherein m is 0 and R⁶ is H, with an amine NR^{a}R^{b}R^{c}, wherein R^{a}, R^{b} and R^{c} are as defined above, or with a metal salt, such as sodium hydroxide, potassium hydroxide or copper acetate.

Compounds of formula I, wherein m is 0 and R⁶ is a group of formula III, may be prepared in analogy to the method described in WO 97/43269 by reacting a compound I, wherein m is 0 and R⁶ is H, with a halogen, especially iodine, in the presence of a base. Suitable bases are, for example, alkali metal hydrides (e.g. sodium hydride, potassium hydride), alkali metal hydroxides (e.g. sodium hydroxide, potassium hydroxyide), alkali metal carbonates (e.g. sodium carbonate, potassium carbonate, caesium carbonate), alkali metal alkoxides (e.g. sodium methoxide, potassium methoxide, sodium ethoxide, potassium ethoxide, potassium tert-butoxide) or organolithium bases (e.g. n-butyl lithium, sec-butyl lithium, tert-butyl lithium, lithium diisopropylamine). The reaction is generally carried out in a suitable solvent. Suitable solvents are, for example, toluene, N-methylpyrrolidinone, ethers (e.g. diethyl ether, tetrahydrofuran, dioxane, 1,2-dimethoxyethane), acetonitrile, N,N-dimethylformamide or dimethylsulfoxide.

Compounds of formula I, wherein m is 0 and R⁶ is —P(=Q)R¹³R¹⁴, may be prepared in analogy to the method described in WO 99/05149.

Compounds of formula II, wherein R^{6a} is different from hydrogen, can be prepared by reacting the NR^{6a} group, wherein R^{6a} is H, in analogy to the above-described conversion of compounds I, wherein R⁶ is H, into compounds, wherein R⁶ is different from H.

Compounds I, wherein m is 1 or 2, can be prepared from respective compounds I, wherein m is 0, by oxidation. Alternatively, compounds I, wherein m is 2, can be prepared from compounds IV by first deprotonating the triazolyl ring and then reacting with a sulfonyl chloride R⁶SO₂Cl. Compounds I, wherein m is 3, can be prepared from compounds IV by first deprotonating the triazolyl ring and then reacting with sulfuric acid chloride or a sulfuric ester chloride of formula R⁶SO₂Cl, wherein R⁶ is selected from hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, where the phenyl moiety in the 2 last-mentioned radicals may be substituted as mentioned above, and a 5- or 6-membered saturated, partially unsaturated or aromatic heterocyclic ring containing 1, 2 or 3 heteroatoms selected from N, O and S, wherein the heterocyclic ring may be substituted as mentioned above.

If individual compounds cannot be prepared via the above-described routes, they can be prepared by derivatization of other compounds I and II or by customary modifications of the synthesis routes described.

The reaction mixtures are worked up in the customary manner, for example by mixing with water, separating the phases, and, if appropriate, purifying the crude products by chromatography, for example on alumina or silica gel. Some of the intermediates and end products may be obtained in the form of colorless or pale brown viscous oils, which are freed or purified from volatile components under reduced pressure and at moderately elevated temperature. If the intermediates and end products are obtained as solids, they may be purified by recrystallization or digestion.

A further aspect of the invention relates to compounds of formula IV

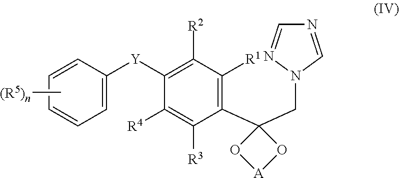

wherein R¹, R², R³, R⁴, R⁵ and n have one of the general or, in particular, one of the preferred meanings given above for compounds I and II, preferably with the proviso that R¹ is not Cl if R², R³ and R⁴ are hydrogen, Y is O, (R⁵)ₙ is 4-Cl, relative to the 1-position of the attachment point of the phenyl ring to the group Y, and A is linear C₂-alkylene which may carry one or two radicals R⁷ selected from C₁-C₄-alkyl or is —(CH₂)₃—; and with the proviso that R³ is not Cl if R¹, R² and R⁴ are hydrogen, Y is O and (R⁵)ₙ is 4-Cl, relative to the 1-position of the attachment point of the phenyl ring to the group Y, and A is linear C₂-alkylene which may carry one or two radicals R⁷ selected from C₁-C₄-alkyl or is —(CH₂)₃—;
more preferably with the proviso that R¹ is not Cl if R², R³ and R⁴ are hydrogen, Y is O and (R⁵)ₙ is 4-Cl, relative to the 1-position of the attachment point of the phenyl ring to the group Y; and with the proviso that R³ is not Cl if R¹, R² and R⁴ are hydrogen, Y is O and (R⁵)ₙ is 4-Cl, relative to the 1-position of the attachment point of the phenyl ring to the group Y.

Compounds IV are on the one side valuable intermediates in the preparation of compounds I and II (see above schemes), but on the other side show a remarkable fungicidal activity, too.

In compounds IV, Y is preferably O or NR⁸ and in particular O.

Particularly preferred compounds IV are compounds of formulae IV.1 to IV.88, wherein the combination of R⁵¹, R⁵², R⁵³, R⁵⁴ and R⁵⁵ corresponds in each case to one row in table A above and the combination of R¹, R², R³ and R⁴ is selected from the meanings given in Tables IV.1 to IV.19:

### Table IV.1

R¹, R², R³ and R⁴ are H

### Table IV.2

R¹ is F and R², R³ and R⁴ are H

### Table IV.3

R¹ is Cl and R², R³ and R⁴ are H

### Table IV.4

R¹ is Br and R², R³ and R⁴ are H

### Table IV.5

R¹ is CH₃ and R², R³ and R⁴ are H

### Table IV.6

R¹ is CF₃ and R², R³ and R⁴ are H

### Table IV.7

R¹ is OCH₃ and R², R³ and R⁴ are H

### Table IV.8

R¹ is OCF₃ and R², R³ and R⁴ are H

### Table IV.9

R² is F and R¹, R³ and R⁴ are H

### Table IV.10

R² is Cl and R¹, R³ and R⁴ are H

### Table IV.11

R² is Br and R¹, R³ and R⁴ are H

### Table IV.12

R² is CH₃ and R¹, R³ and R⁴ are H

### Table IV.13

R² is CF₃ and R¹, R³ and R⁴ are H

### Table IV.14

R² is OCH₃ and R¹, R³ and R⁴ are H

### Table IV.15

R² is OCF₃ and R¹, R³ and R⁴ are H

### Table IV.16

R¹ and R² are F and R³ and R⁴ are H

### Table IV.17

R¹ and R³ are F and R² and R⁴ are H

### Table IV.18

R¹ and R⁴ are F and R² and R³ are H

### Table IV.19

R² and R⁴ are F and R¹ and R³ are H

### Table IV.20

R¹ is Cl, and R⁴ is F and R² and R³ are H

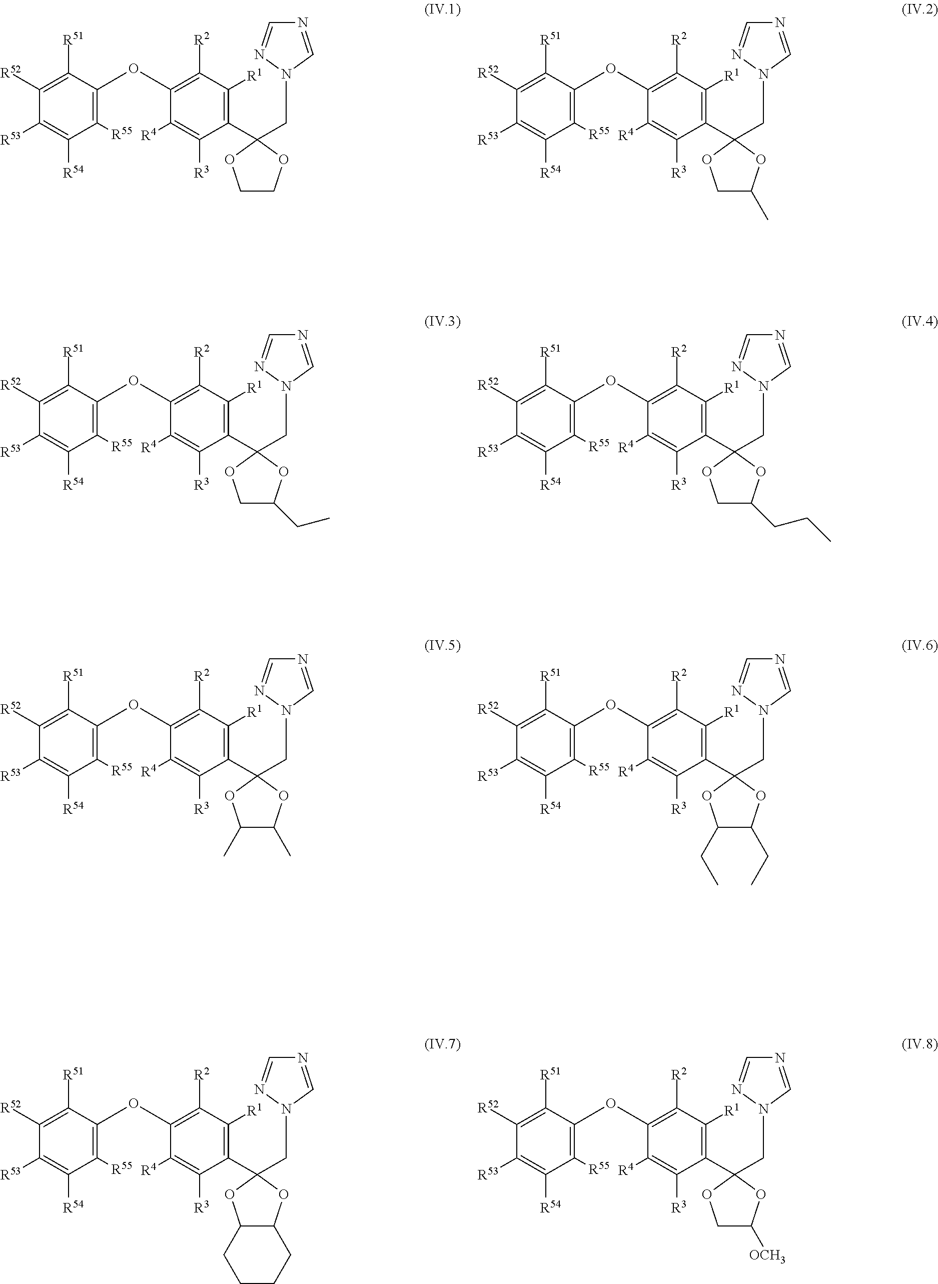

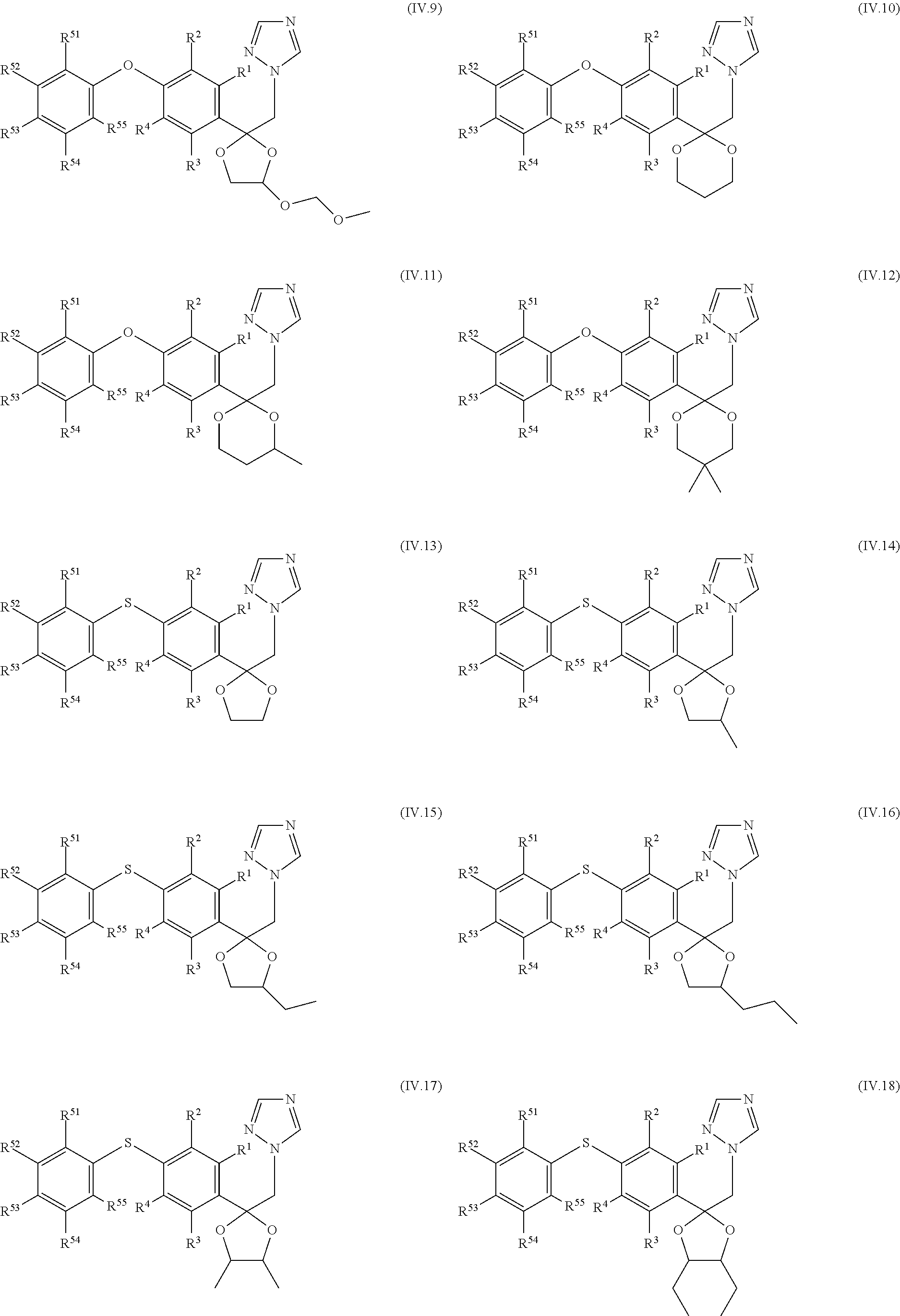

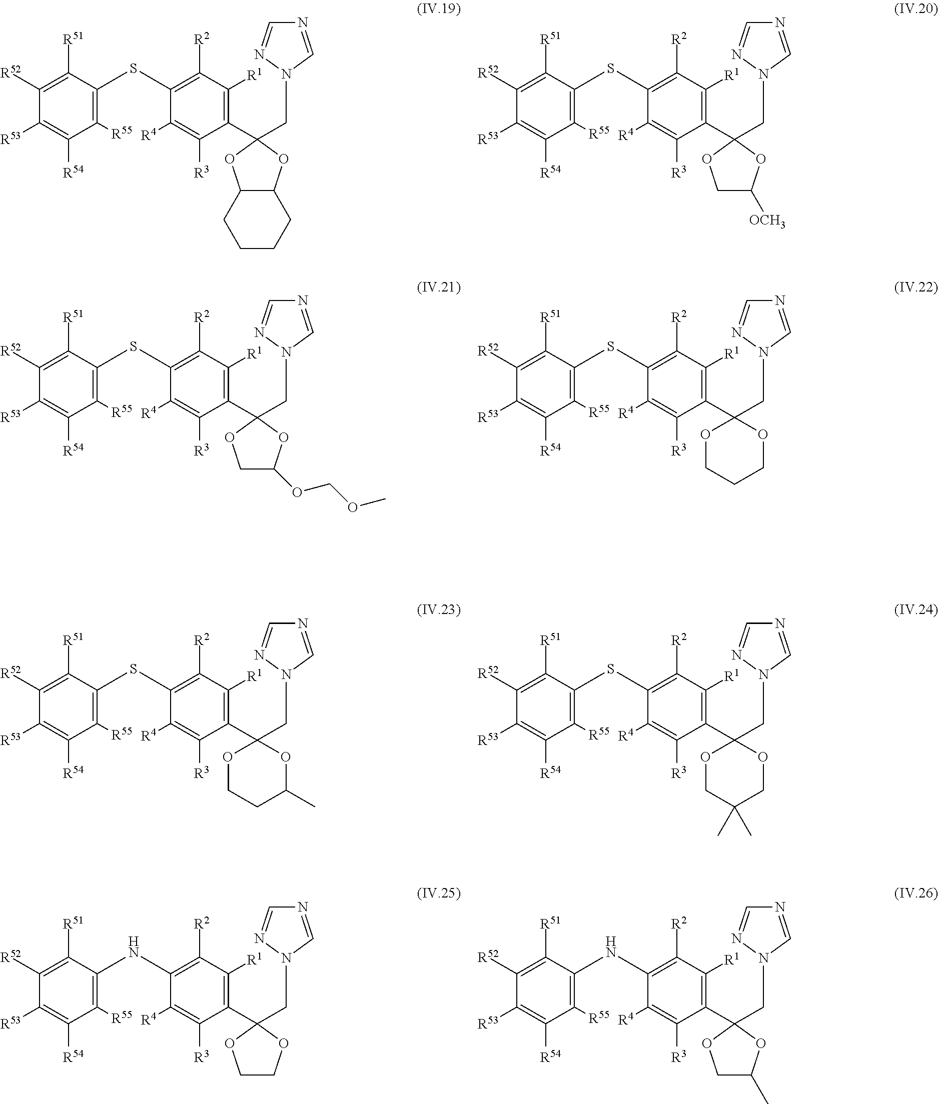

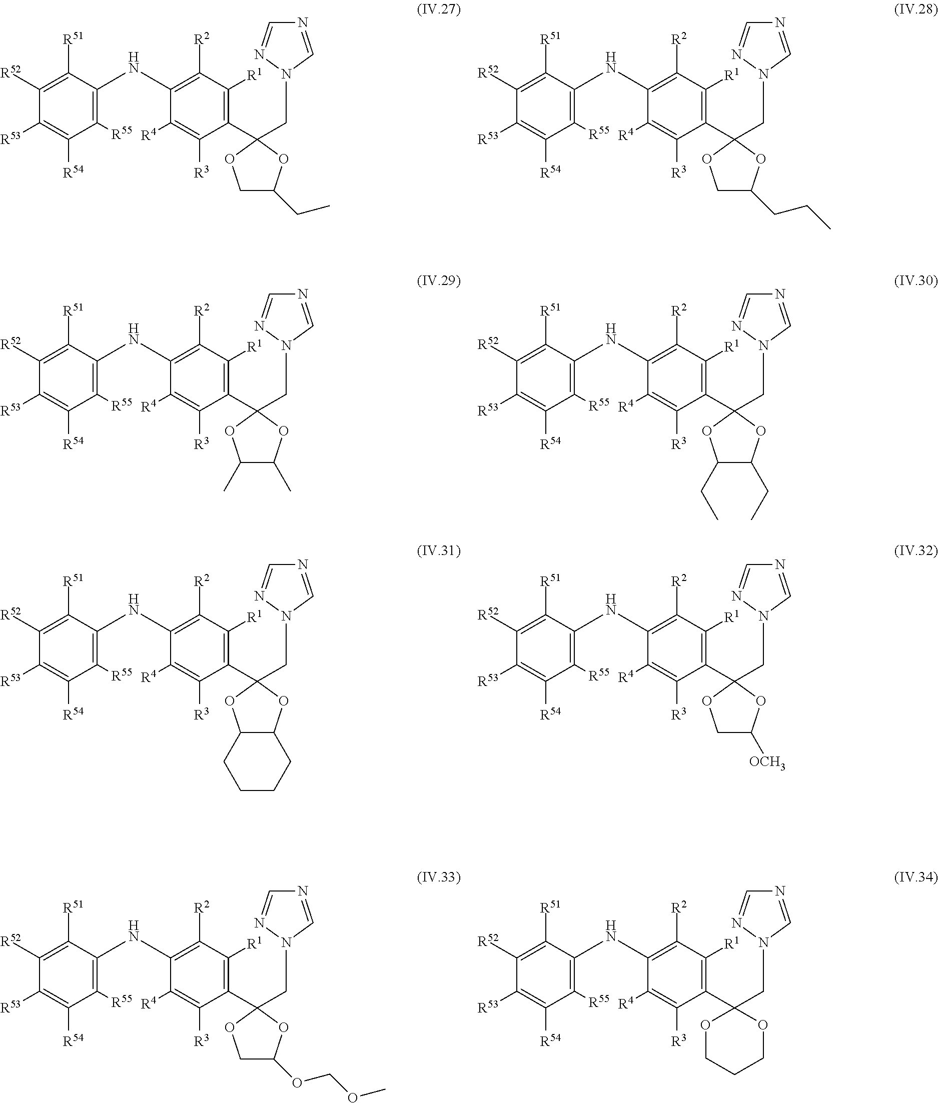

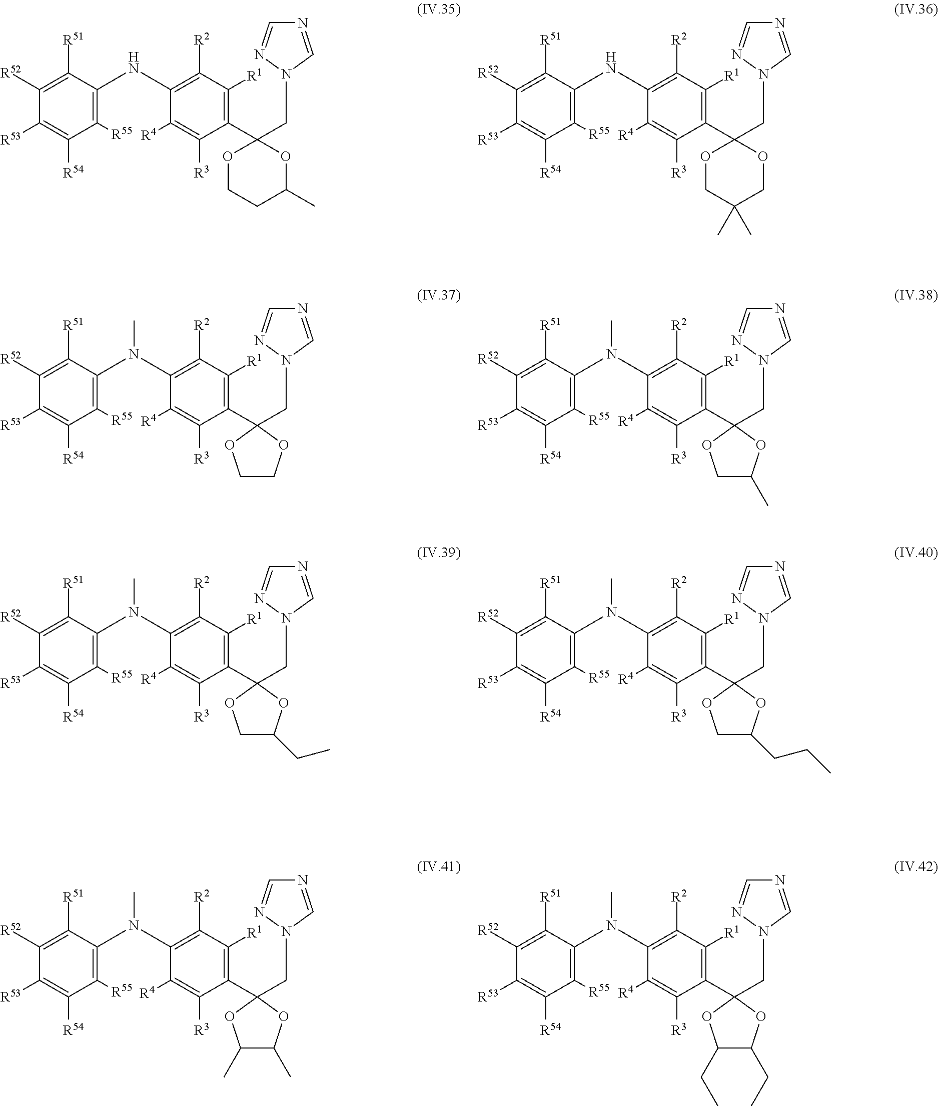

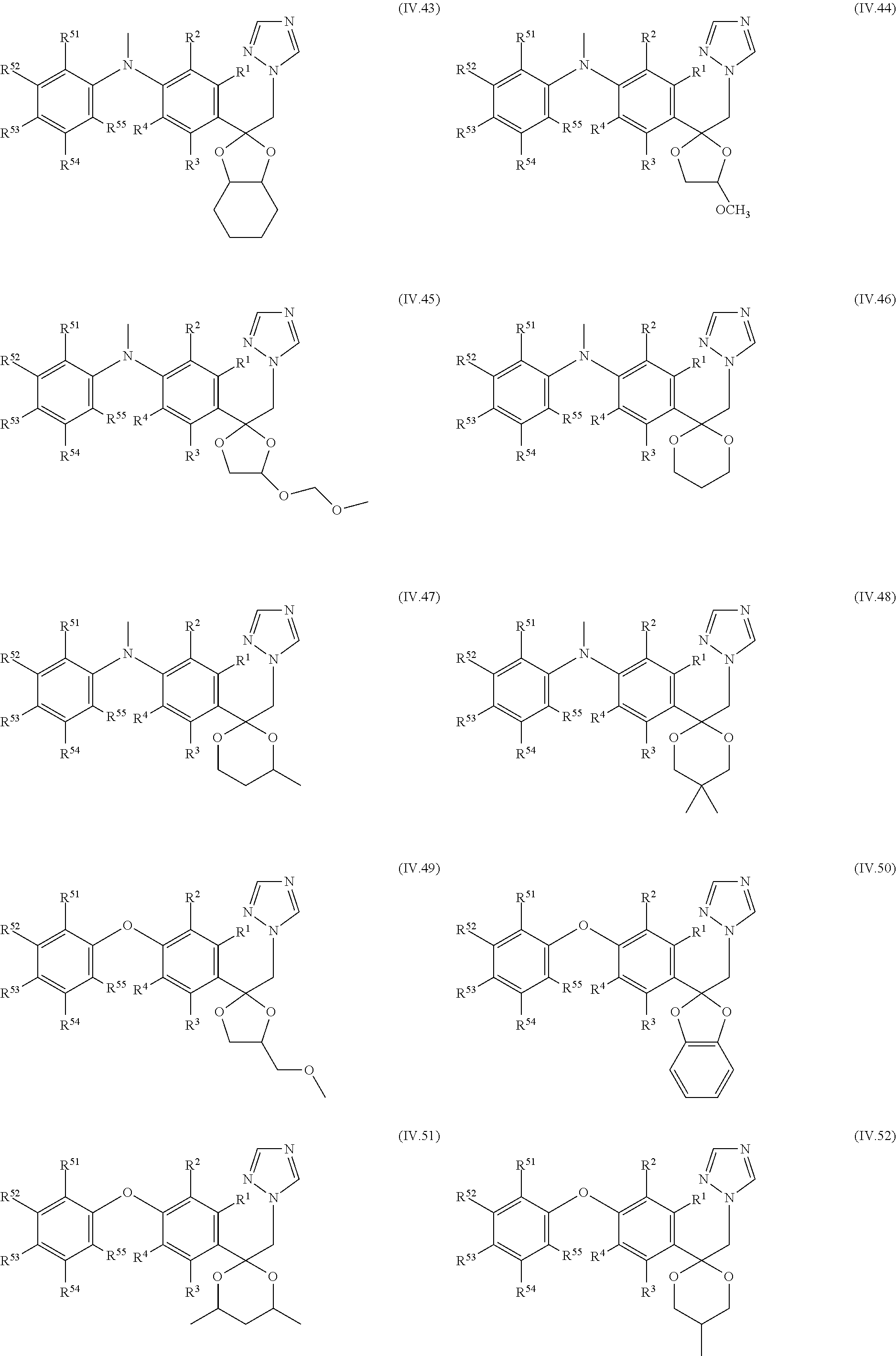

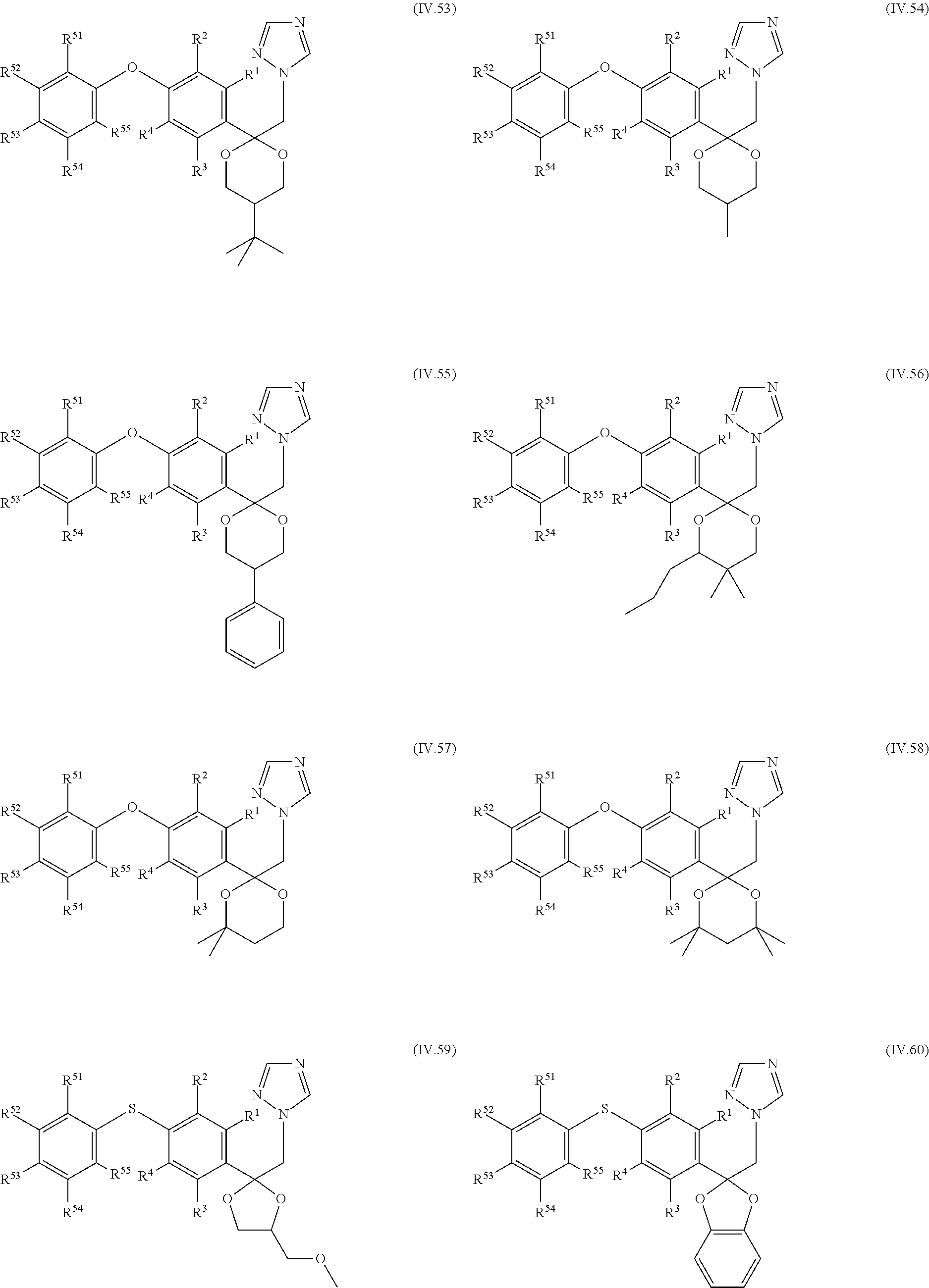

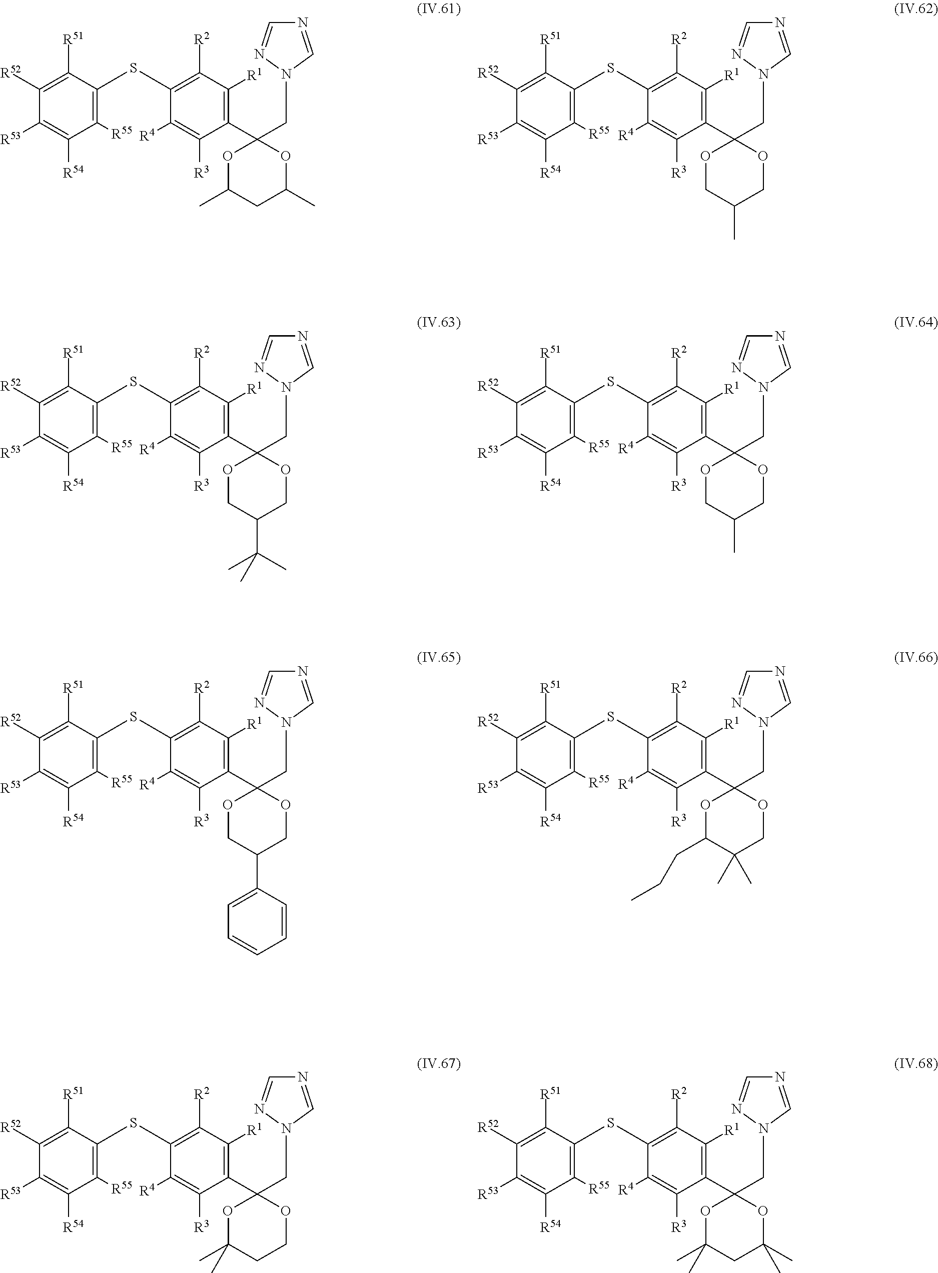

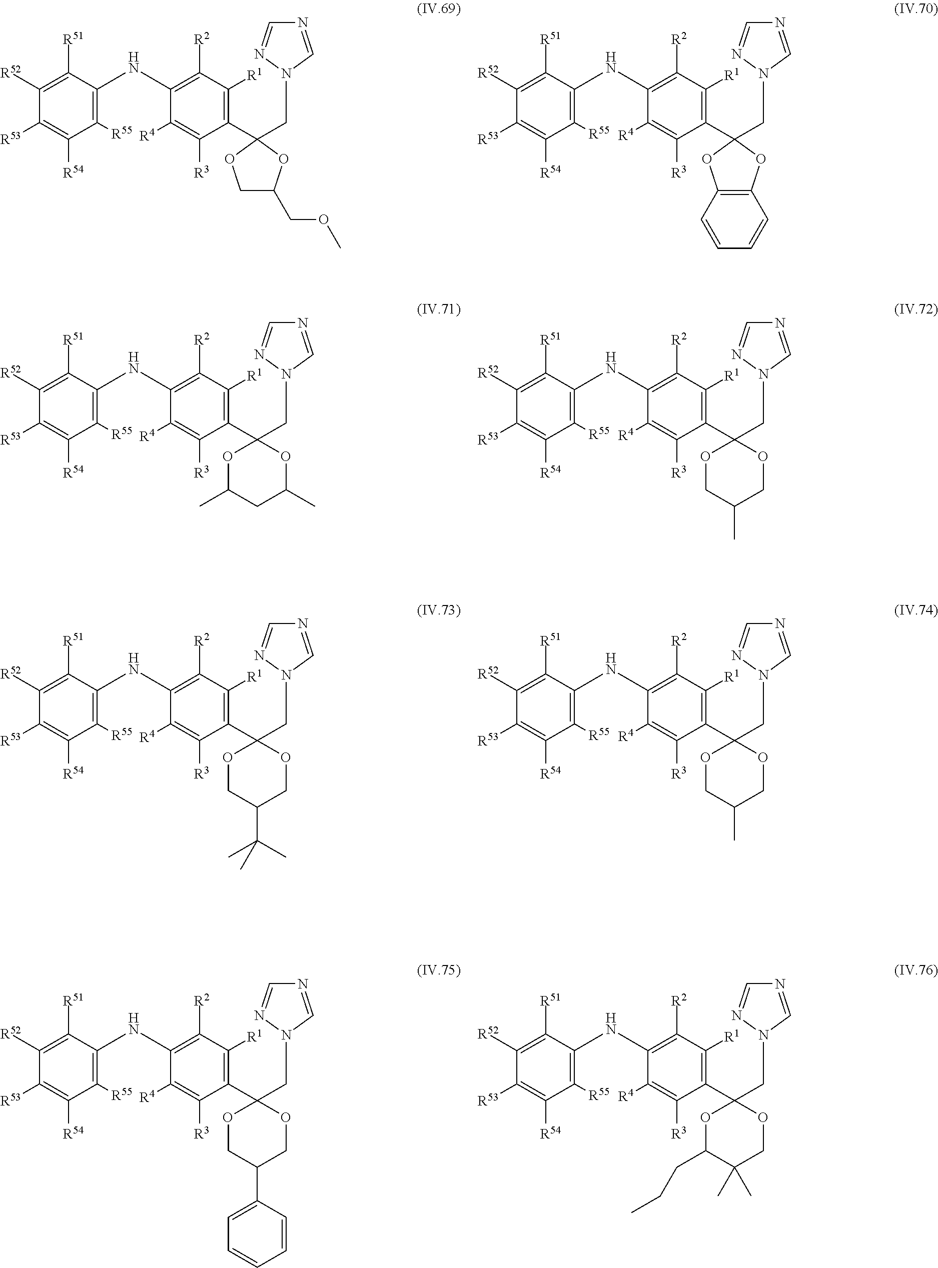

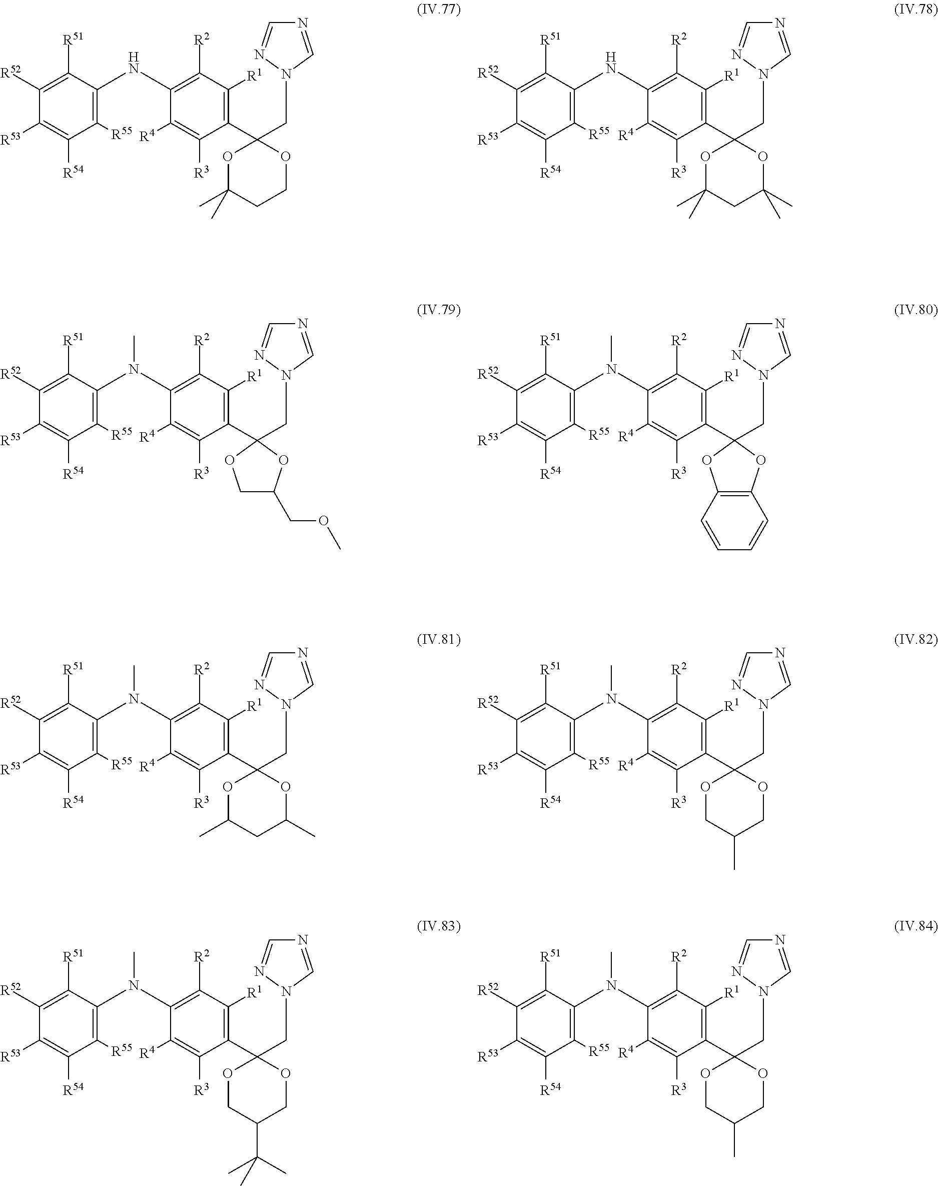

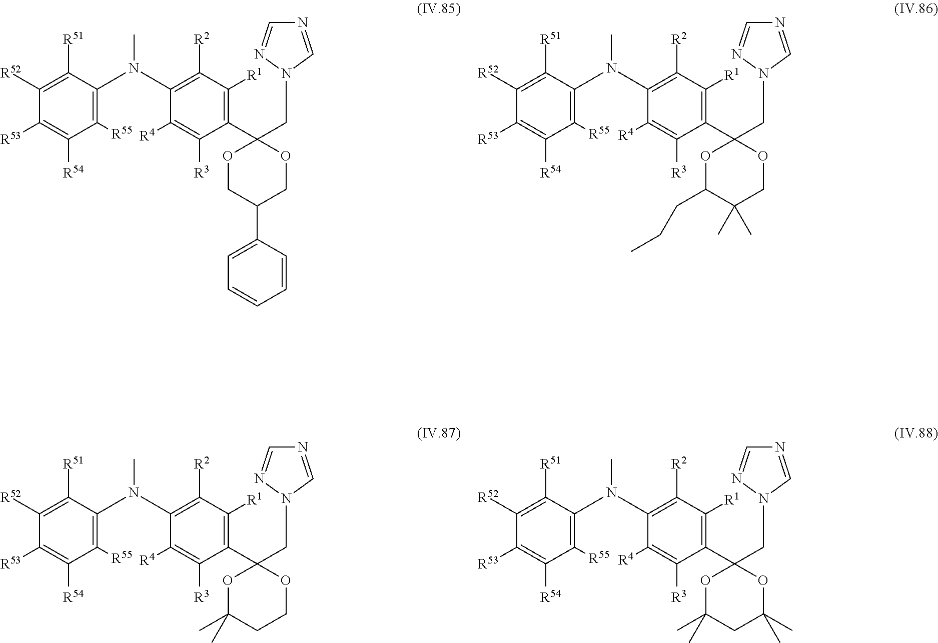

In one embodiment, A in compounds IV is a linear C₂-alkylene bridge which may carry 1, 2, 3 or 4 substituents R⁷, where two substituents R⁷ bound on two adjacent carbon atoms, together with the carbon atoms to which they are bound, may form a cyclopentyl, cyclohexyl or phenyl ring. Preferably, Y is O.

Among these compounds, preference is given to the compounds of formula IVA

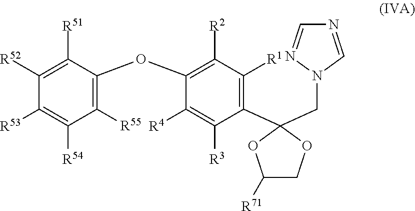

wherein R⁵¹ is F and at least one of R⁵¹, R⁵³, R⁵⁴ or R⁵⁵ is F or Cl.

Among these, preference is given to compounds IVA, wherein R⁵¹ is F and one of R⁵¹, R⁵³, R⁵⁴ or R⁵⁵ is F or Cl. More preference is given to compounds IVA, wherein R⁵¹ is F, one of R⁵¹, R⁵³, R⁵⁴ or R⁵⁵ is F or Cl and the others are hydrogen.

Among these, particular preference is given to compounds IVA, wherein
R¹, R², R³ and R⁴ are hydrogen, R⁵¹ is F, R⁵³ is Cl, R⁵², R⁵⁴ and R⁵⁵ are hydrogen and R⁷¹ is methyl (compound IVA.1);
R¹, R², R³ and R⁴ are hydrogen, R⁵¹ and R⁵³ are F, R⁵², R⁵⁴ and R⁵⁵ are hydrogen and R⁷¹ is methyl (compound IVA.2);
R¹, R², R³ and R⁴ are hydrogen, R⁵¹ and R⁵³ are F, R⁵², R⁵⁴ and R⁵⁵ are hydrogen and R⁷¹ is ethyl (compound IVA.3);
R¹, R², R³ and R⁴ are hydrogen, R⁵¹ is F, R⁵⁵ is Cl, R⁵², R⁵³ and R⁵⁴ are hydrogen and R⁷¹ is methyl (compound IVA.4);
R¹, R², R³ and R⁴ are hydrogen, R⁵¹ is F, R⁵⁵ is Cl, R⁵², R⁵³ and R⁵⁴ are hydrogen and R⁷¹ is ethyl (compound IVA.5);
R¹, R², R³ and R⁴ are hydrogen, R⁵¹ is F, R⁵⁵ is Cl, R⁵², R⁵³ and R⁵⁴ are hydrogen and R⁷¹ is n-propyl (compound IVA.6);
R¹, R², R³ and R⁴ are hydrogen, R⁵¹ and R⁵⁵ are F, R⁵², R⁵³ and R⁵⁴ are hydrogen and R⁷¹ is ethyl (compound IVA.7);
R¹, R², R³ and R⁴ are hydrogen, R⁵¹ and R⁵⁵ are F, R⁵², R⁵³ and R⁵⁴ are hydrogen and R⁷¹ is n-propyl (compound IVA.8);
R¹, R², R³ and R⁴ are hydrogen, R⁵¹ is F, R⁵⁴ is Cl, R⁵², R⁵³ and R⁵⁵ are hydrogen and R⁷¹ is methyl (compound IVA.9);
R¹, R², R³ and R⁴ are hydrogen, R⁵¹ is F, R⁵⁴ is Cl, R⁵², R⁵³ and R⁵⁵ are hydrogen and R⁷¹ is ethyl (compound IVA.10);
R¹, R², R³ and R⁴ are hydrogen, R⁵¹ is F, R⁵⁴ is Cl, R⁵², R⁵³ and R⁵⁵ are hydrogen and R⁷¹ is n-propyl (compound IVA.11);
R¹, R², R³ and R⁴ are hydrogen, R⁵¹ is F, R⁵³ is Cl, R⁵², R⁵⁴ and R⁵⁵ are hydrogen and R⁷¹ is ethyl (compound IVA.12);
R¹, R², R³ and R⁴ are hydrogen, R⁵¹ is F, R⁵³ is Cl, R⁵², R⁵⁴ and R⁵⁵ are hydrogen and R⁷¹ is n-propyl (compound IVA.13);
R¹, R², R³ and R⁴ are hydrogen, R⁵¹ and R⁵⁴ are F, R⁵², R⁵³ and R⁵⁵ are hydrogen and R⁷¹ is methyl (compound IVA.14);
R¹, R², R³ and R⁴ are hydrogen, R⁵¹ and R⁵⁴ are F, R⁵², R⁵³ and R⁵⁵ are hydrogen and R⁷¹ is ethyl (compound IVA.15);
R¹, R², R³ and R⁴ are hydrogen, R⁵¹ and R⁵⁴ are F, R⁵², R⁵³ and R⁵⁵ are hydrogen and R⁷¹ is n-propyl (compound IVA.16);
R¹ is Cl, R², R³ and R⁴ are hydrogen, R⁵¹ and R⁵³ are F, R⁵², R⁵⁴ and R⁵⁵ are hydrogen and R⁷¹ is methyl (compound IVA.17);
R¹ is Cl, R², R³ and R⁴ are hydrogen, R⁵¹ and R⁵⁵ are F, R⁵², R⁵³ and R⁵⁴ are hydrogen and R⁷¹ is methyl (compound IVA.18);
R¹ is methyl, R², R³ and R⁴ are hydrogen, R⁵¹ and R⁵³ are F, R⁵², R⁵⁴ and R⁵⁵ are hydrogen and R⁷¹ is methyl (compound IVA.19);
R¹ is Cl, R², R³ and R⁴ are hydrogen, R⁵¹ is F, R⁵³ is Cl, R⁵², R⁵⁴ and R⁵⁵ are hydrogen and R⁷¹ is methyl (compound IVA.20);
R¹ is methyl, R², R³ and R⁴ are hydrogen, R⁵¹ is F, R⁵³ is Cl, R⁵², R⁵⁴ and R⁵⁵ are hydrogen and R⁷¹ is methyl (compound IVA.21);
R¹ is trifluoromethyl, R², R³ and R⁴ are hydrogen, R⁵¹ is F, R⁵³ is Cl, R⁵², R⁵⁴ and R⁵⁵ are hydrogen and R⁷¹ is methyl (compound IVA.22);
R¹ is Cl, R², R³ and R⁴ are hydrogen, R⁵¹ is F, R⁵⁵ is Cl, R⁵², R⁵³ and R⁵⁴ are hydrogen and R⁷¹ is methyl (compound IVA.23);
R¹ is Cl, R², R³ and R⁴ are hydrogen, R⁵¹ and R⁵⁴ are F, R⁵², R⁵³ and R⁵⁵ are hydrogen and R⁷¹ is methyl (compound IVA.24);
R¹ is methyl, R², R³ and R⁴ are hydrogen, R⁵¹ and R⁵⁴ are F, R⁵², R⁵³ and R⁵⁵ are hydrogen and R⁷¹ is methyl (compound IVA.25);
R¹ is Cl, R⁴ is F, R² and R³ are hydrogen, R⁵¹ is F, R⁵³ is Cl, R⁵², R⁵⁴ and R⁵⁵ are hydrogen and R⁷¹ is methyl (compound IVA.29);
R¹ is Cl, R⁴ is F, R² and R³ are hydrogen, R⁵¹ and R⁵³ are F, R⁵², R⁵⁴ and R⁵⁵ are hydrogen and R⁷¹ is methyl (compound IVA.34).

Alternatively, preference is given to compounds of formula IVA

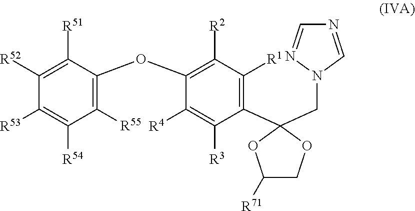

wherein
R¹ is methyl, R², R³ and R⁴ are hydrogen, R⁵¹ and R⁵³ are Cl, R⁵², R⁵⁴ and R⁵⁵ are hydrogen and R⁷¹ is methyl (compound IVA.26);
R¹ is methyl, R², R³ and R⁴ are hydrogen, R⁵² and R⁵⁴ are Cl, R⁵¹, R⁵³ and R⁵⁵ are hydrogen and R⁷¹ is methyl (compound IVA.27);
R¹ is methyl, R², R³ and R⁴ are hydrogen, R⁵¹ is Cl, R⁵², R⁵³, R⁵⁴ and R⁵⁵ are hydrogen and R⁷¹ is methyl (compound IVA.28);
R² is F, R¹, R³ and R⁴ are hydrogen, R⁵³ is Cl, R⁵¹, R⁵², R⁵⁴ and R⁵⁵ are hydrogen and R⁷¹ is methyl (compound IVA.30);
R¹ is F, R², R³ and R⁴ are hydrogen, R⁵¹, R⁵², R⁵³, R⁵⁴ and R⁵⁵ are hydrogen and R⁷¹ is methyl (compound IVA.31);
R¹ is F, R², R³ and R⁴ are hydrogen, R⁵³ is Cl, R⁵¹, R⁵², R⁵⁴ and R⁵⁵ are hydrogen and R⁷¹ is methyl (compound IVA.32);
R² is F, R¹, R³ and R⁴ are hydrogen, R⁵¹, R⁵², R⁵³, R⁵⁴ and R⁵⁵ are hydrogen and R⁷¹ is methyl (compound IVA.33);
R¹ and R⁴ are F, R² and R³ are hydrogen, R⁵³ is Cl, R⁵¹, R⁵², R⁵⁴ and R⁵⁵ are hydrogen and R⁷¹ is methyl (compound IVA.35);
R¹ is Cl, R², R³ and R⁴ are hydrogen, R⁵³ is Cl, R⁵¹, R⁵², R⁵⁴ and R⁵⁵ are hydrogen and R⁷¹ is ethyl (compound IVA.36);
R¹ is Cl, R², R³ and R⁴ are hydrogen, R⁵³ is Cl, R⁵¹, R⁵², R⁵⁴ and R⁵⁵ are hydrogen and R⁷¹ is n-propyl (compound IVA.37);
R² and R³ are F, R¹ and R⁴ are hydrogen, R⁵¹, R⁵², R⁵³, R⁵⁴ and R⁵⁵ are hydrogen and R⁷¹ is methyl (compound IVA.38);
R² and R⁴ are F, R¹ and R³ are hydrogen, R⁵¹, R⁵², R⁵³, R⁵⁴ and R⁵⁵ are hydrogen and R⁷¹ is methyl (compound IVA.39);
R¹ is trifluoromethyl, R², R³ and R⁴ are hydrogen, R⁵³ is Cl, R⁵¹, R⁵², R⁵⁴ and R⁵⁵ are hydrogen and R⁷¹ is methyl (compound IVA.40);
R² is F, R³ is Cl, R¹ and R⁴ are hydrogen, R⁵³ is Cl, R⁵¹, R⁵², R⁵⁴ and R⁵⁵ are hydrogen and R⁷¹ is methyl (compound IVA.41);
R¹ is trifluoromethyl, R², R³ and R⁴ are hydrogen, R⁵¹ and R⁵³ are Cl, R⁵², R⁵⁴ and R⁵⁵ are hydrogen and R⁷¹ is methyl (compound IVA.42);
R¹ is trifluoromethyl, R², R³ and R⁴ are hydrogen, R⁵¹ and R⁵³ are Cl, R⁵², R⁵⁴ and R⁵⁵ are hydrogen and R⁷¹ is hydrogen (compound IVA.43);
R¹ is trifluoromethyl, R², R³ and R⁴ are hydrogen, R⁵¹ is Cl, R⁵², R⁵³, R⁵⁴ and R⁵⁵ are hydrogen and R⁷¹ is hydrogen (compound IVA.44);
R¹ is Cl, R⁴ is F, R² and R³ are hydrogen, R⁵¹ and R⁵³ are Cl, R⁵², R⁵⁴ and R⁵⁵ are hydrogen and R⁷¹ is methyl (compound IVA.45);
R¹ is Cl, R⁴ is F, R² and R³ are hydrogen, R⁵¹ is Cl, R⁵², R⁵³, R⁵⁴ and R⁵⁵ are hydrogen and R⁷¹ is methyl (compound IVA.46).

In sum, particular preference is given to compounds of formula IVA

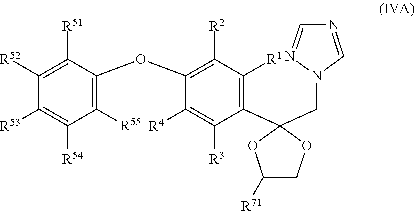

wherein
R¹, R², R³ and R⁴ are hydrogen, R⁵¹ is F, R⁵³ is Cl, R⁵², R⁵⁴ and R⁵⁵ are hydrogen and R⁷¹ is methyl (compound IVA.1);
R¹, R², R³ and R⁴ are hydrogen, R⁵¹ and R⁵³ are F, R⁵², R⁵⁴ and R⁵⁵ are hydrogen and R⁷¹ is methyl (compound IVA.2);
R¹, R², R³ and R⁴ are hydrogen, R⁵¹ and R⁵³ are F, R⁵², R⁵⁴ and R⁵⁵ are hydrogen and R⁷¹ is ethyl (compound IVA.3);
R¹, R², R³ and R⁴ are hydrogen, R⁵¹ is F, R⁵⁵ is Cl, R⁵², R⁵³ and R⁵⁴ are hydrogen and R⁷¹ is methyl (compound IVA.4);
R¹, R², R³ and R⁴ are hydrogen, R⁵¹ is F, R⁵⁵ is Cl, R⁵², R⁵³ and R⁵⁴ are hydrogen and R⁷¹ is ethyl (compound IVA.5);
R¹, R², R³ and R⁴ are hydrogen, R⁵¹ is F, R⁵⁵ is Cl, R⁵², R⁵³ and R⁵⁴ are hydrogen and R⁷¹ is n-propyl (compound IVA.6);
R¹, R², R³ and R⁴ are hydrogen, R⁵¹ and R⁵⁵ are F, R⁵², R⁵³ and R⁵⁴ are hydrogen and R⁷¹ is ethyl (compound IVA.7);
R¹, R², R³ and R⁴ are hydrogen, R⁵¹ and R⁵⁵ are F, R⁵², R⁵³ and R⁵⁴ are hydrogen and R⁷¹ is n-propyl (compound IVA.8);
R¹, R², R³ and R⁴ are hydrogen, R⁵¹ is F, R⁵⁴ is Cl, R⁵², R⁵³ and R⁵⁵ are hydrogen and R⁷¹ is methyl (compound IVA.9);
R¹, R², R³ and R⁴ are hydrogen, R⁵¹ is F, R⁵⁴ is Cl, R⁵², R⁵³ and R⁵⁵ are hydrogen and R⁷¹ is ethyl (compound IVA.10);
R¹, R², R³ and R⁴ are hydrogen, R⁵¹ is F, R⁵⁴ is Cl, R⁵², R⁵³ and R⁵⁵ are hydrogen and R⁷¹ is n-propyl (compound IVA.11);
R¹, R², R³ and R⁴ are hydrogen, R⁵¹ is F, R⁵³ is Cl, R⁵², R⁵⁴ and R⁵⁵ are hydrogen and R⁷¹ is ethyl (compound IVA.12);
R¹, R², R³ and R⁴ are hydrogen, R⁵¹ is F, R⁵³ is Cl, R⁵², R⁵⁴ and R⁵⁵ are hydrogen and R⁷¹ is n-propyl (compound IVA.13);
R¹, R², R³ and R⁴ are hydrogen, R⁵¹ and R⁵⁴ are F, R⁵², R⁵³ and R⁵⁵ are hydrogen and R⁷¹ is methyl (compound IVA.14);
R¹, R², R³ and R⁴ are hydrogen, R⁵¹ and R⁵⁴ are F, R⁵², R⁵³ and R⁵⁵ are hydrogen and R⁷¹ is ethyl (compound IVA.15);
R¹, R², R³ and R⁴ are hydrogen, R⁵¹ and R⁵⁴ are F, R⁵², R⁵³ and R⁵⁵ are hydrogen and R⁷¹ is n-propyl (compound IVA.16);
R¹ is Cl, R², R³ and R⁴ are hydrogen, R⁵¹ and R⁵³ are F, R⁵², R⁵⁴ and R⁵⁵ are hydrogen and R⁷¹ is methyl (compound IVA.17);
R¹ is Cl, R², R³ and R⁴ are hydrogen, R⁵¹ and R⁵⁵ are F, R⁵², R⁵³ and R⁵⁴ are hydrogen and R⁷¹ is methyl (compound IVA.18);
R¹ is methyl, R², R³ and R⁴ are hydrogen, R⁵¹ and R⁵³ are F, R⁵², R⁵⁴ and R⁵⁵ are hydrogen and R⁷¹ is methyl (compound IVA.19);
R¹ is Cl, R², R³ and R⁴ are hydrogen, R⁵¹ is F, R⁵³ is Cl, R⁵², R⁵⁴ and R⁵⁵ are hydrogen and R⁷¹ is methyl (compound IVA.20);
R¹ is methyl, R², R³ and R⁴ are hydrogen, R⁵¹ is F, R⁵³ is Cl, R⁵², R⁵⁴ and R⁵⁵ are hydrogen and R⁷¹ is methyl (compound IVA.21);
R¹ is trifluoromethyl, R², R³ and R⁴ are hydrogen, R⁵¹ is F, R⁵³ is Cl, R⁵², R⁵⁴ and R⁵⁵ are hydrogen and R⁷¹ is methyl (compound IVA.22);
R¹ is Cl, R², R³ and R⁴ are hydrogen, R⁵¹ is F, R⁵⁵ is Cl, R⁵², R⁵³ and R⁵⁴ are hydrogen and R⁷¹ is methyl (compound IVA.23);
R¹ is Cl, R², R³ and R⁴ are hydrogen, R⁵¹ and R⁵⁴ are F, R⁵², R⁵³ and R⁵⁵ are hydrogen and R⁷¹ is methyl (compound IVA.24);
R¹ is methyl, R², R³ and R⁴ are hydrogen, R⁵¹ and R⁵⁴ are F, R⁵², R⁵³ and R⁵⁵ are hydrogen and R⁷¹ is methyl (compound IVA.25);
R¹ is methyl, R², R³ and R⁴ are hydrogen, R⁵¹ and R⁵³ are Cl, R⁵², R⁵⁴ and R⁵⁵ are hydrogen and R⁷¹ is methyl (compound IVA.26);
R¹ is methyl, R², R³ and R⁴ are hydrogen, R⁵² and R⁵⁴ are Cl, R⁵¹, R⁵³ and R⁵⁵ are hydrogen and R⁷¹ is methyl (compound IVA.27);
R¹ is methyl, R², R³ and R⁴ are hydrogen, R⁵¹ is Cl, R⁵², R⁵³, R⁵⁴ and R⁵⁵ are hydrogen and R⁷¹ is methyl (compound IVA.28);
R¹ is Cl, R⁴ is F, R² and R³ are hydrogen, R⁵¹ is F, R⁵³ is Cl, R⁵², R⁵⁴ and R⁵⁵ are hydrogen and R⁷¹ is methyl (compound IVA.29);
R² is F, R¹, R³ and R⁴ are hydrogen, R⁵³ is Cl, R⁵¹, R⁵², R⁵⁴ and R⁵⁵ are hydrogen and R⁷¹ is methyl (compound IVA.30);
R¹ is F, R², R³ and R⁴ are hydrogen, R⁵¹, R⁵², R⁵³, R⁵⁴ and R⁵⁵ are hydrogen and R⁷¹ is methyl (compound IVA.31);
R¹ is F, R², R³ and R⁴ are hydrogen, R⁵³ is Cl, R⁵¹, R⁵², R⁵⁴ and R⁵⁵ are hydrogen and R⁷¹ is methyl (compound IVA.32);
R² is F, R¹, R³ and R⁴ are hydrogen, R⁵¹, R⁵², R⁵³, R⁵⁴ and R⁵⁵ are hydrogen and R⁷¹ is methyl (compound IVA.33);
R¹ is Cl, R⁴ is F, R² and R³ are hydrogen, R⁵¹ and R⁵³ are F, R⁵², R⁵⁴ and R⁵⁵ are hydrogen and R⁷¹ is methyl (compound IVA.34);
R¹ and R⁴ are F, R² and R³ are hydrogen, R⁵³ is Cl, R⁵¹, R⁵², R⁵⁴ and R⁵⁵ are hydrogen and R⁷¹ is methyl (compound IVA.35);
R¹ is Cl, R², R³ and R⁴ are hydrogen, R⁵³ is Cl, R⁵¹, R⁵², R⁵⁴ and R⁵⁵ are hydrogen and R⁷¹ is ethyl (compound IVA.36);
R¹ is Cl, R², R³ and R⁴ are hydrogen, R⁵³ is Cl, R⁵¹, R⁵², R⁵⁴ and R⁵⁵ are hydrogen and R⁷¹ is n-propyl (compound IVA.37);
R² and R³ are F, R¹ and R⁴ are hydrogen, R⁵¹, R⁵², R⁵³, R⁵⁴ and R⁵⁵ are hydrogen and R⁷¹ is methyl (compound IVA.38);
R² and R⁴ are F, R¹ and R³ are hydrogen, R⁵¹, R⁵², R⁵³, R⁵⁴ and R⁵⁵ are hydrogen and R⁷¹ is methyl (compound IVA.39);
R¹ is trifluoromethyl, R², R³ and R⁴ are hydrogen, R⁵³ is Cl, R⁵¹, R⁵², R⁵⁴ and R⁵⁵ are hydrogen and R⁷¹ is methyl (compound IVA.40);
R² is F, R³ is Cl, R¹ and R⁴ are hydrogen, R⁵³ is Cl, R⁵¹, R⁵², R⁵⁴ and R⁵⁵ are hydrogen and R⁷¹ is methyl (compound IVA.41);
R¹ is trifluoromethyl, R², R³ and R⁴ are hydrogen, R⁵¹ and R⁵³ are Cl, R⁵², R⁵⁴ and R⁵⁵ are hydrogen and R⁷¹ is methyl (compound IVA.42);
R¹ is trifluoromethyl, R², R³ and R⁴ are hydrogen, R⁵¹ and R⁵³ are Cl, R⁵², R⁵⁴ and R⁵⁵ are hydrogen and R⁷¹ is hydrogen (compound IVA.43);
R¹ is trifluoromethyl, R², R³ and R⁴ are hydrogen, R⁵¹ is Cl, R⁵², R⁵³, R⁵⁴ and R⁵⁵ are hydrogen and R⁷¹ is hydrogen (compound IVA.44);
R¹ is Cl, R⁴ is F, R² and R³ are hydrogen, R⁵¹ and R⁵³ are Cl, R⁵², R⁵⁴ and R⁵⁵ are hydrogen and R⁷¹ is methyl (compound IVA.45);
R¹ is Cl, R⁴ is F, R² and R³ are hydrogen, R⁵¹ is Cl, R⁵², R⁵³, R⁵⁴ and R⁵⁵ are hydrogen and R⁷¹ is methyl (compound IVA.46).

Moreover, preference is given to compounds of formulae IVB and IVC

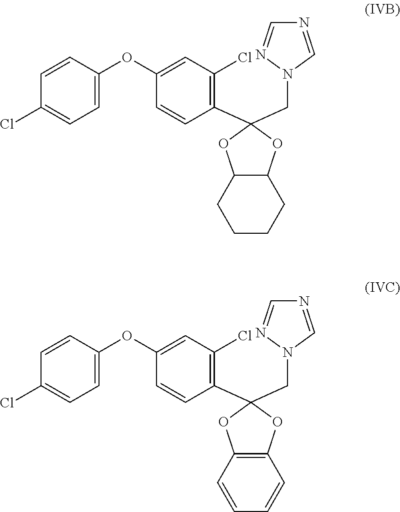

In an alternative embodiment, in compounds IV, A is a linear C₄-C₅-alkylene bridge which may be substituted by 1, 2, 3 or 4 substituents R⁷. Y is preferably O. As regards preferred meanings of the remaining substituents, reference is made to what has been said above for compounds I and II.

It is however particularly preferred that A in compounds IV is a linear C₃-alkylene bridge which may be substituted by 1, 2, 3, 4, 5 or 6, preferably 1, 2, 3 or 4 radicals R⁷. R⁷ is preferably selected from C₁-C₄-alkyl and phenyl. Y is preferably O.

Among these, preference is given to compounds IV, wherein
A is a linear C₃-alkylene bridge which may be substituted by 1, 2, 3 or 4 substituents R⁷;
R¹ is selected from fluorine, chlorine, methyl and trifluoromethyl;
R², R³ and R⁴ are hydrogen;
(R⁵)ₙ is 4-Cl, relative to the 1-position of the attachment point of the phenyl ring to the group Y;
each R⁷ is independently selected from C₁-C₄-alkyl and phenyl;
with the provisos that
R¹ is not Cl if Y is O and A is —(CH₂)₃—;
R¹ is not methyl if Y is O and A is —(CH₂)₃— or —CH(CH₃)CH₂CH₂—.

Particular preference is given to compounds of formula IVD

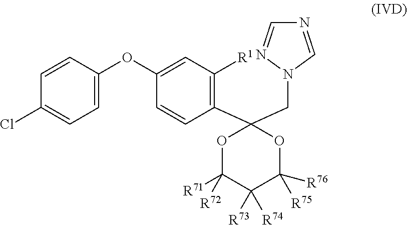

wherein
R¹ is Cl, R⁷¹ is methyl and R⁷², R⁷³, R⁷⁴, R⁷⁵ and R⁷⁶ are hydrogen (compound IVD.1);
R¹ is Cl, R⁷¹ and R⁷⁶ are methyl and R⁷², R⁷³, R⁷⁴ and R⁷⁵ are hydrogen (compound IVD.2);
R¹ is Cl, R⁷³ is tert-butyl and R⁷¹, R⁷², R⁷⁴, R⁷⁵ and R⁷⁶ are hydrogen (compound IVD.3);
R¹ is Cl, R⁷³ is phenyl and R⁷¹, R⁷², R⁷⁴, R⁷⁵ and R⁷⁶ are hydrogen (compound IVD.4);
R¹ is Cl, R⁷³ is methyl and R⁷¹, R⁷², R⁷⁴, R⁷⁵ and R⁷⁶ are hydrogen (compound IVD.5);
R¹ is Cl, R⁷³ and R⁷⁴ are methyl and R⁷¹, R⁷², R⁷⁵ and R⁷⁶ are hydrogen (compound IVD.6);
R¹ is Cl, R⁷¹ is n-propyl, R⁷³ and R⁷⁴ are methyl and R⁷², R⁷⁵ and R⁷⁶ are hydrogen (compound IVD.7);
R¹ is Cl, R⁷¹ and R⁷² are methyl and R⁷³, R⁷⁴, R⁷⁵ and R⁷⁶ are hydrogen (compound IVD.8);
R¹ is Cl, R⁷¹, R⁷², R⁷⁵ and R⁷⁶ are methyl and R⁷³ and R⁷⁴ are hydrogen (compound IVD.9);
R¹ is methyl and R⁷¹, R⁷², R⁷³, R⁷⁴, R⁷⁵ and R⁷⁶ are hydrogen (compound IVD.10);
R¹ is methyl, R⁷¹ is methyl and R⁷², R⁷³, R⁷⁴, R⁷⁵ and R⁷⁶ are hydrogen (compound IVD.11);
R¹ is methyl, R⁷¹ and R⁷² are methyl and R⁷³, R⁷⁴, R⁷⁵ and R⁷⁶ are hydrogen (compound IVD.12);
R¹ is methyl, R⁷¹, R⁷², R⁷⁵ and R⁷⁶ are methyl and R⁷³ and R⁷⁴ are hydrogen (compound IVD.13);
R¹ is methyl, R⁷¹ and R⁷⁶ are methyl and R⁷², R⁷³, R⁷⁴ and R⁷⁵ are hydrogen (compound IVD.14);
R¹ is methyl, R⁷³ is tert-butyl and R⁷¹, R⁷², R⁷⁴, R⁷⁵ and R⁷⁶ are hydrogen (compound IVD.15);
R¹ is methyl, R⁷³ is phenyl and R⁷¹, R⁷², R⁷⁴, R⁷⁵ and R⁷⁶ are hydrogen (compound IVD.16);
R¹ is methyl, R⁷³ is methyl and R⁷¹, R⁷², R⁷⁴, R⁷⁵ and R⁷⁶ are hydrogen (compound IVD.17);
R¹ is methyl, R⁷³ and R⁷⁴ are methyl and R⁷¹, R⁷², R⁷⁵ and R⁷⁶ are hydrogen (compound IVD.18);
R¹ is methyl, R⁷¹ is n-propyl, R⁷³ and R⁷⁴ are methyl and R⁷², R⁷⁵ and R⁷⁶ are hydrogen (compound IVD.19);
R¹ is trifluoromethyl and R⁷¹, R⁷², R⁷³, R⁷⁴, R⁷⁵ and R⁷⁶ are hydrogen (compound IVD.20);
R¹ is trifluoromethyl, R⁷¹ is methyl and R⁷², R⁷³, R⁷⁴, R⁷⁵ and R⁷⁶ are hydrogen (compound IVD.21);
R¹ is trifluoromethyl, R⁷¹ and R⁷⁶ are methyl and R⁷², R⁷³, R⁷⁴ and R⁷⁵ are hydrogen (compound IVD.22);
R¹ is trifluoromethyl, R⁷³ is tert-butyl and R⁷¹, R⁷², R⁷⁴, R⁷⁵ and R⁷⁶ are hydrogen (compound IVD.23);
R¹ is trifluoromethyl, R⁷³ is phenyl and R⁷¹, R⁷², R⁷⁴, R⁷⁵ and R⁷⁶ are hydrogen (compound IVD.24);
R¹ is trifluoromethyl, R⁷³ is methyl and R⁷¹, R⁷², R⁷⁴, R⁷⁵ and R⁷⁶ are hydrogen (compound IVD.25);
R¹ is trifluoromethyl, R⁷³ and R⁷⁴ are methyl and R⁷¹, R⁷², R⁷⁵ and R⁷⁶ are hydrogen (compound IVD.26);
R¹ is trifluoromethyl, R⁷¹ is n-propyl, R⁷³ and R⁷⁴ are methyl and R⁷², R⁷⁵ and R⁷⁶ are hydrogen (compound IVD.27);
R¹ is trifluoromethyl, R⁷¹ and R⁷² are methyl and R⁷³, R⁷⁴, R⁷⁵ and R⁷⁶ are hydrogen (compound IVD.28);
R¹ is trifluoromethyl, R⁷¹, R⁷², R⁷⁵ and R⁷⁶ are methyl and R⁷³ and R⁷⁴ are hydrogen (compound IVD.29).

The invention further refers to an agricultural composition comprising at least one compound of formula I, II and/or IV as defined above or an agriculturally acceptable salt thereof and a liquid or solid carrier. Suitable carriers, as well as auxiliaries and further active compounds which may also be contained in the composition of the invention are defined below.

The compounds I and II as well as IV and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically effective and they can be used in crop protection as foliar fungicides, fungicides for seed dressing and soil fungicides. Moreover, they are suitable for controlling harmful fungi, which inter alia occur in wood or roots of plants.

The compounds I, II and IV and the compositions according to the invention are particularly important in the control of a multitude of phytopathogenic fungi on various cultivated plants, such as cereals, e.g. wheat, rye, barley, triticale, oats or rice; beet, e.g. sugar beet or fodder beet; fruits, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp orjute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; natural rubber plants or ornamental and forestry plants, such as flowers, shrubs, broad-leaved trees or evergreens, e.g. conifers; and on the plant propagation material, such as seeds, and the crop material of these plants.

Preferably, compounds I, II and IV and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I, II and IV and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://www.bio.org/speeches/pubs/er/agri_products.asp). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e.g. by glycosylation or polymer additions such as prenylated, acetylated or farnesylated moieties or PEG moieties.

Plants that have been modified by breeding, mutagenesis or genetic engineering, e.g. have been rendered tolerant to applications of specific classes of herbicides, such as hydroxyphenylpyruvate dioxygenase (HPPD) inhibitors; acetolactate synthase (ALS) inhibitors, such as sulfonyl ureas (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e.g. U.S. Pat. No. 6,222,100, WO 01/82685, WO 00/026390, WO 97/41218, WO 98/002526, WO 98/02527, WO 04/106529, WO 05/20673, WO 03/014357, WO 03/13225, WO 03/14356, WO 04/16073); enolpyruvylshikimate-3-phosphate synthase (EPSPS) inhibitors, such as glyphosate (see e.g. WO 92/00377); glutamine synthetase (GS) inhibitors, such as glufosinate (see e.g. EP-A 242 236, EP-A 242 246) or oxynil herbicides (see e.g. U.S. Pat. No. 5,559,024) as a result of conventional methods of breeding or genetic engineering. Several cultivated plants have been rendered tolerant to herbicides by conventional methods of breeding (mutagenesis), e.g. Clearfield® summer rape (Canola, BASF SE, Germany) being tolerant to imidazolinones, e.g. imazamox. Genetic engineering methods have been used to render cultivated plants, such as soybean, cotton, corn, beets and rape, tolerant to herbicides such as glyphosate and glufosinate, some of which are commercially available under the trade names RoundupReady® (glyphosate-tolerant, Monsanto, U.S.A.) and LibertyLink® (glufosinatetolerant, Bayer CropScience, Germany).

Furthermore, plants are also covered that, by the use of recombinant DNA techniques, are capable to synthesize one or more insecticidal proteins, especially those known from the bacterial genus Bacillus, particularly from Bacillus thuringiensis, such as δ-endotoxins, e.g. CryIA(b), CryIA(c), CryIF, CryIF(a2), CryIIA(b), CryIIIA, CryIIIB(b1) or Cry9c; vegetative insecticidal proteins (VIP), e.g. VIP1, VIP2, VIP3 or VIP3A; insecticidal proteins of bacteria colonizing nematodes, e.g. Photorhabdus spp, or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e.g. WO 02/015701). Further examples of such toxins or genetically modified plants capable of synthesizing such toxins are disclosed, e.g., in EP-A 374 753, WO 93/007278, WO 95/34656, EP-A 427 529, EP-A 451 878, WO 03/18810 und WO 03/52073. The methods for producing such genetically modified plants are generally known to the person skilled in the art and are described, e.g., in the publications mentioned above. These insecticidal proteins contained in the genetically modified plants impart to the plants producing these proteins tolerance to harmful pests from all taxonomic groups of arthropods, especially to beetles (Coleoptera), two-winged insects (Diptera), and moths (Lepidoptera) and to nematodes (Nematoda). Genetically modified plants capable to synthesize one or more insecticidal proteins are, e.g., described in the publications mentioned above, and some of them are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Her-culex® R W (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); BtXtra®, NatureGard®, KnockOut®, BiteGard®, Protecta®, Bt11 (e.g. Agrisure®CB) and Bt176 from Syngenta Seeds SAS, France, (corn cultivars producing the Cry1Ab toxin and PAT enyzme), MIR604 from Syngenta Seeds SAS, France (corn cultivars producing a modified version of the Cry3A toxin, c.f. WO 03/018810), MON 863 from Monsanto Europe S. A., Belgium (corn cultivars producing the Cry3Bb1 toxin), IPC 531 from Monsanto Europe S. A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that, by the use of recombinant DNA techniques, are capable to synthesize one or more proteins to increase the resistance or tolerance of those plants to bacterial, viral or fungal pathogens. Examples of such proteins are the so-called “pathogenesis-related proteins” (PR proteins, see, e.g. EP-A 392 225), plant disease resistance genes (e.g. potato cultivars, which express resistance genes acting against Phytophthora infestans derived from the Mexican wild potato Solanum bulbocastanum) or T4-lysozym (e.g. potato cultivars capable of synthesizing these proteins with increased resistance against bacteria such as Erwinia amylvora). The methods for producing such genetically modified plants are generally known to the person skilled in the art and are described, e.g., in the publications mentioned above.

Furthermore, plants are also covered that, by the use of recombinant DNA techniques, are capable to synthesize one or more proteins to increase the productivity (e.g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that, by the use of recombinant DNA techniques, contain a modified amount of substances of content or new substances of content, specifically to improve human or animal nutrition, e.g. oil crops that produce health-promoting long-chain omega-3 fatty acids or unsaturated omega-9 fatty acids (e.g. Nexera® rape, DOW Agro Sciences, Canada).

Furthermore, plants are also covered that, by the use of recombinant DNA techniques, contain a modified amount of substances of content or new substances of content, specifically to improve raw material production, e.g. potatoes that produce increased amounts of amylopectin (e.g. Amflora® potato, BASF SE, Germany).

The compounds I, II and IV and compositions thereof, respectively, are particularly suitable for controlling the following plant diseases: Albugo spp. (white rust) on ornamentals, vegetables (e.g. A. candida) and sunflowers (e.g. A. tragopogonis); Alternaria spp. (Alternaria leaf spot) on vegetables, rape (A. brassicola or brassicae), sugar beets (A. tenuis), fruits, rice, soybeans, potatoes (e.g. A. solani or A. alternata), tomatoes (e.g. A. solani or A. alternata) and wheat; Aphanomyces spp, on sugar beets and vegetables; Ascochyta spp, on cereals and vegetables, e.g. A. tritici (anthracnose) on wheat and A. hordei on barley; Bipolaris and Drechslera spp. (teleomorph: Cochliobolus spp.), e.g. Southern leaf blight (D. maydis) or Northern leaf blight (B. zeicola) on corn, e.g. spot blotch (B. sorokiniana) on cereals and e.g. B. oryzae on rice and turfs; Blumeria (formerly Erysiphe) graminis (powdery mildew) on cereals (e.g. on wheat or barley); Botrytis cinerea (teleomorph: Botryotinia fuckeliana: grey mold) on fruits and berries (e.g. strawberries), vegetables (e.g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e.g. C. ulmi (Dutch elm disease) on elms; Cercospora spp. (Cercospora leaf spots) on corn (e.g. Gray leaf spot: C. zeae-maydis), rice, sugar beets (e.g. C. beticola), sugar cane, vegetables, coffee, soybeans (e.g. C. sojina or C. kikuchii) and rice; Cladosporium spp, on tomatoes (e.g. C. fulvum: leaf mold) and cereals, e.g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e.g. C. sativus, anamorph: B. sorokiniana) and rice (e.g. C. miyabeanus, anamorph: H. oryzae); Colletotrichum (teleomorph: Glomerella) spp. (anthracnose) on cotton (e.g. C. gossypii), corn (e.g. C. graminicola: Anthracnose stalk rot), soft fruits, potatoes (e.g. C. coccodes black dot), beans (e.g. C. lindemuthianum) and soybeans (e.g. C. truncatum or C. gloeosporioides); Corticium spp., e.g. C. sasakii (sheath blight) on rice; Corynespora cassiicola (leaf spots) on soybeans and ornamentals; Cycloconium spp., e.g. C. oleaginum on olive trees; Cylindrocarpon spp. (e.g. fruit tree canker or young vine decline, teleomorph: Nectria or Neonectria spp.) on fruit trees, vines (e.g. C. liriodendri, teleomorph: Neonectria liriodendri. Black Foot Disease) and ornamentals; Dematophora (teleomorph: Rosellinia) necatrix (root and stem rot) on soybeans; Diaporthe spp., e.g. D. phaseolorum (damping off) on soybeans; Drechslera (syn. Helminthosporium, teleomorph: Pyrenophora) spp. on corn, cereals, such as barley (e.g. D. teres, net blotch) and wheat (e.g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeo-acremonium chlamydosporum), Phaeoacremonium aleophilum and/or Botryosphaeria obtusa; Elsinoe spp, on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e.g. E. pisi), such as cucurbits (e.g. E. cichoracearum), cabbages, rape (e.g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp, on corn (e.g. E. turcicum); Fusarium (teleomorph: Gibberella) spp. (wilt, root or stem rot) on various plants, such as F. graminearum or F. culmorum (root rot, scab or head blight) on cereals (e.g. wheat or barley), F. oxysporum on tomatoes, F. solanion soybeans and F. verticilloides on corn; Gaeumannomyces graminis (take-all) on cereals (e.g. wheat or barley) and corn; Gibberella spp, on cereals (e.g. G. zeae) and rice (e.g. G. fujikuroi. Bakanae disease); Glomerella cingulata on vines, pome fruits and other plants and G. gossypii on cotton; Grain-staining complex on rice; Guignardia bidwellii (black rot) on vines; Gymnosporangium spp, on rosaceous plants and junipers, e.g. G. sabinae (rust) on pears; Helminthosporium spp. (syn. Drechslera, teleomorph: Cochliobolus) on corn, cereals and rice; Hemileia spp., e.g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e.g. wheat or barley); Microsphaera diffusa (powdery mildew) on soybeans; Monilinia spp., e.g. M. laxa, M. fructicola and M. fructigena (bloom and twig blight, brown rot) on stone fruits and other rosaceous plants; Mycosphaerella spp. on cereals, bananas, soft fruits and ground nuts, such as e.g. M. graminicola (anamorph: Septoria tritici, Septoria blotch) on wheat or M. fijiensis (black Sigatoka disease) on bananas; Peronospora spp. (downy mildew) on cabbage (e.g. P. brassicae), rape (e.g. P. parasitica), onions (e.g. P. destructor), tobacco (P. tabacina) and soybeans (e.g. P. manshurica); Phakopsora pachyrhizi and P. meibomiae (soybean rust) on soybeans; Phialophora spp. e.g. on vines (e.g. P. tracheiphila and P. tetraspora) and soybeans (e.g. P. gregata: stem rot); Phoma lingam (root and stem rot) on rape and cabbage and P. betae (root rot, leaf spot and damping-off) on sugar beets; Phomopsis spp. on sunflowers, vines (e.g. P. viticola: can and leaf spot) and soybeans (e.g. stem rot: P. phaseoli teleomorph: Diaporthe phaseolorum); Physoderma maydis (brown spots) on corn; Phytophthora spp. (wilt, root, leaf, fruit and stem root) on various plants, such as paprika and cucurbits (e.g. P. capsici), soybeans (e.g. P. megasperma, syn. P. sojae), potatoes and tomatoes (e.g. P. infestans: late blight) and broad-leaved trees (e.g. P. ramorum: sudden oak death); Plasmodiophora brassicae (club root) on cabbage, rape, radish and other plants; Plasmopara spp., e.g. P. viticola (grapevine downy mildew) on vines and P. halstedii on sunflowers; Podosphaera spp. (powdery mildew) on rosaceous plants, hop, pome and soft fruits, e.g. P. leucotricha on apples; Polymyxa spp., e.g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e.g. wheat or barley; Pseudoperonospora (downy mildew) on various plants, e.g. P. cubensis on cucurbits or P. humili ion hop; Pseudopezicula trachephila (red fire disease or, rotbrenner’, anamorph: Phialophora) on vines; Puccinia spp. (rusts) on various plants, e.g. P. triticina (brown or leaf rust), P. striiformis (stripe or yellow rust), P. hordei(dwarf rust), P. graminis (stem or black rust) or P. recondita (brown or leaf rust) on cereals, such as e.g. wheat, barley or rye, and asparagus (e.g. P. asparagi); Pyrenophora (anamorph: Drechslera) triticirepentis (tan spot) on wheat or P. teres (net blotch) on barley; Pyricularia spp., e.g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e.g. P. ultimum or P. aphanidermatum); Ramularia spp., e.g. R. collo-cygni (Ramularia leaf spots, Physiological leaf spots) on barley and R. beticola on sugar beets; Rhizoctonia spp, on cotton, rice, potatoes, turf, corn, rape, potatoes, sugar beets, vegetables and various other plants, e.g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocadium oryzae and S. attenuatum (sheath rot) on rice; Sclerotinia spp. (stem rot or white mold) on vegetables and field crops, such as rape, sunflowers (e.g. S. scerotiorum) and soybeans (e.g. S. rolfsii or S. scerotiorum); Septoria spp, on various plants, e.g. S. glycines (brown spot) on soybeans, S. tritici(Septoria blotch) on wheat and S. (syn. Stagonospora) nodorum (Stagonospora blotch) on cereals; Uncinula (syn. Erysiphe) necator(powdery mildew, anamorph: Oidium tuckeri) on vines; Setospaeria spp. (leaf blight) on corn (e.g. S. turcicum, syn. Helminthosporium turcicum) and turf; Sphacerotheca spp. (smut) on corn, (e.g. S. reiliana: head smut), sorghum und sugar cane; Sphaerotheca fuliginea (powdery mildew) on cucurbits; Spongospora subterranea (powdery scab) on potatoes and thereby transmitted viral diseases; Stagonospora spp. on cereals, e.g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e.g. T. deformans (leaf curl disease) on peaches and T. pruni (plum pocket) on plums; Thielaviopsis spp. (black root rot) on tobacco, pome fruits, vegetables, soybeans and cotton, e.g. T. basicola (syn. Chalara elegans); Tilletia spp. (common bunt or stinking smut) on cereals, such as e.g. T. tritici (syn. T caries, wheat bunt) and T. controversa (dwarf bunt) on wheat; Typhula incarnata (grey snow mold) on barley or wheat; Urocystis spp., e.g. U. occulta (stem smut) on rye; Uromyces spp. (rust) on vegetables, such as beans (e.g. U. appendiculatus, syn. U. phaseoi) and sugar beets (e.g. U. betae); Ustilago spp. (loose smut) on cereals (e.g. U. nuda and U. avaenae), corn (e.g. U. maydis: corn smut) and sugar cane; Venturia spp. (scab) on apples (e.g. V. inaequalis) and pears; and Verticiium spp. (wilt) on various plants, such as fruits and ornamentals, vines, soft fruits, vegetables and field crops, e.g. V. dahliae on strawberries, rape, potatoes and tomatoes.

The compounds I, II and IV and compositions thereof, respectively, are also suitable for controlling harmful fungi in the protection of stored products or harvest and in the protection of materials. The term “protection of materials” is to be understood to denote the protection of technical and non-living materials, such as adhesives, glues, wood, paper and paperboard, textiles, leather, paint dispersions, plastics, coiling lubricants, fiber or fabrics, against the infestation and destruction by harmful microorganisms, such as fungi and bacteria. As to the protection of wood and other materials, the particular attention is paid to the following harmful fungi: Ascomycetes such as Ophiostoma spp., Ceratocystis spp., Aureobasidium pullulans, Scerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp, and Tyromyces spp., Deuteromycetes such as Aspergillus spp., Cladosporium spp., Penicillium spp., Trichorma spp., Alternaria spp., Paecilomyces spp, and Zygomycetes such as Mucor spp., and in addition in the protection of stored products and harvest the following yeast fungi are worthy of note: Candida spp, and Saccharomyces cerevisae.

The compounds I, II and IV and compositions thereof, respectively, may be used for improving the health of a plant. The invention also relates to a method for improving plant health by treating a plant, its propagation material and/or the locus where the plant is growing or is to grow with an effective amount of compounds I, II and/or IV and compositions thereof, respectively.

The term “plant health” is to be understood to denote a condition of the plant and/or its products which is determined by several indicators alone or in combination with each other such as yield (e.g. increased biomass and/or increased content of valuable ingredients), plant vigor [e.g. improved plant growth and/or greener leaves (“greening effect”)], quality (e.g. improved content or composition of certain ingredients) and tolerance to abiotic and/or biotic stress. The above identified indicators for the health condition of a plant may be interdependent or may result from each other.

The compounds of formula I, II and IV can be present in different crystal modifications whose biological activity may differ. They are likewise subject matter of the present invention.

The compounds I, II and IV are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I, II and/or IV as such or a composition comprising at least one compound I, II and/or IV prophylactically either at or before planting or transplanting.

The invention also relates to agrochemical compositions comprising a solvent or solid carrier and at least one compound I, II and/or IV and to the use for controlling harmful fungi.

An agrochemical composition comprises a fungicidally effective amount of a compound I, II and/or IV. The term “effective amount” denotes an amount of the composition or of the compounds I, II and/or IV, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, II and IV and salts thereof can be converted into customary types of agrochemical compositions, e.g. solutions, emulsions, suspensions, dusts, powders, pastes and granules. The composition type depends on the particular intended purpose; in each case, it should ensure a fine and uniform distribution of the compound according to the invention.

Examples for composition types are suspensions (SC, OD, FS), emulsifiable concentrates (EC), emulsions (EW, EO, ES), pastes, pastilles, wettable powders or dusts (WP, SP, SS, WS, DP, DS) or granules (GR, FG, GG, MG), which can be water-soluble or wettable, as well as gel formulations for the treatment of plant propagation materials such as seeds (GF).

Usually the composition types (e.g. SC, OD, FS, EC, WG, SG, WP, SP, SS, WS, GF) are employed diluted. Composition types such as DP, DS, GR, FG, GG and MG are usually used undiluted.

The compositions are prepared in a known manner (cf. U.S. Pat. No. 3,060,084, EP-A 707 445 (for liquid concentrates), Browning: “Agglomeration”, Chemical Engineering, Dec. 4, 1967, 147-48, Perry's Chemical Engineer's Handbook, 4^{th} Ed., McGraw-Hill, New York, 1963, pp. 8-57 et seq., WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (J. Wiley & Sons, New York, 1961), Hance et al.: Weed Control Handbook (8^{th} Ed., Blackwell Scientific, Oxford, 1989) and Mollet, H. and Grubemann, A.: Formulation technology (Wiley VCH Verlag, Weinheim, 2001).

The agrochemical compositions may also comprise auxiliaries which are customary in agrochemical compositions. The auxiliaries used depend on the particular application form and active substance, respectively.

Examples for suitable auxiliaries are solvents, solid carriers, dispersants or emulsifiers (such as further solubilizers, protective colloids, surfactants and adhesion agents), organic and inorganic thickeners, bactericides, anti-freezing agents, anti-foaming agents, if appropriate colorants and tackifiers or binders (e.g. for seed treatment formulations).

Suitable solvents are water, organic solvents such as mineral oil fractions of medium to high boiling point, such as kerosene or diesel oil, furthermore coal tar oils and oils of vegetable or animal origin, aliphatic, cyclic and aromatic hydrocarbons, e.g. toluene, xylene, paraffin, tetrahydronaphthalene, alkylated naphthalenes or their derivatives, alcohols such as methanol, ethanol, propanol, butanol and cyclohexanol, glycols, ketones such as cyclohexanone and gamma-butyrolactone, fatty acid dimethylamides, fatty acids and fatty acid esters and strongly polar solvents, e.g. amines such as N-methylpyrrolidone.

Solid carriers are mineral earths such as silicates, silica gels, talc, kaolins, limestone, lime, chalk, bole, loess, clays, dolomite, diatomaceous earth, calcium sulfate, magnesium sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers.

Suitable surfactants (adjuvants, wtters, tackifiers, dispersants or emulsifiers) are alkali metal, alkaline earth metal and ammonium salts of aromatic sulfonic acids, such as ligninsoulfonic acid (Borresperse® types, Borregard, Norway) phenolsulfonic acid, naphthalenesulfonic acid (Morwet® types, Akzo Nobel, U.S.A.), dibutylnaphthalenesulfonic acid (Nekal® types, BASF, Germany), and fatty acids, alkylsulfonates, alkylarylsulfonates, alkyl sulfates, laurylether sulfates, fatty alcohol sulfates, and sulfated hexa-, hepta- and octadecanolates, sulfated fatty alcohol glycol ethers, furthermore condensates of naphthalene or of naphthalenesulfonic acid with phenol and formaldehyde, polyoxy-ethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and proteins, denatured proteins, polysaccharides (e.g. methylcellulose), hydrophobically modified starches, polyvinyl alcohols (Mowiol® types, Clariant, Switzerland), polycarboxylates (Sokolan® types, BASF, Germany), polyalkoxylates, polyvinylamines (Lupasol® types, BASF, Germany), polyvinylpyrrolidone and the copolymers thereof.

Examples for thickeners (i.e. compounds that impart a modified flowability to compositions, i.e. high viscosity under static conditions and low viscosity during agitation) are polysaccharides and organic and anorganic clays such as Xanthan gum (Kelzan®, CP Kelco, U.S.A.), Rhodopol® 23 (Rhodia, France), Veegum® (R. T. Vanderbilt, U.S.A.) or Attaclay® (Engelhard Corp., NJ, USA).

Bactericides may be added for preservation and stabilization of the composition. Examples for suitable bactericides are those based on dichlorophene and benzylalcohol hemi formal (Proxel® from ICI or Acticide® RS from Thor Chemie and Kathon® MK from Rohm & Haas) and isothiazolinone derivatives such as alkylisothiazolinones and benzisothiazolinones (Acticide® MBS from Thor Chemie).

Examples for suitable anti-freezing agents are ethylene glycol, propylene glycol, urea and glycerin.

Examples for anti-foaming agents are silicone emulsions (such as e.g. Silikon® SRE, Wacker, Germany or Rhodorsil®, Rhodia, France), long chain alcohols, fatty acids, salts of fatty acids, fluoroorganic compounds and mixtures thereof.

Suitable colorants are pigments of low water solubility and water-soluble dyes. Examples to be mentioned und the designations rhodamin B, C. I. pigment red 112, C. I. solvent red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples for tackifiers or binders are polyvinylpyrrolidons, polyvinylacetates, polyvinyl alcohols and cellulose ethers (Tylose®, Shin-Etsu, Japan).

Powders, materials for spreading and dusts can be prepared by mixing or concomitantly grinding the compounds I and, if appropriate, further active substances, with at least one solid carrier.

Granules, e.g. coated granules, impregnated granules and homogeneous granules, can be prepared by binding the active substances to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers.

Examples for composition types are:

1. Composition Types for Dilution with Water

### i) Water-Soluble Concentrates (SL, LS)

10 parts by weight of a compound I according to the invention are dissolved in 90 parts by weight of water or in a water-soluble solvent. As an alternative, wetting agents or other auxiliaries are added. The active substance dissolves upon dilution with water. In this way, a composition having a content of 10% by weight of active substance is obtained.

### ii) Dispersible Concentrates (DC)

20 parts by weight of a compound I according to the invention are dissolved in 70 parts by weight of cyclohexanone with addition of 10 parts by weight of a dispersant, e.g. polyvinylpyrrolidone. Dilution with water gives a dispersion. The active substance content is 20% by weight.

iii) Emulsifiable Concentrates (EC)

15 parts by weight of a compound I according to the invention are dissolved in 75 parts by weight of xylene with addition of calcium dodecylbenzenesulfonate and castor oil ethoxylate (in each case 5 parts by weight). Dilution with water gives an emulsion. The composition has an active substance content of 15% by weight.

### iv) Emulsions (EW, EO, ES)

25 parts by weight of a compound I according to the invention are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (Ultraturrax) and made into a homogeneous emulsion. Dilution with water gives an emulsion. The composition has an active substance content of 25% by weight.

### v) Suspensions (SC, OD, FS)

In an agitated ball mill, 20 parts by weight of a compound I according to the invention are comminuted with addition of 10 parts by weight of dispersants and wetting agents and 70 parts by weight of water or an organic solvent to give a fine active substance suspension. Dilution with water gives a stable suspension of the active substance. The active substance content in the composition is 20% by weight,

### vi) Water-Dispersible Granules and Water-Soluble Granules (WG, SG)

50 parts by weight of a compound I according to the invention are ground finely with addition of 50 parts by weight of dispersants and wetting agents and prepared as water-dispersible or water-soluble granules by means of technical appliances (e.g. extrusion, spray tower, fluidized bed). Dilution with water gives a stable dispersion or solution of the active substance. The composition has an active substance content of 50% by weight.

### vi) Water-Dispersible Powders and Water-Soluble Powders (WP, SP, SS, WS)

75 parts by weight of a compound I according to the invention are ground in a rotor-stator mill with addition of 25 parts by weight of dispersants, wetting agents and silica gel. Dilution with water gives a stable dispersion or solution of the active substance. The active substance content of the composition is 75% by weight.

viii) Gel (GF)

In an agitated ball mill, 20 parts by weight of a compound I according to the invention are comminuted with addition of 10 parts by weight of dispersants, 1 part by weight of a gelling agent wetters and 70 parts by weight of water or of an organic solvent to give a fine suspension of the active substance. Dilution with water gives a stable suspension of the active substance, whereby a composition with 20% (w/w) of active substance is obtained.

### 2. Composition Types to be Applied Undiluted

### ix) Dustable Powders (DP, DS)

5 parts by weight of a compound I according to the invention are ground finely and mixed intimately with 95 parts by weight of finely divided kaolin. This gives a dustable composition having an active substance content of 5% by weight.

### x) Granules (GR, FG, GG, MG)

0.5 parts by weight of a compound I according to the invention is ground finely and associated with 99.5 parts by weight of carriers. Current methods are extrusion, spray-drying or the fluidized bed. This gives granules to be applied undiluted having an active substance content of 0.5% by weight.

### xi) ULV Solutions (UL)

10 parts by weight of a compound I according to the invention are dissolved in 90 parts by weight of an organic solvent, e.g. xylene. This gives a composition to be applied undiluted having an active substance content of 10% by weight.

The agrochemical compositions generally comprise between 0.01 and 95%, preferably between 0.1 and 90%, most preferably between 0.5 and 90%, by weight of active substance. The active substances are employed in a purity of from 90% to 100%, preferably from 95% to 100% (according to NMR spectrum).

Water-soluble concentrates (LS), flowable concentrates (FS), powders for dry treatment (DS), water-dispersible powders for slurry treatment (WS), water-soluble powders (SS), emulsions (ES) emulsifiable concentrates (EC) and gels (GF) are usually employed for the purposes of treatment of plant propagation materials, particularly seeds. These compositions can be applied to plant propagation materials, particularly seeds, diluted or undiluted. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying or treating agrochemical compounds and compositions thereof, respectively, on to plant propagation material, especially seeds, are known in the art, and include dressing, coating, pelleting, dusting, soaking and infurrow application methods of the propagation material. In a preferred embodiment, the compounds or the compositions thereof, respectively, are applied on to the plant propagation material by a method such that germination is not induced, e.g. by seed dressing, pelleting, coating and dusting.

In a preferred embodiment, a suspension-type (FS) composition is used for seed treatment. Typically, a FS composition may comprise 1-800 g/l of active substance, 1-200 g/l Surfactant, 0 to 200 g/l antifreezing agent, 0 to 400 g/l of binder, 0 to 200 g/l of a pigment and up to 1 liter of a solvent, preferably water.

The active substances can be used as such or in the form of their compositions, e.g. in the form of directly sprayable solutions, powders, suspensions, dispersions, emulsions, oil dispersions, pastes, dustable products, materials for spreading, or granules, by means of spraying, atomizing, dusting, spreading, brushing, immersing or pouring. The application forms depend entirely on the intended purposes; it is intended to ensure in each case the finest possible distribution of the active substances according to the invention.

Aqueous application forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active substance concentrations in the ready-to-use preparations can be varied within relatively wide ranges. In general, they are from 0.0001 to 10%, preferably from 0.001 to 1% by weight of active substance.

The active substances may also be used successfully in the ultra-low-volume process (ULV), it being possible to apply compositions comprising over 95% by weight of active substance, or even to apply the active substance without additives.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, in particular from 0.1 to 0.75 kg per ha.

In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seed) are generally required.

When used in the protection of materials or stored products, the amount of active substance applied depends on the kind of application area and on the desired effect.

Amounts customarily applied in the protection of materials are, e.g., 0.001 g to 2 kg, preferably 0.005 g to 1 kg, of active substance per cubic meter of treated material.

Various types of oils, wetters, adjuvants, herbicides, bactericides, other fungicides and/or pesticides may be added to the active substances or the compositions comprising them, if appropriate not until immediately prior to use (tank mix). These agents can be admixed with the compositions according to the invention in a weight ratio of 1:100 to 100:1, preferably 1:10 to 10:1.

Adjuvants which can be used are in particular organic modified polysiloxanes such as Break Thru S 240®; alcohol alkoxylates such as Atplus 245®, Atplus MBA 1303®, Plurafac LF 300® and Lutensol ON 30®; EO/PO block polymers, e.g. Pluronic RPE 2035® and Genapol B®; alcohol ethoxylates such as Lutensol XP 80®; and dioctyl sulfosuccinate sodium such as Leophen RA®.

The compositions according to the invention can, in the use form as fungicides, also be present together with other active substances, e.g. with herbicides, insecticides, growth regulators, fungicides or else with fertilizers, as pre-mix or, if appropriate, not until immediately prior to use (tank mix).

Mixing the compounds I, II and/or IV or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of active substances, in conjunction with which the compounds according to the invention can be used, is intended to illustrate the possible combinations but does not limit them:

A) strobilurins
azoxystrobin, dimoxystrobin, enestroburin, fluoxastrobin, kresoxim-methyl, metominostrobin, orysastrobin, picoxystrobin, pyraclostrobin, pyribencarb, trifloxystrobin, 2-(2-(6-(3-chloro-2-methyl-phenoxy)-5-fluoro-pyrimidin-4-yloxy)phenyl)-2-methoxyimino-N-methyl-acetamide, 3-methoxy-2-(2-(N-(4-methoxyphenyl)-cyclopropane-carboximidoylsulfanylmethyl)-phenyl)-acrylic acid methyl ester, methyl (2-chloro-5-[1-(3-methylbenzyloxyimino)ethyl]benzyl)carbamate and 2-(2-(3-(2,6-dichlorophenyl)-1-methyl-allylideneaminooxymethyl)-phenyl)-2-methoxyimino-N-methyl-acetamide;
   B) carboxamides
carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penthiopyrad, sedaxane, tecloftalam, thifluzamide, tiadinil, 2-amino-4-methylthiazole-5-carboxanilide, 2-chloro-N-(1,1,3-trimethyl-indan-4-yl)-nicotinamide, N-(3′,4′,5′-trifluorobiphenyl-2-yl)-3-difluoromethyl-1-methyl-1H-pyrazole-4-carboxamide, N-(4′-trifluoromethylthiobiphenyl-2-yl)-3-difluoromethyl-1-methyl-1H-pyrazole-4-carboxamide, N-(2-(1,3-dimethyl-butyl)-phenyl)-1,3-dimethyl-5-fluoro-1H-pyrazole-4-carboxamide and N-(2-(1,3,3-trimethyl-butyl)-phenyl)-1,3-dimethyl-5-fluoro-1H-pyrazole-4-carboxamide;
carboxylic morpholides: dimethomorph, flumorph, pyrimorph;
benzoic acid amides: flumetover, fluopicolide, fluopyram, zoxamide, N-(3-Ethyl-3,5,5-trimethyl-cyclohexyl)-3-formylamino-2-hydroxy-benzamide;
other carboxamides: carpropamid, dicyclomet, mandiproamid, oxytetracyclin, silthiofarm and N-(6-methoxy-pyridin-3-yl)cyclopropanecarboxylic acid amide;
   C) azoles
triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-(4-chloro-phenyl)-2-([1,2,4]triazol-1-yl)-cycloheptanol;
imidazoles: cyazofamid, imazalil, pefurazoate, prochloraz, triflumizol;
benzimidazoles: benomyl, carbendazim, fuberidazole, thiabendazole;
others: ethaboxam, etridiazole, hymexazole and 2-(4-chloro-phenyl)-N-[4-(3,4-dimethoxy-phenyl)-isoxazol-5-yl]-2-prop-2-ynyloxy-acetamide;
   D) heterocyclic Compounds
pyridines: fluazinam, pyrifenox, 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine, 2,3,5,6-tetra-chloro-4-methanesulfonyl-pyridine, 3,4,5-trichloropyridine-2,6-di-carbonitrile, N-(1-(5-bromo-3-chloro-pyridin-2-yl)-ethyl)-2,4-dichloronicotinamide, N-[(5-bromo-3-chloro-pyridin-2-yl)-methyl]-2,4-dichloro-nicotinamide;
pyrimidines: bupirimate, cyprodinil, diflumetorim, fenarimol, ferimzone, mepanipyrim, nitrapyrin, nuarimol, pyrimethanil;
piperazines: triforine;
pyrroles: fenpiclonil, fludioxonil;
morpholines: aldimorph, dodemorph, dodemorph-acetate, fenpropimorph, tridemorph;
piperidines: fenpropidin;
dicarboximides: fluoroimid, iprodione, procymidone, vinclozolin;
non-aromatic 5-membered heterocycles: famoxadone, fenamidone, flutianil, octhilinone, probenazole, 5-amino-2-isopropyl-3-oxo-4-ortho-tolyl-2,3-dihydropyrazole-1-carbothioic acid S-allyl ester;
others: acibenzolar-S-methyl, amisulbrom, anilazin, blasticidin-S, captafol, captan, chinomethionat, dazomet, debacarb, diclomezine, difenzoquat, difenzoquatmethylsulfate, fenoxanil, Folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxy-pyrimidin-2-yl)-2-methyl-1H-benzoimidazole, 5-chloro-7-(4-methylpiperidin-1-yl)-6-(2,4,6-trifluorophenyl)-[1,2,4]triazolo[1,5-a]pyrimidine and 5-ethyl-6-octyl-[1,2,4]triazolo[1,5-a]pyrimidine-7-ylamine;
   E) carbamates
thio- and dithiocarbamates: ferbam, mancozeb, maneb, metam, methasulphocarb, metiram, propineb, thiram, zineb, ziram;
carbamates: benthiavalicarb, diethofencarb, iprovalicarb, propamocarb, propamocarb hydrochlorid, valiphenal and N-(1-(1-(4-cyano-phenyl)ethanesulfonyl)-but-2-yl) carbamic acid-(4-fluorophenyl) ester;

### F) Other Active Substances

guanidines: guanidine, dodine, dodine free base, guazatine, guazatine-acetate, iminoctadine, iminoctadine-triacetate, iminoctadine-tris(albesilate);
antibiotics: kasugamycin, kasugamycin hydrochloride-hydrate, streptomycin, polyoxine, validamycin A;
nitrophenyl derivates: binapacryl, dinobuton, dinocap, nitrthal-isopropyl, tecnazen,
organometal compounds: fentin salts, such as fentin-acetate, fentin chloride or fentin hydroxide;
sulfur-containing heterocyclyl compounds: dithianon, isoprothiolane;
organophosphorus compounds: edifenphos, fosetyl, fosetyl-aluminum, iprobenfos, phosphorous acid and its salts, pyrazophos, tolclofos-methyl;
organochlorine compounds: chlorothalonil, dichlofluanid, dichlorophen, flusulfamide, hexachlorobenzene, pencycuron, pentachlorphenole and its salts, phthalide, quintozene, thiophanate-methyl, tolylfluanid, N-(4-chloro-2-nitro-phenyl)-N-ethyl-4-methyl-benzenesulfonamide;
inorganic active substances: Bordeaux mixture, copper acetate, copper hydroxide, copper oxychloride, basic copper sulfate, sulfur;
others: biphenyl, bronopol, cyflufenamid, cymoxanil, diphenylamin, metrafenone, mildiomycin, oxin-copper, prohexadione-calcium, spiroxamine, tolylfluanid, N(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethylphenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2-methyl-4-(3-trimethylsilanyl-propoxy)phenyl)-N-ethyl-N-methyl formamidine,
2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, acetic acid 6-tert-butyl-8-fluoro-2,3-dimethyl-quinolin-4-yl ester and methoxy-acetic acid 6-tert-butyl-8-fluoro-2,3-dimethyl-quinolin-4-yl ester.
   G) growth Regulators
abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid, trinexapac-ethyl and uniconazole;

### H) Herbicides

acetamides: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor;
amino acid derivatives: bilanafos, glyphosate, glufosinate, sulfosate;
aryloxyphenoxypropionates: clodinafop, cyhalofop-butyl, fenoxaprop, fluazifop, haloxyfop, metamifop, propaquizafop, quizalofop, quizalofop-P-tefuryl;
Bipyridyls: diquat, paraquat;
(thio)carbamates: asulam, butylate, carbetamide, desmedipham, dimepiperate, eptam (EPTC), esprocarb, molinate, orbencarb, phenmedipham, prosulfocarb, pyributicarb, thiobencarb, triallate;
cyclohexanediones: butroxydim, clethodim, cycloxydim, profoxydim, sethoxydim, tepraloxydim, tralkoxydim;
dinitroanilines: benfluralin, ethalfluralin, oryzalin, pendimethalin, prodiamine, trifluralin;
diphenyl ethers: acifluorfen, aclonifen, bifenox, diclofop, ethoxyfen, fomesafen, lactofen, oxyfluorfen;
hydroxybenzonitriles: bomoxynil, dichlobenil, ioxynil;
imidazolinones: imazamethabenz, imazamox, imazapic, imazapyr, imazaquin, imazethapyr;
phenoxy acetic acids: clomeprop, 2,4-dichlorophenoxyacetic acid (2,4-D), 2,4-DB, dichlorprop, MCPA, MCPA-thioethyl, MCPB, Mecoprop;
pyrazines: chloridazon, flufenpyr-ethyl, fluthiacet, norflurazon, pyridate;
pyridines: aminopyralid, clopyralid, diflufenican, dithiopyr, fluridone, fluoroxypyr, picloram, picolinafen, thiazopyr;
sulfonyl ureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propyl-imidazo[1,2-b]pyridazin-3-yl)sulfonyl)-3-(4,6-dimethoxy-pyrimidin-2-yl)urea;
triazines: ametryn, atrazine, cyanazine, dimethametryn, ethiozin, hexazinone, metamitron, metribuzin, prometryn, simazine, terbuthylazine, terbutryn, triaziflam;
ureas: chlorotoluron, daimuron, diuron, fluometuron, isoproturon, linuron, methabenzthiazuron, tebuthiuron;
other acetolactate synthase inhibitors: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam;
others: amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, fluorochloridone, flurtamone, indanofan, isoxaben, isoxaflutole, lenacil, propanil, propyzamide, quinclorac, quinmerac, mesotrione, methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxy-ethoxymethyl)-6-trifluoromethyl-pyridine-3-carbonyl]-bicyclo[3.2.1]oct-3-en-2-one, (3-[2-chloro-4-fluoro-5-(3-methyl-2,6-dioxo-4-trifluoromethyl-3,6-dihydro-2H-pyrimidin-1-yl)phenoxy]-pyridin-2-yloxy)-acetic acid ethyl ester, 6-amino-5-chloro-2-cyclopropylpyrimidine-4-carboxylic acid methyl ester, 6-chloro-3-(2-cyclopropyl-6-methylphenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)pyridine-2-carboxylic acid methyl ester, and 4-amino-3-chloro-6-(4-chloro-3-dimethylamino-2-fluoro-phenyl)-pyridine-2-carboxylic acid methyl ester.

### I) Insecticides

organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, bifenthrin, cyfluthrin, cyhalothrin, cyphenothrin,
cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;
insect growth regulators: a) chitin synthesis inhibitors: benzoylureas: chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;
nicotinic receptor agonists/antagonists compounds: clothianidin, dinotefuran, imidacloprid, thiamethoxam, nitenpyram, acetamiprid, thiacloprid, 1-(2-chlorothiazol-5-ylmethyl)-2-nitrimino-3,5-dimethyl-[1,3,5]triazinane;
GABA antagonist compounds: endosulfan, ethiprole, fipronil, vaniliprole, pyrafluprole, pyriprole, 5-amino-1-(2,6-dichloro-4-methyl-phenyl)-4-sulfinamoyl-1H-pyrazole-3-carbothioic acid amide;
macrocyclic lactone insecticides: abamectin, emamectin, milbemectin, lepimectin, spinosad, spinetoram;
mitochondrial electron transport inhibitor (METI) I acaricides: fenazaquin, pyridaben, tebufenpyrad, tolfenpyrad, flufenerim;
METI II and III compounds: acequinocyl, fluacyprim, hydramethylnon;
Uncouplers: chlorfenapyr;
oxidative phosphorylation inhibitors: cyhexatin, diafenthiuron, fenbutatin oxide, propargite;
moulting disruptor compounds: cryomazine;
mixed function oxidase inhibitors: piperonyl butoxide;
sodium channel blockers: indoxacarb, metaflumizone;
others: benclothiaz, bifenazate, cartap, flonicamid, pyridalyl, pymetrozine, sulfur, thiocyclam, flubendiamide, chlorantraniliprole, cyazypyr (HGW86), cyenopyrafen, flupyrazofos, cyflumetofen, amidoflumet, imicyafos, bistrifluoron, and pyrifluquinazon.

The present invention furthermore relates to agrochemical compositions comprising a mixture of at least one compound I, II and/or IV (component 1) and at least one further active substance useful for plant protection, e.g. selected from the groups A) to I) (component 2), in particular one further fungicide, e.g. one or more fungicide from the groups A) to F), as described above, and if desired one suitable solvent or solid carrier. Those mixtures are of particular interest, since many of them at the same application rate show higher efficiencies against harmful fungi. Furthermore, combating harmful fungi with a mixture of compounds I, II and/or IV and at least one fungicide from groups A) to F), as described above, is more efficient than combating those fungi with individual compounds I, II or IV or individual fungicides from groups A) to F). By applying compounds I, II and/or IV together with at least one active substance from groups A) to I) a synergistic effect can be obtained, i.e. more then simple addition of the individual effects is obtained (synergistic mixtures).

According to this invention, applying the compounds I, II and/or IV together with at least one further active substance is to be understood to denote that at least one compound of formula I, II and/or IV and at least one further active substance occur simultaneously at the site of action (i.e. the harmful fungi to be controlled or their habitats such as infected plants, plant propagation materials, particularly seeds, surfaces, materials or the soil as well as plants, plant propagation materials, particularly seeds, soil, surfaces, materials or rooms to be protected from fungal attack) in a fungicidally effective amount. This can be obtained by applying the compounds I, II and/or IV and at least one further active substance simultaneously, either jointly (e.g. as tank-mix) or separately, or in succession, wherein the time interval between the individual applications is selected to ensure that the active substance applied first still occurs at the site of action in a sufficient amount at the time of application of the further active substance(s). The order of application is not essential for working of the present invention.

In binary mixtures, i.e. compositions according to the invention comprising one compound I, II or IV (component 1) and one further active substance (component 2), e.g. one active substance from groups A) to I), the weight ratio of component 1 and component 2 generally depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:3 to 3:1.

In ternary mixtures, i.e. compositions according to the invention comprising one compound I (component 1) and a first further active substance (component 2) and a second further active substance (component 3), e.g. two active substances from groups A) to 1), the weight ratio of component 1 and component 2 depends from the properties of the active substances used, preferably it is in the range of from 1:50 to 50:1 and particularly in the range of from 1:10 to 10:1, and the weight ratio of component 1 and component 3 preferably is in the range of from 1:50 to 50:1 and particularly in the range of from 1:10 to 10:1.

The components can be used individually or already partially or completely mixed with one another to prepare the composition according to the invention. It is also possible for them to be packaged and used further as combination composition such as a kit of parts.

In one embodiment of the invention, the kits may include one or more, including all, components that may be used to prepare a subject agrochemical composition. E. g., kits may include one or more fungicide component(s) and/or an adjuvant component and/or an insecticide component and/or a growth regulator component and/or a herbicide. One or more of the components may already be combined together or preformulated.

In those embodiments where more than two components are provided in a kit, the components may already be combined together and as such are packaged in a single container such as a vial, bottle, can, pouch, bag or canister. In other embodiments, two or more components of a kit may be packaged separately, i.e., not preformulated. As such, kits may include one or more separate containers such as vials, cans, bottles, pouches, bags or canisters, each container containing a separate component for an agrochemical composition. In both forms, a component of the kit may be applied separately from or together with the further components or as a component of a combination composition according to the invention for preparing the composition according to the invention.

The user applies the composition according to the invention usually from a predosage device, a knapsack sprayer, a spray tank or a spray plane. Here, the agrochemical composition is made up with water and/or buffer to the desired application concentration, it being possible, if appropriate, to add further auxiliaries, and the ready-to-use spray liquor or the agrochemical composition according to the invention is thus obtained. Usually, 50 to 500 liters of the ready-to-use spray liquor are applied per hectare of agricultural useful area, preferably 100 to 400 liters.

According to one embodiment, individual components of the composition according to the invention such as parts of a kit or parts of a binary or ternary mixture may be mixed by the user himself in a spray tank and further auxiliaries may be added, if appropriate (tank mix).

In a further embodiment, either individual components of the composition according to the invention or partially premixed components, e.g. components comprising compounds I, II and/or IV and/or active substances from the groups A) to I), may be mixed by the user in a spray tank and further auxiliaries and additives may be added, if appropriate (tank mix).

In a further embodiment, either individual components of the composition according to the invention or partially premixed components, e.g. components comprising compounds I, II and/or IV and/or active substances from the groups A) to I), can be applied jointly (e.g. after tank mix) or consecutively.

Preference is also given to mixtures comprising a compound I, II and/or IV (component 1) and at least one active substance selected from the strobilurines of group A) (component 2) and particularly selected from azoxystrobin, dimoxystrobin, fluoxastrobin, kresoxim-methyl, orysastrobin, picoxystrobin, pyraclostrobin and trifloxystrobin.

Preference is also given to mixtures comprising a compound I, II and/or IV (component 1) and at least one active substance selected from the carboxamides of group B) (component 2) and particularly selected from bixafen, boscalid, sedaxane, fenhexamid, metalaxyl, isopyrazam, mefenoxam, ofurace, dimethomorph, flumorph, fluopicolid (picobenzamid), zoxamide, carpropamid, mandipropamid and N-(3′,4′,5′-trifluorobiphenyl-2-yl)-3-difluoromethyl-1-methyl-1H-pyrazole-4-carboxamide.

Preference is given to mixtures comprising a compound of formula I, II and/or IV (component 1) and at least one active substance selected from the azoles of group C) (component 2) and particularly selected from cyproconazole, difenoconazole, epoxiconazole, fluquinconazole, flusilazole, flutriafol, metconazole, myclobutanil, penconazole, propiconazole, prothioconazole, triadimefon, triadimenol, tebuconazole, tetraconazole, triticonazole, prochloraz, cyazofamid, benomyl, carbendazim and ethaboxam.

Preference is also given to mixtures comprising a compound I, II and/or IV (component 1) and at least one active substance selected from the heterocyclic compounds of group D) (component 2) and particularly selected from fluazinam, cyprodinil, fenarimol, mepanipyrim, pyrimethanil, triforine, fludioxonil, dodemorph, fenpropimorph, tridemorph, fenpropidin, iprodione, vinclozolin, famoxadone, fenamidone, probenazole, proquinazid, acibenzolar-5-methyl, captafol, folpet, fenoxanil, quinoxyfen and 5-ethyl-6-octyl-[1,2,4]triazolo[1,5-a]pyrimidine-7-ylamine.

Preference is also given to mixtures comprising a compound I, II and/or IV (component 1) and at least one active substance selected from the carbamates of group E) (component 2) and particularly selected from mancozeb, metiram, propineb, thiram, iprovalicarb, benthiavalicarb and propamocarb.

Preference is also given to mixtures comprising a compound I, II and/or IV (component 1) and at least one active substance selected from the fungicides given in group F) (component 2) and particularly selected from dithianon, fentin salts, such as fentin acetate, fosetyl, fosetyl-aluminium, H₃PO₃ and salts thereof, chlorthalonil, dichlofluanid, thiophanat-methyl, copper acetate, copper hydroxide, copper oxychloride, copper sulfate, sulfur, cymoxanil, metrafenone and spiroxamine.

Accordingly, the present invention furthermore relates to compositions comprising one compound I, II and/or IV (component 1) and one further active substance (component 2), which further active substance is selected from the column “Component 2” of the lines B-1 to B-346 of Table B.

A further embodiment relates to the compositions B-1 to B-346 listed in Table B, where a row of Table B corresponds in each case to a fungicidal composition comprising one of the in the present specification individualized compounds of formula I, II or IV (component 1) and the respective further active substance from groups A) to I) (component 2) stated in the row in question. Preferably, the compositions described comprise the active substances in synergistically effective amounts.

TABLE B
Composition comprising one indivivalized compound I, II or IV and one further active substance
from groups A) to I)

| Mixture | Component 1 | Component 2 |
|---|---|---|
| B-1 | one individualized compound I, II or IV | Azoxystrobin |
| B-2 | one individualized compound I, II or IV | Dimoxystrobin |
| B-3 | one individualized compound I, II or IV | Enestroburin |
| B-4 | one individualized compound I, II or IV | Fluoxastrobin |
| B-5 | one individualized compound I, II or IV | Kresoxim-methyl |
| B-6 | one individualized compound I, II or IV | Metominostrobin |
| B-7 | one individualized compound I, II or IV | Orysastrobin |
| B-8 | one individualized compound I, II or IV | Picoxystrobin |
| B-9 | one individualized compound I, II or IV | Pyraclostrobin |
| B-10 | one individualized compound I, II or IV | Pyribencarb |
| B-11 | one individualized compound I, II or IV | Trifloxystrobin |
| B-12 | one individualized compound I, II or IV | 2-(2-(6-(3-Chloro-2-methyl- |
| | | phenoxy)-5-fluoro-pyrimidin-4- |
| | | yloxy)-phenyl)-2-methoxyimino- |
| | | N-methyl-acetamide |
| B-13 | one individualized compound I, II or IV | 2-(ortho-((2,5-Dimethylphenyl- |
| | | oxymethylen)phenyl)-3-methoxy- |
| | | acrylsauremethylester |
| B-14 | one individualized compound I, II or IV | 3-Methoxy-2-(2-(N-(4-methoxy- |
| | | phenyl)-cyclopropanecarbox- |
| | | imidoylsulfanylmethyl)-phenyl)- |
| | | acrylic acid methyl ester |
| B-15 | one individualized compound I, II or IV | 2-(2-(3-(2,6-dichlorophenyl)-1- |
| | | methyl-allylideneaminooxy- |
| | | methyl)-phenyl)-2-methoxyimino- |
| | | N-methyl-acetamide |
| B-16 | one individualized compound I, II or IV | Benalaxyl |
| B-17 | one individualized compound I, II or IV | Benalaxyl-M |
| B-18 | one individualized compound I, II or IV | Benodanil |
| B-19 | one individualized compound I, II or IV | Bixafen |
| B-20 | one individualized compound I, II or IV | Boscalid |
| B-21 | one individualized compound I, II or IV | Carboxin |
| B-22 | one individualized compound I, II or IV | Fenfuram |
| B-23 | one individualized compound I, II or IV | Fenhexamid |
| B-24 | one individualized compound I, II or IV | Flutolanil |
| B-25 | one individualized compound I, II or IV | Furametpyr |
| B-26 | one individualized compound I, II or IV | Isopyrazam |
| B-27 | one individualized compound I, II or IV | Isotianil |
| B-28 | one individualized compound I, II or IV | Kiralaxyl |
| B-29 | one individualized compound I, II or IV | Mepronil |
| B-30 | one individualized compound I, II or IV | Metalaxyl |
| B-31 | one individualized compound I, II or IV | Metalaxyl-M |
| B-32 | one individualized compound I, II or IV | Ofurace |
| B-33 | one individualized compound I, II or IV | Oxadixyl |
| B-34 | one individualized compound I, II or IV | Oxycarboxin |
| B-35 | one individualized compound I, II or IV | Penthiopyrad |
| B-36 | one individualized compound I, II or IV | Sedaxane |
| B-37 | one individualized compound I, II or IV | Tecloftalam |
| B-38 | one individualized compound I, II or IV | Thifluzamide |
| B-39 | one individualized compound I, II or IV | Tiadinil |
| B-40 | one individualized compound I, II or IV | 2-Amino-4-methyl-thiazole-5- |
| | | carboxylic acid anilide |
| B-41 | one individualized compound I, II or IV | 2-Chloro-N-(1,1,3-trimethyl- |
| | | indan-4-yl)-nicotinamide |
| B-42 | one individualized compound I, II or IV | N-(3′,4′,5′-trifluorobiphenyl-2-yl)- |
| | | 3-difluoromethyl-1-methyl-1H- |
| | | pyrazole-4-carboxamide |
| B-43 | one individualized compound I, II or IV | N-(4′-trifluoromethylthiobiphenyl- |
| | | 2-yl)-3-difluoromethyl-1-methyl- |
| | | 1H-pyrazole-4-carboxamide |
| B-44 | one individualized compound I, II or IV | N-(2-(1,3-dimethyl-butyl)- |
| | | phenyl)-1,3-dimethyl-5-fluoro- |
| | | 1H-pyrazole-4-carboxamide |
| B-45 | one individualized compound I, II or IV | N-(2-(1,3,3-trimethyl-butyl)- |
| | | phenyl)-1,3-dimethyl-5-fluoro- |
| | | 1H-pyrazole-4-carboxamide |
| B-46 | one individualized compound I, II or IV | Dimethomorph |
| B-47 | one individualized compound I, II or IV | Flumorph |
| B-48 | one individualized compound I, II or IV | Pyrimorph |
| B-49 | one individualized compound I, II or IV | Flumetover |
| B-50 | one individualized compound I, II or IV | Fluopicolide |
| B-51 | one individualized compound I, II or IV | Fluopyram |
| B-52 | one individualized compound I, II or IV | Zoxamide |
| B-53 | one individualized compound I, II or IV | N-(3-Ethyl-3,5,5-trimethyl- |
| | | cyclohexyl)-3-formylamino-2- |
| | | hydroxy-benzamide |
| B-54 | one individualized compound I, II or IV | Carpropamid |
| B-55 | one individualized compound I, II or IV | Diclocymet |
| B-56 | one individualized compound I, II or IV | Mandipropamid |
| B-57 | one individualized compound I, II or IV | Oxytetracyclin |
| B-58 | one individualized compound I, II or IV | Silthiofam |
| B-59 | one individualized compound I, II or IV | N-(6-methoxy-pyridin-3-yl) cyclopropanecarboxylic |
| | | acid amide |
| B-60 | one individualized compound I, II or IV | Azaconazole |
| B-61 | one individualized compound I, II or IV | Bitertanol |
| B-62 | one individualized compound I, II or IV | Bromuconazole |
| B-63 | one individualized compound I, II or IV | Cyproconazole |
| B-64 | one individualized compound I, II or IV | Difenoconazole |
| B-65 | one individualized compound I, II or IV | Diniconazole |
| B-66 | one individualized compound I, II or IV | Diniconazole-M |
| B-67 | one individualized compound I, II or IV | Epoxiconazole |
| B-68 | one individualized compound I, II or IV | Fenbuconazole |
| B-69 | one individualized compound I, II or IV | Fluquinconazole |
| B-70 | one individualized compound I, II or IV | Flusilazole |
| B-71 | one individualized compound I, II or IV | Flutriafol |
| B-72 | one individualized compound I, II or IV | Hexaconazol |
| B-73 | one individualized compound I, II or IV | Imibenconazole |
| B-74 | one individualized compound I, II or IV | Ipconazole |
| B-75 | one individualized compound I, II or IV | Metconazole |
| B-76 | one individualized compound I, II or IV | Myclobutanil |
| B-77 | one individualized compound I, II or IV | Oxpoconazol |
| B-78 | one individualized compound I, II or IV | Paclobutrazol |
| B-79 | one individualized compound I, II or IV | Penconazole |
| B-80 | one individualized compound I, II or IV | Propiconazole |
| B-81 | one individualized compound I, II or IV | Prothioconazole |
| B-82 | one individualized compound I, II or IV | Simeconazole |
| B-83 | one individualized compound I, II or IV | Tebuconazole |
| B-84 | one individualized compound I, II or IV | Tetraconazole |
| B-85 | one individualized compound I, II or IV | Triadimefon |
| B-86 | one individualized compound I, II or IV | Triadimenol |
| B-87 | one individualized compound I, II or IV | Triticonazole |
| B-88 | one individualized compound I, II or IV | Uniconazole |
| B-89 | one individualized compound I, II or IV | 1-(4-Chloro-phenyl)-2- |
| | | ([1,2,4]triazol-1-yl)-cycloheptanol |
| B-90 | one individualized compound I, II or IV | Cyazofamid |
| B-91 | one individualized compound I, II or IV | Imazalil |
| B-92 | one individualized compound I, II or IV | Imazalil-sulfate |
| B-93 | one individualized compound I, II or IV | Pefurazoate |
| B-94 | one individualized compound I, II or IV | Prochloraz |
| B-95 | one individualized compound I, II or IV | Triflumizole |
| B-96 | one individualized compound I, II or IV | Benomyl |
| B-97 | one individualized compound I, II or IV | Carbendazim |
| B-98 | one individualized compound I, II or IV | Fuberidazole |
| B-99 | one individualized compound I, II or IV | Thiabendazole |
| B-100 | one individualized compound I, II or IV | Ethaboxam |
| B-101 | one individualized compound I, II or IV | Etridiazole |
| B-102 | one individualized compound I, II or IV | Hymexazole |
| B-103 | one individualized compound I, II or IV | 2-(4-Chloro-phenyl)-N-[4-(3,4- |
| | | dimethoxy-phenyl)-isoxazol-5- |
| | | yl]-2-prop-2-ynyloxy-acetamide |
| B-104 | one individualized compound I, II or IV | Fluazinam |
| B-105 | one individualized compound I, II or IV | Pyrifenox |
| B-106 | one individualized compound I, II or IV | 3-[5-(4-Chloro-phenyl)-2,3- |
| | | dimethyl-isoxazolidin-3-yl]- |
| | | pyridine |
| B-107 | one individualized compound I, II or IV | 3-[5-(4-Methyl-phenyl)-2,3-dimethyl- |
| | | isoxazolidin-3-yl]-pyridine |
| B-108 | one individualized compound I, II or IV | 2,3,5,6-Tetrachloro-4- |
| | | methanesulfonyl-pyridine |
| B-109 | one individualized compound I, II or IV | 3,4,5-Trichloro-pyridine-2,6- |
| | | dicarbonitrile |
| B-110 | one individualized compound I, II or IV | N-(1-(5-Bromo-3-chloro-pyridin- |
| | | 2-yl)-ethyl)-2,4-dichloro- |
| | | nicotinamide |
| B-111 | one individualized compound I, II or IV | N-((5-Bromo-3-chloro-pyridin-2- |
| | | yl)-methyl)-2,4-dichloro- |
| | | nicotinamide |
| B-112 | one individualized compound I, II or IV | Bupirimate |
| B-113 | one individualized compound I, II or IV | Cyprodinil |
| B-114 | one individualized compound I, II or IV | Diflumetorim |
| B-115 | one individualized compound I, II or IV | Fenarimol |
| B-116 | one individualized compound I, II or IV | Ferimzone |
| B-117 | one individualized compound I, II or IV | Mepanipyrim |
| B-118 | one individualized compound I, II or IV | Nitrapyrin |
| B-119 | one individualized compound I, II or IV | Nuarimol |
| B-120 | one individualized compound I, II or IV | Pyrimethanil |
| B-121 | one individualized compound I, II or IV | Triforine |
| B-122 | one individualized compound I, II or IV | Fenpiclonil |
| B-123 | one individualized compound I, II or IV | Fludioxonil |
| B-124 | one individualized compound I, II or IV | Aldimorph |
| B-125 | one individualized compound I, II or IV | Dodemorph |
| B-126 | one individualized compound I, II or IV | Dodemorph-acetate |
| B-127 | one individualized compound I, II or IV | Fenpropimorph |
| B-128 | one individualized compound I, II or IV | Tridemorph |
| B-129 | one individualized compound I, II or IV | Fenpropidin |
| B-130 | one individualized compound I, II or IV | Fluoroimid |
| B-131 | one individualized compound I, II or IV | Iprodione |
| B-132 | one individualized compound I, II or IV | Procymidone |
| B-133 | one individualized compound I, II or IV | Vinclozolin |
| B-134 | one individualized compound I, II or IV | Famoxadone |
| B-135 | one individualized compound I, II or IV | Fenamidone |
| B-136 | one individualized compound I, II or IV | Flutianil |
| B-137 | one individualized compound I, II or IV | Octhilinone |
| B-138 | one individualized compound I, II or IV | Probenazole |
| B-139 | one individualized compound I, II or IV | 5-Amino-2-iso-propyl-4-ortho- |
| | | tolyl-2,3-dihydro-pyrazole-1- |
| | | carbothioic acid S-allyl ester |
| B-140 | one individualized compound I, II or IV | Acibenzolar-S-methyl |
| B-141 | one individualized compound I, II or IV | Amisulbrom |
| B-142 | one individualized compound I, II or IV | Anilazin |
| B-143 | one individualized compound I, II or IV | Blasticidin-S |
| B-144 | one individualized compound I, II or IV | Captafol |
| B-145 | one individualized compound I, II or IV | Captan |
| B-146 | one individualized compound I, II or IV | Chinomethionat |
| B-147 | one individualized compound I, II or IV | Dazomet |
| B-148 | one individualized compound I, II or IV | Debacarb |
| B-149 | one individualized compound I, II or IV | Diclomezine |
| B-150 | one individualized compound I, II or IV | Difenzoquat, |
| B-151 | one individualized compound I, II or IV | Difenzoquat-methylsulfate |
| B-152 | one individualized compound I, II or IV | Fenoxanil |
| B-153 | one individualized compound I, II or IV | Folpet |
| B-154 | one individualized compound I, II or IV | Oxolinsaure |
| B-155 | one individualized compound I, II or IV | Piperalin |
| B-156 | one individualized compound I, II or IV | Proquinazid |
| B-157 | one individualized compound I, II or IV | Pyroquilon |
| B-158 | one individualized compound I, II or IV | Quinoxyfen |
| B-159 | one individualized compound I, II or IV | Triazoxid |
| B-160 | one individualized compound I, II or IV | Tricyclazole |
| B-161 | one individualized compound I, II or IV | 2-Butoxy-6-iodo-3-propyl- |
| | | chromen-4-one |
| B-162 | one individualized compound I, II or IV | 5-Chloro-1-(4,6-dimethoxy- |
| | | pyrimidin-2-yl)-2-methyl-1H- |
| | | benzoimidazole |
| B-163 | one individualized compound I, II or IV | 5-Chloro-7-(4-methyl-piperidin-1- |
| | | yl)-6-(2,4,6-trifluoro-phenyl)- |
| | | [1,2,4]triazolo[1,5-a]pyrimidine |
| B-164 | one individualized compound I, II or IV | 5-ethyl-6-octyl-[1,2,4]triazolo[1,5- |
| | | a]pyrimidine-7-ylamine |
| B-165 | one individualized compound I, II or IV | Ferbam |
| B-166 | one individualized compound I, II or IV | Mancozeb |
| B-167 | one individualized compound I, II or IV | Maneb |
| B-168 | one individualized compound I, II or IV | Metam |
| B-169 | one individualized compound I, II or IV | Methasulphocarb |
| B-170 | one individualized compound I, II or IV | Metiram |
| B-171 | one individualized compound I, II or IV | Propineb |
| B-172 | one individualized compound I, II or IV | Thiram |
| B-173 | one individualized compound I, II or IV | Zineb |
| B-174 | one individualized compound I, II or IV | Ziram |
| B-175 | one individualized compound I, II or IV | Diethofencarb |
| B-176 | one individualized compound I, II or IV | Benthiavalicarb |
| B-177 | one individualized compound I, II or IV | Iprovalicarb |
| B-178 | one individualized compound I, II or IV | Propamocarb |
| B-179 | one individualized compound I, II or IV | Propamocarb hydrochlorid |
| B-180 | one individualized compound I, II or IV | Valiphenal |
| B-181 | one individualized compound I, II or IV | N-(1-(1-(4-cyanophenyl)ethane- |
| | | sulfonyl)-but-2-yl) carbamic acid- |
| | | (4-fluorophenyl) ester |
| B-182 | one individualized compound I, II or IV | Dodine |
| B-183 | one individualized compound I, II or IV | Dodine free base |
| B-184 | one individualized compound I, II or IV | Guazatine |
| B-185 | one individualized compound I, II or IV | Guazatine-acetate |
| B-186 | one individualized compound I, II or IV | Iminoctadine |
| B-187 | one individualized compound I, II or IV | Iminoctadine-triacetate |
| B-188 | one individualized compound I, II or IV | Iminoctadine-tris(albesilate) |
| B-189 | one individualized compound I, II or IV | Kasugamycin |
| B-190 | one individualized compound I, II or IV | Kasugamycin-hydrochloride- |
| | | hydrate |
| B-191 | one individualized compound I, II or IV | Polyoxine |
| B-192 | one individualized compound I, II or IV | Streptomycin |
| B-193 | one individualized compound I, II or IV | Validamycin A |
| B-194 | one individualized compound I, II or IV | Binapacryl |
| B-195 | one individualized compound I, II or IV | Dicloran |
| B-196 | one individualized compound I, II or IV | Dinobuton |
| B-197 | one individualized compound I, II or IV | Dinocap |
| B-198 | one individualized compound I, II or IV | Nitrothal-isopropyl |
| B-199 | one individualized compound I, II or IV | Tecnazen |
| B-200 | one individualized compound I, II or IV | Fentin salts |
| B-201 | one individualized compound I, II or IV | Dithianon |
| B-202 | one individualized compound I, II or IV | Isoprothiolane |
| B-203 | one individualized compound I, II or IV | Edifenphos |
| B-204 | one individualized compound I, II or IV | Fosetyl, Fosetyl-aluminium |
| B-205 | one individualized compound I, II or IV | Iprobenfos |
| B-206 | one individualized compound I, II or IV | Phosphorous acid (H₃PO₃) and |
| | | derivatives |
| B-207 | one individualized compound I, II or IV | Pyrazophos |
| B-208 | one individualized compound I, II or IV | Tolclofos-methyl |
| B-209 | one individualized compound I, II or IV | Chlorothalonil |
| B-210 | one individualized compound I, II or IV | Dichlofluanid |
| B-211 | one individualized compound I, II or IV | Dichlorophen |
| B-212 | one individualized compound I, II or IV | Flusulfamide |
| B-213 | one individualized compound I, II or IV | Hexachlorbenzene |
| B-214 | one individualized compound I, II or IV | Pencycuron |
| B-215 | one individualized compound I, II or IV | Pentachlorophenol and salts |
| B-216 | one individualized compound I, II or IV | Phthalide |
| B-217 | one individualized compound I, II or IV | Quintozene |
| B-218 | one individualized compound I, II or IV | Thiophanate Methyl |
| B-219 | one individualized compound I, II or IV | Tolylfluanid |
| B-220 | one individualized compound I, II or IV | N-(4-chloro-2-nitro-phenyl)-N- |
| | | ethyl-4-methyl- |
| | | benzenesulfonamide |
| B-221 | one individualized compound I, II or IV | Bordeaux mixture |
| B-222 | one individualized compound I, II or IV | Copper acetate |
| B-223 | one individualized compound I, II or IV | Copper hydroxide |
| B-224 | one individualized compound I, II or IV | Copper oxychloride |
| B-225 | one individualized compound I, II or IV | basic Copper sulfate |
| B-226 | one individualized compound I, II or IV | Sulfur |
| B-227 | one individualized compound I, II or IV | Biphenyl |
| B-228 | one individualized compound I, II or IV | Bronopol |
| B-229 | one individualized compound I, II or IV | Cyflufenamid |
| B-230 | one individualized compound I, II or IV | Cymoxanil |
| B-231 | one individualized compound I, II or IV | Diphenylamin |
| B-232 | one individualized compound I, II or IV | Metrafenone |
| B-233 | one individualized compound I, II or IV | Mildiomycin |
| B-234 | one individualized compound I, II or IV | Oxin-copper |
| B-235 | one individualized compound I, II or IV | Prohexadione calcium |
| B-236 | one individualized compound I, II or IV | Spiroxamine |
| B-237 | one individualized compound I, II or IV | Tolylfluanid |
| B-238 | one individualized compound I, II or IV | N-(Cyclopropylmethoxyimino-(6- |
| | | difluoromethoxy-2,3-difluoro- |
| | | phenyl)-methyl)-2-phenyl acetamide |
| B-239 | one individualized compound I, II or IV | N′-(4-(4-chloro-3-trifluoromethyl- |
| | | phenoxy)-2,5-dimethyl-phenyl)- |
| | | N-ethyl-N-methyl formamidine |
| B-240 | one individualized compound I, II or IV | N′-(4-(4-fluoro-3-trifluoromethyl- |
| | | phenoxy)-2,5-dimethyl-phenyl)- |
| | | N-ethyl-N-methyl formamidine |
| B-241 | one individualized compound I, II or IV | N′-(2-methyl-5-trifluoromethyl-4- |
| | | (3-trimethylsilanyl-propoxy)- |
| | | phenyl)-N-ethyl-N-methyl |
| | | formamidine |
| B-242 | one individualized compound I, II or IV | N′-(5-difluoromethyl-2-methyl-4- |
| | | (3-trimethylsilanyl-propoxy)- |
| | | phenyl)-N-ethyl-N-methyl |
| | | formamidine |
| B-243 | one individualized compound I, II or IV | 2-{1-[2-(5-Methyl-3- |
| | | trifluoromethyl-pyrazole-1-yl)- |
| | | acetyl]-piperidin-4-yl}-thiazole-4- |
| | | carboxylic acid methyl-(1,2,3,4- |
| | | tetrahydro-naphthalen-1-yl)- |
| | | amide |
| B-244 | one individualized compound I, II or IV | 2-{1-[2-(5-Methyl-3-trifluoro- |
| | | methyl-pyrazole-1-yl)-acetyl]- |
| | | piperidin-4-yl}-thiazole-4- |
| | | carboxylic acid methyl-(R)- |
| | | 1,2,3,4-tetrahydro-naphthalen-1- |
| | | yl-amide |
| B-245 | one individualized compound I, II or IV | Acetic acid 6-tert-butyl-8-fluoro- |
| | | 2,3-dimethyl-quinolin-4-yl ester |
| B-246 | one individualized compound I, II or IV | Methoxy-acetic acid 6-tert-butyl- |
| | | 8-fluoro-2,3-dimethyl-quinolin-4- |
| | | yl ester |
| B-247 | one individualized compound I, II or IV | Carbaryl |
| B-248 | one individualized compound I, II or IV | Carbofuran |
| B-249 | one individualized compound I, II or IV | Carbosulfan |
| B-250 | one individualized compound I, II or IV | Methomylthiodicarb |
| B-251 | one individualized compound I, II or IV | Bifenthrin |
| B-252 | one individualized compound I, II or IV | Cyfluthrin |
| B-253 | one individualized compound I, II or IV | Cypermethrin |
| B-254 | one individualized compound I, II or IV | alpha-Cypermethrin |
| B-255 | one individualized compound I, II or IV | zeta-Cypermethrin |
| B-256 | one individualized compound I, II or IV | Deltamethrin |
| B-257 | one individualized compound I, II or IV | Esfenvalerate |
| B-258 | one individualized compound I, II or IV | Lambda-cyhalothrin |
| B-259 | one individualized compound I, II or IV | Permethrin |
| B-260 | one individualized compound I, II or IV | Tefluthrin |
| B-261 | one individualized compound I, II or IV | Diflubenzuron |
| B-262 | one individualized compound I, II or IV | Flufenoxuron |
| B-263 | one individualized compound I, II or IV | Lufenuron |
| B-264 | one individualized compound I, II or IV | Teflubenzuron |
| B-265 | one individualized compound I, II or IV | Spirotetramate |
| B-266 | one individualized compound I, II or IV | Clothianidin |
| B-267 | one individualized compound I, II or IV | Dinotefuran |
| B-268 | one individualized compound I, II or IV | Imidacloprid |
| B-269 | one individualized compound I, II or IV | Thiamethoxam |
| B-270 | one individualized compound I, II or IV | Acetamiprid |
| B-271 | one individualized compound I, II or IV | Thiacloprid |
| B-272 | one individualized compound I, II or IV | Endosulfan |
| B-273 | one individualized compound I, II or IV | Fipronil |
| B-274 | one individualized compound I, II or IV | Abamectin |
| B-275 | one individualized compound I, II or IV | Emamectin |
| B-276 | one individualized compound I, II or IV | Spinosad |
| B-277 | one individualized compound I, II or IV | Spinetoram |
| B-278 | one individualized compound I, II or IV | Hydramethylnon |
| B-279 | one individualized compound I, II or IV | Chlorfenapyr |
| B-280 | one individualized compound I, II or IV | Fenbutatin oxide |
| B-281 | one individualized compound I, II or IV | Indoxacarb |
| B-282 | one individualized compound I, II or IV | Metaflumizone |
| B-283 | one individualized compound I, II or IV | Flonicamid |
| B-284 | one individualized compound I, II or IV | Lubendiamide |
| B-285 | one individualized compound I, II or IV | Chlorantraniliprole |
| B-286 | one individualized compound I, II or IV | Cyazypyr (HGW86) |
| B-287 | one individualized compound I, II or IV | Cyflumetofen |
| B-288 | one individualized compound I, II or IV | Acetochlor |
| B-289 | one individualized compound I, II or IV | Dimethenamid |
| B-290 | one individualized compound I, II or IV | metolachlor |
| B-291 | one individualized compound I, II or IV | Metazachlor |
| B-292 | one individualized compound I, II or IV | Glyphosate |
| B-293 | one individualized compound I, II or IV | Glufosinate |
| B-294 | one individualized compound I, II or IV | Sulfosate |
| B-295 | one individualized compound I, II or IV | Clodinafop |
| B-296 | one individualized compound I, II or IV | Fenoxaprop |
| B-297 | one individualized compound I, II or IV | Fluazifop |
| B-298 | one individualized compound I, II or IV | Haloxyfop |
| B-299 | one individualized compound I, II or IV | Paraquat |
| B-300 | one individualized compound I, II or IV | Phenmedipham |
| B-301 | one individualized compound I, II or IV | Clethodim |
| B-302 | one individualized compound I, II or IV | Cycloxydim |
| B-303 | one individualized compound I, II or IV | Profoxydim |
| B-304 | one individualized compound I, II or IV | Sethoxydim |
| B-305 | one individualized compound I, II or IV | Tepraloxydim |
| B-306 | one individualized compound I, II or IV | Pendimethalin |
| B-307 | one individualized compound I, II or IV | Prodiamine |
| B-308 | one individualized compound I, II or IV | Trifluralin |
| B-309 | one individualized compound I, II or IV | Acifluorfen |
| B-310 | one individualized compound I, II or IV | Bromoxynil |
| B-311 | one individualized compound I, II or IV | Imazamethabenz |
| B-312 | one individualized compound I, II or IV | Imazamox |
| B-313 | one individualized compound I, II or IV | Imazapic |
| B-314 | one individualized compound I, II or IV | Imazapyr |
| B-315 | one individualized compound I, II or IV | Imazaquin |
| B-316 | one individualized compound I, II or IV | Imazethapyr |
| B-317 | one individualized compound I, II or IV | 2,4-Dichlorophenoxyacetic acid |
| | | (2,4-D) |
| B-318 | one individualized compound I, II or IV | Chloridazon |
| B-319 | one individualized compound I, II or IV | Clopyralid |
| B-320 | one individualized compound I, II or IV | Fluroxypyr |
| B-321 | one individualized compound I, II or IV | Picloram |
| B-322 | one individualized compound I, II or IV | Picolinafen |
| B-323 | one individualized compound I, II or IV | Bensulfuron |
| B-324 | one individualized compound I, II or IV | Chlorimuron-ethyl |
| B-325 | one individualized compound I, II or IV | Cyclosulfamuron |
| B-326 | one individualized compound I, II or IV | Iodosulfuron |
| B-327 | one individualized compound I, II or IV | Mesosulfuron |
| B-328 | one individualized compound I, II or IV | Metsulfuron-methyl |
| B-329 | one individualized compound I, II or IV | Nicosulfuron |
| B-330 | one individualized compound I, II or IV | Rimsulfuron |
| B-331 | one individualized compound I, II or IV | Triflusulfuron |
| B-332 | one individualized compound I, II or IV | Atrazine |
| B-333 | one individualized compound I, II or IV | Hexazinone |
| B-334 | one individualized compound I, II or IV | Diuron |
| B-335 | one individualized compound I, II or IV | Florasulam |
| B-336 | one individualized compound I, II or IV | Pyroxasulfone |
| B-337 | one individualized compound I, II or IV | Bentazone |
| B-338 | one individualized compound I, II or IV | Cinidon-ethlyl |
| B-339 | one individualized compound I, II or IV | Cinmethylin |
| B-340 | one individualized compound I, II or IV | Dicamba |
| B-341 | one individualized compound I, II or IV | Diflufenzopyr |
| B-342 | one individualized compound I, II or IV | Quinclorac |
| B-343 | one individualized compound I, II or IV | Quinmerac |
| B-344 | one individualized compound I, II or IV | Mesotrione |
| B-345 | one individualized compound I, II or IV | Saflufenacil |
| B-346 | one individualized compound I, II or IV | Topramezone |

The active substances referred to as component 2, their preparation and their activity against harmful fungi is known (cf.: http://www.alanwood.net/pesticides/); these substances are commercially available. The compounds described by IUPAC nomenclature, their preparation and their fungicidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624).

The mixtures of active substances can be prepared as compositions comprising besides the active ingredients at least one inert ingredient by usual means, e.g. by the means given for the compositions of compounds I, II and/or IV.

Concerning usual ingredients of such compositions reference is made to the explanations given for the compositions containing compounds I, II and/or IV.

The mixtures of active substances according to the present invention are suitable as fungicides, as are the compounds of formula I, II ad IV. They are distinguished by an outstanding effectiveness against a broad spectrum of phytopathogenic fungi, especially from the classes of the Ascomycetes, Basidiomycetes, Deuteromycetes and Peronosporomycetes (syn. Oomycetes). In addition, it is referred to the explanations regarding the fungicidal activity of the compounds and the compositions containing compounds I, II and/or IV respectively.

The compounds I, II and IV and pharmaceutically acceptable salts thereof are also suitable for treating diseases in men and animals, especially as antimycotics, for treating cancer and for treating virus infections. The term “antimycotic”, as distinguished from the term “fungicide”, refers to a medicament for combating zoopathogenic or humanpathogenic fungi, i.e. for combating fungi in animals, especially in mammals (including humans) and birds.

Thus, a further aspect of the present invention relates to a medicament comprising at least one compound of the formulae I, II and/or IV and/or at least one pharmaceutically acceptable salt thereof and a pharmaceutically acceptable carrier.

Suitable pharmaceutically acceptable salts are especially physiologically tolerated salts of the compound I, in particular the acid addition salts with physiologically acceptable acids. Examples of suitable organic and inorganic acids are hydrochloric acid, hydrobromic acid, phosphoric acid, sulfuric acid, C₁-C₄-alkylsulfonic acids, such as methanesulfonic acid, aromatic sulfonic acids, such as benzenesulfonic acid and toluenesulfonic acid, oxalic acid, maleic acid, fumaric acid, lactic acid, tartaric acid, adipic acid and benzoic acid. Further suitable acids are described, for example, in Fortschritte der Arzneimittelforschung, Volume 10, pages 224 ff., Birkhäuser Verlag, Basle and Stuttgart, 1966, the entire contents of which is expressly incorporated herein by way of reference.

Suitable carriers are, for example, solvents, carriers, excipients, binders and the like customarily used for pharmaceutical formulations, which are described below in an exemplary manner for individual types of administration.

A further aspect of the present invention relates to the use of compounds I, II and IV or of pharmaceutically acceptable salts thereof for preparing an antimycotic medicament; i.e. for preparing a medicament for the treatment and/or prophylaxis of infections with humanpathogenic and/or zoopathogenic fungi. Another aspect of the present invention relates to the use of compounds of formulae I, II and/or IV or of pharmaceutically acceptable salts thereof for preparing a medicament for the treatment of cancer. Another aspect of the present invention relates to the use of compounds of formulae I, II and/or IV or of pharmaceutically acceptable salts thereof for preparing a medicament for the treatment or prophylaxis of virus infections.

The compounds of formulae I, II and IV and/or their pharmaceutically acceptable salts are suitable for the treatment, inhibiton or control of growth and/or propagation of tumor cells and the disorders associated therewith. Accordingly, they are suitable for cancer therapy in warm-blooded vertebrates, for example mammals and birds, in particular man, but also other mammals, in particular useful and domestic animals, such as dogs, cats, pigs, ruminants (cattle, sheep, goats, bison, etc.), horses and birds, such as chicken, turkey, ducks, geese, guineafowl and the like.

The compounds of formulae I, II and IV and/or their pharmaceutically acceptable salts are suitable for the therapy of cancer or cancerous disorders of the following organs:
breast, lung, intestine, prostate, skin (melanoma), kidney, bladder, mouth, larynx, oesophagus, stomach, ovaries, pancreas, liver and brain or CNS.

The compounds of formulae I, II and IV and/or their pharmaceutically acceptable salts are suitable for the treatment of virus infections in warm-blooded vertebrates, for example mammals and birds, in particular man, but also other mammals, in particular useful and domestic animals, such as dogs, cats, pigs, ruminants (cattle, sheep, goats, bison, etc.), horses and birds, such as chicken, turkey, ducks, geese, guineafowl and the like. They are suitable for treating virus infections like retrovirus infections such as HIV and HTLV, influenza virus infection, rhinovirus infections, herpes and the like.

The compounds according to the invention can be administered in a customary manner, for example orally, intravenously, intramuscularly or subcutaneously. For oral administration, the active compound can be mixed, for example, with an inert diluent or with an edible carrier; it can be embedded into a hard or soft gelatin capsule, it can be compressed to tablets or it can be mixed directly with the food/feed. The active compound can be mixed with excipients and administered in the form of indigestible tablets, buccal tablets, pastilles, pills, capsules, suspensions, potions, syrups and the like. Such preparations should contain at least 0.1% of active compound. The composition of the preparation may, of course, vary. It usually comprises from 2 to 60% by weight of active compound, based on the total weight of the preparation in question (dosage unit). Preferred preparations of the compound I according to the invention comprise from 10 to 1000 mg of active compound per oral dosage unit.

The tablets, pastilles, pills, capsules and the like may furthermore comprise the following components: binders, such as traganth, gum arabic, corn starch or gelatin, excipients, such as dicalcium phosphate, disintegrants, such as corn starch, potato starch, alginic acid and the like, glidants, such as magnesium stearate, sweeteners, such as sucrose, lactose or saccharin, and/or flavors, such as peppermint, vanilla and the like. Capsules may furthermore comprise a liquid carrier. Other substances which modify the properties of the dosage unit may also be used. For example, tablets, pills and capsules may be coated with schellack, sugar or mixtures thereof. In addition to the active compound, syrups or potions may also comprise sugar (or other sweeteners), methyl- or propylparaben as preservative, a colorant and/or a flavor. The components of the active compound preparations must, of course, be pharmaceutically pure and nontoxic at the quantities employed. Furthermore, the active compounds can be formulated as preparations with a controlled release of active compound, for example as delayed-release preparations.

The active compounds can also be administered parenterally or intraperitoneally. Solutions or suspensions of the active compounds or their salts can be prepared with water using suitable wetting agents, such as hydroxypropylcellulose. Dispersions can also be prepared using glycerol, liquid polyethylene glycols and mixtures thereof in oils. Frequently, these preparations furthermore comprise a preservative to prevent the growth of microorganisms.

Preparations intended for injections comprise sterile aqueous solutions and dispersions and also sterile powders for preparing sterile solutions and dispersions. The preparation has to be sufficiently liquid for injection. It has to be stable under the preparation and storage conditions and it has to be protected against contamination by microorganisms. The carrier may be a solvent or a dispersion medium, for example, water, ethanol, a polyol (for example glycerol, propylene glycol or liquid polyethylene glycol), a mixture thereof and/or a vegetable oil.

The invention is further illustrated by the following, non-limiting examples.

### I. SYNTHESIS EXAMPLES

Log P is the partition coefficient between water and octanol.

### I.1 Synthesis of Compounds IV

### 1. 1-{2-[2-Chloro-4-(4-chloro-phenoxy)-phenyl]-4-methyl-[1,3]dioxan-2-ylmethyl}-1H-[1,2,4]triazole (Compound IVD.1)

### 1.1 Synthesis of 1-[2-chloro-4-(4-chlorophenoxy)]acetophenone

4-Chlorophenol (193.5 g, 1.49 mol) was dissolved in DMF (1 litre) and potassium carbonate (247.1 g, 1.79 mol) was added. 2-Chloro-4-fluoroacetophenone (259.8 g, 1.49 mol) was added and the mixture stirred at 115° C. for 8 hours. The mixture was cooled to room temperature and water (2 litres) and saturated aqueous sodium chloride solution (500 ml) was added and the organic components extracted with MTBE (methyl-tert-butyl ether) (4 litres), washed with further saturated aqueous sodium chloride solution (2×500 ml) and dried over sodium sulfate. Evaporation of the solvents gave the diaryl ether as a brown oil (420 g, 90%), which was used without further purification.

### 1.2 Synthesis of 1-[2-chloro-4-(4-chlorophenoxy)]-2-bromoacetophenone

Bromine (12.6 g, 4.1 ml, 78.6 mmol) was added to dropwise to a solution of the diaryl ether from step 1.1 (34.0 g, 54.4 mmol) in diethyl ether (250 ml) at room temperature. The reaction mixture was stirred at room temperature for 30 minutes, then water (1 litre) and saturated sodium hydrogen carbonate solution (700 ml) was added. The organic components were extracted with MTBE (800 ml) and the extracts washed with saturated sodium hydrogen carbonate solution, then saturated sodium chloride solution and dried over sodium sulfate. Evaporation of the solvents gave the bromoacetophenone as a brown oil (36 g, 73%).

### 1.3 Synthesis of 1-[2-chloro-4-(4-chlorophenoxy)]-2-[1,2,4]triazol-1-yl-acetophenone

1,2,4-Triazole (22.9 g, 324 mmol) was added portionwise to a mixture of sodium hydride (7.8 g, 324 mmol) and THF (900 ml) and the mixture stirred at room temperature for 15 minutes. A solution of bromoacetophenone from step 1.2 (90 g, 250 mmol) in THF (300 ml) was added dropwise and the resultant mixture stirred at room temperature overnight. Water (1 litre) and saturated sodium chloride solution (500 ml) was added and the organic components extracted with MTBE (1.5 litres). The organic extracts were washed with further saturated sodium chloride solution (500 ml), dried over sodium sulfate and the solvents evaporated. Trituration with a mixture of diisopropyl ether (1 litre) and MTBE (200 ml) gave the ketone as an orange coloured solid (72.3 g, 79%).

### 1.4 Synthesis of 1-{2-[2-chloro-4-(4-chloro-phenoxy)-phenyl]-4-methyl-[1,3]dioxan-2-ylmethyl}-1H-[1,2,4]triazole

A mixture of the ketone from step 1.3 (0.5 g, 1.36 mmol), 1,3-butanediol (0.2 g, 2.20 mmol), p-toluenesulfonic acid (0.1 g) and toluene (60 ml) was heated to reflux and the water formed in the reaction was removed using a Dean-Stark trap. After 60 hours heating, additional diol (0.4 g) and acid (0.1 g) was added and the heating continued for a further 60 hours. The reaction mixture was cooled to room temperature and added to a saturated aqueous solution of K₂CO₃ (50 ml) and the organic components extracted with ethyl acetate (3×100 ml), washed with saturated sodium chloride solution and dried over sodium sulfate. Evaporation of the solvents followed by purification by column chromatography gave the title compound as a brown solid (0.26 g, 46%).

LCMS: 3.840 minutes

### 2. 1-{2-[2-Chloro-4-(2,4-dichloro-phenoxy)-5-fluoro-phenyl]-4-methyl-[1,3]dioxolan-2-ylmethyl}-1H-[1,2,4]triazole (Compound IVA.45)

1-[2-Chloro-5-fluoro-4-(2,4-dichlorophenoxy)]-2-bromoacetophenone was prepared in analogy to 1-[2-chloro-4-(4-chlorophenoxy)]-2-bromoacetophenone (see above steps 1.1 and 1.2).

### 2.1 Synthesis of 2-bromomethyl-2-[2-chloro-4-(2,4-dichloro-phenoxy)-5-fluoro-phenyl]-4-methyl-[1,3]dioxolane

A mixture of 1-[2-chloro-5-fluoro-4-(2,4-dichlorophenoxy)]-2-bromoacetophenone (2.6 g, 6.3 mmol), 1,2-propanediol (0.73 g, 9.6 mmol), p-toluenensulfonic acid (152 mg) and toluene (20 ml) was heated to reflux and the water formed in the reaction was removed using a Dean-Stark trap. After 18 hours the reaction was cooled to room temperature, aqueous NaHCO₃ was added and the organic components extracted with MTBE (methyl-tert-butyl ether). The organic extracts were dried over sodium sulfate, and the solvents evaporated to give the ketal as a brown oil, which was used without further purification.

### 2.2 Synthesis of 1-{2-[2-chloro-4-(2,4-dichloro-phenoxy)-5-fluoro-phenyl]-4-methyl-[1,3]dioxolan-2-ylmethyl}-1H-[1,2,4]triazole

A mixture of the ketal from step 2.1 above (3.5 g, 7.44 mmol), 1,2,4-triazole (0.565 g, 8.18 mmol), caesium carbonate (3.64 g, 11.12 mmol) and DMF (37 ml) was stirred at 100° C. for 60 hours. The reaction mixture was cooled to room temperature, water was added and the organic components extracted with MTBE. The organic extracts were washed with an aqueous LiCl solution and dried over sodium sulfate. Evaporation of the solvents and purification by column chromatography gave the title compound as a yellow oil (2.43 g, 71%).

LCMS: 3.913 and 3.934 (2 diastereomers)

The following compounds were synthesized in analogy to the above examples 1 or 2:

### 3. 1-{2-[4-(4-Chloro-2-fluoro-phenoxy)-phenyl]-4-methyl-[1,3]dioxolan-2-ylmethyl}-1H-[1,2,4]triazole (Compound IVA. 1)

Melting point: 113-115° C.

### 4. 1-{2-[4-(2,4-Difluoro-phenoxy)-phenyl]-4-methyl-[1,3]dioxolan-2-ylmethyl}-1H-[1,2,4]triazole (Compound IVA.2)

Melting point: 83-86° C.

### 5. 1-{2-[4-(2,4-Difluoro-phenoxy)-phenyl]-4-ethyl-[1,3]dioxolan-2-ylmethyl}-1H-[1,2,4]triazole (Compound IVA.3)

Melting point: 112-114° C.

### 6. 1-{2-[4-(6-Chloro-2-fluoro-phenoxy)-phenyl]-4-methyl-[1,3]dioxolan-2-ylmethyl}-1H-[1,2,4]triazole (Compound IVA.4)

Melting point: 112-114° C.

### 7. 1-{2-[4-(6-Chloro-2-fluoro-phenoxy)-phenyl]-4-ethyl-[1,3]dioxolan-2-ylmethyl}-1H-[1,2,4]triazole (Compound IVA.5)

LCMS: 3.424 minutes

### 8. 1-{2-[4-(6-Chloro-2-fluoro-phenoxy)-phenyl]-4-propyl-[1,3]dioxolan-2-ylmethyl}-1H-[1,2,4]triazole (Compound IVA.6)

LCMS: 3.611 minutes

### 9. 1-{2-[4-(2,6-Difluoro-phenoxy)-phenyl]-4-ethyl-[1,3]dioxolan-2-ylmethyl}-1H-[1,2,4]triazole (Compound IVA.7)

Melting point: 90-95° C.

### 10. 1-{2-[4-(2,6-Difluoro-phenoxy)-phenyl]-4-propyl-[1,3]dioxolan-2-ylmethyl}-1H-[1,2,4]triazole (Compound IVA.8)

LCMS: 3.482 minutes

### 11. 1-{2-[4-(5-Chloro-2-fluoro-phenoxy)-phenyl]-4-methyl-[1,3]dioxolan-2-ylmethyl}-1H-[1,2,4]triazole (Compound IVA.9)

LCMS: 3.320 minutes

### 12. 1-{2-[4-(5-Chloro-2-fluoro-phenoxy)-phenyl]-4-ethyl-[1,3]dioxolan-2-ylmethyl}-1H-[1,2,4]triazole (Compound IVA.10)

LCMS: 3.51 minutes

### 13. 1-{2-[4-(5-Chloro-2-fluoro-phenoxy)-phenyl]-4-propyl-[1,3]dioxolan-2-ylmethyl}-1H-[1,2,4]triazole (Compound IVA. 11)

LCMS: 3.70 minutes

### 14. 1-{2-[4-(4-Chloro-2-fluoro-phenoxy)-phenyl]-4-ethyl-[1,3]dioxolan-2-ylmethyl}-1H-[1,2,4]triazole (Compound IVA.12)

LCMS: 3.652 minutes

### 15. 1-{2-[4-(4-Chloro-2-fluoro-phenoxy)-phenyl]-4-propyl-[1,3]dioxolan-2-ylmethyl}-1H-[1,2,4]triazole (Compound IVA. 13)

LCMS: 3.718 minutes

### 16. 1-{2-[4-(2,5-Difluoro-phenoxy)-phenyl]-4-methyl-[1,3]dioxolan-2-ylmethyl}-1H-[1,2,4]triazole (Compound IVA.14)

LCMS: 3.104 minutes

### 17. 1-{2-[4-(2,5-Difluoro-phenoxy)-phenyl]-4-ethyl-[1,3]dioxolan-2-ylmethyl}-1H-[1,2,4]triazole (Compound IVA.15)

LCMS: 3.303 minutes

### 18. 1-{2-[4-(2,5-Difluoro-phenoxy)-phenyl]-4-propyl-[1,3]dioxolan-2-ylmethyl}-1H-[1,2,4]triazole (Compound IVA.16)

LCMS: 3.494 minutes

### 19. 1-{2-[2-Chloro-4-(2,4-difluoro-phenoxy)-phenyl]-4-methyl-[1,3]dioxolan-2-ylmethyl}-1H-[1,2,4]triazole (Compound IVA.17)

LCMS: 3.499 minutes

### 20. 1-{2-[2-Chloro-4-(2,6-difluoro-phenoxy)-phenyl]-4-methyl-[1,3]dioxolan-2-ylmethyl}-1H-[1,2,4]triazole (Compound IVA.18)

LCMS: 3.298 minutes

### 21. 1-{2-[4-(2,4-Difluoro-phenoxy)-2-methyl-phenyl]-4-methyl-[1,3]dioxolan-2-ylmethyl}-1H-[1,2,4]triazole (Compound IVA.19)

LCMS: 3.362 minutes

### 22. 1-{2-[2-Chloro-4-(4-chloro-2-fluoro-phenoxy)-phenyl]-4-methyl-[1,3]dioxolan-2-ylmethyl}-1H-[1,2,4]triazole (Compound IVA.20)

LCMS: 3.884 minutes

### 23. 1-{2-[4-(4-chloro-2-fluoro-phenoxy)-2-methyl-phenyl]-4-methyl-[1,3]dioxolan-2-ylmethyl}-1H-[1,2,4]triazole (Compound IVA.21)

LCMS: 3.706 minutes

### 24. 1-{2-[4-(4-chloro-2-fluoro-phenoxy)-2-trifluoromethyl-phenyl]-4-methyl-[1,3]dioxolan-2-ylmethyl}-1H-[1,2,4]triazole (Compound IVA.22)

LCMS: 3.816 minutes

### 25. 1-{2-[2-Chloro-4-(6-chloro-2-fluoro-phenoxy)-phenyl]-4-methyl-[1,3]dioxolan-2-ylmethyl}-1H-[1,2,4]triazole (Compound IVA23)

LCMS: 3.394 minutes

### 26. 1-{2-[2-Chloro-4-(2,5-difluoro-phenoxy)-phenyl]-4-methyl-[1,3]dioxolan-2-ylmethyl}-1H-[1,2,4]triazole (Compound IVA.24)

Melting point: 119.9° C.

### 27. 1-{2-[4-(2,5-Difluoro-phenoxy)-2-methyl-phenyl]-4-methyl-[1,3]dioxolan-2-ylmethyl}-1H-[1,2,4]triazole (Compound IVA.25)

LCMS: 3.420 minutes

### 28. 1-{2-[4-(2,4-Dichloro-phenoxy)-2-methyl-phenyl]-4-methyl-[1,3]dioxolan-2-ylmethyl}-1H-[1,2,4]triazole (Compound IVA.26)

LCMS: 3.849 minutes

### 29. 1-{2-[4-(3,5-Dichloro-phenoxy)-2-methyl-phenyl]-4-methyl-[1,3]dioxolan-2-ylmethyl}-1H-[1,2,4]triazole (Compound IVA.27)

LCMS: 3.904 minutes

### 30. 1-{2-[4-(2-Chloro-phenoxy)-2-methyl-phenyl]-4-methyl-[1,3]dioxolan-2-ylmethyl}-1H-[1,2,4]triazole (Compound IVA. 128

LCMS: 3.588 minutes

### 31. 1-{2-[2-Chloro-4-(4-chloro-2-fluoro-phenoxy)-5-fluoro-phenyl]-4-methyl-[1,3]dioxolan-2-ylmethyl}-1H-[1,2,4]triazole (Compound IVA.29)

LCMS: 3.722 minutes

### 32. 1-{2-[4-(4-Chloro-phenoxy)-3-fluoro-phenyl]-4-methyl-[1,3]dioxolan-2-ylmethyl}-1H-[1,2,4]triazole (Compound IVA.30)

Log P: 4.2

### 33. 1-{2-[2-Fluoro-4-phenoxy-phenyl]-4-methyl-[1,3]dioxolan-2-ylmethyl}-1H-[1,2,4]triazole (Compound IVA.31)

### 34. 1-{2-[4-(4-Chloro-phenoxy)-2-fluoro-phenyl]-4-methyl-[1,3]dioxolan-2-ylmethyl}-1H-[1,2,4]triazole (Compound IVA.32)

### 35. 1-{2-[3-Fluoro-4-phenoxy-phenyl]-4-methyl-[1,3]dioxolan-2-ylmethyl}-1H-[1,2,4]triazole (Compound IVA.33)

Melting point: 108-110° C.

### 36. 1-{2-[2-Chloro-4-(2,4-difluoro-phenoxy)-5-fluoro-phenyl]-4-methyl-[1,3]dioxolan-2-ylmethyl}-1H-[1,2,4]triazole (Compound IVA.34)

LCMS: 3.458 minutes

### 37. 1-{2-[4-(4-Chloro-phenoxy)-2,5-difluoro-phenyl]-4-methyl-[1,3]dioxolan-2-ylmethyl}-1H-[1,2,4]triazole (Compound IVA.35)

Melting point: 86-88° C.

### 38. 1-{2-[2-Chloro-4-(4-chloro-phenoxy)-phenyl]-4-ethyl-[1,3]dioxolan-2-ylmethyl}-1H-[1,2,4]triazole (Compound IVA.36)

Log P-5.0

### 39. 1-{2-[2-Chloro-4-(4-chloro-phenoxy)-phenyl]-4-propyl-[1,3]dioxolan-2-ylmethyl}-1H-[1,2,4]triazole (Compound IVA.37)

Log P-5.4

### 40. 1-{2-[3,6-Difluoro-4-phenoxy-phenyl]-4-methyl-[1,3]dioxolan-2-ylmethyl}-1H-[1,2,4]triazole (Compound IVA.38)

Melting point: 102-104° C.

### 41. 1-{2-[3,5-Difluoro-4-phenoxy-phenyl]-4-methyl-[1,3]dioxolan-2-ylmethyl}-1H-[1,2,4]triazole (Compound IVA.39)

Log P-3.7

### 42. 1-{2-[4-(4-Chloro-phenoxy)-2-trifluoromethyl-phenyl]-4-methyl-[1,3]dioxolan-2-ylmethyl}-1H-[1,2,4]triazole (Compound IVA.40)

LCMS: 3.961 minutes

### 43. 1-{2-[2-Chloro-4-(4-chloro-phenoxy)-5-fluoro-phenyl]-4-methyl-[1,3]dioxolan-2-ylmethyl}-1H-[1,2,4]triazole (Compound IVA.41)

LCMS—3.793 minutes

### 44. 1-{2-[4-(2,4-Dichloro-phenoxy)-2-trifluoromethyl-phenyl]-4-methyl-[1,3]dioxolan-2-ylmethyl}-1H-[1,2,4]triazole (Compound IVA.42)

LCMS: 4.079 minutes

### 45. 1-{2-[4-(2,4-Dichloro-phenoxy)-2-trifluoromethyl-phenyl]-[1,3]dioxolan-2-ylmethyl}-1H-[1,2,4]triazole (Compound IVA.43)

LCMS: 3.850 minutes

### 46. 1-{2-[4-(2-Chloro-phenoxy)-2-trifluoromethyl-phenyl]-[1,3]dioxolan-2-ylmethyl}-1H-[1,2,4]triazole (Compound IVA.44)

LCMS: 3.568 minutes

### 47. 1-{2-[2-Chloro-4-(2,4-dichloro-phenoxy)-5-fluoro-phenyl]-4-methyl-[1,3]dioxolan-2-ylmethyl}-1H-[1,2,4]triazole (Compound IVA.45)

LCMS: 3.913 minutes

### 48. 1-{2-[2-Chloro-4-(2-chloro-phenoxy)-5-fluoro-phenyl]-4-methyl-[1,3]dioxolan-2-ylmethyl}-1H-[1,2,4]triazole (Compound IVA.46)

LCMS: 3.559 minutes

### 49. 1-{2-[2-Chloro-4-(4-chloro-phenoxy)-phenyl]-hexahydrobenzo[1,3]dioxolan-2-ylmethyl}-1H-[1,2,4]triazole (Compound IVB)

LCMS: 4.068 minutes

### 50. 1-{2-[2-Chloro-4-(4-chloro-phenoxy)-phenyl]-benzo[1,3]dioxolan-2-ylmethyl}-1H-[1,2,4]triazole (Compound IVC)

LCMS: 4.010 minutes

### 51. 1-{2-[2-chloro-4-(4-chloro-phenoxy)-phenyl]-4,6-dimethyl-[1,3]dioxan-2-ylmethyl}-1H-[1,2,4]triazole (Compound IVD.2)

LCMS: 4.058 minutes

### 52. 1-{2-[2-chloro-4-(4-chloro-phenoxy)-phenyl]-5-tert-butyl-[1,3]dioxan-2-ylmethyl}-1H-[1,2,4]triazole (Compound IVD.3)

LCMS: 4.325 minutes

### 53. 1-{2-[2-chloro-4-(4-chloro-phenoxy)-phenyl]-5-phenyl-[1,3]dioxan-2-ylmethyl}-1H-[1,2,4]triazole (Compound IVD.4)

LCMS: 4.152 minutes

### 54. 1-{2-[2-chloro-4-(4-chloro-phenoxy)-phenyl]-5-methyl-[1,3]dioxan-2-ylmethyl}-1H-[1,2,4]triazole (Compound IVD.5)

LCMS: 3.799 minutes

### 55. 1-{2-[2-chloro-4-(4-chloro-phenoxy)-phenyl]-5,5-dimethyl-[1,3]dioxan-2-ylmethyl}-1H-[1,2,4]triazole (Compound IVD.6)

LCMS: 3.951 minutes

### 56. 1-{2-[2-chloro-4-(4-chloro-phenoxy)-phenyl]-5,5-dimethyl-4-propyl-[1,3]dioxan-2-ylmethyl}-1H-[1,2,4]triazole (Compound IVD.7)

LCMS: 4.743 minutes

### 57. 1-{2-[4-(4-Chloro-phenoxy)-2-methylphenyl]-[1,3]dioxan-2-ylmethyl}-1H-[1,2,4]triazole (Compound IVD.10)

LCMS: 3.573 minutes

### 58. 1-{2-[4-(4-Chloro-phenoxy)-2-methylphenyl]-4-methyl-[1,3]dioxan-2-ylmethyl}-1H-[1,2,4]triazole (Compound IVD.11)

LCMS: 3.929 minutes

### 59. 1-{2-[4-(4-Chloro-phenoxy)-2-methylphenyl]-5-tert-butyl-[1,3]dioxan-2-ylmethyl}-1H-[1,2,4]triazole (Compound IVD.15)

LCMS: 4.251 minutes

### 60. 1-{2-[4-(4-Chloro-phenoxy)-2-methylphenyl]-5-methyl-[1,3]dioxan-2-ylmethyl}-1H-[1,2,4]triazole (Compound IVD.17)

LCMS: 3.818 minutes

### 61. 1-{2-[4-(4-Chloro-phenoxy)-2-methylphenyl]-5,5-dimethyl-[1,3]dioxan-2-ylmethyl}-1H-[1,2,4]triazole (Compound IVD.18)

LCMS: 4.091 minutes

### 1.2 Synthesis of compounds I/II

### 62. 2-{2-[2-Chloro-4-(4-chloro-phenoxy)-phenyl]-4-methyl-[1,3]dioxan-2-yl-methyl}-2H-[1,2,4]triazole-3-thiol (Compound I.C1.1)

Butyllithium (1.57 mmol of a 1.6M solution in hexane) was added dropwise to a solution of the triazole from example 1 above (300 mg, 0.71 mmol) in THF (6 ml) at −20° C. The mixture was warmed to room temperature and stirred for 30 minutes then cooled again to −70° C. Sulfur (27 mg, 0.86 mmol) was then added and the mixture stirred at −70° C. for one hour. At −70° C. a saturated solution of ammonium chloride (2 ml) was added and the mixture warmed to room temperature and further water (50 ml) was added. The organic components were extracted with MTBE (100 ml), the organic extracts washed with a saturated solution of ammonium chloride and dried over sodium sulfate. Evaporation of the solvents followed by purification by reverse phase chromatography gave the titled thiotriazole (130 mg, 38%) as a brown solid.

LCMS: 3.841 minutes

The following compounds were synthesized in analogy to the above example 63:

### 63. 2-{2-[4-(4-Chloro-phenoxy)-2,5-difluoro-phenyl]-4-methyl-[1,3]dioxolan-2-ylmethyl}-2H-[1,2,4]triazole-3-thiol (compound I.C1.2)

Log P-3.2

### 64. 2-{2-[2,5-Difluoro-4-phenoxy-phenyl]-4-methyl-[1,3]dioxolan-2-yl-methyl}-2H-[1,2,4]triazole-3-thiol (compound I.C1.3)

Log P-2.9

### 65. 2-{2-[2-Chloro-4-(4-chloro-phenoxy)-phenyl]-4-propyl-[1,3]dioxolan-2-yl-methyl}-2H-[1,2,4]triazole-3-thiol (compound I.C1.4)

Melting point: 105-106° C.

### 66. 2-{2-[2-Chloro-4-(4-chloro-phenoxy)-phenyl]-4-methoxymethyl-[1,3]dioxolan-2-ylmethyl}-2H-[1,2,4]triazole-3-thiol (compound I.C1.5)

Melting point: 153-155° C.

### 67. 2-{2-[3-Fluoro-4-phenoxy-phenyl]-4-methyl-[1,3]dioxolan-2-yl-methyl}-2H-[1,2,4]triazole-3-thiol (compound I.C1.6)

Melting point: 62-64° C.

### 68. 2-{2-[3,5-Difluoro-4-phenoxy-phenyl]-4-methyl-[1,3]dioxolan-2-yl-methyl}-2H-[1,2,4]triazole-3-thiol (compound I.C1.7)

Melting point: 139-141° C.

### 69. 2-{2-[4-(4-Chloro-phenoxy)-3-fluoro-phenyl]-4-methyl-[1,3]dioxolan-2-yl-methyl}-2H-[1,2,4]triazole-3-thiol (compound I.C1.8)

Log P-3.6

### 70. 2-{2-[4-(4-Chloro-phenoxy)-2-fluoro-phenyl]-4-methyl-[1,3]dioxolan-2-yl-methyl}-2H-[1,2,4]triazole-3-thiol (compound I.C1.9)

Log P-3.5

### 71. 2-{2-[4-(4-Chloro-phenoxy)-2-methyl-phenyl]-4-methyl-[1,3]dioxolan-2-yl-methyl}-2H-[1,2,4]triazole-3-thiol (compound I.C1.10)

LCMS—3.645 minutes

### II. EXAMPLES OF THE ACTION AGAINST HARMFUL FUNGI

The fungicidal action of the compounds of the formulae I and II was demonstrated by the following experiments:

### A) Green House Tests

The spray solutions were prepared in several steps:

The stock solution were prepared: a mixture of acetone and/or dimethylsulfoxide and the wetting agent/emulsifier Wettol, which is based on ethoxylated alkylphenoles, in a relation (volume) solvent-emulsifier of 99 to 1 was added to 25 mg of the compound to give a total of 10 ml. Water was then added to total volume of 100 ml. This stock solution was diluted with the described solvent-emulsifier-water mixture to the given concentration.

1. Curative Control of Soy Bean Rust on Soy Beans Caused by Phakopsora pachyrhizi

Leaves of pot-grown soy bean seedlings were inoculated with spores of Phakopsora pachyrhizi. To ensure the success of the artificial inoculation, the plants were transferred to a humid chamber with a relative humidity of about 95% and 20 to 24° C. for 24 h. The next day the plants were cultivated for 2 days in a greenhouse chamber at 23-27° C. and a relative humidity between 60 and 80%. Then the plants were sprayed to run-off with an aqueous suspension, containing the concentration of active ingredient or their mixture as described below. The plants were allowed to air-dry. Then the trial plants were cultivated for 14 days in a greenhouse chamber at 23-27° C. and a relative humidity between 60 and 80%. The extent of fungal attack on the leaves was visually assessed as % diseased leaf area. The plants which had been treated with an aqueous active compound preparation comprising 300 ppm of the active compounds of examples 1, 3, 4, 5, 6, 7, 8, 9, 10, 11, 12, 13, 14, 15, 16, 18, 19, 20, 21, 22, 24, 29, 30, 31, 32, 33, 34, 38, 39, 40, 41, 42, 43, 46, 48, 49, 50, 51, 52, 62, 63, 64, 65, 66, 67, 68 and 69 showed an infection of at most 15%, whereas the untreated plants were 60% infected.

2. Preventative Control of Leaf Blotch on Wheat Caused by Septoria tritici

Leaves of pot-grown wheat seedling were sprayed to run-off with an aqueous suspension of the active compound or their mixture, prepared as described. The plants were allowed to air-dry. At the following day the plants were inoculated with an aqueous spore suspension of Septoria tritici. Then the trial plants were immediately transferred to a humid chamber at 18-22° C. and a relative humidity close to 100%. After 4 days the plants were transferred to a chamber with 18-22° C. and a relative humidity close to 70%. After 4 weeks the extent of fungal attack on the leaves was visually assessed as % diseased leaf area. The plants which had been treated with an aqueous active compound preparation comprising 300 ppm of the active compounds of examples 5, 6, 7, 8, 11, 14, 15, 16, 19, 21, 22, 23, 24, 35, 37, 38, 39, 40, 44, 62, 63, 64, 65 and 67 showed an infection of at most 15%, whereas the untreated plants were 90% infected.

3. Preventative Control of Brown Rust on Wheat Caused by Puccinia recondita

The first two developed leaves of pot-grown wheat seedling were sprayed to run-off with an aqueous suspension, containing the concentration of active ingredient or their mixture as described below. The next day the plants were inoculated with spores of Puccinia recondita. To ensure the success the artificial inoculation, the plants were transferred to a humid chamber without light and a relative humidity of 95 to 99% and to 24° C. for 24 h. Then the trial plants were cultivated for 6 days in a greenhouse chamber at 20-24° C. and a relative humidity between 65 and 70%. The extent of fungal attack on the leaves was visually assessed as % diseased leaf area. The plants which had been treated with an aqueous active compound preparation comprising 300 ppm of the active compounds of examples 1, 25, 26, 27, 28, 41, 42, 43, 49, 50, 51, 65 and 66 showed an infection of at most 15%, whereas the untreated plants were 90% infected.

1-32. (canceled)

33. A compound of formula I or II 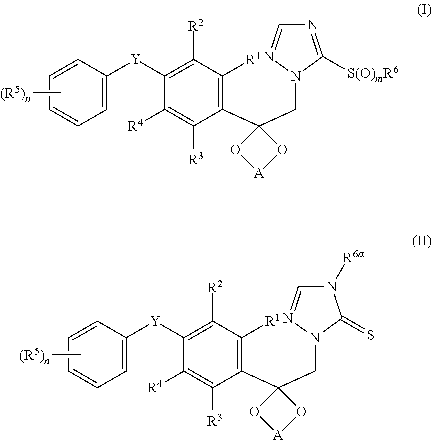
wherein
A is a linear C₁-C₅-alkylene bridge which may be substituted by 1, 2, 3, 4, 5 or 6 substituents R⁷;
Y is O, S or NR⁸;
R¹, R², R³ and R⁴, independently of each other, are selected from the group consisting of hydrogen, halogen, OH, SH, NO₂, CN, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₈-cycloalkyl, C₃-C₈-halocycloalkyl, C₁-C₄-alkoxy, C₁-C₄-haloalkoxy, C₁-C₄-alkenyloxy, C₁-C₄-haloalkenyloxy, C₁-C₄-alkynyloxy, C₁-C₄-haloalkynyloxy, C₃-C₈-cycloalkoxy, C₃-C₈-halocycloalkoxy, C₁-C₄-alkylthio, C₁-C₄-haloalkylthio, C₁-C₄-alkenylylthio, C₁-C₄-haloalkynylylthio, phenyl, phenyl-C₁-C₄-alkyl, phenyl-C₁-C₄-alkoxy, phenoxy, phenylthio, wherein the phenyl moiety in the 5 last-mentioned radicals may carry 1, 2, 3, 4 or 5 substituents R⁹; 3-, 4-, 5-, 6- or 7-membered saturated, partially unsaturated or fully unsaturated heterocyclic ring containing 1, 2 or 3 heteroatoms selected from the group consisting of N, O and S as ring members, wherein the heterocyclic ring may carry 1, 2 or 3 substituents R⁹; COR¹⁰, COOR¹⁰, CONR¹⁵R¹⁶, NR¹⁵R¹⁶ and S(O)ₚR¹⁰, wherein the aliphatic moieties in the above radicals may carry 1, 2 or 3 substituents R¹⁸ and wherein the cycloaliphatic moieties in the above radicals may carry 1, 2 or 3 substituents R¹⁹; or
R¹ and R² or R³ and R⁴, together with the carbon atom to which they are bound, form a partly unsaturated or fully unsaturated 5-, 6- or 7-membered carbocyclic ring or a partly unsaturated or fully unsaturated 5-, 6- or 7-membered heterocyclic ring containing 1, 2 or 3 heteroatoms selected from the group consisting of O, S and N as ring members; wherein the carbocyclic or heterocyclic ring may carry 1, 2 or 3 substituents R⁹;
each R⁵ is independently selected from the group consisting of halogen, OH, SH, NO₂, CN, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₈-cycloalkyl, C₃-C₈-halocycloalkyl, C₁-C₄-alkoxy, C₁-C₄-haloalkoxy, C₁-C₄-alkenyloxy, C₁-C₄-haloalkenyloxy, C₁-C₄-alkynyloxy, C₁-C₄-haloalkynyloxy, C₃-C₈-cycloalkoxy, C₃-C₈-halocycloalkoxy, C₁-C₄-alkylthio, C₁-C₄-haloalkylthio, C₁-C₄-alkenylylthio, C₁-C₄-haloalkynylylthio, phenyl, phenyl-C₁-C₄-alkyl, phenyl-C₁-C₄-alkoxy, phenoxy, phenylthio, wherein the phenyl moiety in the 5 last-mentioned radicals may carry 1, 2, 3, 4 or 5 substituents R⁹; 3-, 4-, 5-, 6- or 7-membered saturated, partially unsaturated or fully unsaturated heterocyclic ring containing 1, 2 or 3 heteroatoms selected from the group consisting of N, O and S as ring members, wherein the heterocyclic ring may carry 1, 2 or 3 substituents R⁹; COR¹⁰, COOR¹⁰, CONR¹⁵R¹⁶, NR¹⁵R¹⁶ and S(O)ₚR¹⁰, wherein the aliphatic moieties in the above radicals may carry 1, 2 or 3 substituents R¹⁸ and wherein the cycloaliphatic moieties in the above radicals may carry 1, 2 or 3 substituents R¹⁹; or
two radicals R⁵ bound on adjacent carbon atoms, together with the carbon atom to which they are bound, form a partly unsaturated or fully unsaturated 5-, 6- or 7-membered carbocyclic ring or a partly unsaturated or fully unsaturated 5-, 6- or 7-membered heterocyclic ring containing 1, 2 or 3 heteroatoms selected from the group consisting of O, S and N as ring members; wherein the carbocyclic or heterocyclic ring may carry 1, 2 or 3 substituents R⁹;
R⁶ is selected from the group consisting of hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, wherein the phenyl moiety in the 2 last-mentioned radicals may carry 1, 2, 3, 4 or 5 substituents R¹¹, and a 5- or 6-membered saturated, partially unsaturated or aromatic heterocyclic ring containing 1, 2 or 3 heteroatoms selected from the group consisting of N, O and S as ring members, wherein the heterocyclic ring may carry 1, 2 or 3 substituents R¹¹; or, in case m is 0, may also be selected from the group consisting of —C(═O)R¹², —C(═S)R¹², —S(O)₂R¹², —CN, —P(═O)R¹³R¹⁴, M and a group of the formula III 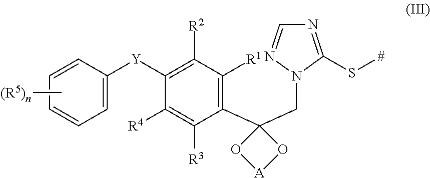
wherein
A, Y, R¹, R², R³, R⁴, R⁵ and n are as defined for formulae I and II; and
# is the attachment point to the remainder of the molecule;
R^{6a} is selected from the group consisting of hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, wherein the phenyl moiety in the 2 last-mentioned radicals may carry 1, 2, 3, 4 or 5 substituents R¹¹, and a 5- or 6-membered saturated, partially unsaturated or aromatic heterocyclic ring containing 1, 2 or 3 heteroatoms selected from the group consisting of N, O and S as ring members, wherein the heterocyclic ring may carry 1, 2 or 3 substituents R¹¹, —C(═O)R¹², —C(═S)R², —S(O)₂R¹², —CN, —P(═O)R¹³R¹⁴ and M;
each R⁷ is independently selected from the group consisting of halogen, OH, SH, NR¹⁵R¹⁶, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, C₁-C₄-alkylthio and C₁-C₄-haloalkylthio, wherein the aliphatic moieties in the above radicals may carry 1, 2 or 3 substituents R¹⁸; or
two radicals R⁷ bound on two adjacent carbon atoms, together with the carbon atoms to which they are bound, form a 3-, 4-, 5-, 6- or 7-membered saturated, partly unsaturated or fully unsaturated carbocyclic ring or a 3-, 4-, 5-, 6- or 7-membered saturated, partly unsaturated or fully unsaturated heterocyclic ring containing 1, 2, or 3 heteroatoms selected from the group consisting of O, S and N as ring members, wherein the carbocyclic or heterocyclic ring may carry 1, 2 or 3 substituents R⁹;
R⁸ is selected from the group consisting of hydrogen, CN, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, phenyl, phenyl-C₁-C₄-alkyl, wherein the phenyl moiety in the 2 last-mentioned radicals may carry 1, 2, 3, 4 or 5 substituents R⁹; COR¹⁰, COOR¹⁰, CONR¹⁵R¹⁶ and S(O)ₚR¹⁰;
each R⁹ is independently selected from the group consisting of halogen, OH, SH, NR¹⁵R¹⁶, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, C₁-C₄-alkylthio and C₁-C₄-haloalkylthio, wherein the aliphatic moieties in the above radicals may carry 1, 2 or 3 substituents R¹⁸;
each R¹⁰ is independently selected from the group consisting of hydrogen, C₁-C₄-alkyl, C₁-C₄-haloalkyl, C₂-C₄-alkenyl, C₂-C₄-haloalkenyl, C₁-C₄-aminoalkyl, phenyl, phenyl-C₁-C₄-alkyl, wherein the phenyl moiety in the 2 last-mentioned radicals may carry 1, 2, 3, 4 or 5 substituents R⁹, and a 5- or 6-membered saturated, partially unsaturated or aromatic heterocyclic ring containing 1, 2 or 3 heteroatoms selected from the group consisting of N, O and S as ring members, wherein the heterocyclic ring may carry 1, 2 or 3 substituents R⁹;
each R¹¹ is independently selected from the group consisting of halogen, OH, SH, NR¹⁵R¹⁶, CN, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₁-C₄-alkoxy, C₁-C₄-haloalkoxy, C₁-C₄-alkylthio and C₁-C₄-haloalkylthio, wherein the aliphatic moieties in the above radicals may carry 1, 2 or 3 substituents R¹⁸;
R¹² is selected from the group consisting of hydrogen, C₁-C₁₀-alkyl, C₁-C₁₀-haloalkyl, C₁-C₁₀-alkoxy, C₁-C₁₀-haloalkoxy, C₁-C₁₀-aminoalkyl, C₃-C₁₀-cycloalkyl, C₃-C₁₀-halocycloalkyl, phenyl, phenyl-C₁-C₄-alkyl, wherein the phenyl moiety in the 2 last-mentioned radicals may carry 1, 2, 3, 4 or 5 substituents R¹¹, and a 5- or 6-membered saturated, partially unsaturated or aromatic heterocyclic ring containing 1, 2 or 3 heteroatoms selected from the group consisting of N, O and S as ring members, wherein the heterocyclic ring may carry 1, 2 or 3 substituents R¹¹, and NR¹⁵R¹⁶;
R¹³ and R¹⁴, independently of each other, are selected from the group consisting of C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, C₁-C₁₀-alkoxy, C₁-C₁₀-haloalkoxy, C₁-C₄-alkoxy-C₁-C₁₀-alkyl, C₁-C₄-alkoxy-C₁-C₁₀-alkoxy, C₁-C₁₀-alkylthio, C₁-C₁₀-haloalkylthio, C₂-C₁₀-alkenyloxy, C₂-C₁₀-alkenylthio, C₂-C₁₀-alkynyloxy, C₂-C₁₀-alkynylthio, C₃-C₁₀-cycloalkoxy, C₃-C₁₀-cycloalkylthio, phenyl, phenyl-C₁-C₄-alkyl, phenylthio, phenyl-C₁-C₄-alkoxy, and NR¹⁵R¹⁶;
each R¹⁵ is independently selected from the group consisting of hydrogen and C₁-C₈-alkyl;
each R¹⁶ is independently selected from the group consisting of hydrogen, C₁-C₈-alkyl, phenyl, and phenyl-C₁-C₄-alkyl;
or R¹⁵ and R¹⁶ together form a linear C₄- or C₅-alkylene bridge or a group —CH₂CH₂OCH₂CH₂— or —CH₂CH₂NR¹⁷CH₂CH₂—;
each R¹⁷ is independently selected from the group consisting of hydrogen and C₁-C₄-alkyl;
each R⁸ is independently selected from the group consisting of nitro, CN, OH, SH, COR¹⁰, COOR¹⁰, CONR¹⁵R¹⁶; NR¹⁵R¹⁶, C₃-C₆-cycloalkyl, C₃-C₆-halocycloalkyl, C₁-C₄-alkoxy, C₁-C₄-haloalkoxy, C₃-C₆-cycloaloxy, phenyl and phenoxy;
each R¹⁹ is independently selected from the group consisting of nitro, CN, OH, SH, COR¹⁰, COOR¹⁰, CONR¹⁵R¹⁶; NR¹⁵R¹⁶, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy, C₃-C₆-cycloaloxy, phenyl and phenoxy;
Q is O or S;
M is a metal cation equivalent or an ammonium cation of formula (NR^{a}R^{b}R^{c}R^{d})⁺, wherein R^{a}, R^{b}, R^{c} and R^{d}, independently of each other, are selected from the group consisting of hydrogen, C₁-C₁₀-alkyl, phenyl and benzyl, wherein the phenyl moiety in the 2 last-mentioned radicals may carry 1, 2 or 3 substituents independently selected from the group consisting of halogen, CN, nitro, C₁-C₄-alkyl, C₁-C₄-haloalkyl, C₁-C₄-alkoxy, C₁-C₄-haloalkoxy and NR¹⁵R¹⁶;
m is 0, 1 or 2;
n is 0, 1, 2, 3, 4 or 5; and
p is 1 or 2;
with the proviso that R¹ is not Cl if R², R³ and R⁴ are hydrogen, Y is O, (R⁵)ₙ is 4-Cl, relative to the 1-position of the attachment point of the phenyl ring to the group Y, and A is a linear C₂-alkylene group which may be substituted by 1 or 2 C₁-C₄-alkyl groups or is —(CH₂)₃; and
with the proviso that R³ is not Cl if R¹, R² and R⁴ are hydrogen, Y is O, (R⁵)ₙ is 4-Cl, relative to the 1-position of the attachment point of the phenyl ring to the group Y, and A is a linear C₂-alkylene group which may be substituted by 1 or 2 C₁-C₄-alkyl groups or is —(CH₂)₃;
and the agriculturally acceptable salts thereof.

34. The compound of claim 33,
with the proviso that R¹ is not Cl if R², R³ and R⁴ are hydrogen, Y is O and (R⁵)ₙ is 4-Cl, relative to the 1-position of the attachment point of the phenyl ring to the group Y; and
with the proviso that R³ is not Cl if R¹, R² and R⁴ are hydrogen, Y is O and (R⁵)ₙ is 4-Cl, relative to the 1-position of the attachment point of the phenyl ring to the group Y.

35. The compound of claim 33, wherein R², R³ and R⁴ are hydrogen and R¹ is selected from the group consisting of fluorine, bromine, OH, SH, NO₂, CN, C₁-C₄-alkyl, C₁-C₄-haloalkyl, C₁-C₄-alkoxy-C₁-C₄-alkyl, C₁-C₄-alkoxy, C₁-C₄-alkoxy-C₁-C₄-alkoxy, and C₁-C₄-haloalkoxy.

36. The compound of claim 35, wherein R², R³ and R⁴ are hydrogen and R¹ is selected from the group consisting of fluorine and bromine.

37. The compound of claim 35, wherein R², R³ and R⁴ are hydrogen and R¹ is selected from the group consisting of C₁-C₄-alkyl, C₁-C₄-haloalkyl, C₁-C₄-alkoxy and C₁-C₄-haloalkoxy.

38. The compound of claim 35, wherein at least two of R¹, R², R³ and R⁴ are not hydrogen.

39. The compound of claim 35, wherein at least two of R¹, R², R³ and R⁴ are not hydrogen and are simultaneously selected from the group consisting of fluorine, chlorine, bromine, C₁-C₄-alkyl, C₁-C₄-haloalkyl, C₁-C₄-alkoxy and C₁-C₄-haloalkoxy.

40. The compound of claim 33, wherein n is 1 and R⁵ is selected from the group consisting of 2-Cl and 3-Cl.

41. The compound of claim 33, wherein R⁵ is selected from the group consisting of fluorine, bromine, OH, SH, NO₂, CN, C₁-C₄-alkyl, C₁-C₄-haloalkyl, C₁-C₄-alkoxy-C₁-C₄-alkyl, C₁-C₄-alkoxy, C₁-C₄-alkoxy-C₁-C₄-alkoxy, and C₁-C₄-haloalkoxy.

42. The compound of claim 33, wherein n is 0, 2, 3 or 4.

43. The compound of claim 33, wherein R¹² is selected from the group consisting of C₁-C₄-alkyl, C₁-C₂-haloalkyl, C₁-C₄-alkoxy, C₁-C₂-haloalkoxy, phenyl, phenoxy and NR¹⁵R¹⁶, wherein R¹⁵ is hydrogen and R¹⁶ is selected from the group consisting of hydrogen, C₁-C₄-alkyl and phenyl, or both R¹⁵ and R¹⁶ are C₁-C₄-alkyl.

44. The compound of claim 33, wherein R⁶ is selected from the group consisting of hydrogen, C₁-C₄-alkyl, —C(═O)R¹², —S(O)₂R¹², —CN, M and a group of the formula III.

45. The compound of claim 33, wherein R^{6a} is selected from the group consisting of hydrogen, C₁-C₄-alkyl, —S(O)₂R¹², and —C(═O)R¹².

46. The compound of claim 33, wherein A is a linear C₂- or C₃-alkyene bridge, wherein 1 or 2 hydrogen atoms of the alkylene bridge may be replaced by 1 or 2 substituents R⁷, wherein each R⁷ is independently selected from the group consisting of C₁-C₄-alkyl, C₁-C₄-haloalkyl, C₁-C₄-alkoxy, C₁-C₄-alkoxy-C₁-C₄-alkoxy and C₁-C₄-haloalkoxy or two substituents R⁷ bound on adjacent carbon atoms, together with the carbon atoms to which they are bound, form a cyclopentyl or cyclohexyl ring.

47. The compound of claim 33, wherein m is 0.

48. A compound of formula IV 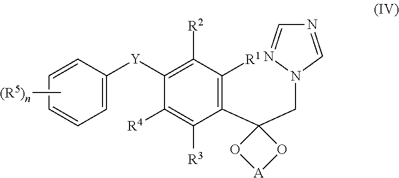
wherein A, Y, R¹, R², R³, R⁴, R⁵ and n are as defined in claim 33.

49. The compound of claim 48, wherein Y is O or NR⁸.

50. The compound of claim 49, wherein Y is O.

51. The compound of claim 48, wherein
A is a linear C₃-alkylene bridge which may be substituted by 1, 2, 3 or 4 substituents R⁷;
R¹ is selected from the group consisting of fluorine, chlorine, methyl and trifluoromethyl;
R², R³ and R⁴ are hydrogen;
(R⁵)ₙ is 4-Cl, relative to the 1-position of the attachment point of the phenyl ring to the group Y;
each R⁷ is independently selected from the group consisting of C₁-C₄-alkyl and phenyl; with the provisos that
R¹ is not Cl if Y is O and A is —(CH₂)₃—;
R¹ is not methyl if Y is O and A is —(CH₂)₃— or —CH(CH₃)CH₂CH₂—.

52. The compound of claim 51, of formula IVD 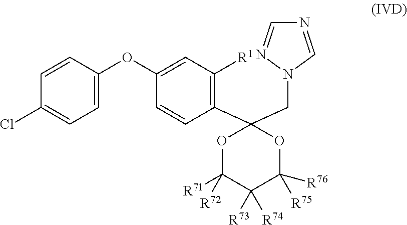
wherein
a. R¹ is Cl, R⁷¹ is methyl and R⁷², R⁷³, R⁷⁴, R⁷⁵ and R⁷⁶ are hydrogen;
b. R¹ is Cl, R⁷¹ and R⁷⁶ are methyl and R⁷², R⁷³, R⁷⁴ and R⁷⁵ are hydrogen;
c. R¹ is Cl, R⁷³ is tert-butyl and R⁷¹, R⁷², R⁷⁴, R⁷⁵ and R⁷⁶ are hydrogen;
d. R¹ is Cl, R⁷³ is phenyl and R⁷¹, R⁷², R⁷⁴, R⁷⁵ and R⁷⁶ are hydrogen;
e. R¹ is Cl, R⁷³ is methyl and R⁷¹, R⁷², R⁷⁴, R⁷⁵ and R⁷⁶ are hydrogen;
f. R¹ is Cl, R⁷³ and R⁷⁴ are methyl and R⁷¹, R⁷², R⁷⁵ and R⁷⁶ are hydrogen;
g. R¹ is Cl, R⁷¹ is n-propyl, R⁷³ and R⁷⁴ are methyl and R⁷², R⁷ and R⁷⁶ are hydrogen;
h. R¹ is Cl, R⁷¹ and R⁷² are methyl and R⁷³, R⁷⁴, R⁷⁵ and R⁷⁶ are hydrogen;
i. R¹ is Cl, R⁷¹, R⁷², R⁷⁵ and R⁷⁶ are methyl and R⁷³ and R⁷⁴ are hydrogen;
j. R¹ is methyl and R⁷¹, R⁷², R⁷³, R⁷⁴, R⁷⁵ and R⁷⁶ are hydrogen;
k. R¹ is methyl, R⁷¹ is methyl and R⁷², R⁷³, R⁷⁴, R⁷⁵ and R⁷⁶ are hydrogen;
l. R¹ is methyl, R⁷¹ and R⁷² are methyl and R⁷³, R⁷⁴, R⁷⁵ and R⁷⁶ are hydrogen
m. R¹ is methyl, R⁷¹, R⁷², R⁷⁵ and R⁷⁶ are methyl and R⁷³ and R⁷⁴ are hydrogen;
n. R¹ is methyl, R⁷¹ and R⁷⁶ are methyl and R⁷², R⁷³, R⁷⁴ and R⁷⁵ are hydrogen;
o. R¹ is methyl, R⁷³ is tert-butyl and R⁷¹, R⁷², R⁷⁴, R⁷⁵ and R⁷⁶ are hydrogen;
p. R¹ is methyl, R⁷³ is phenyl and R⁷¹, R⁷², R⁷⁴, R⁷⁵ and R⁷⁶ are hydrogen;
q. R¹ is methyl, R⁷³ is methyl and R⁷¹, R⁷², R⁷⁴, R⁷⁵ and R⁷⁶ are hydrogen;
r. R¹ is methyl, R⁷³ and R⁷⁴ are methyl and R⁷¹, R⁷², R⁷⁵ and R⁷⁶ are hydrogen;
s. R¹ is methyl, R⁷¹ is n-propyl, R⁷³ and R⁷⁴ are methyl and R⁷², R⁷⁵ and R⁷⁶ are hydrogen;
t. R¹ is trifluoromethyl and R⁷¹, R⁷², R⁷³, R⁷⁴, R⁷⁵ and R⁷⁶ are hydrogen;
u. R¹ is trifluoromethyl, R⁷¹ is methyl and R⁷², R⁷³, R⁷⁴, R⁷⁵ and R⁷⁶ are hydrogen;
v. R¹ is trifluoromethyl, R⁷¹ and R⁷⁶ are methyl and R⁷², R⁷³, R⁷⁴ and R⁷⁵ are hydrogen;
w. R¹ is trifluoromethyl, R⁷³ is tert-butyl and R⁷¹, R⁷², R⁷⁴, R⁷⁵ and R⁷⁶ are hydrogen;
x. R¹ is trifluoromethyl, R⁷³ is phenyl and R⁷¹, R⁷², R⁷⁴, R⁷⁵ and R⁷⁶ are hydrogen;
y. R¹ is trifluoromethyl, R⁷³ is methyl and R⁷¹, R⁷², R⁷⁴, R⁷⁵ and R⁷⁶ are hydrogen;
z. R¹ is trifluoromethyl, R⁷³ and R⁷⁴ are methyl and R⁷¹, R⁷², R⁷⁵ and R⁷⁶ are hydrogen;
aa. R¹ is trifluoromethyl, R⁷¹ is n-propyl, R⁷³ and R⁷⁴ are methyl and R⁷², R⁷⁵ and R⁷⁶ are hydrogen;
bb. R¹ is trifluoromethyl, R⁷¹ and R⁷² are methyl and R⁷³, R⁷⁴, R⁷⁵ and R⁷⁶ are hydrogen;
cc. R¹ is trifluoromethyl, R⁷¹, R⁷², R⁷⁵ and R⁷⁶ are methyl and R⁷³ and R⁷⁴ are hydrogen.

53. The compound of claim 48, wherein
A is a linear C₄-C₈-alkylene bridge which may be substituted by 1, 2, 3 or 4 substituents R⁷.

54. The compound of claim 48, of formula IVA 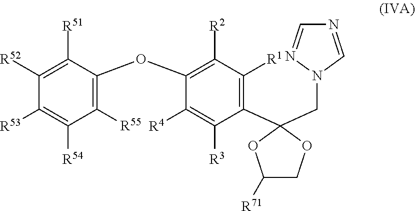
wherein R⁵¹ is F and at least one of R⁵¹, R⁵³, R⁵⁴ or R⁵⁵ is F or Cl.

55. The compound of claim 54, of formula IVA, wherein
dd. R¹, R², R³ and R⁴ are hydrogen, R⁵¹ is F, R⁵³ is Cl, R⁵², R⁵⁴ and R⁵⁵ are hydrogen and R⁷¹ is methyl;
ee. R¹, R², R³ and R⁴ are hydrogen, R⁵¹ and R⁵³ are F, R⁵², R⁵⁴ and R⁵⁵ are hydrogen and R⁷¹ is methyl;
ff. R¹, R², R³ and R⁴ are hydrogen, R⁵¹ and R⁵³ are F, R⁵², R⁵⁴ and R⁵⁵ are hydrogen and R⁷¹ is ethyl;
gg. R¹, R², R³ and R⁴ are hydrogen, R⁵¹ is F, R⁵⁵ is Cl, R⁵², R⁵³ and R⁵⁴ are hydrogen and R⁷¹ is methyl;
hh. R¹, R², R³ and R⁴ are hydrogen, R⁵¹ is F, R⁵⁵ is Cl, R⁵², R⁵³ and R⁵⁴ are hydrogen and R⁷¹ is ethyl;
ii. R¹, R², R³ and R⁴ are hydrogen, R⁵¹ is F, R⁵⁵ is Cl, R⁵², R⁵³ and R⁵⁴ are hydrogen and R⁷¹ is n-propyl;
jj. R¹, R², R³ and R⁴ are hydrogen, R⁵¹ and R⁵⁵ are F, R⁵², R⁵³ and R⁵⁴ are hydrogen and R⁷¹ is ethyl;
kk. R¹, R², R³ and R⁴ are hydrogen, R⁵¹ and R⁵⁵ are F, R⁵², R⁵³ and R⁵⁴ are hydrogen and R⁷¹ is n-propyl;
ll. R¹, R², R³ and R⁴ are hydrogen, R⁵¹ is F, R⁵⁴ is Cl, R⁵², R⁵³ and R⁵⁵ are hydrogen and R⁷¹ is methyl;
mm. R¹, R², R³ and R⁴ are hydrogen, R⁵¹ is F, R⁵⁴ is Cl, R⁵², R⁵³ and R⁵⁵ are hydrogen and R⁷¹ is ethyl;
nn. R¹, R², R³ and R⁴ are hydrogen, R⁵¹ is F, R⁵⁴ is Cl, R⁵², R⁵³ and R⁵⁵ are hydrogen and R⁷¹ is n-propyl;
oo. R¹, R², R³ and R⁴ are hydrogen, R⁵¹ is F, R⁵³ is Cl, R⁵², R⁵⁴ and R⁵⁵ are hydrogen and R⁷¹ is ethyl;
pp. R¹, R², R³ and R⁴ are hydrogen, R⁵¹ is F, R⁵³ is Cl, R⁵², R⁵⁴ and R⁵⁵ are hydrogen and R⁷¹ is n-propyl;
qq. R¹, R², R³ and R⁴ are hydrogen, R⁵¹ and R⁵⁴ are F, R⁵², R⁵³ and R⁵⁵ are hydrogen and R⁷¹ is methyl;
rr. R¹, R², R³ and R⁴ are hydrogen, R⁵¹ and R⁵⁴ are F, R⁵², R⁵³ and R⁵⁵ are hydrogen and R⁷¹ is ethyl;
ss. R¹, R², R³ and R⁴ are hydrogen, R⁵¹ and R⁵⁴ are F, R⁵², R⁵³ and R⁵⁵ are hydrogen and R⁷¹ is n-propyl;
tt. R¹ is Cl, R², R³ and R⁴ are hydrogen, R⁵¹ and R⁵³ are F, R⁵², R⁵⁴ and R⁵⁵ are hydrogen and R⁷¹ is methyl;
uu. R¹ is Cl, R², R³ and R⁴ are hydrogen, R⁵¹ and R⁵⁵ are F, R⁵², R⁵³ and R⁵⁴ are hydrogen and R⁷¹ is methyl;
vv. R¹ is methyl, R², R³ and R⁴ are hydrogen, R⁵¹ and R⁵³ are F, R⁵², R⁵⁴ and R⁵⁵ are hydrogen and R⁷¹ is methyl;
ww. R¹ is Cl, R², R³ and R⁴ are hydrogen, R⁵¹ is F, R⁵³ is Cl, R⁵², R⁵⁴ and R⁵⁵ are hydrogen and R⁷¹ is methyl;
xx. R¹ is methyl, R², R³ and R⁴ are hydrogen, R⁵¹ is F, R⁵³ is Cl, R⁵², R⁵⁴ and R⁵⁵ are hydrogen and R⁷¹ is methyl;
yy. R¹ is trifluoromethyl, R², R³ and R⁴ are hydrogen, R⁵¹ is F, R⁵³ is Cl, R⁵², R⁵⁴ and R⁵⁵ are hydrogen and R⁷¹ is methyl;
zz. R¹ is Cl, R², R³ and R⁴ are hydrogen, R⁵¹ is F, R⁵⁵ is Cl, R⁵², R⁵³ and R⁵⁴ are hydrogen and R⁷¹ is methyl;
aaa. R¹ is Cl, R², R³ and R⁴ are hydrogen, R⁵¹ and R⁵⁴ are F, R⁵², R⁵³ and R⁵⁵ are hydrogen and R⁷¹ is methyl;
bbb. R¹ is methyl, R², R³ and R⁴ are hydrogen, R⁵¹ and R⁵⁴ are F, R⁵², R⁵³ and R⁵⁵ are hydrogen and R⁷¹ is methyl;
ccc. R¹ is Cl, R⁴ is F, R² and R³ are hydrogen, R⁵¹ is F, R⁵³ is Cl, R⁵², R⁵⁴ and R⁵⁵ are hydrogen and R⁷¹ is methyl;
ddd. R¹ is Cl, R⁴ is F, R² and R³ are hydrogen, R⁵¹ and R⁵³ are F, R⁵², R⁵⁴ and R⁵⁵ are hydrogen and R⁷¹ is methyl.

56. The compound of claim 48, of formula IVA 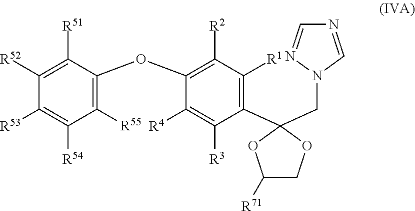
wherein
eee. R¹ is methyl, R², R³ and R⁴ are hydrogen, R⁵¹ and R⁵³ are Cl, R⁵², R⁵⁴ and R⁵⁵ are hydrogen and R⁷¹ is methyl;
fff. R¹ is methyl, R², R³ and R⁴ are hydrogen, R⁵² and R⁵⁴ are Cl, R⁵¹, R⁵³ and R⁵⁵ are hydrogen and R⁷¹ is methyl;
ggg. R¹ is methyl, R², R³ and R⁴ are hydrogen, R⁵¹ is Cl, R⁵², R⁵³, R⁵⁴ and R⁵⁵ are hydrogen and R⁷¹ is methyl;
hhh. R² is F, R¹, R³ and R⁴ are hydrogen, R⁵³ is Cl, R⁵¹, R⁵², R⁵⁴ and R⁵⁵ are hydrogen and R⁷¹ is methyl;
iii. R¹ is F, R², R³ and R⁴ are hydrogen, R⁵¹, R⁵², R⁵³, R⁵⁴ and R⁵⁵ are hydrogen and R⁷¹ is methyl;
jjj. R¹ is F, R², R³ and R⁴ are hydrogen, R⁵³ is Cl, R⁵¹, R⁵², R⁵⁴ and R⁵⁵ are hydrogen and R⁷¹ is methyl;
kkk. R² is F, R¹, R³ and R⁴ are hydrogen, R⁵¹, R⁵², R⁵³, R⁵⁴ and R⁵⁵ are hydrogen and R⁷¹ is methyl;
lll. R¹ and R⁴ are F, R² and R³ are hydrogen, R⁵³ is Cl, R⁵¹, R⁵², R⁵⁴ and R⁵⁵ are hydrogen and R⁷¹ is methyl;
mmm. R¹ is Cl, R², R³ and R⁴ are hydrogen, R⁵³ is Cl, R⁵¹, R⁵², R⁵⁴ and R⁵⁵ are hydrogen and R⁷¹ is ethyl;
nnn. R¹ is Cl, R², R³ and R⁴ are hydrogen, R⁵³ is Cl, R⁵¹, R⁵², R⁵⁴ and R⁵⁵ are hydrogen and R⁷¹ is n-propyl;
ooo. R² and R³ are F, R¹ and R⁴ are hydrogen, R⁵¹, R⁵², R⁵³, R⁵⁴ and R⁵⁵ are hydrogen and R⁷¹ is methyl;
ppp. R² and R⁴ are F, R¹ and R³ are hydrogen, R⁵¹, R⁵², R⁵³, R⁵⁴ and R⁵⁵ are hydrogen and R⁷¹ is methyl;
qqq. R¹ is trifluoromethyl, R², R³ and R⁴ are hydrogen, R⁵³ is Cl, R⁵¹, R⁵², R⁵⁴ and R⁵⁵ are hydrogen and R⁷¹ is methyl;
rrr. R² is F, R³ is Cl, R¹ and R⁴ are hydrogen, R⁵³ is Cl, R⁵¹, R⁵², R⁵⁴ and R⁵⁵ are hydrogen and R⁷¹ is methyl;
sss. R¹ is trifluoromethyl, R², R³ and R⁴ are hydrogen, R⁵¹ and R⁵³ are Cl, R⁵², R⁵⁴ and R⁵⁵ are hydrogen and R⁷¹ is methyl;
ttt. R¹ is trifluoromethyl, R², R³ and R⁴ are hydrogen, R⁵¹ and R⁵³ are Cl, R⁵², R⁵⁴ and R⁵⁵ are hydrogen and R⁷¹ is hydrogen;
uuu. R¹ is trifluoromethyl, R², R³ and R⁴ are hydrogen, R⁵¹ is Cl, R⁵², R⁵³, R⁵⁴ and R⁵⁵ are hydrogen and R⁷¹ is hydrogen;
vvv. R¹ is Cl, R⁴ is F, R² and R³ are hydrogen, R⁵¹ and R⁵³ are Cl, R⁵², R⁵⁴ and R⁵⁵ are hydrogen and R⁷¹ is methyl;
www. R¹ is Cl, R⁴ is F, R² and R³ are hydrogen, R⁵¹ is Cl, R⁵², R⁵³, R⁵⁴ and R⁵⁵ are hydrogen and R⁷¹ is methyl.

57. The compound of claim 48, of formula IVB or IVC

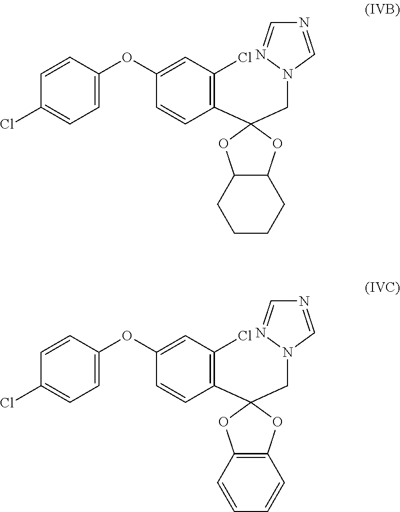

58. An agricultural composition comprising at least one compound of claim 33 or an agriculturally acceptable salt thereof and a liquid or solid carrier.

59. A method for controlling harmful fungi, wherein the fungi, their habitat or the materials or plants to be protected against fungal attack, or the soil or propagation material are treated with an effective amount of at least compound of claim 33.

60. A seed treated with at least compound of claim 33, in an amount of from 0.1 g to 10 kg per 100 kg of seeds.

61. A pharmaceutical composition comprising at least one compound of claim 33 or a pharmaceutically acceptable salt thereof and at least one pharmaceutically acceptable carrier.

62. A method for treating cancer or virus infections or for combating zoopathogenic or humanpathogenic fungi, which comprises treating an individual in need thereof with at least one compound of in claim 33, or a pharmaceutically acceptable salt thereof.
